(19) United States
(12) Patent Application Publication
(10) Pub. No.: US 2024/0335443 A1
(43) Pub. Date: Oct. 10, 2024

(54) IMIDAZOLOPYRIDAZINE OR PYRAZOLOPYRIMIDINE COMPOUNDS AND COMPOSITIONS

(71) Applicant: Anrui Biomedical Technology (Guangzhou) Co., Ltd., Guangzhou, Guangdong (CN)

(72) Inventors: Dai Cheng, Guangzhou (CN); Sen Zeng, Guangzhou (CN); Qiming Yue, Guangzhou (CN); Zeqiang Xie, Guangzhou (CN)

(21) Appl. No.: 18/273,011

(22) PCT Filed: Jan. 18, 2022
(86) PCT No.: PCT/CN2022/072472

§ 371 Date: Jul. 18, 2023

(30) Foreign Application Priority Data
Jan. 19, 2021 (WO) PCT/CN2021/072752

(51) Int. Cl. A61K 31/5025; A61K 31/519; C07D 487/04; C07D 519/00

(52) U.S. Cl. CPC A61K 31/5025; A61K 31/519; C07D 487/04; C07D 519/00

(57) ABSTRACT

Provided herein are novel compounds (e.g., Formula I or II), pharmaceutical compositions, and methods of using related to Tyrosine kinase 2 (TYK2). The compounds herein are typically TYK2 inhibitors, which can be used for treating a variety of diseases or disorders, such as an autoimmune disorder or an inflammatory disorder, e.g., psoriasis, psoriatic arthritis, Crohn's disease, ulcerative colitis, inflammatory bowel disease, and/or systemic lupus erythematosus.

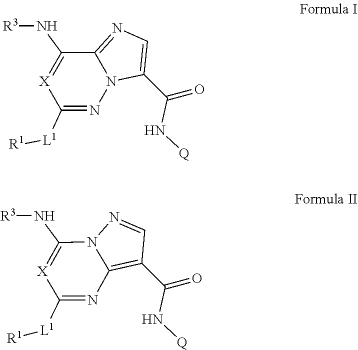

### CROSS REFERENCE TO RELATED APPLICATIONS

This application claims priority of International Application No. PCT/CN2021/072752, filed Jan. 19, 2021, the content of which is incorporated herein by reference in its entirety for all purposes.

In various embodiments, the present disclosure generally relates to novel compounds, compositions comprising the same, methods of preparing and methods of using the same, e.g., for inhibiting tyrosine-protein kinase 2 (TYK2) and/or for treating or preventing various diseases or disorders described herein.

### BACKGROUND

Tyrosine kinase 2 (TYK2) is a member of the Janus kinase (JAK) family of nonreceptor tyrosine kinases. It has been shown that TYK2 is critical in regulating the signal transduction cascade downstream of receptors for IL-12, IL-23 and type I interferons (e.g., IFN-alpha or IFN-beta) both in mice and in human. TYK2 mediates the receptor-induced phosphorylation of members of the Signal Transducer and Activation of Transcription (STAT) family of transcription factors, an essential signal that leads to the dimerization of STAT proteins and the transcription of STAT-dependent pro-inflammatory genes.

Various diseases or disorders such as autoimmune diseases, inflammatory diseases, etc. are known to be associated/mediated by TYK2. Clinically, a TYK2 inhibitor, BMS-986165, is currently in phase III trial for treating psoriasis. BMS-986165 is also in various trials for treating other diseases such as Crohn's disease, psoriatic arthritis, systemic lupus erythematosus, ulcerative colitis, and inflammatory bowel disease. New TYK2 inhibitors are needed to provide therapeutic benefits to a wide variety of patients in need thereof.

### BRIEF SUMMARY

In various embodiments, the present disclosure is based in part on the discovery of newly designed heteroaryl compounds as TYK2 inhibitors, which can provide various advantages over existing TYK2 inhibitors, such as better potency, pharmacokinetic profile, and/or in vivo activities. The compounds and compositions herein are useful for treating various diseases or disorders, such as an autoimmune disorder or an inflammatory disorder, e.g., psoriasis, psoriatic arthritis, Crohn's disease, ulcerative colitis, inflammatory bowel disease, and/or systemic lupus erythematosus.

Some embodiments of the present disclosure are directed to a compound of Formula I or II, or a pharmaceutically acceptable salt thereof,

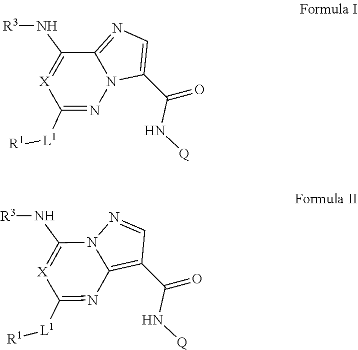

wherein the variables are defined herein. In some embodiments, the compound of Formula I can have a sub-formula of I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A, as defined herein. In some embodiments, the compound of Formula II can have a sub-formula of II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A, as defined herein. In some embodiments, the present disclosure also provides specific compounds selected from any of the specific compounds disclosed in Table 1A or 1B herein, or any of Compound Nos. 1-107, or a pharmaceutically acceptable salt thereof.

In some embodiments, the present disclosure provides a pharmaceutical composition comprising one or more compounds of the present disclosure and optionally a pharmaceutically acceptable excipient. The pharmaceutical composition can be typically formulated for oral administration.

In some embodiments, the present disclosure also provides a method of inhibiting TYK2 in a subject or biological sample. In some embodiments, the method comprises contacting the subject or biological sample with an effective amount of one or more compounds of the present disclosure, e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof, or a pharmaceutical composition comprising the same.

In some embodiments, the present disclosure provides a method of treating or preventing a TYK2-mediated disease or disorder in a subject in need thereof. In some embodiments, the method comprises administering to the subject an effective amount of one or more compounds of the present disclosure or the pharmaceutical composition herein. In some embodiments, the method comprises administering to the subject an effective amount of a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, 11-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof, or a pharmaceutical composition comprising the same. In some embodiments, the TYK2-mediated disease or disorder is an autoimmune disease or disorder, an inflammatory disease or disorder, a proliferative disease or disorder, an endocrine disease or disorder, a neurological disease or disorder, and/or a disease or disorder associated with transplantation. In some embodiments, the TYK2 mediate disease or disorder is psoriasis, psoriatic arthritis, Crohn's disease, ulcerative colitis, inflammatory bowel disease, and/or systemic lupus erythematosus. In some embodiments, the administering is an oral administration. In some embodiments, the method herein further comprises administering to the subject an additional therapeutic agent.

It is to be understood that both the foregoing summary and the following detailed description are exemplary and explanatory only, and are not restrictive of the invention herein.

### DETAILED DESCRIPTION

In various embodiments, the present disclosure provides compounds and compositions that are useful for inhibiting TYK2 and/or treating or preventing various diseases or disorders described herein.

### Compounds

In some embodiments, the present disclosure provides a compound of Formula I or II, or a pharmaceutically acceptable salt thereof:

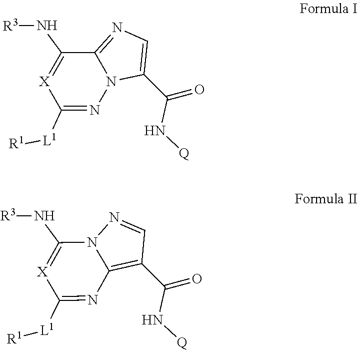

wherein:
L¹ is a bond, NR¹⁰, O, optionally substituted C₁₋₄ alkylene or optionally substituted C₁₋₄ heteroalkyelene;
R¹ is an optionally substituted carbocyclic ring, optionally substituted heterocyclic ring, optionally substituted aryl or optionally substituted heteroaryl ring;
X is N or CR², wherein R² is hydrogen, hydroxyl, halogen, optionally substituted C₁₋₆ alkyl, optionally substituted C₁₋₆ heteroalkyl, optionally substituted C₁₋₆ alkoxy, optionally substituted C₃₋₆ cycloalkyl, or optionally substituted 4-8 membered heterocyclyl;
R³ is hydrogen, optionally substituted C₁₋₆ alkyl, optionally substituted C₁₋₆ heteroalkyl, optionally substituted C₃₋₆ cycloalkyl, or optionally substituted 4-8 membered heterocyclyl; or R² and R³, together with the intervening atoms, form a 5-8 membered heterocyclic or a 5 or 6 membered heteroaryl ring, each of which is optionally substituted and has one ring nitrogen atom as required by Formula I or II, and optionally 1-2 additional ring heteroatoms independently selected from nitrogen, oxygen, and sulfur;
Q is 6-14 membered heterocyclyl or 5-10 membered heteroaryl, each of which is optionally substituted, or Q is

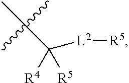

wherein R⁴ and R⁵ are each independently hydrogen or an optionally substituted C₁₋₆ alkyl, or R⁴ and R⁵ are joined to form a 3-8 membered carbocyclic or heterocyclic ring, each of which is optionally substituted;
L² is a bond, NR^{11A}, O, optionally substituted C₁₋₄ alkylene or optionally substituted C₁₋₄ heteroalkylene;
R⁶ is hydrogen, a 3-8 membered heterocyclic ring, or a 5 or 6 membered heteroaryl ring, each of which is optionally substituted; and
wherein:
R¹⁰ and R^{11A} are each independently hydrogen, optionally substituted C₁₋₆ alkyl, optionally substituted C₃₋₆ cycloalkyl, optionally substituted aryl (e.g, phenyl), optionally substituted heteroaryl (e.g., 5 or 6 membered heteroaryl), or optionally substituted 4-8 membered heterocyclyl.

In some embodiments, the compound of Formula I or II (including any of the applicable sub-formulae as described herein) can comprise one or more asymmetric centers and/or axial chirality, and thus can exist in various stereoisomeric forms, e.g., enantiomers and/or diastereomers. In some embodiments, the compound of Formula I or II can exist in the form of an individual enantiomer and/or diastereomer, as applicable, or a mixture of stereoisomers, including racemic mixtures and mixtures enriched in one or more stereoisomers. In some embodiments, when applicable, the compound of Formula I or II (including any of the applicable sub-formulae as described herein) can exist as an isolated individual enantiomer substantially free (e.g., with less than 20%, less than 10%, less than 5%, less than 1%, by weight, by supercritical fluid chromatography (SFC) or high performance liquid chromatography (HPLC) area, or both, or with a non-detectable amount) of the other enantiomer. In some embodiments, the compound of Formula I or II (including any of the applicable sub-formulae as described herein) can have an enantiomeric excess (“ee”) of greater than 70%, such as having greater than 80% ee, greater than 90% ee, greater than 95% ee, greater than 98% ee, greater than 99% ee, or the other enantiomer is non-detectable. In some embodiments, the compound of Formula I or II (including any of the applicable sub-formulae as described herein) can have a diastereomeric excess (“de”) of greater than 70%, such as having greater than 80% de, greater than 90% de, greater than 95% de, greater than 98% de, greater than 99% de, or the other diastereomer(s) is non-detectable. In some embodiments, when applicable, the compound of Formula I or II (including any of the applicable sub-formulae as described herein) can also exist as a mixture of stereoisomers in any ratio, such as a racemic mixture.

In some embodiments, the compound has a Formula I.

In some embodiments, the compound has a Formula II.

It should be apparent to those skilled in the art that in certain cases, the compound of Formula I or II may exist as a mixture of tautomers. The present disclosure is not limited to any specific tautomer. Rather, the present disclosure encompasses any and all of such tautomers whether or not explicitly drawn or referred to.

X in Formula I or II can be N or CR², wherein R² is defined herein. Typically, X in Formula I or II is CH or N. For example, in some embodiments, the compound of Formula I or II can be characterized as having a subformula of Formula I-1, I-2, I-3, II-1, II-2, or II-3:

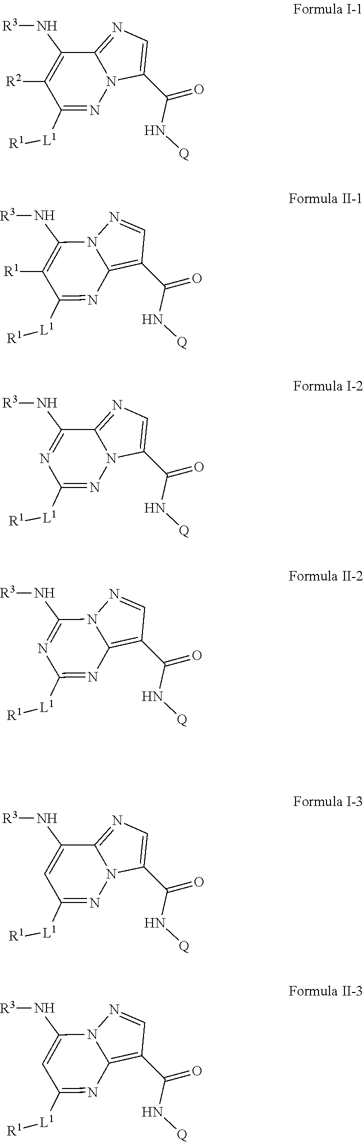

wherein L¹, R¹, R², R³, and Q include any of those described herein in any combination.

In some preferred embodiments, Q in Formula I or II (e.g., I-1, I-2, I-3, II-1, II-2, or II-3) is an optionally substituted fused bicyclic heterocyclyl, preferably, a 6-10 membered fused bicyclic heterocyclyl, which is optionally substituted. For example, in some embodiments, Q in Formula I or II (e.g., I-1, I-2, I-3, II-1, II-2, or II-3) is an optionally substituted fused 4,4-bicyclic heterocyclyl, fused 4,5-bicyclic heterocyclyl, fused 4,6-bicyclic heterocyclyl, fused 5,6-bicyclic heterocyclyl, fused 5,5-bicyclic heterocyclyl, or fused 6,6-bicyclic heterocyclyl. Either of the two rings of the bicyclic heterocyclyl can contain a ring heteroatom, and either of the two rings can be attached to the NH group of

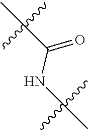 [SMILES: *NC(*)=O]

in Formula I or II. In some embodiments, the fused bicyclic heterocyclyl is not substituted. In some embodiments, the fused bicyclic heterocyclyl is substituted with one or more substituents, preferably 1 or 2 substituents. In such embodiments, the substituent(s) can be a substituent(s) of either of the two rings, and each substituent can be independently selected, for example, each substituent can be independently F, Cl, CN, OH, oxo (as valency permits), C₁₋₄ alkyl optionally substituted with F, cyclopropyl, cyclobutyl, or C₁₋₄ alkoxy optionally substituted with F.

In some embodiments, Q in Formula I or II (e.g., I-1, I-2, I-3, II-1, II-2, or II-3) is an optionally substituted fused 4,4-bicyclic heterocyclyl, wherein one of the 4-membered rings of the bicyclic heterocyclyl is a carbocyclic ring and is attached to the NH group of

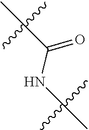 [SMILES: *NC(*)=O]

in Formula I or II, wherein the other 4-membered ring of the bicyclic heterocyclyl is a heterocyclic ring having one or two ring heteroatoms independently selected from N, O, and S. For example, in some embodiments, Q in Formula I or II (e.g., I-1, I-2, I-3, II-1, II-2, or II-3) can be

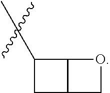 [SMILES: *C1CC2COC12]

In some embodiments, Q in Formula I or II (e.g., I-1, I-2, I-3, II-1, II-2, or II-3) is an optionally substituted fused 4,5-bicyclic or 4,6-bicyclic heterocyclyl, wherein the 4-membered ring of the bicyclic heterocyclyl is a carbocyclic ring and is attached to the NH group of

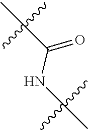 [SMILES: *NC(*)=O]

in Formula I or II, wherein the 5- or 6-membered ring of the bicyclic heterocyclyl is a heterocyclic ring having one or two ring heteroatoms independently selected from N, O, and S. For example, in some embodiments, Q in Formula I or II (e.g., I-1, I-2, I-3, II-1, II-2, or II-3) can be

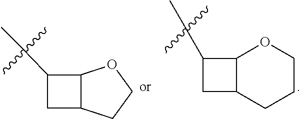 [SMILES: *C1CC2CCOC12 ; *C1CC2CCCOC12]

In some embodiments, Q in Formula I or II (e.g., I-1, I-2, I-3, II-1, II-2, or II-3) is an optionally substituted fused 4,5-bicyclic or 4,6-bicyclic heterocyclyl, wherein the 4-membered ring of the bicyclic heterocyclyl is a carbocyclic ring and is attached to the NH group of

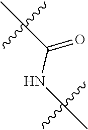 [SMILES: *NC(*)=O]

in Formula I or II, wherein the 5- or 6-membered ring of the bicyclic heterocyclyl is a heteroaryl ring having 1-3 ring heteroatom independently selected from N, O, and S. For example, in some embodiments, Q in Formula I or II (e.g., I-1, I-2, I-3, II-1, II-2, or II-3) can be selected from:

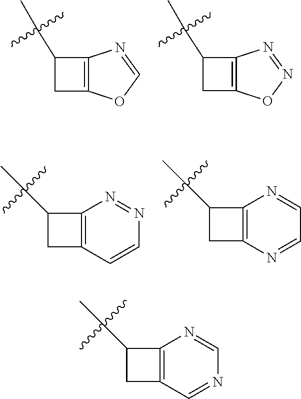 [SMILES: *C1Cc2ocnc21 ; *C1Cc2onnc21 ; *C1Cc2ccnnc21 ; *C1Cc2nccnc21 ; *C1Cc2cncnc21]

In some embodiments, Q in Formula I or II (e.g., I-1, I-2, I-3, II-1, II-2, or II-3) is an optionally substituted fused 4,5-bicyclic or 4,6-bicyclic heterocyclyl, wherein the 4-membered ring of the bicyclic heterocyclyl is a heterocyclic ring having one or two ring heteroatoms independently selected from N, O, and S and is attached to the NH group of

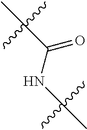 [SMILES: *NC(*)=O]

in Formula I or II, wherein the 5- or 6-membered ring of the bicyclic heterocyclyl is a carbocyclyl, phenyl, or heterocyclic ring having one or two ring heteroatoms independently selected from N, O, and S.

In some embodiments, Q in Formula I or II (e.g., I-1, I-2, I-3, II-1, II-2, or II-3) is an optionally substituted fused 4,5-bicyclic or 4,6-bicyclic heterocyclyl, wherein the 4-membered ring of the bicyclic heterocyclyl is a heterocyclic ring having one or two ring heteroatoms independently selected from N, O, and S and is attached to the NH group of

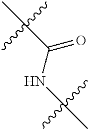 [SMILES: *NC(*)=O]

in Formula I or II, wherein the 5- or 6-membered ring of the bicyclic heterocyclyl is a heteroaryl ring having 1-4 ring heteroatoms independently selected from N, O, and S.

In some embodiments, Q in Formula I or II (e.g., I-1, I-2, I-3, II-1, II-2, or II-3) is an optionally substituted fused 5,5-bicyclic heterocyclyl, wherein one of the 5-membered rings of the bicyclic heterocyclyl is a carbocyclic ring and is attached to the NH group of

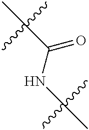 [SMILES: *NC(*)=O]

in Formula I or II, wherein the other 5-membered ring of the bicyclic heterocyclyl is a heteroaryl ring having 1-3 ring heteroatom independently selected from N, O, and S. For example, in some embodiments, Q in Formula I or II (e.g., I-1, I-2, I-3, II-1, II-2, or II-3) can be selected from:

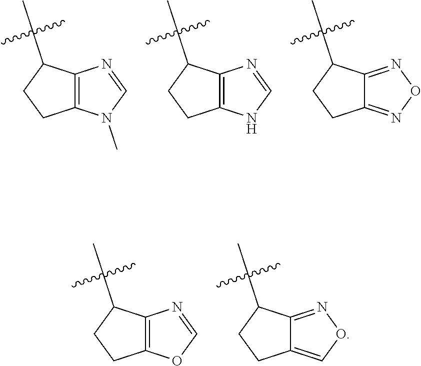

In some embodiments, Q in Formula I or II (e.g., I-1, I-2, I-3, II-1, II-2, or II-3) can be

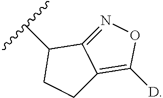

In some embodiments, Q in Formula I or II (e.g., I-1, I-2, I-3, II-1, II-2, or II-3) is an optionally substituted fused 5,5-bicyclic heterocyclyl, wherein one of the 5-membered rings of the bicyclic heterocyclyl is a heterocyclic ring having one or two ring heteroatoms independently selected from N, O, and S and is attached to the NH group of

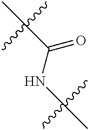 [SMILES: *NC(*)=O]

in Formula I or II, wherein the other 5-membered ring of the bicyclic heterocyclyl is a heteroaryl ring having 1-4 ring heteroatom independently selected from N, O, and S. For example, in some embodiments, Q in Formula I or II (e.g., I-1, I-2, I-3, II-1, II-2, or II-3) can be selected from:

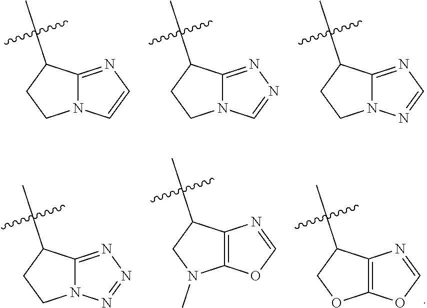

In some embodiments, Q in Formula I or II (e.g., I-1, I-2, I-3, II-1, II-2, or II-3) is an optionally substituted fused 5,5-bicyclic heterocyclyl, wherein one of the 5-membered rings of the bicyclic heterocyclyl is a carbocyclic ring and is attached to the NH group of

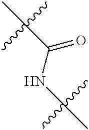 [SMILES: *NC(*)=O]

in Formula I or II, wherein the other 5-membered ring of the bicyclic heterocyclyl is a heterocyclic ring having one or two ring heteroatoms selected from N, O, and S. For example, in some embodiments, Q in Formula I or II (e.g., I-1, I-2, I-3, II-1, II-2, or II-3) can be

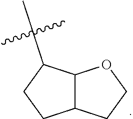 [SMILES: *C1CCC2CCOC12]

In some embodiments, Q in Formula I or II (e.g., I-1, I-2, I-3, II-1, II-2, or II-3) is an optionally substituted fused 5,5-bicyclic heterocyclyl, wherein one of the 5-membered rings of the bicyclic heterocyclyl is a heteroaryl ring having 1-3 ring heteroatom independently selected from N, O, and S and is attached to the NH group of

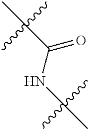 [SMILES: *NC(*)=O]

in Formula I or II, wherein the other 5-membered ring of the bicyclic heterocyclyl is a carbocyclic ring or heterocyclic ring having one or two ring heteroatoms independently selected from N, O, and S.

In some embodiments, Q in Formula I or II (e.g., I-1, I-2, I-3, II-1, II-2, or II-3) is an optionally substituted fused 5,5-bicyclic heterocyclyl, wherein one of the 5-membered rings of the bicyclic heterocyclyl is a heterocyclic ring having one or two ring heteroatoms independently selected from N, O, and S and is attached to the NH group of

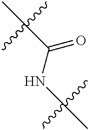 [SMILES: *NC(*)=O]

in Formula I or II, wherein the other 5-membered ring of the bicyclic heterocyclyl is carbocyclic ring, phenyl, or heterocyclic ring having one or two ring heteroatoms independently selected from N, O, and S.

In some embodiments, Q in Formula I or II (e.g., I-1, I-2, I-3, II-1, II-2, or II-3) is an optionally substituted fused 4,5-bicyclic or 4,6-bicyclic heterocyclyl, wherein the 5- or 6-membered ring of the bicyclic heterocyclyl is a carbocyclic ring and is attached to the NH group of

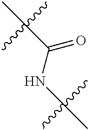 [SMILES: *NC(*)=O]

in Formula I or II, wherein the 4-membered ring of the bicyclic heterocyclyl is a carbocyclic ring or heterocyclic ring having one or two ring heteroatoms independently selected from N, O, and S. For example, in some embodiments, Q in Formula I or II (e.g., I-1, I-2, I-3, II-1, II-2, or II-3) can be

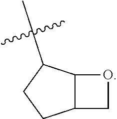 [SMILES: *C1CCC2COC12]

In some embodiments, Q in Formula I or II (e.g., I-1, I-2, I-3, II-1, II-2, or II-3) is an optionally substituted fused 4,5-bicyclic or 4,6-bicyclic heterocyclyl, wherein the 5- or 6-membered ring of the bicyclic heterocyclyl is a heterocyclic ring having 1-2 ring heteroatom independently selected from N, O, and S and is attached to the NH group of

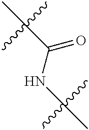 [SMILES: *NC(*)=O]

in Formula I or II, wherein the 4-membered ring of the bicyclic heterocyclyl is a carbocyclic ring or heterocyclic ring having one or two ring heteroatoms independently selected from N, O, and S.

In some embodiments, Q in Formula I or II (e.g., I-1, I-2, I-3, II-1, II-2, or II-3) is an optionally substituted fused 4,5-bicyclic or 4,6-bicyclic heterocyclyl, wherein the 5- or 6-membered ring of the bicyclic heterocyclyl is a heteroaryl ring having 1-4 ring heteroatom independently selected from N, O, and Sand is attached to the NH group of

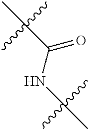 [SMILES: *NC(*)=O]

in Formula I or II, wherein the 4-membered ring of the bicyclic heterocyclyl is a carbocyclic ring or heterocyclic ring having one or two ring heteroatoms independently selected from N, O, and S.

In some embodiments, Q in Formula I or II (e.g., I-1, I-2, I-3, II-1, II-2, or II-3) is an optionally substituted fused 5,6-bicyclic heterocyclyl, wherein the 5-membered ring of the bicyclic heterocyclyl is a carbocyclyl ring and is attached to the NH group of

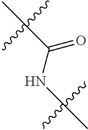 [SMILES: *NC(*)=O]

in Formula I or II, wherein the 6-membered ring of the bicyclic heterocyclyl is a heterocyclic ring having one or two ring heteroatoms independently selected from N, O, and S. For example, in some embodiments, Q in Formula I or II (e.g., I-1, I-2, I-3, II-1, II-2, or II-3) can be

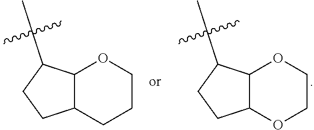 [SMILES: *C1CCC2CCCOC12 ; *C1CCC2OCCOC12]

In some embodiments, Q in Formula I or II (e.g., I-1, I-2, I-3, II-1, II-2, or II-3) is an optionally substituted fused 5,6-bicyclic heterocyclyl, wherein the 5-membered ring of the bicyclic heterocyclyl is a carbocyclyl ring and is attached to the NH group of

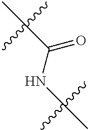 [SMILES: *NC(*)=O]

in Formula I or II, wherein the 6-membered ring of the bicyclic heterocyclyl is a heteroaryl ring having 1-3 ring nitrogens. For example, in some embodiments, Q in Formula I or II (e.g., I-1, I-2, I-3, II-1, II-2, or II-3) can be selected from:

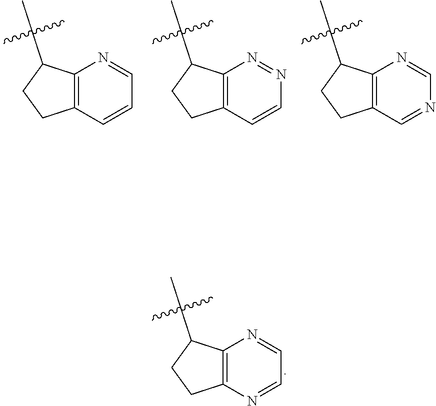

In some embodiments, Q in Formula I or II (e.g., I-1, I-2, I-3, II-1, II-2, or II-3) is an optionally substituted fused 5,6-bicyclic heterocyclyl, wherein the 5-membered ring of the bicyclic heterocyclyl is a heterocyclyl ring having 1-2 ring heteroatom independently selected from N, O, and S and is attached to the NH group of

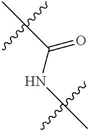 [SMILES: *NC(*)=O]

in Formula I or II, wherein the 6-membered ring of the bicyclic heterocyclyl is a heteroaryl ring having 1-3 ring nitrogens. For example, in some embodiments, Q in Formula I or II (e.g., I-1, I-2, I-3, II-1, II-2, or II-3) can be selected from:

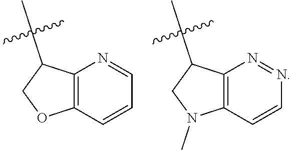

In some embodiments, Q in Formula I or II (e.g., I-1, I-2, I-3, II-1, II-2, or II-3) is an optionally substituted fused 5,6-bicyclic heterocyclyl, wherein the 5-membered ring of the bicyclic heterocyclyl is a heteroaryl ring having 1-4 ring heteroatom independently selected from N, O, and S and is attached to the NH group of

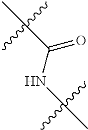 [SMILES: *NC(*)=O]

in Formula I or II, wherein the 6-membered ring of the bicyclic heterocyclyl is a carbocyclic ring or heterocyclic ring having one or two ring heteroatoms independently selected from N, O, and S.

In some embodiments, Q in Formula I or II (e.g., I-1, I-2, I-3, II-1, II-2, or II-3) is an optionally substituted fused 5,6-bicyclic heterocyclyl, wherein the 5-membered ring of the bicyclic heterocyclyl is a heterocyclic ring having one or two ring heteroatoms independently selected from N, O, and S and is attached to the NH group of

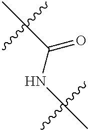 [SMILES: *NC(*)=O]

in Formula I or II, wherein the 6-membered ring of the bicyclic heterocyclyl is a carbocyclic ring, phenyl, or heterocyclic ring having one or two ring heteroatoms independently selected from N, O, and S.

In some embodiments, Q in Formula I or II (e.g., I-1, I-2, I-3, II-1, II-2, or II-3) is an optionally substituted fused 6,6-bicyclic heterocyclyl, wherein one of the 6-membered rings of the bicyclic heterocyclyl is a carbocyclic ring and is attached to the NH group of

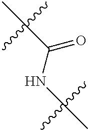 [SMILES: *NC(*)=O]

in Formula I or II, wherein the other 6-membered ring of the bicyclic heterocyclyl is a heteroaryl ring having 1-3 ring heteroatom independently selected from N, O, and S.

In some embodiments, Q in Formula I or II (e.g., I-1, I-2, I-3, II-1, II-2, or II-3) is an optionally substituted fused 6,6-bicyclic heterocyclyl, wherein one of the 6-membered rings of the bicyclic heterocyclyl is a heterocyclic ring having one or two ring heteroatoms

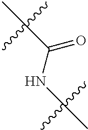 [SMILES: *NC(*)=O]

independently selected from N, O, and S and is attached to the NH group of in Formula I or II, wherein the other 6-membered ring of the bicyclic heterocyclyl is a heteroaryl ring having 1-3 ring nitrogens.

In some embodiments, Q in Formula I or II (e.g., I-1, I-2, I-3, II-1, II-2, or II-3) is an optionally substituted fused 6,6-bicyclic heterocyclyl, wherein one of the 6-membered rings of the bicyclic heterocyclyl is a carbocyclic ring and is attached to the NH group of

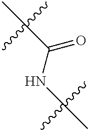 [SMILES: *NC(*)=O]

in Formula I or II, wherein the other 6-membered ring of the bicyclic heterocyclyl is a heterocyclic ring having one or two ring heteroatoms selected from N, O, and S.

In some embodiments, Q in Formula I or II (e.g., I-1, I-2, I-3, II-1, II-2, or II-3) is an optionally substituted fused 6,6-bicyclic heterocyclyl, wherein one of the 6-membered rings of the bicyclic heterocyclyl is a heteroaryl ring having 1-3 ring nitrogens and is attached to the NH group of

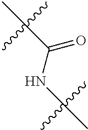 [SMILES: *NC(*)=O]

in Formula I or II, wherein the other 6-membered ring of the bicyclic heterocyclyl is a carbocyclic ring or heterocyclic ring having one or two ring heteroatoms independently selected from N, O, and S.

In some embodiments, Q in Formula I or II (e.g., I-1, I-2, I-3, II-1, II-2, or II-3) is an optionally substituted fused 6,6-bicyclic heterocyclyl, wherein one of the 6-membered rings of the bicyclic heterocyclyl is a heterocyclyl ring having 1-2 ring heteroatoms independently selected from N, O, and S and is attached to the NH group of

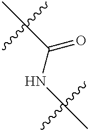 [SMILES: *NC(*)=O]

in Formula I or II, wherein the other 6-membered ring of the bicyclic heterocyclyl is a carbocyclic ring, phenyl, or heterocyclic ring having one or two ring heteroatoms independently selected from N, O, and S.

In some embodiments, Q in Formula I or II (e.g., I-1, I-2, I-3, II-1, II-2, or II-3) can be any of the fused bicyclic heterocyclyl or heteroaryl ring described herein, which comprises ring A and ring B fused together, represented by

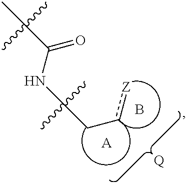

wherein the ring atom Z in ring B is a heteroatom such as O or N, wherein ring A can be a carbocyclyl, phenyl, heterocyclyl, or heteroaryl, typically 4-7 membered, such as those discussed herein, and ring B can be a heterocyclyl or heteroaryl, typically 4-7 membered, such as those discussed herein, wherein each of ring A and ring B can be optionally substituted. To be clear, the two ring atoms of ring A that bond to the NH and Z, respectively, are carbon atoms. The two shared ring atoms of ring A and ring B can be two carbons or one carbon (bonded to Z) and one nitrogen.

In some specific embodiments, Q in Formula I or II (e.g., I-1, I-2, I-3, II-1, II-2, or II-3) can be characterized as having the structure of F-1, F-2, or F-3:

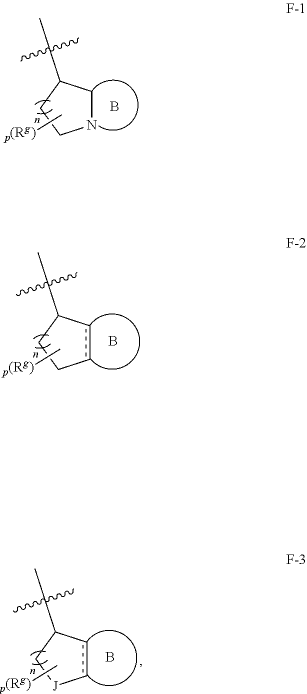

wherein:
ring B in F-1 is an optionally substituted 5-membered heteroaryl having 1-4 ring heteroatoms independently selected from nitrogen, oxygen, and sulfur, or an optionally substituted 4-7 membered such as 4-, 5- or 6-membered heterocyclyl having 1-2 ring heteroatoms independently selected from nitrogen, oxygen, and sulfur,
ring B in F-2 or F-3 is an optionally substituted 5- or 6-membered heteroaryl having 1-3 ring heteroatoms independently selected from nitrogen, oxygen, and sulfur, or an optionally substituted 4-7 membered such as 4-, 5- or 6-membered heterocyclyl having 1-2 ring heteroatoms independently selected from nitrogen, oxygen, and sulfur,
J is O or NR¹¹, wherein R¹¹ is a C₁₋₄ alkyl optionally substituted with 1-3 R^{s1},
n is 0, 1, or 2,
p is 0, 1, 2, or 3, as valency permits,
R^{g} at each occurrence is independently OH, halogen (e.g., F or Cl), CN, oxo (as valency permits), C₁₋₄ alkyl optionally substituted with one or more (e.g., 1, 2, or 3) R^{s2}, C₁₋₄ heteroalkyl optionally substituted with one or more (e.g., 1, 2, or 3) R^{s2}, C₃₋₆ cycloalkyl optionally substituted with one or more (e.g., 1, 2, or 3) R^{s2}, or 4-7 membered heterocyclyl optionally substituted with one or more (e.g., 1, 2, or 3) R^{s2},
wherein R^{s1} at each occurrence is independently F, OH, oxo (as valency permits), methoxy, or methyl; and
R^{s2} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F. To be clear, in F-1, F-2, or F-3, when p is 1, 2, or 3, it should be understood that R⁹ is attached to the ring that is fused to ring B, and ring B itself may be optionally substituted with one or more (e.g., 1 or 2) R^{S3}, wherein R^{S3} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.

Various heterocycles and heteroaryls are suitable ring B in F-1, F-2, or F-3. In some embodiments, ring B in F-1, F-2, or F-3 is a 5-membered heteroaryl having 1-4 ring heteroatoms independently selected from nitrogen, oxygen, and sulfur, such as imidazole, pyrazole, oxazole, isoxazole, oxadiazole (e.g., 1,2,5-oxadiazole), thiadiazole, triazole, or tetrazole, as applicable, wherein the 5-membered heteroaryl is optionally substituted, e.g., with one or more (e.g., 1 or 2) R^{S3}, wherein R^{S3} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F. For example, in some embodiments, ring B in F-1 can be selected from:

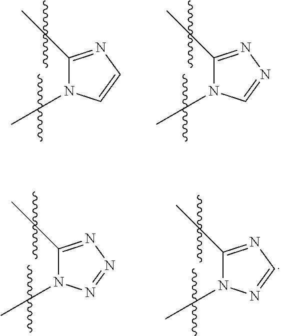 [SMILES: *c1nccn1* ; *c1nncn1* ; *c1nnnn1* ; *c1ncnn1*]

In some embodiments, ring B in F-2 or F-3 can be selected from:

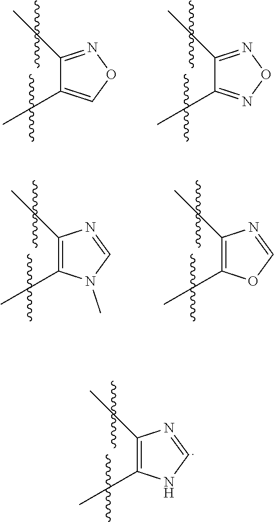

In some embodiments, ring B in F-2 or F-3 is a 6-membered heteroaryl having 1-3 ring nitrogen atoms, such as pyridine, pyrimidine, pyridazine, pyrazine, or triazine, as applicable, wherein the 6-membered heteroaryl is optionally substituted, e.g., with one or more (e.g., 1 or 2) R^{s3}, wherein R^{s3} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F. For example, in some embodiments, ring B in F-2 or F-3 can be selected from:

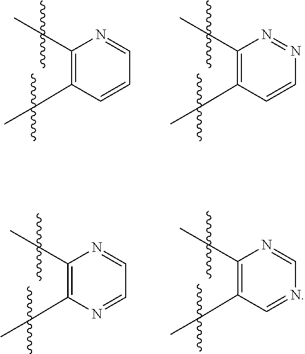 [SMILES: *c1cccnc1* ; *c1ccnnc1* ; *c1nccnc1* ; *c1cncnc1*]

In some embodiments, ring B in F-1, F-2, or F-3 can also be a 4-7 membered heterocyclyl having 1-3 ring heteroatoms independently selected from N, O, and S, wherein the 4-7 membered heterocyclyl is optionally substituted, e.g., with one or more (e.g., 1 or 2) R^{s3}, wherein R^{s3} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F. For example, in some embodiments, in F-2 and F-3, ring B can be selected from

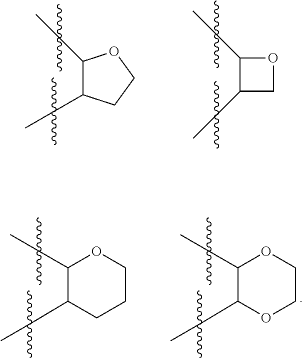 [SMILES: *C1CCOC1* ; *C1COC1* ; *C1CCCOC1* ; *C1OCCOC1*]

In some embodiments, in F-1, ring B can be

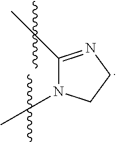 [SMILES: *C1=NCCN1*]

The ring that is fused to ring B in F-1, F-2, or F-3 is typically a 4-6 membered ring. For example, in some embodiments, n in F-1, F-2, or F-3 is 0. In some embodiments, n in F-1, F-2, or F-3 is 1. In some embodiments, n in F-1, F-2, or F-3 can also be 2.

Typically, the ring that is fused to ring B in F-1, F-2, or F-3 is not substituted, i.e., p is 0. In some embodiments, p can also be 1, 2, or 3. For example, in some embodiments, p in F-1, F-2, or F-3 can be 1 or 2. In some embodiments, when present, R⁹ at each occurrence can be independently OH, F, Cl, CN, oxo (as valency permits), C₁₋₄ alkyl optionally substituted with F, cyclopropyl, cyclobutyl, or C₁₋₄ alkoxy optionally substituted with F.

Typically, in F-3, J is O or NCH₃.

In some embodiments, F-1, F-2, or F-3 can be represented by the following structures, respectively:

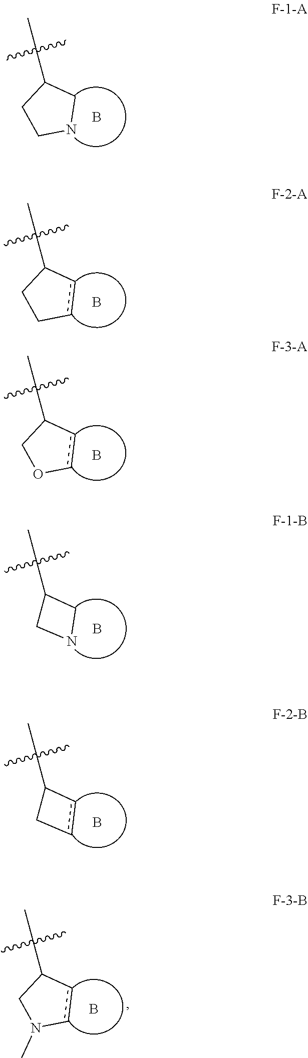

wherein ring B include any of those described herein for F-1, F-2, or F-3, respectively.

In any of the embodiments described herein, unless otherwise specified or contrary from context, in Formula I or II (e.g., I-1, I-2, I-3, II-1, II-2, or II-3), Q can be selected from:

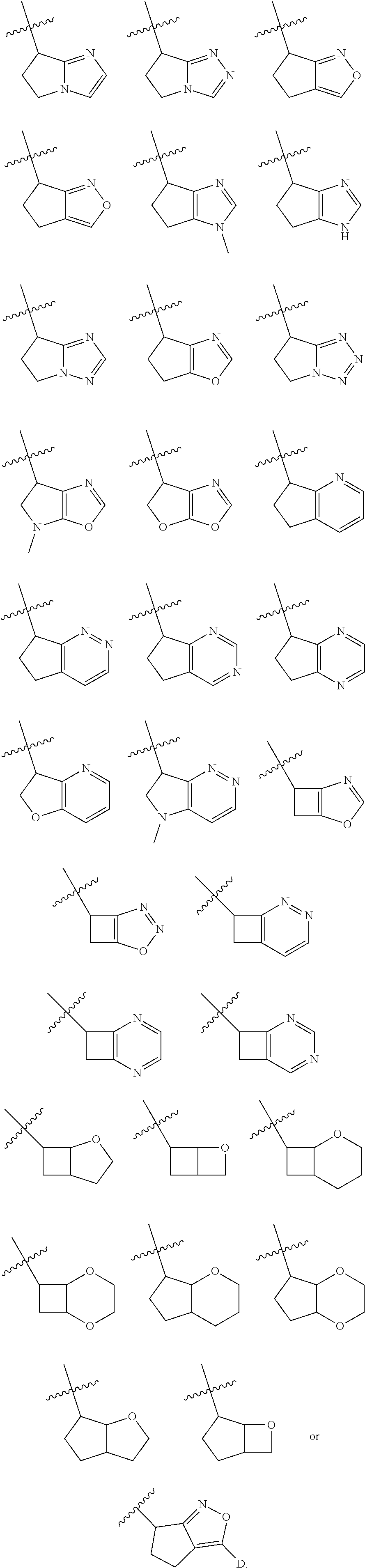

In some preferred embodiments, in Formula I or II (e.g., I-1, I-2, I-3, II-1, II-2, or II-3), Q can be selected from:

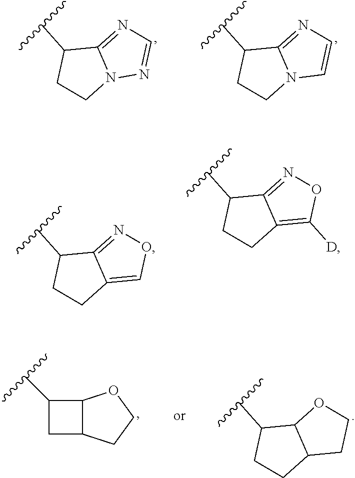

As would be understood by those skilled in the art, in some embodiments, Q can have one or more asymmetric centers. The present disclosure is not particularly limited to any particular stereoisomers and can include individual stereoisomers, mixtures of stereoisomers enriched in one or more stereoisomers, and mixtures of stereoisomers in any ratio. For example, in some embodiments, Q in Formula I or II (e.g., I-1, I-2, I-3, II-1, II-2, or II-3) can exist predominantly in the configuration of the as-drawn stereoisomer selected from:

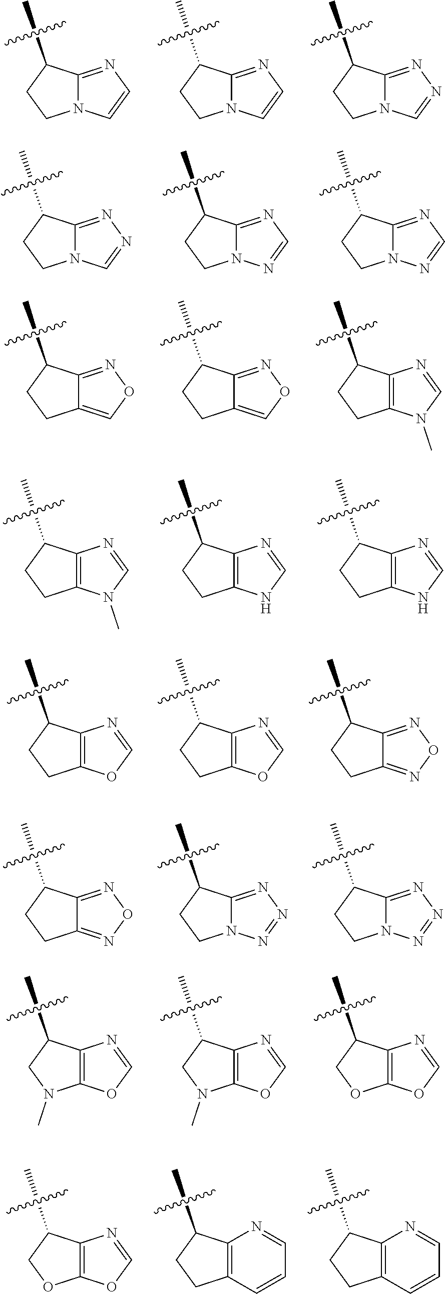

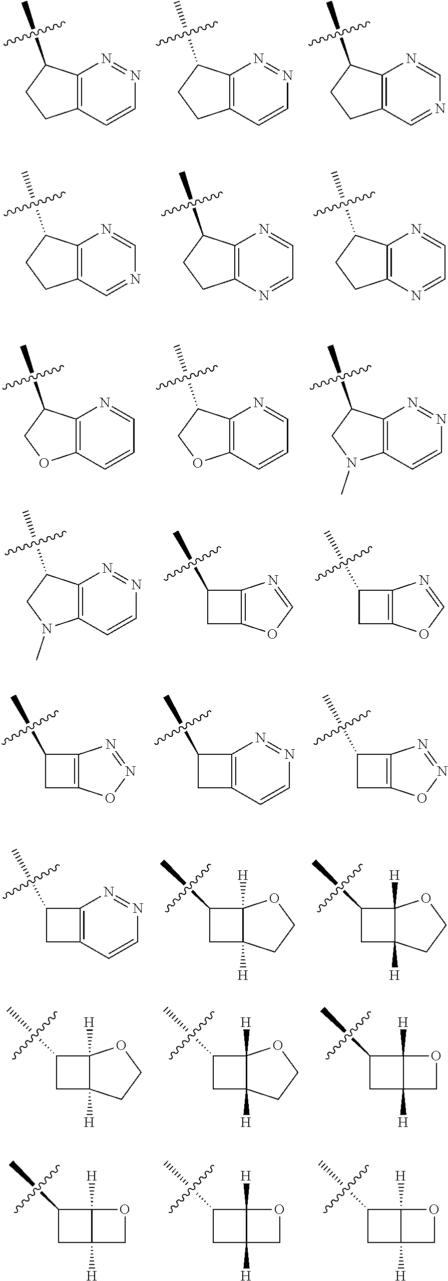

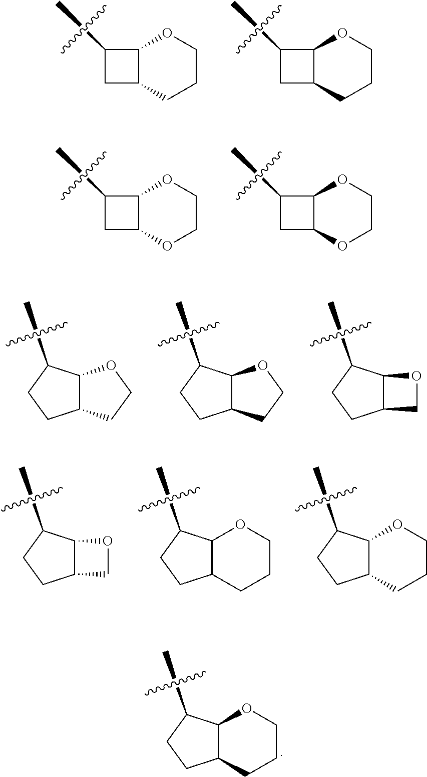

For example, in some preferred embodiments, in Formula I or II (e.g., I-1, I-2, I-3, II-1, II-2, or II-3), Q can be

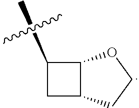

For the avoidance of doubt, when compounds of Formula I or II are said to have such Q group with the stereochemistry drawn, unless otherwise specified or otherwise contrary from context, it should be understood that in some embodiments, with respect to the as-drawn stereochemical center(s), the compound of Formula I or II (e.g., I-1, I-2, I-3, II-1, II-2, or II-3) can exist predominantly as the as-drawn stereoisomer, such as with less than 20%, less than 10%, less than 5%, less than 1%, by weight, by SFC or HPLC area, or both, or with a non-detectable amount of the other stereoisomer(s).

In some specific embodiments, the compound of Formula I or II can be characterized as having a subformula according to any of the following:

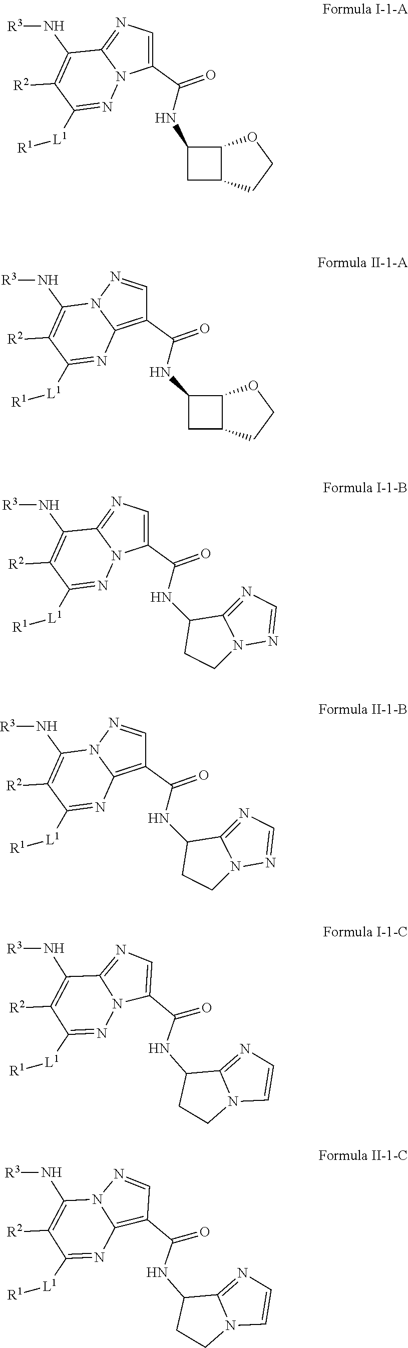

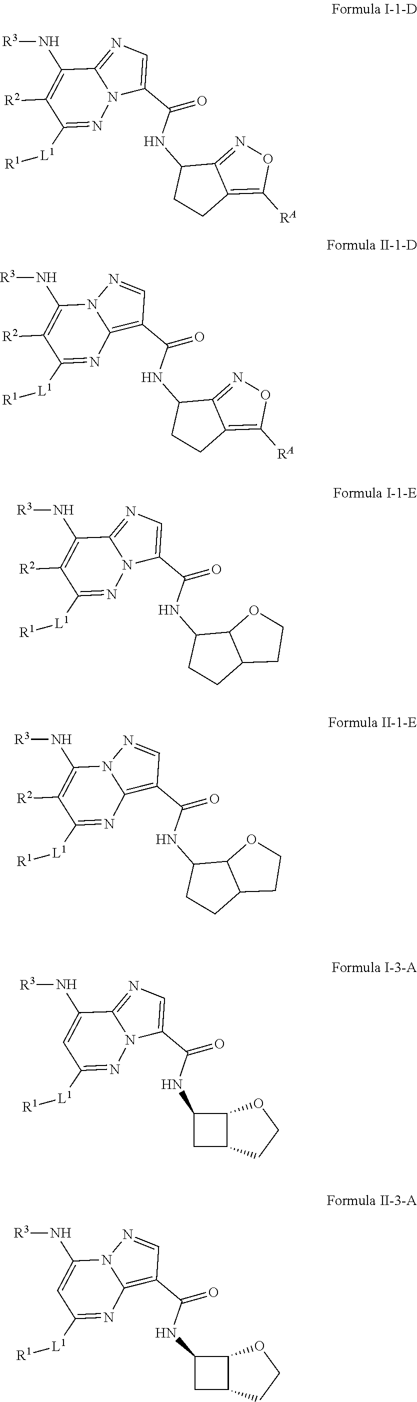

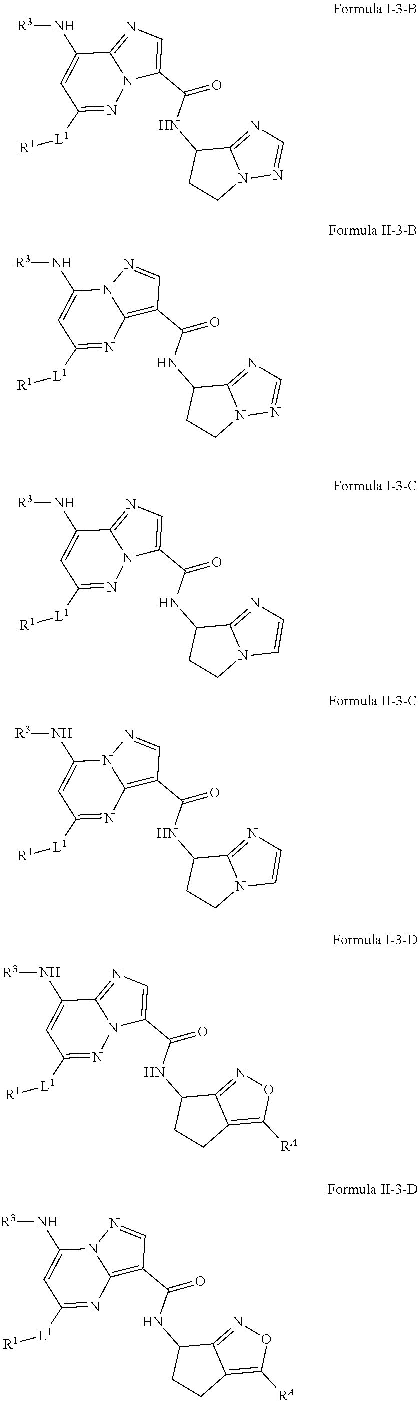

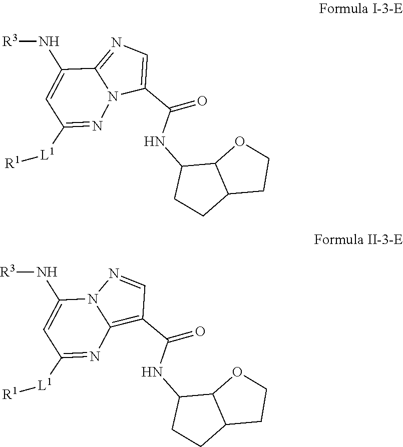

wherein R^{A} is hydrogen or deuterium, wherein L¹, R¹, R², and R³ include any of those described herein in any combination.

In some preferred embodiments, Q in Formula I or II (e.g., I-1, I-2, I-3, II-1, II-2, or II-3) is

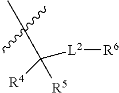

as defined herein. For example, in some embodiments, the compound of Formula I or II can have a subformula according to Formula I-4 or II-4:

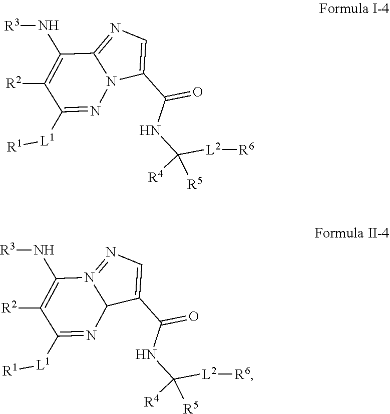

wherein L¹, L², R¹, R², R³, R⁴, R⁵, and R⁶ include any of those described herein in any combination.

Typically, L² in Formula I-4 or II-4 can be a bond, NR^{11A}, optionally substituted C₁₋₄ alkylene or optionally substituted C₁₋₄ heteroalkyelene. Typically, L² is a bond, and R⁶ is directly bonded to the carbon where both R⁴ and R⁵ are attached to. However, L² is not limited to a bond. For example, in some embodiments, L² can be a C₁₋₄ alkylene (e.g., CH₂).

In some embodiments, R⁴ and R⁵ in Formula I-4 or II-4 are each independently hydrogen or a C₁₋₄ alkyl optionally substituted with one or more substituents independently selected from F, hydroxyl, or C₁₋₄ alkoxy. For example, in some embodiments, both R⁴ and R⁵ can be hydrogen. In some embodiments, one of R⁴ and R⁵ is hydrogen and the other of R⁴ and R⁵ is a C₁₋₄ alkyl, such as methyl. In some embodiments, both R⁴ and R⁵ are C₁₋₄ alkyl.

In some embodiments, R⁴ and R⁵ in Formula I-4 or II-4 are joined to form a C₃₋₆ cycloalkyl ring, e.g., cyclopropyl or cyclobutyl, which is optionally substituted with one or more (e.g., 1 or 2) substituents independently selected from C₁₋₄ alkyl, F, hydroxyl, or C₁₋₄ alkoxy, wherein two substituents together with the C₃₋₆ cycloalkyl ring can form a 5-10 membered spiro, bridged, or fused bicyclic ring, which optionally includes one or more ring heteroatoms independently selected from nitrogen, oxygen, and sulfur. For example, in some embodiments, R⁴ and R⁵ can be joined to form an unsubstituted cyclopropyl or cyclobutyl. In some embodiments, R⁴ and R⁵ can be joined to form cyclopropyl or cyclobutyl, which is further substituted with 1 or more, typically, 1 or 2 substituents independently selected from C₁₋₄ alkyl, F, hydroxyl, or C₁₋₄ alkoxy. In some embodiments, R⁴ and R⁵ can be joined to form a C₃₋₆ cycloalkyl ring, wherein two substituents of the C₃₋₆ cycloalkyl ring together with the C₃₋₆ cycloalkyl ring can form a 5-10 membered spiro, bridged, or fused bicyclic ring, which optionally includes one or more ring heteroatoms independently selected from nitrogen, oxygen, and sulfur. To be clear, in such embodiments, the 5-10 membered spiro, bridged, or fused bicyclic ring can be further optionally substituted at any suitable ring atoms, e.g., with one or more (e.g., 1 or 2) substituents independently selected from C₁₋₄ alkyl, F, hydroxyl, or C₁₋₄ alkoxy.

In some embodiments, R⁴ and R⁵ in Formula I-4 or II-4 can also be joined to form a 3-8 membered heterocyclic ring having one or more ring heteroatoms independently selected from nitrogen, oxygen, and sulfur, which is optionally substituted, for example, with one or more (e.g., 1 or 2) substituents independently selected from oxo, imino (═NH), methyl imino (═N(Me)), C₁₋₄ alkyl, 4-8 membered heterocyclyl, 5 or 6 membered heteroaryl, F, hydroxyl, or C₁₋₄ alkoxy. For example, in some embodiments, R⁴ and R⁵ in Formula I or II can also be joined to form a 5 or 6 membered heterocyclic ring having 1-3 ring heteroatoms independently selected from nitrogen, oxygen, and sulfur, which is optionally substituted with one or more (e.g., 1 or 2) substituents independently selected from oxo, imino (═NH), methyl imino (═N(Me)), 5 or 6 membered heteroaryl, C₁₋₄ alkyl, F, hydroxyl, or C₁₋₄ alkoxy, wherein the alkyl, heteroaryl, or alkoxy can be optionally substituted, for example, with C₁₋₄ alkyl, F, hydroxyl, or C₁₋₄ alkoxy. In some embodiments, two substituents of the 3-8 membered heterocyclic ring together with the 3-8 membered heterocyclic ring can form a 5-10 membered spiro, bridged, or fused bicyclic heterocyclic ring having one or more ring heteroatoms independently selected from nitrogen, oxygen, and sulfur.

In some particular embodiments, the compound of Formula I-4 or II-4 can be characterized as having one of the following formulae:

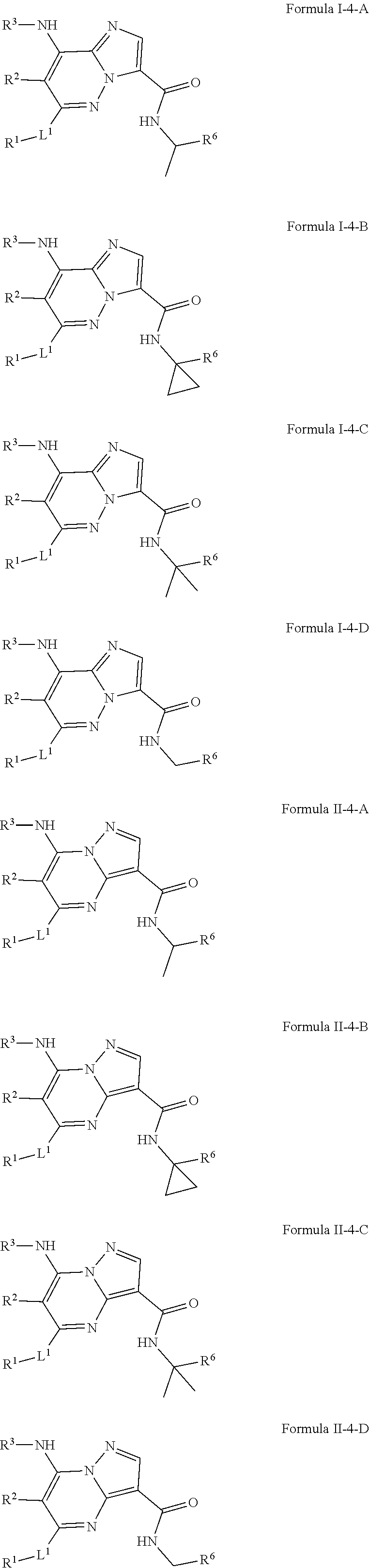

wherein L¹, R¹, R², R³, and R⁶ include any of those described herein in any combination.

Various groups are suitable as R⁶ in Formula I or II (e.g., Formula I-4 or II-4 or any of the sub-formulae described herein). In some embodiments, R⁶ can be hydrogen. However, typically, R⁶ is a 4-6 membered heterocyclic ring or a 5 or 6 membered heteroaryl ring. For example, in some embodiments, R⁶ is 4-6 membered heterocyclic ring having one or more ring heteroatoms independently selected from nitrogen, oxygen, and sulfur, which is saturated or partially unsaturated, which is optionally substituted with one or more substituents independently selected from oxo, F, hydroxyl, C₁₋₄ alkyl, and C₁₋₄ alkoxy. In some embodiments, R⁶ is a 5 or 6 membered heteroaryl having one or more (e.g., 1, 2, or 3) ring heteroatoms independently selected from nitrogen, oxygen, and sulfur, which is optionally substituted with one or more substituents independently selected from F, Cl, CN, hydroxyl, C₁₋₄ alkyl, and C₁₋₄ alkoxy.

In some embodiments, R⁶ can be selected from:

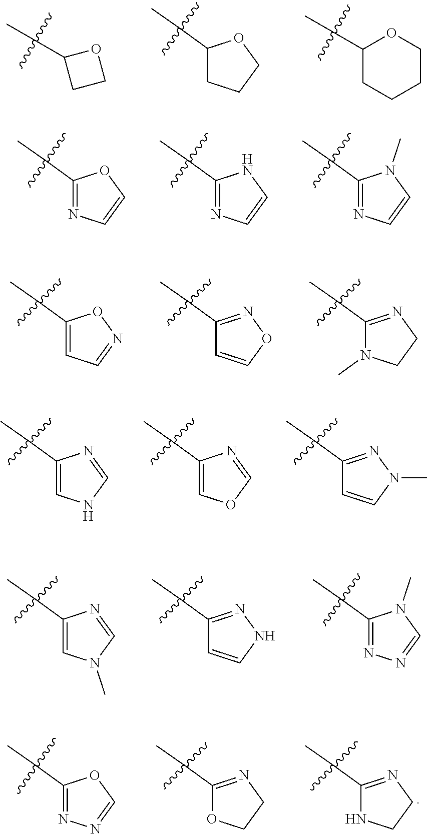

The stereochemistry of R⁶, when applicable, is also not particularly limited. For example, in some embodiments, R⁶ can be selected from the following stereoisomers:

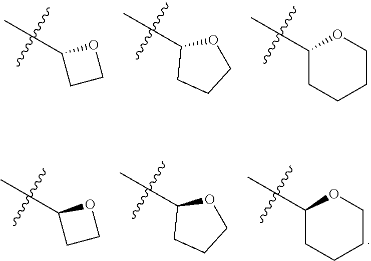 [SMILES: *[C@H]1CCO1 ; *[C@H]1CCCO1 ; *[C@H]1CCCCO1 ; *[C@@H]1CCO1 ; *[C@@H]1CCCO1 ; *[C@@H]1CCCCO1]

The combination of R⁴, R⁵, L², and R⁶ are not particularly limited. For example, in some embodiments, the

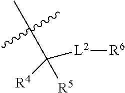

in Formula I-4 or II-4 can be selected from

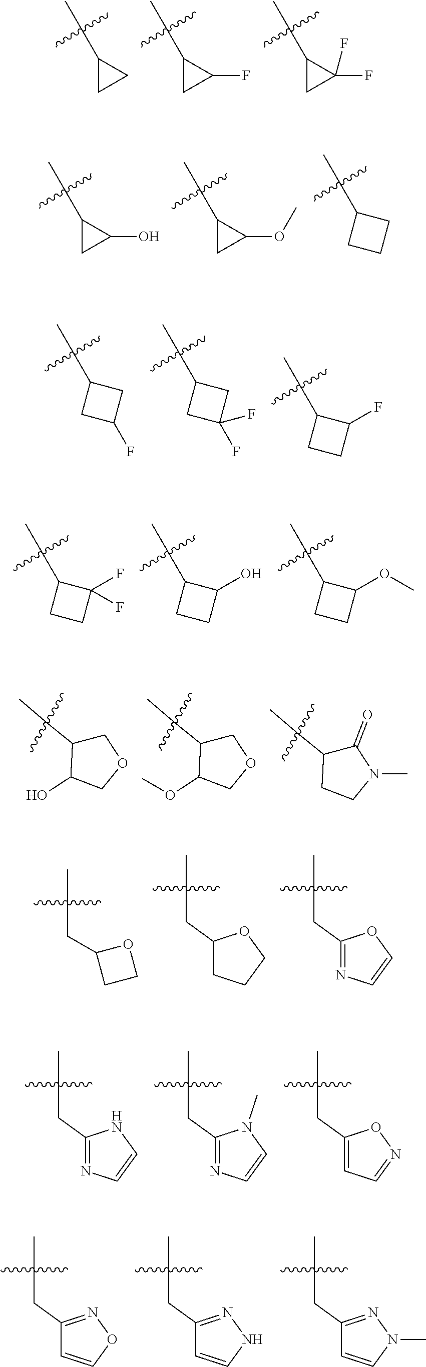

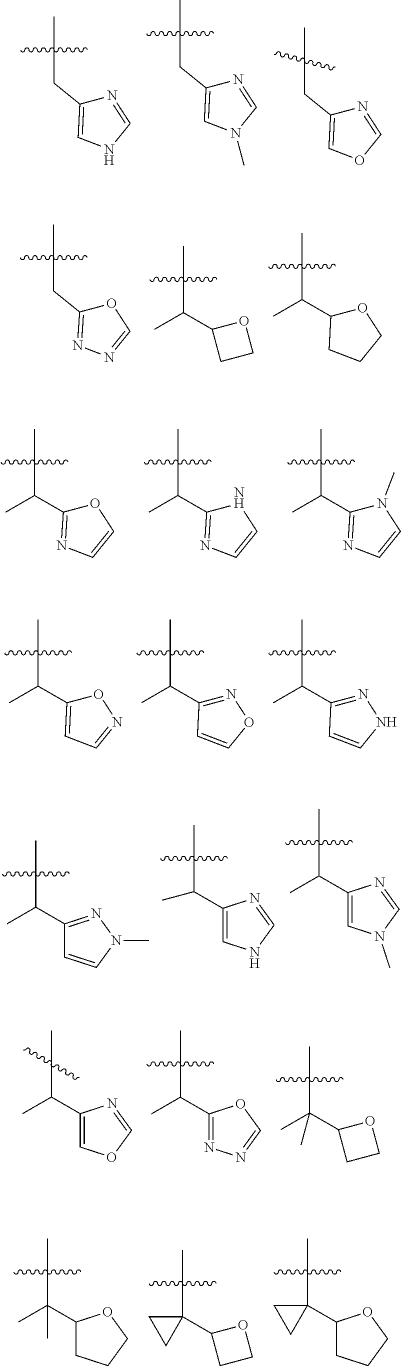

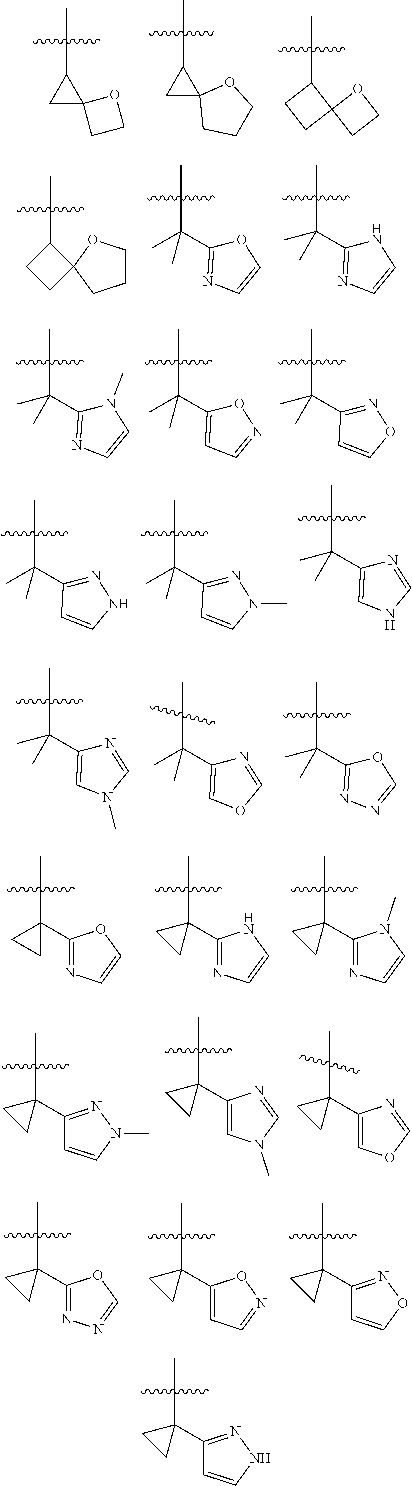

In some preferred embodiments, the

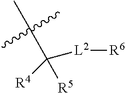

### in Formula I-4 or II-4 can be

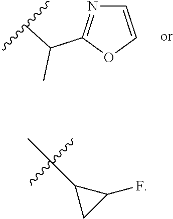

In some embodiments, the

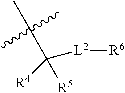

in Formula I-4 or II-4 can be selected from

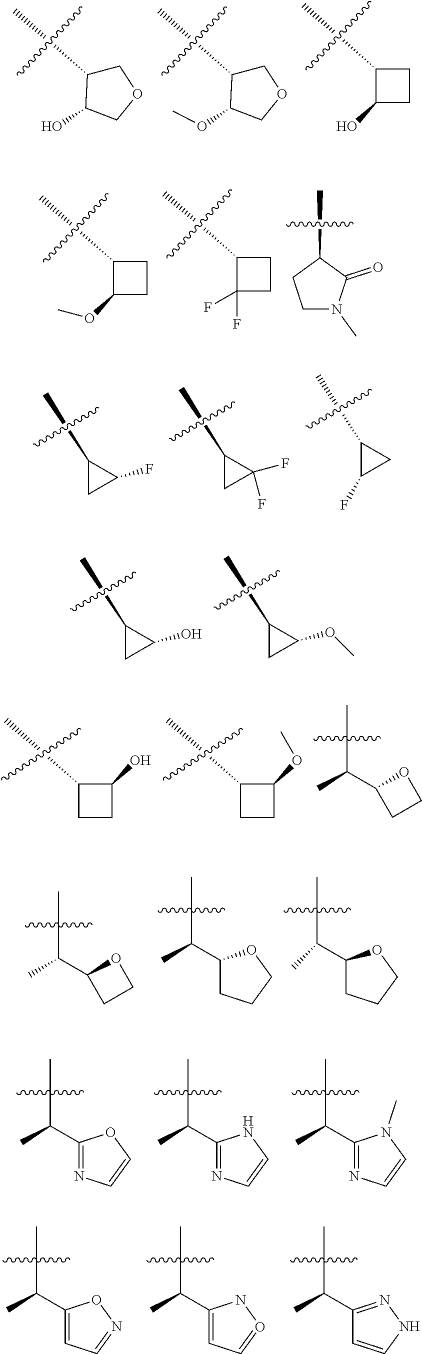

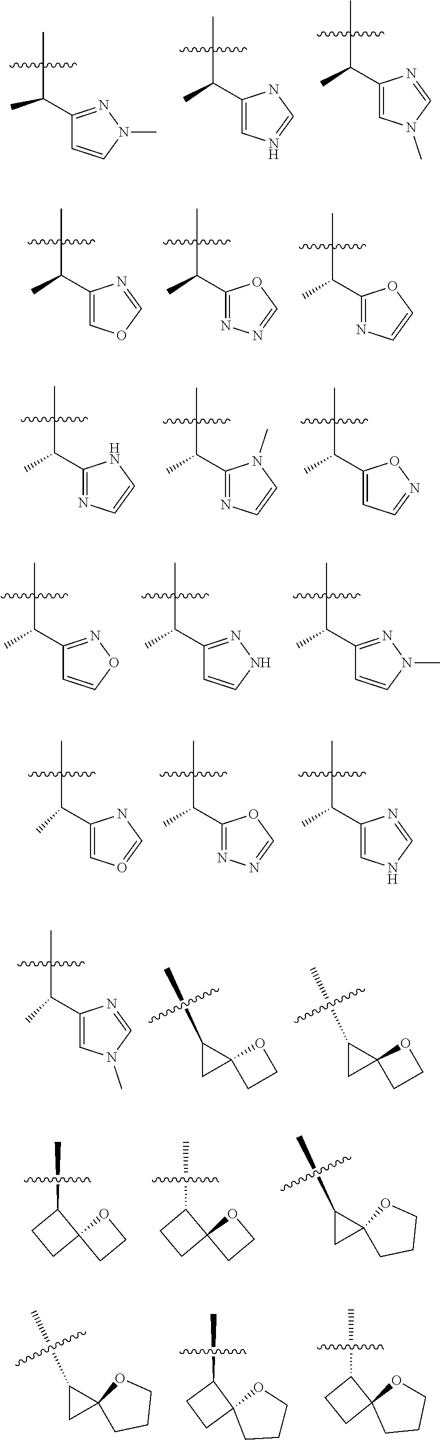

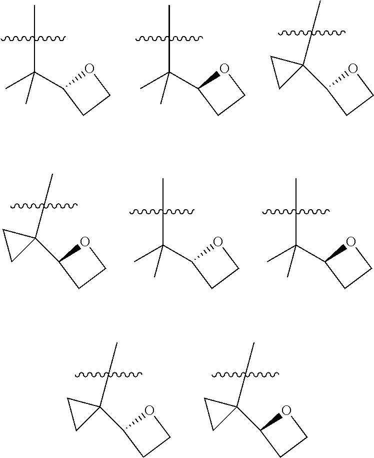

As discussed herein, when the stereochemistry of

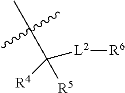

in Formula I-4 or II-4 is drawn, unless otherwise specified or contrary from context, it should be understood that in some embodiments, with respect to the as-drawn stereochemical center(s), the compound of Formula I-4 or II-4 (e.g., any of its subformulae herein) can exist predominantly as the as-drawn stereoisomer, such as with less than 20%, less than 10%, less than 5%, less than 1%, by weight, by SFC or HPLC area, or both, or with a non-detectable amount of the other stereoisomer(s).

In some preferred embodiments, the compound of Formula I-4 or II-4 can have a structure according to any of the following subformulae:

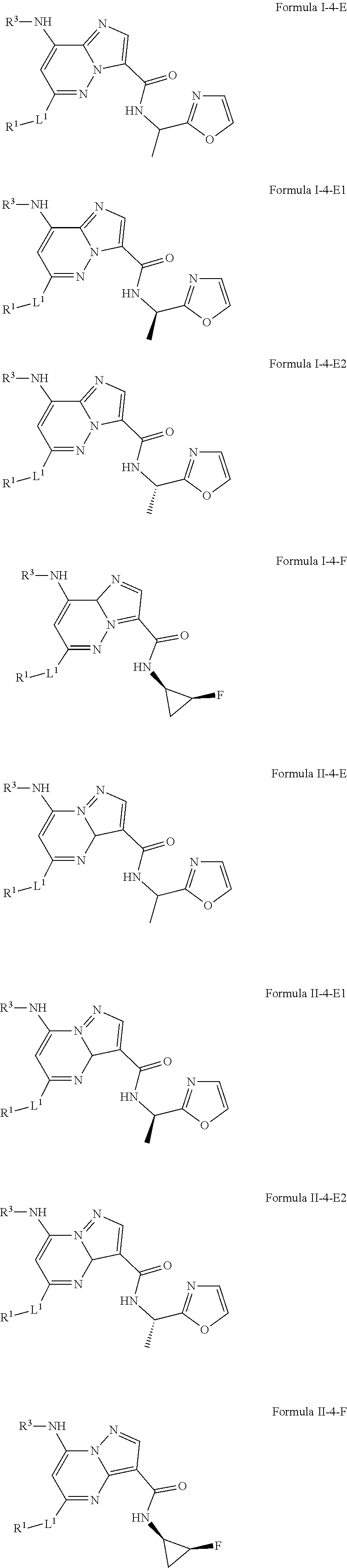

wherein L¹, R¹, and R³ include any of those described herein in any combinations.

Typically, L¹ in Formula I or II (e.g., I-1, I-2, I-3, I-4, II-1, II-2, II-3 or II-4, or any subformulae thereof) can be a bond, NR¹⁰, O, optionally substituted C₁₋₄ alkylene or optionally substituted C₁₋₄ heteroalkylene. For example, in some embodiments, L¹ is a bond, and R¹ is directly bonded to the bicyclic heteroaryl core structure in Formula I or II. In some embodiments, L¹ can also be NR¹⁰, preferably, NH. In some embodiments, L¹ can also be 0.

R¹ in Formula I or II (e.g., I-1, I-2, I-3, I-4, II-1, II-2, II-3 or II-4, or any subformulae thereof) is typically an optionally substituted 5 or 6 membered heteroaryl, optionally substituted heterocyclyl, an optionally substituted phenyl, an optionally substituted bicyclic heteroaryl. For example, in some embodiments, in Formula I or II (e.g., I-1, I-2, I-3, I-4, II-1, II-2, II-3 or II-4, or any subformulae thereof), R¹ is an optionally substituted phenyl, optionally substituted 5- or 6-membered heteroaryl ring having 1-4 ring heteroatoms independently selected from nitrogen, oxygen, and sulfur, optionally substituted 8-10 membered bicyclic heteroaryl having 1-4 ring heteroatoms independently selected from nitrogen, oxygen, and sulfur, or optionally substituted 8-10 membered heterocycle having 1-4 ring heteroatoms independently selected from nitrogen, oxygen, and sulfur.

For example, in some embodiments, R¹ in Formula I or II (e.g., I-1, I-2, I-3, I-4, II-1, II-2, II-3 or II-4, or any subformulae thereof) is an optionally substituted phenyl, 5- or 6-membered heterocyclic or heteroaryl ring selected from:

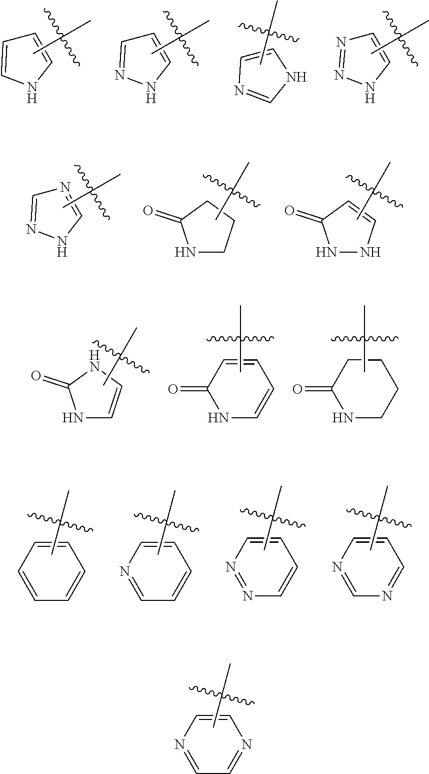

wherein each of which is optionally substituted with one or more (e.g., 1, 2 or 3) substituents each independently:
1) halogen (e.g., F, or Cl), cyano (CN), —C(O)(C₁₋₄ alkyl), —C(O)NH₂, —COOH, —C(O)—N(H)(C₁₋₄ alkyl), —C(O)—N(C₁₋₄ alkyl)(C₁₋₄ alkyl), —N(H)—C(O)—(C₁₋₄ alkyl), or —N(C₁₋₄ alkyl)-C(O)—(C₁₋₄ alkyl),
2) C₁₋₆ alkyl, —O—C₁₋₆ alkyl, —N(H)—C₁₋₆ alkyl, or —(C₁₋₄ heteroalkylene)-C₁₋₆ alkyl, wherein each of the C₁₋₆ alkyl is optionally substituted, e.g., with one or more (e.g., 1, 2 or 3) substituents independently selected from oxo, C₁₋₄ heteroalkyl, hydroxyl, N(C₁₋₄ alkyl)(C₁₋₄ alkyl), N(H)(C₁₋₄ alkyl), —C(O)—N(C₁₋₄ alkyl)(C₁₋₄ alkyl), —C(O)NH₂, —COOH, —C(O)—N(H)(C₁₋₄ alkyl), —N(H)—C(O)—(C₁₋₄ alkyl), —N(C₁₋₄ alkyl)-C(O)—(C₁₋₄ alkyl), —C(O)-G¹, F, C₁₋₄ alkoxy optionally substituted with 1-3 F, 5- or 6-membered heteroaryl having 1-3 ring heteroatoms independently selected from nitrogen, oxygen, and sulfur, and 4-8 membered saturated heterocyclyl having 1-3 (e.g., 1 or 2) ring heteroatoms independently selected from nitrogen, oxygen, and sulfur, wherein the 5- or 6-membered heteroaryl or 4-8 membered saturated heterocyclyl is optionally substituted, e.g., with 1-3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
3) C₃₋₁₀ cycloalkyl, —(C₁₋₄ alkylene)-C₃₋₁₀ cycloalkyl, —O—C₃₋₁₀ cycloalkyl, —N(H)—C₃₋₁₀ cycloalkyl, or —(C₁₋₄ heteroalkylene)-C₃₋₁₀ cycloalkyl, each of the C₃₋₁₀ cycloalkyl is monocyclic or polycyclic, preferably a monocyclic C₃₋₆ cycloalkyl, which is optionally substituted, e.g., with one or more (e.g., 1 or 2) substituents independently selected from C₁₋₄ alkyl optionally substituted with 1-3 F, C₁₋₄ heteroalkyl, hydroxyl, F, and C₁₋₄ alkoxy optionally substituted with 1-3 F, wherein two of the optional substituents of the C₃₋₁₀ cycloalkyl can be joined to form a ring structure, such as a 4-8 membered (e.g., 4-6 membered) saturated heterocyclic ring containing 1 or 2 ring heteroatoms independently selected from nitrogen, oxygen, and sulfur, wherein the 4-8 membered (e.g., 4-6 membered) saturated heterocyclic ring is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
4) 4-8 membered heterocyclyl, —(C₁₋₄ alkylene)-(4-8 membered heterocyclyl), —O-(4-8 membered heterocyclyl), —N(H)-(4-8 membered heterocyclyl), or —(C₁₋₄ heteroalkylene)-(4-8 membered heterocyclyl), wherein each of the 4-8 membered heterocyclyl is a heterocyclyl having 1-3 (e.g., 1 or 2) ring heteroatoms independently selected from nitrogen, oxygen, and sulfur, which is optionally substituted, e.g., with 1-3 substituents independently selected from oxo, F, C₁₋₄ alkyl optionally substituted with 1-3 F, C₁₋₄ heteroalkyl, hydroxyl, G¹, and C₁₋₄ alkoxy optionally substituted with 1-3 F, wherein two optional substituents of the 4-8 membered heterocyclyl can be joined together to form a ring structure, e.g., a spirocycloalkyl,
5) 5 or 6 membered heteroaryl, —(C₁₋₄ alkylene)-(5 or 6 membered heteroaryl), —O-(5 or 6 membered heteroaryl), —N(H)-(5 or 6 membered heteroaryl), or —(C₁₋₄ heteroalkylene)-(5 or 6 membered heteroaryl), wherein each of the 5 or 6 membered heteroaryl is a heteroaryl having 1-4 ring heteroatoms independently selected from nitrogen, oxygen, and sulfur, which is optionally substituted, e.g., with one or more (e.g., 1 or 2) substituents independently selected from C₁₋₄ alkyl optionally substituted with 1-3 F, C₁₋₄ heteroalkyl, hydroxyl, F, Cl, CN, and C₁₋₄ alkoxy optionally substituted with 1-3 F, wherein two optional substituents of the 5 or 6 membered heteroaryl can be joined together to form a ring structure, or
6) phenyl, —(C₁₋₄ alkylene)-phenyl, —O-phenyl, —N(H)-phenyl, or —(C₁₋₄ heteroalkylene)-phenyl, each of the phenyl is optionally substituted, e.g., with 1-3 substituents independently selected from F, Cl, CN, C₁₋₄ alkyl optionally substituted with 1-3 F, C₁₋₄ heteroalkyl, hydroxyl, and C₁₋₄ alkoxy optionally substituted with 1-3 F, wherein two optional substituents of the phenyl can be joined together to form a ring structure, and wherein G¹ is a 4-8 membered (e.g., 4-6 membered) saturated heterocyclyl containing 1 or 2 ring heteroatoms independently selected from nitrogen, oxygen, and sulfur, wherein the heterocyclyl is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F, and wherein two of the optional substituents of the phenyl, 5- or 6-membered heterocyclic or heteroaryl ring as drawn above can be joined together to form a ring structure (e.g., spiro, fused, or bridged ring structure).
It should be understood that the phenyl, 5- or 6-membered heterocyclic or 5 or 6 membered heteroaryl as drawn above can be attached to the remainder of the molecule through any available attaching points, see exemplified attachments herein. The substitution pattern of the phenyl, 5- or 6-membered heterocyclic or 5 or 6 membered heteroaryl as drawn above is also not particularly limited. Any of the hydrogen of an available N—H or C—H of the phenyl, 5- or 6-membered heterocyclic or 5 or 6 membered heteroaryl can be replaced with a permissible substituent. The term polycyclic should be understood as including bicyclic structures, such as a fused, bridged, or spiro bicyclic structure. It should be further understood that when two C₁₋₄ alkyl groups appear in the same structural moiety, such as in an expression “N(C₁₋₄ alkyl)(C₁₋₄ alkyl),” the two C₁₋₄ alkyl groups can be the same or different, for example, both NMe₂ and N(Me)(Et) are within the meaning of “N(C₁₋₄ alkyl)(C₁₋₄ alkyl)”.
In some embodiments, the phenyl, 5- or 6-membered heterocyclic or 5- or 6-membered heteroaryl ring as drawn above is substituted with one substituent. In some embodiments, the phenyl, 5- or 6-membered heterocyclic or 5- or 6-membered heteroaryl ring as drawn above is substituted with two substituents. In some embodiments, the phenyl, 5- or 6-membered heterocyclic or 5- or 6-membered heteroaryl ring as drawn above is substituted with three substituents.

In some embodiments, two substituents of the phenyl, 5- or 6-membered heterocyclic or 5- or 6-membered heteroaryl ring as drawn above can be joined together to form a ring structure, such as a spiro, fused, or bridged ring structure. For example, in some embodiments, R¹ is a phenyl or 5- or 6-membered heteroaryl ring selected from:

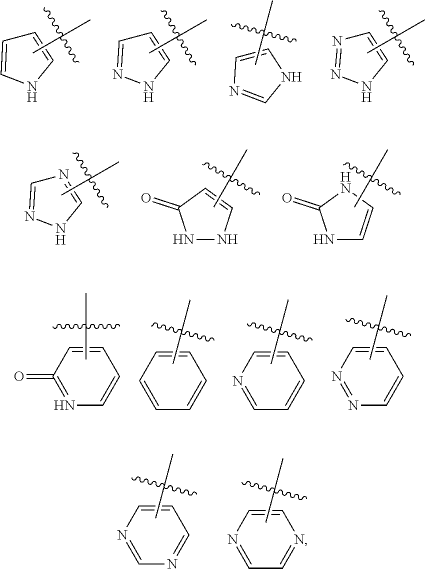

wherein two substituents on two adjacent ring atoms of the phenyl or 5- or 6-membered heteroaryl ring are joined together to form a ring structure, such as a 5 or 6-membered heterocyclic ring having 1-2 ring heteroatoms independently selected from nitrogen and oxygen. In some embodiments, R¹ is a 5- or 6-membered heterocyclic ring selected from:

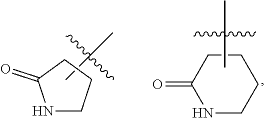

wherein two substituents on adjacent ring atoms or the same ring carbon can be joined together to form a ring structure, such as a C₃₋₆ cycloalkyl, 5 or 6-membered heterocyclic ring having 1-2 ring heteroatoms independently selected from nitrogen and oxygen, or 5, or 6 membered heteroaryl having 1-3 ring heteroatoms independently selected from nitrogen and oxygen. To be clear, in such embodiments, the phenyl, 5- or 6-membered heterocyclic or 5- or 6-membered heteroaryl ring can be further optionally substituted, and the spiro, fused, or bridged ring structure can also be optionally substituted.

In some embodiments, R¹ in Formula I or II (e.g., I-1, I-2, I-3, I-4, II-1, II-2, II-3 or II-4, or any subformulae thereof) can be selected from:

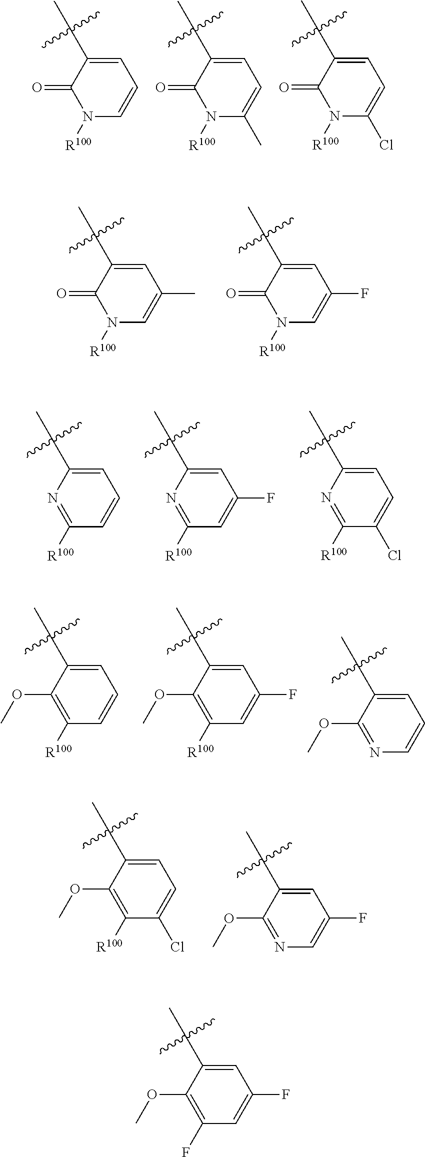

wherein each R¹⁰⁰ is independently selected from:
1) C₁₋₆ alkyl, which is optionally substituted, e.g., with one or more (e.g., 1, 2 or 3) substituents independently selected from oxo, C₁₋₄ heteroalkyl, hydroxyl, N(C₁₋₄ alkyl)(C₁₋₄ alkyl), N(H)(C₁₋₄ alkyl), —C(O)—N(C₁₋₄ alkyl)(C₁₋₄ alkyl), —C(O)—N(H)(C₁₋₄ alkyl), —C(O)NH₂, —COOH, —N(H)—C(O)—(C₁₋₄ alkyl), —N(C₁₋₄ alkyl)-C(O)—(C₁₋₄ alkyl), —C(O)-G¹, F, C₁₋₄ alkoxy optionally substituted with 1-3 F, 5- or 6-membered heteroaryl having 1-3 ring heteroatoms independently selected from nitrogen, oxygen, and sulfur, and 4-8 membered saturated heterocyclyl having 1-3 (e.g., 1 or 2) ring heteroatoms independently selected from nitrogen, oxygen, and sulfur, wherein the 5- or 6-membered heteroaryl or 4-8 membered saturated heterocyclyl is optionally substituted, e.g., with 1-3 substituents independently selected from F, C₁₋₄ alkyl optionally substituted with 1-3 F, C₁₋₄ heteroalkyl, hydroxyl, and C₁₋₄ alkoxy optionally substituted with 1-3 F,
2) C₃₋₆ cycloalkyl, which is optionally substituted, e.g., with one or more (e.g., 1 or 2) substituents independently selected from C₁₋₄ alkyl optionally substituted with 1-3 F, C₁₋₄ heteroalkyl, hydroxyl, F, 5- or 6-membered heteroaryl having 1-3 ring heteroatoms independently selected from nitrogen, oxygen, and sulfur, 4-8 membered saturated heterocyclyl having 1-3 (e.g., 1 or 2) ring heteroatoms independently selected from nitrogen, oxygen, and sulfur, and C₁₋₄ alkoxy optionally substituted with 1-3 F, wherein two of the optional substituents of the C₃₋₆ cycloalkyl can be joined to form a ring structure, such as a 4-8 membered (e.g., 4-6 membered) saturated heterocyclic ring containing 1 or 2 ring heteroatoms independently selected from nitrogen, oxygen, and sulfur, wherein the 5- or 6-membered heteroaryl or 4-8 membered (e.g., 4-6 membered) saturated heterocyclic ring is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F, 3) 4-8 membered heterocyclyl having 1-3 (e.g., 1 or 2) ring heteroatoms independently
selected from nitrogen, oxygen, and sulfur, which is optionally substituted, e.g., with 1-3 substituents independently selected from oxo, F, C₁₋₄ alkyl optionally substituted with 1-3 F, C₁₋₄ heteroalkyl, hydroxyl, G¹, and C₁₋₄ alkoxy optionally substituted with 1-3 F,
4) 5 or 6 membered heteroaryl having 1-4 ring heteroatoms independently selected from nitrogen, oxygen, and sulfur, which is optionally substituted, e.g., with one or more (e.g., 1 or 2) substituents independently selected from C₁₋₄ alkyl optionally substituted with 1-3 F, C₁₋₄ heteroalkyl, hydroxyl, F, Cl, CN, and C₁₋₄ alkoxy optionally substituted with 1-3 F, and
5) phenyl, which is optionally substituted, e.g., with 1-3 substituents independently selected from F, Cl, CN, C₁₋₄ alkyl optionally substituted with 1-3 F, C₁₋₄ heteroalkyl, hydroxyl, and C₁₋₄ alkoxy optionally substituted with 1-3 F,
wherein G¹ is a 4-8 membered (e.g., 4-6 membered) saturated heterocyclyl containing 1 or 2 ring heteroatoms independently selected from nitrogen, oxygen, and sulfur, wherein the heterocyclyl is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
or R¹⁰⁰ is a 7-10 membered spiro, bridged, or fused bicyclic ring structure containing 1 or 2 ring heteroatoms independently selected from nitrogen, oxygen, and sulfur, which is optionally substituted.

In some embodiments, each R¹⁰⁰ for the above formulae can be independently selected from:
1) C₁₋₄ alkyl optionally substituted with one or more (e.g., 1, 2 or 3) substituents independently selected from F, C₁₋₄ alkyl optionally substituted with 1-3 F, C₁₋₄ heteroalkyl, hydroxyl, —C(O)—N(C₁₋₄ alkyl)(C₁₋₄ alkyl), —C(O)—N(H)(C₁₋₄ alkyl), —C(O)NH₂, —COOH, and C₁₋₄ alkoxy optionally substituted with 1-3 F, for example, R¹⁰⁰ is methyl, ethyl, or isopropyl,
2) C₃₋₆ cycloalkyl, such as cyclopropyl, cyclobutyl, cyclopentyl, or cyclohexyl, optionally substituted with one or more, such as 1, 2, or 3 substituents independently selected from F, C₁₋₄ alkyl optionally substituted with 1-3 F, OH, and C₁₋₄ alkoxy optionally substituted with 1-3 F (e.g., OMe);
3) 4-8 membered (e.g., 4-6 membered) saturated monocyclic heterocyclyl containing 1 or 2 ring heteroatoms independently selected from N and O, such as oxetanyl, azetidinyl, tetrahydrofuranyl, tetrahydropyranyl, pyrrolidinyl, piperazinyl, morpholinyl, etc., wherein the heterocyclyl is optionally substituted with one or more, such as 1, 2, or 3 substituents independently selected from oxo, F, C₁₋₄ alkyl optionally substituted with 1-3 F, hydroxyl, and C₁₋₄ alkoxy optionally substituted with 1-3 F (e.g., methoxy); and
4) 5 or 6 membered heteroaryl having 1-4 ring heteroatoms independently selected from nitrogen, oxygen, and sulfur, such as pyridinyl (e.g., 2-, 3-, or 4-pyridinyl), pyrimidinyl, pyridazinyl, pyrazinyl, oxazolyl, isoxazolyl, etc., which is optionally substituted with one or more, such as 1, 2, or 3 substituents independently selected from F, C₁₋₄ alkyl optionally substituted with 1-3 F (e.g., methyl), and C₁₋₄ alkoxy optionally substituted with 1-3 F (e.g., methoxy).

In some embodiments, each R¹⁰⁰ for the above formulae can be a 7, 8, or 9 membered spiro, bridged, or fused bicyclic ring structure containing 1 or 2 ring heteroatoms independently selected from nitrogen, oxygen, and sulfur, such as a saturated 7, 8, or 9 membered bicyclic ring structure containing a ring oxygen. The bicyclic structure is optionally substituted with one or more, such as 1, 2, or 3 substituents independently selected from F, C₁₋₄ alkyl optionally substituted with 1-3 F (e.g., methyl), and C₁₋₄ alkoxy optionally substituted with 1-3 F (e.g., methoxy). For example, R¹⁰⁰ can be a 8 or 9-membered spiro bicyclic structure such as

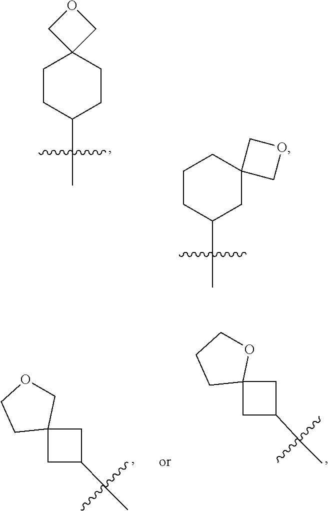 [SMILES: *C1CCC2(CC1)COC2 ; *C1CCCC2(COC2)C1 ; *C1CC2(CCOC2)C1 ; *C1CC2(CCCO2)C1]

a 7-membered fused bicyclic structure such as

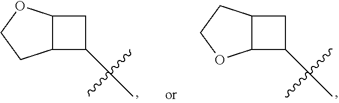 [SMILES: *C1CC2OCCC12 ; *C1CC2CCOC12]

or 8-membered bridged bicyclic structure such as

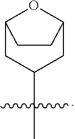 [SMILES: *C1CC2CCC(C1)O2]

In some embodiments, R¹ in Formula I or II (e.g., I-1, I-2, I-3, I-4, II-1, II-2, II-3 or II-4, or any subformulae thereof) can be selected from:

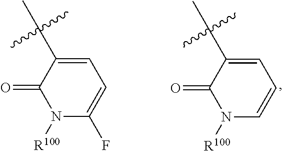

wherein R¹⁰⁰ can be any of those defined herein.

In any of the embodiments described herein, unless otherwise specified or contrary from context, R¹⁰⁰ can be selected from:

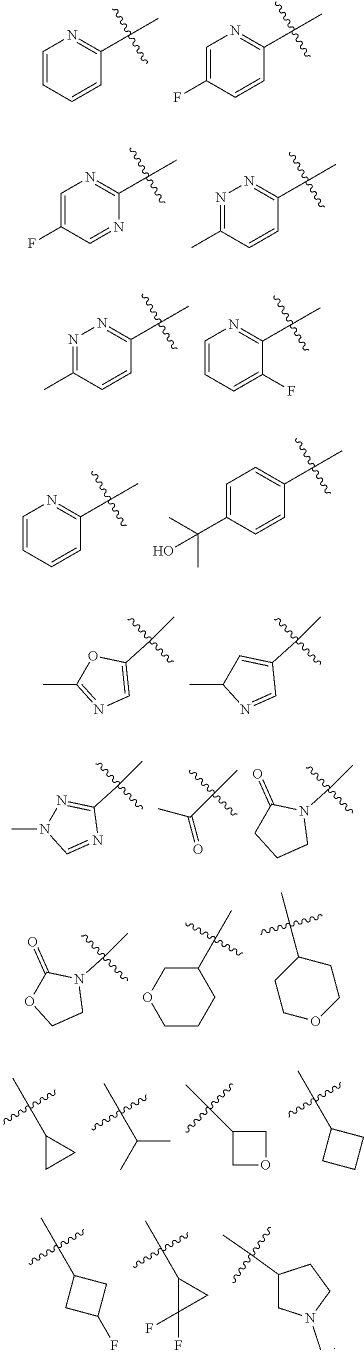

In any of the embodiments described herein, unless otherwise specified or contrary from context, R¹⁰⁰ can be

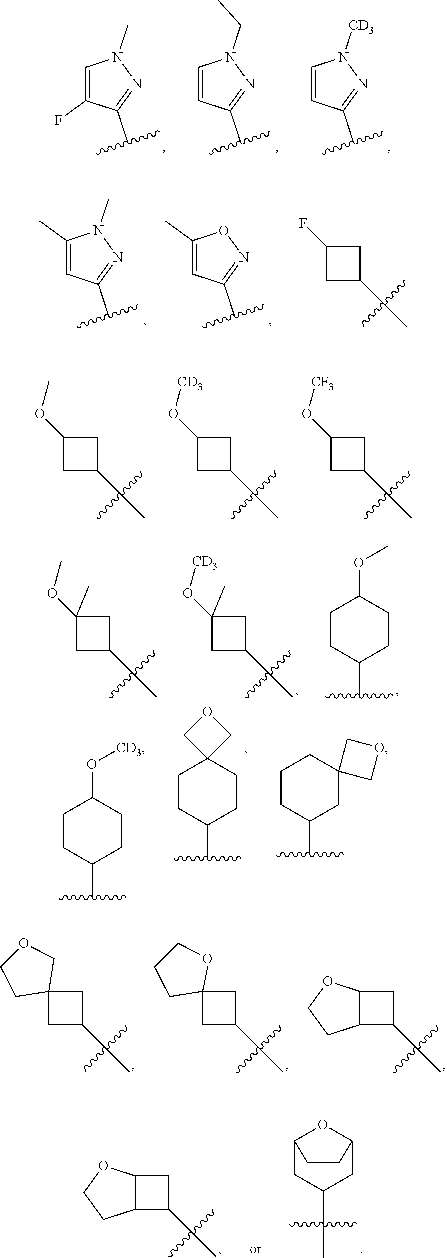

As would be apparent to those skilled in the art, compounds of Formula I or II having some of the R¹⁰⁰ group drawn above can have one or more asymmetric centers. As discussed herein, the present disclosure is not limited to any particular possible stereoisomer, but rather encompasses individual stereoisomers such as enantiomers and/or diastereomers and mixtures of stereoisomers in any ratio, such as racemic mixtures.

For example, in any of the embodiments described herein, unless otherwise specified or contrary from context, R¹⁰⁰ can also be selected from:

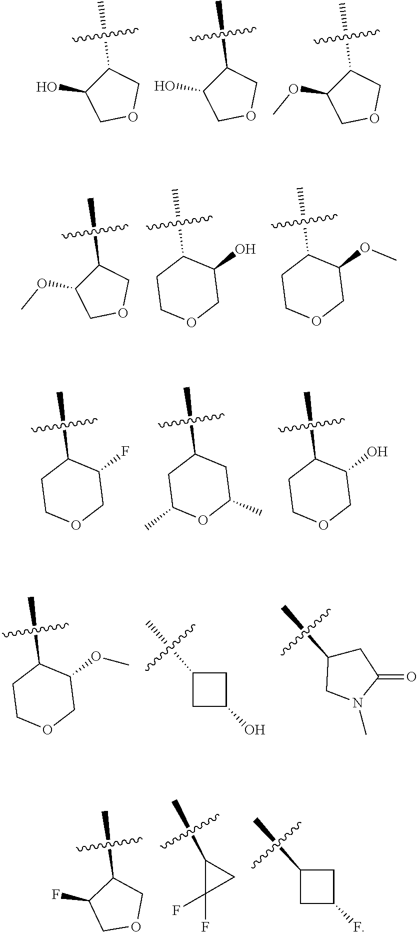

In any of the embodiments described herein, unless otherwise specified or contrary from context, R¹⁰⁰ can also be selected from:

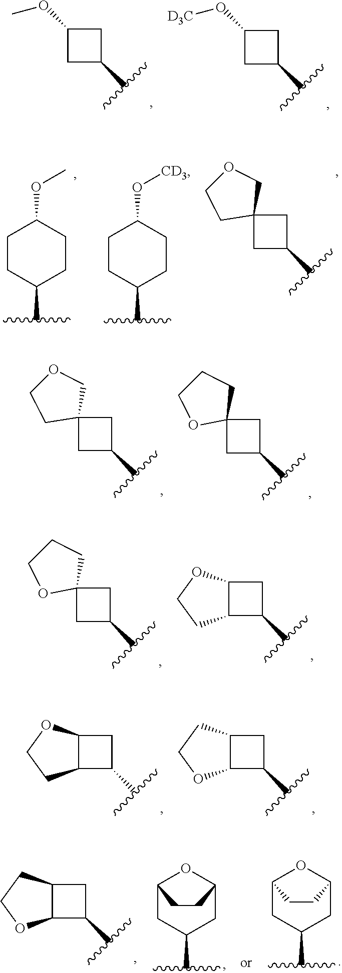

For the avoidance of doubt, when compounds of Formula I or II is said to have such R¹⁰⁰ with the stereochemistry drawn, unless otherwise specified or otherwise contrary from context, it should be understood that in some embodiments, with respect to the as-drawn stereochemical center(s), the compound of Formula I or II (e.g., any of its subformulae herein) can exist predominantly as the as-drawn stereoisomer, such as with less than 20%, less than 10%, less than 5%, less than 1%, by weight, by SFC or HPLC area, or both, or with a non-detectable amount of the other stereoisomer(s). Stereochemical features drawn for other R¹⁰⁰ definitions should be understood similarly.

In any of the embodiments described herein, unless otherwise specified or contrary from context, R¹⁰⁰ can also be selected from:

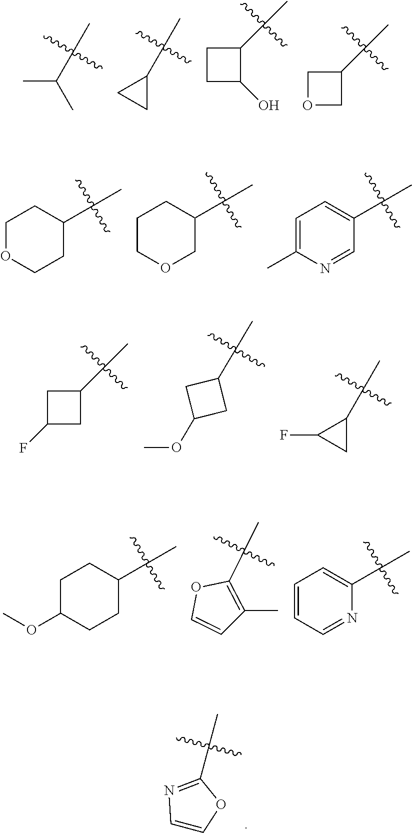

In any of the embodiments described herein, unless otherwise specified or contrary from context, R¹⁰⁰ can also be selected from:

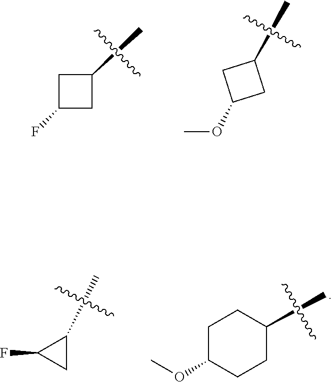

In some particular embodiments, R¹ in Formula I or II (e.g., I-1, I-2, I-3, I-4, II-1, II-2, II-3 or II-4, or any subformulae thereof) can have a structure of P-1:

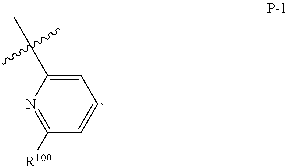

wherein R¹⁰⁰ can be any of those described herein. However, in some specific embodiments, R¹⁰⁰ in P-1 is C₁₋₄ alkyl optionally substituted with one or more (e.g., 1, 2 or 3) substituents independently selected from F, C₁₋₄ alkyl optionally substituted with 1-3 F, C₁₋₄ heteroalkyl, hydroxyl, and C₁₋₄ alkoxy optionally substituted with 1-3 F. For example, in some embodiments, R¹⁰⁰ in P-1 is methyl, ethyl, or isopropyl.

Suitable groups for R¹ in Formula I or II (e.g., I-1, I-2, I-3, I-4, II-1, II-2, II-3 or II-4, or any subformulae thereof) also include optionally substituted phenyl groups. For example, in some embodiments, R¹ can be a phenyl, which is optionally substituted, e.g., with one or more (e.g., 1-3) substituents each independently
1) halogen (e.g., F, or Cl), cyano (CN), —C(O)(C₁₋₄ alkyl), —C(O)NH₂, —COOH, —C(O)—N(H)(C₁₋₄ alkyl), —C(O)—N(C₁₋₄ alkyl)(C₁₋₄ alkyl), —N(H)—C(O)—(C₁₋₄ alkyl), or —N(C₁₋₄ alkyl)-C(O)—(C₁₋₄ alkyl),
2) C₁₋₆ alkyl, —O—C₁₋₆ alkyl, —N(H)—C₁₋₆ alkyl, or —(C₁₋₄ heteroalkylene)-C₁₋₆ alkyl, each of the C₁₋₆ alkyl is optionally substituted, e.g., with one or more (e.g., 1, 2 or 3) substituents independently selected from oxo, C₁₋₄ heteroalkyl, hydroxyl, N(C₁₋₄ alkyl)(C₁₋₄ alkyl), N(H)(C₁₋₄ alkyl), —C(O)—N(C₁₋₄ alkyl)(C₁₋₄ alkyl), —C(O)—N(H)(C₁₋₄ alkyl), —C(O)NH₂, —COOH, —N(H)—C(O)—(C₁₋₄ alkyl), —N(C₁₋₄ alkyl)-C(O)—(C₁₋₄ alkyl), —C(O)-G¹, F, C₁₋₄ alkoxy optionally substituted with 1-3 F, and 4-8 membered saturated heterocyclyl having 1-3 (e.g., 1 or 2) ring heteroatoms independently selected from nitrogen, oxygen, and sulfur, wherein the 4-8 membered saturated heterocyclyl is optionally substituted, e.g., with 1-3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
3) C₃₋₁₀ cycloalkyl, —(C₁₋₄ alkylene)-C₃₋₁₀ cycloalkyl, —O—C₃₋₁₀ cycloalkyl, —N(H)—C₃₋₁₀ cycloalkyl, or —(C₁₋₄ heteroalkylene)-C₃₋₁₀ cycloalkyl, each of the C₃₋₁₀ cycloalkyl is monocyclic or polycyclic, preferably a monocyclic C₃₋₆ cycloalkyl, which is optionally substituted, e.g., with one or more (e.g., 1 or 2) substituents independently selected from C₁₋₄ alkyl optionally substituted with 1-3 F, C₁₋₄ heteroalkyl, hydroxyl, F, and C₁₋₄ alkoxy optionally substituted with 1-3 F, wherein two of the optional substituents can be joined to form a ring structure, such as a 4-8 membered (e.g., 4-6 membered) saturated heterocyclic ring containing 1 or 2 ring heteroatoms independently selected from nitrogen, oxygen, and sulfur, wherein the 4-8 membered (e.g., 4-6 membered) saturated heterocyclic ring is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
4) 4-8 membered heterocyclyl, —(C₁₋₄ alkylene)-(4-8 membered heterocyclyl), —O-(4-8 membered heterocyclyl), —N(H)-(4-8 membered heterocyclyl), or —(C₁₋₄ heteroalkylene)-(4-8 membered heterocyclyl), wherein each of the 4-8 membered heterocyclyl is a heterocyclyl having 1-3 (e.g., 1 or 2) ring heteroatoms independently selected from nitrogen, oxygen, and sulfur, which is optionally substituted, e.g., with 1-3 substituents independently selected from oxo, F, C₁₋₄ alkyl optionally substituted with 1-3 F, C₁₋₄ heteroalkyl, hydroxyl, G¹, and C₁₋₄ alkoxy optionally substituted with 1-3 F, wherein two optional substituents can be joined together to form a ring structure,
5) 5 or 6 membered heteroaryl, —(C₁₋₄ alkylene)-(5 or 6 membered heteroaryl), —O-(5 or 6 membered heteroaryl), —N(H)-(5 or 6 membered heteroaryl), or —(C₁₋₄ heteroalkylene)-(5 or 6 membered heteroaryl), wherein each of the 5 or 6 membered heteroaryl is a heteroaryl having 1-4 ring heteroatoms independently selected from nitrogen, oxygen, and sulfur, which is optionally substituted, e.g., with one or more (e.g., 1 or 2) substituents independently selected from C₁₋₄ alkyl optionally substituted with 1-3 F, C₁₋₄ heteroalkyl, hydroxyl, F, Cl, CN, and C₁₋₄ alkoxy optionally substituted with 1-3 F, wherein two optional substituents can be joined together to form a ring structure, or
6) phenyl, —(C₁₋₄ alkylene)-phenyl, —O-phenyl, —N(H)-phenyl, or —(C₁₋₄ heteroalkylene)-phenyl, each of the phenyl is optionally substituted, e.g., with 1-3 substituents independently selected from F, Cl, CN, C₁₋₄ alkyl optionally substituted with 1-3 F, C₁₋₄ heteroalkyl, hydroxyl, and C₁₋₄ alkoxy optionally substituted with 1-3 F, wherein two optional substituents can be joined together to form a ring structure, and wherein G¹ is a 4-8 membered (e.g., 4-6 membered) saturated heterocyclyl containing 1 or 2 ring heteroatoms independently selected from nitrogen, oxygen, and sulfur, wherein the heterocyclyl is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F, and wherein two of the optional substituents of the phenyl together with the intervening atoms can be joined together to form a 5-8 membered ring structure.

In some embodiments, R¹ in Formula I or II (e.g., I-1, I-2, I-3, I-4, II-1, II-2, II-3 or II-4, or any subformulae thereof) can be an optionally substituted phenyl, wherein two optional substituents of the phenyl together with the intervening atoms are joined together to form a 5 or 6 membered monocyclic heterocyclic structure with 1 or 2 ring heteroatoms independently selected from O and N, wherein the monocyclic heterocyclic structure is optionally substituted with one or more, typically, 1, 2 or 3 substituents independently selected from oxo, F, C₁₋₄ alkyl optionally substituted with 1-3 F, C₁₋₄ heteroalkyl, hydroxyl, and C₁₋₄ alkoxy. In some embodiments, R¹ in Formula I or II (e.g., I-1, I-2, I-3, I-4, II-1, II-2, II-3 or II-4, or any subformulae thereof) can be an optionally substituted phenyl, wherein two optional substituents of the phenyl together with the intervening atoms are joined together to form a 5 or 6 membered heteroaryl structure with 1-3 ring heteroatoms independently selected from S, O and N, wherein the heteroaryl structure is optionally substituted with one or more, typically, 1, 2 or 3 substituents independently selected from oxo, F, C₁₋₄ alkyl optionally substituted with 1-3 F, C₁₋₄ heteroalkyl, hydroxyl, and C₁₋₄ alkoxy. As discussed herein, in such embodiments, both the phenyl and the 5 or 6 membered monocyclic heterocyclic structure or 5 or 6 membered heteroaryl structure can be further optionally substituted. In some embodiments, R¹ can be a chromanyl group, e.g., 8-chromanyl.

In some specific embodiments, R¹ in Formula I or II (e.g., I-1, I-2, I-3, I-4, II-1, II-2, II-3 or II-4, or any subformulae thereof) can be a substituted phenyl described herein, which can be selected from:

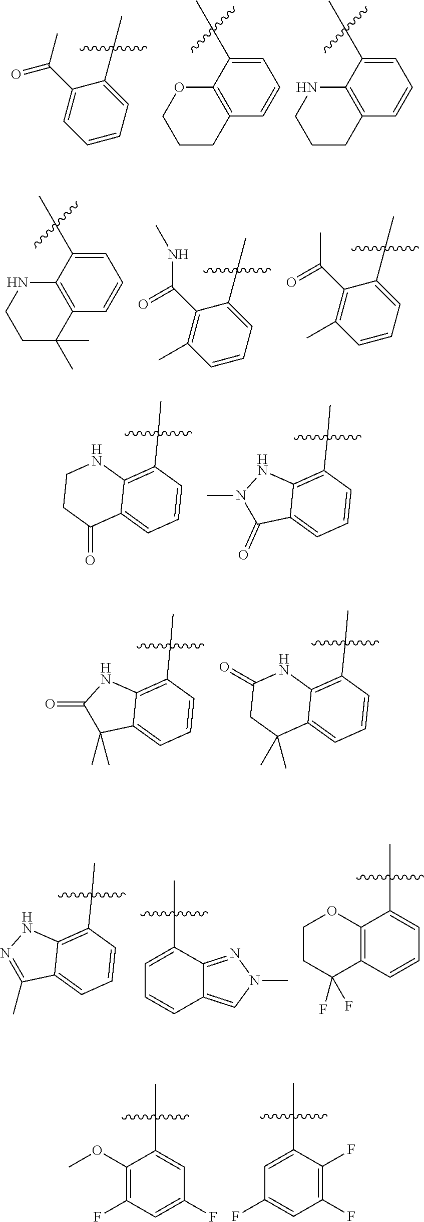

In some specific embodiments, R¹ in Formula I or II (e.g., I-1, I-2, I-3, I-4, II-1, II-2, II-3 or II-4, or any subformulae thereof) can be

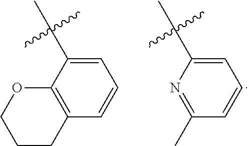

In some embodiments, R¹ in Formula I or II (e.g., I-1, I-2, I-3, I-4, II-1, II-2, II-3 or II-4, or any subformulae thereof) can also be an optionally substituted bicyclic heteroaryl (e.g., 5,5-bicyclic heteroaryl, 5,6-bicyclic heteroaryl, or 6,6-bicyclic heteroaryl) having 1-4 ring heteroatoms independently selected from nitrogen, oxygen, and sulfur. Suitable bicyclic heteroaryls include any of those described herein. Typically, the bicyclic heteroaryl is a 5,6-bicyclic heteroaryl having 1-3 ring heteroatoms independently selected from nitrogen, oxygen, and sulfur. Preferably, at most one of the ring heteroatoms of the bicyclic heteroaryl is oxygen or sulfur.

In some embodiments, R¹ in Formula I or II (e.g., I-1, I-2, I-3, I-4, II-1, II-2, II-3 or II-4, or any subformulae thereof) can be a 5,6-bicyclic heteroaryl ring selected from:

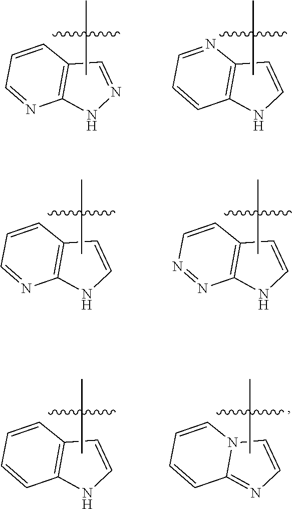

or a tautomeric form thereof, wherein each of which is optionally substituted. To be clear, the 5,6-bicyclic heteroaryl ring as drawn above or a tautomeric form thereof can link to the remainder of Formula I or II through any of the available attaching points in either of the two rings. In some embodiments, each of the 5,6-bicyclic heteroaryl ring as drawn above or a tautomeric form thereof can be substituted with one or more (e.g., 1 or 2) substituents each independently:
1) halogen (e.g., F, or Cl), CN, —C(O)(C₁₋₄ alkyl), —C(O)NH₂, —COOH, —C(O)—N(H)(C₁₋₄ alkyl), —C(O)—N(C₁₋₄ alkyl)(C₁₋₄ alkyl), —N(H)—C(O)—(C₁₋₄ alkyl), or —N(C₁₋₄ alkyl)-C(O)—(C₁₋₄ alkyl),
2) C₁₋₆ alkyl, —O—C₁₋₆ alkyl, —N(H)—C₁₋₆ alkyl, or —(C₁₋₄ heteroalkylene)-C₁₋₆ alkyl, each of the C₁₋₆ alkyl is optionally substituted, e.g., with one or more (e.g., 1, 2 or 3) substituents independently selected from oxo, C₁₋₄ heteroalkyl, hydroxyl, N(C₁₋₄ alkyl)(C₁₋₄ alkyl), N(H)(C₁₋₄ alkyl), —C(O)—N(C₁₋₄ alkyl)(C₁₋₄ alkyl), —C(O)—N(H)(C₁₋₄ alkyl), —C(O)NH₂, —COOH, —N(H)—C(O)—(C₁₋₄ alkyl), —N(C₁₋₄ alkyl)-C(O)—(C₁₋₄ alkyl), —C(O)-G¹, F, C₁₋₄ alkoxy optionally substituted with 1-3 F, and 4-8 membered saturated heterocyclyl having 1-3 (e.g., 1 or 2) ring heteroatoms independently selected from nitrogen, oxygen, and sulfur, wherein the 4-8 membered saturated heterocyclyl is optionally substituted, e.g., with 1-3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
3) C₃₋₁₀ cycloalkyl, —(C₁₋₄ alkylene)-C₃₋₁₀ cycloalkyl, —O—C₃₋₁₀ cycloalkyl, —N(H)—C₃₋₁₀ cycloalkyl, or —(C₁₋₄ heteroalkylene)-C₃₋₁₀ cycloalkyl, each of the C₃₋₁₀ cycloalkyl is monocyclic or polycyclic, preferably monocyclic C₃₋₆ cycloalkyl, which is optionally substituted, e.g., with one or more (e.g., 1 or 2) substituents independently selected from C₁₋₄ alkyl optionally substituted with 1-3 F, C₁₋₄ heteroalkyl, hydroxyl, F, and C₁₋₄ alkoxy optionally substituted with 1-3 F, wherein two of the optional substituents of the cycloalkyl can be joined to form a ring structure, such as a 4-8 membered (e.g., 4-6 membered) saturated heterocyclic ring containing 1 or 2 ring heteroatoms independently selected from nitrogen, oxygen, and sulfur, wherein the heterocyclic ring is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
4) 4-8 membered heterocyclyl, —(C₁₋₄ alkylene)-(4-8 membered heterocyclyl), —O-(4-8 membered heterocyclyl), —N(H)-(4-8 membered heterocyclyl), or —(C₁₋₄ heteroalkylene)-(4-8 membered heterocyclyl), wherein each of the 4-8 membered heterocyclyl is a heterocyclyl having 1-3 (e.g., 1 or 2) ring heteroatoms independently selected from nitrogen, oxygen, and sulfur, which is optionally substituted, e.g., with 1-3 substituents independently selected from oxo, F, C₁₋₄ alkyl optionally substituted with 1-3 F, C₁₋₄ heteroalkyl, hydroxyl, G¹, and C₁₋₄ alkoxy optionally substituted with 1-3 F, wherein two optional substituents of the heterocyclyl can be joined together to form a ring structure,
5) 5 or 6 membered heteroaryl, —(C₁₋₄ alkylene)-(5 or 6 membered heteroaryl), —O-(5 or 6 membered heteroaryl), —N(H)-(5 or 6 membered heteroaryl), or —(C₁₋₄ heteroalkylene)- (5 or 6 membered heteroaryl), wherein each of the 5 or 6 membered heteroaryl is a heteroaryl having 1-4 ring heteroatoms independently selected from nitrogen, oxygen, and sulfur, which is optionally substituted, e.g., with one or more (e.g., 1 or 2) substituents independently selected from C₁₋₄ alkyl optionally substituted with 1-3 F, C₁₋₄ heteroalkyl, hydroxyl, F, Cl, CN, and C₁₋₄ alkoxy optionally substituted with 1-3 F, wherein two optional substituents of the heteroaryl can be joined together to form a ring structure, or
6) phenyl, —(C₁₋₄ alkylene)-phenyl, —O-phenyl, —N(H)-phenyl, or —(C₁₋₄ heteroalkylene)-phenyl, each of the phenyl is optionally substituted, e.g., with 1-3 substituents independently selected from F, Cl, CN, C₁₋₄ alkyl optionally substituted with 1-3 F, C₁₋₄ heteroalkyl, hydroxyl, and C₁₋₄ alkoxy optionally substituted with 1-3 F, wherein two optional substituents of the phenyl can be joined together to form a ring structure, and wherein G¹ is a 4-8 membered (e.g., 4-6 membered) saturated heterocyclyl containing 1 or 2 ring heteroatoms independently selected from nitrogen, oxygen, and sulfur, wherein the heterocyclyl is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F.

In some embodiments, R¹ in Formula I or II (e.g., I-1, I-2, I-3, I-4, II-1, II-2, II-3 or II-4, or any subformulae thereof) can be selected from:

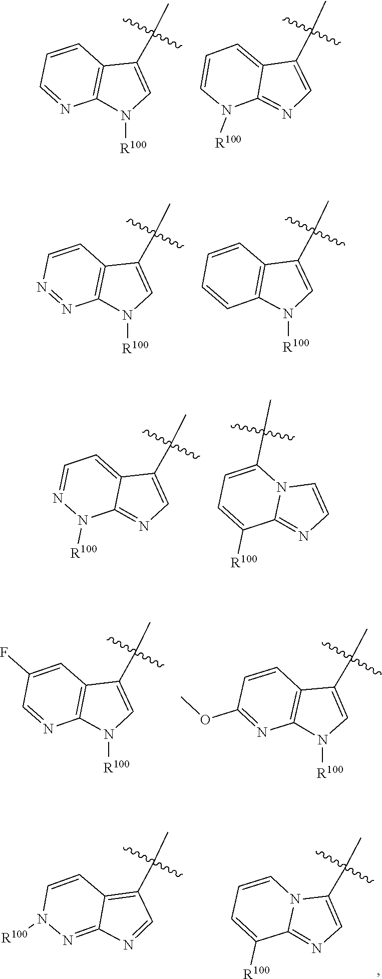

wherein each R¹⁰⁰ is independently selected from:
1) C₁₋₆ alkyl, which is optionally substituted, e.g., with one or more (e.g., 1, 2 or 3) substituents independently selected from oxo, C₁₋₄ heteroalkyl, hydroxyl, N(C₁₋₄ alkyl)(C₁₋₄ alkyl), N(H)(C₁₋₄ alkyl), —C(O)—N(C₁₋₄ alkyl)(C₁₋₄ alkyl), —C(O)—N(H)(C₁₋₄ alkyl), —C(O)NH₂, —COOH, —N(H)—C(O)—(C₁₋₄ alkyl), —N(C₁₋₄ alkyl)-C(O)—(C₁₋₄ alkyl), —C(O)-G¹, F, C₁₋₄ alkoxy optionally substituted with 1-3 F, and 4-8 membered saturated heterocyclyl having 1-3 (e.g., 1 or 2) ring heteroatoms independently selected from nitrogen, oxygen, and sulfur, wherein the 4-8 membered saturated heterocyclyl is optionally substituted, e.g., with 1-3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
2) C₃₋₆ cycloalkyl, which is optionally substituted, e.g., with one or more (e.g., 1 or 2) substituents independently selected from C₁₋₄ alkyl optionally substituted with 1-3 F, C₁₋₄ heteroalkyl, hydroxyl, F, and C₁₋₄ alkoxy optionally substituted with 1-3 F, wherein two of the optional substituents of the cycloalkyl can be joined to form a ring structure, such as a 4-8 membered (e.g., 4-6 membered) saturated heterocyclic ring containing 1 or 2 ring heteroatoms independently selected from nitrogen, oxygen, and sulfur, wherein the heterocyclic ring is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
3) 4-8 membered heterocyclyl having 1-3 (e.g., 1 or 2) ring heteroatoms independently selected from nitrogen, oxygen, and sulfur, which is optionally substituted, e.g., with 1-3 substituents independently selected from oxo, F, C₁₋₄ alkyl optionally substituted with 1-3 F, C₁₋₄ heteroalkyl, hydroxyl, G¹, and C₁₋₄ alkoxy optionally substituted with 1-3 F,
4) 5 or 6 membered heteroaryl having 1-4 ring heteroatoms independently selected from nitrogen, oxygen, and sulfur, which is optionally substituted, e.g., with one or more (e.g., 1 or 2) substituents independently selected from C₁₋₄ alkyl optionally substituted with 1-3 F, Cl, CN, C₁₋₄ heteroalkyl, hydroxyl, F, and C₁₋₄ alkoxy optionally substituted with 1-3 F, and
5) phenyl, which is optionally substituted, e.g., with 1-3 substituents independently selected from F, Cl, CN, C₁₋₄ alkyl optionally substituted with 1-3 F, C₁₋₄ heteroalkyl, hydroxyl, and C₁₋₄ alkoxy optionally substituted with 1-3 F, and
wherein G¹ is a 4-8 membered (e.g., 4-6 membered) saturated heterocyclyl containing 1 or 2 ring heteroatoms independently selected from nitrogen, oxygen, and sulfur, wherein the heterocyclyl is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy.

In some embodiments, R¹ in Formula I or II (e.g., I-1, I-2, I-3, I-4, II-1, II-2, II-3 or II-4, or any subformulae thereof) can be selected from:

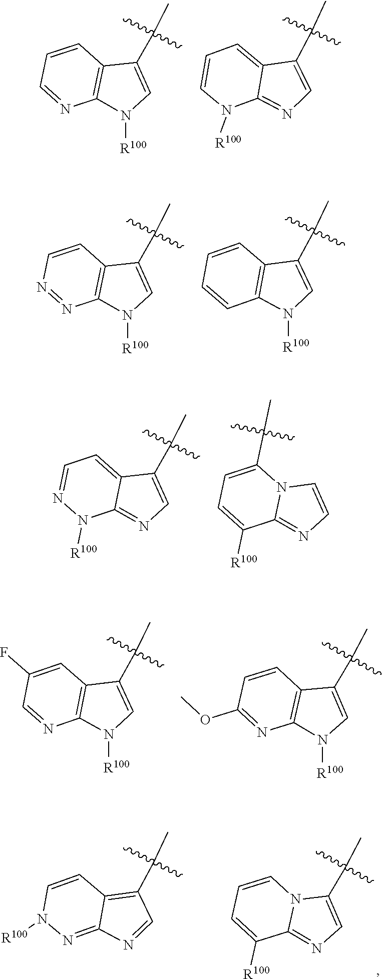

wherein each R¹⁰⁰ is independently selected from:
1) C₁₋₄ alkyl optionally substituted with one or more (e.g., 1, 2 or 3) substituents independently selected from F, C₁₋₄ alkyl optionally substituted with 1-3 F, C₁₋₄ heteroalkyl, hydroxyl, —C(O)—N(C₁₋₄ alkyl)(C₁₋₄ alkyl), —C(O)—N(H)(C₁₋₄ alkyl), —C(O)NH₂, —COOH, —N(H)—C(O)—(C₁₋₄ alkyl), —N(C₁₋₄ alkyl)-C(O)—(C₁₋₄ alkyl), and C₁₋₄ alkoxy optionally substituted with 1-3 F, for example, R¹⁰⁰ is methyl, ethyl, or isopropyl,
2) C₃₋₆ cycloalkyl, such as cyclopropyl, cyclobutyl, cyclopentyl, or cyclohexyl, optionally substituted with one or more, such as 1, 2, or 3 substituents independently selected from F, C₁₋₄ alkyl optionally substituted with 1-3 F, OH, and C₁₋₄ alkoxy optionally substituted with 1-3 F (e.g., OMe);
3) 4-8 membered (e.g., 4-6 membered) saturated monocyclic heterocyclyl containing 1 or 2 ring heteroatoms independently selected from N and O, such as oxetanyl, azetidinyl, tetrahydrofuranyl, tetrahydropyranyl, pyrrolidinyl, piperazinyl, morpholinyl, etc., wherein the heterocyclyl is optionally substituted with one or more, such as 1, 2, or 3 substituents independently selected from oxo, F, C₁₋₄ alkyl optionally substituted with 1-3 F, hydroxyl, and C₁₋₄ alkoxy optionally substituted with 1-3 F (e.g., methoxy); and
4) 5 or 6 membered heteroaryl having 1-4 ring heteroatoms independently selected from nitrogen, oxygen, and sulfur, such as pyridinyl (e.g., 2-, 3-, or 4-pyridinyl), pyrymidinyl, pyridazinyl, pyrazinyl, oxazolyl, isoxazolyl, etc., which is optionally substituted with one or more, such as 1, 2, or 3 substituents independently selected from F, Cl, CN, C₁₋₄ alkyl optionally substituted with 1-3 F (e.g., methyl), and C₁₋₄ alkoxy optionally substituted with 1-3 F (e.g., methoxy).

In some embodiments, R¹ in Formula I or II (e.g., I-1, I-2, I-3, I-4, II-1, II-2, II-3 or II-4, or any subformulae thereof) can be selected from:

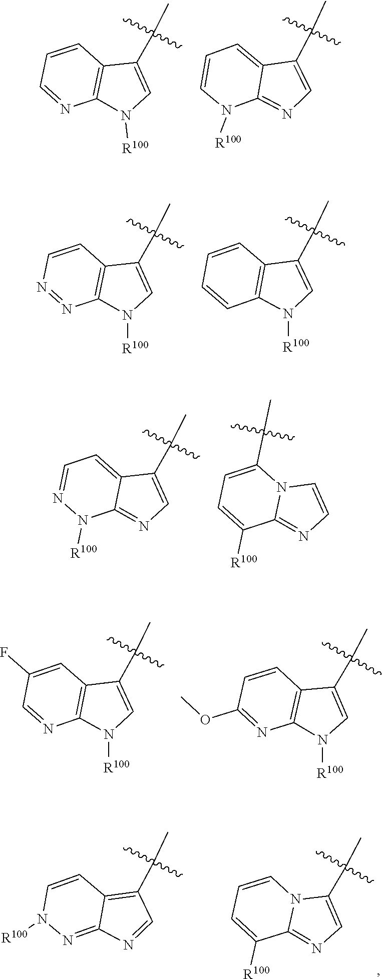

wherein R¹⁰⁰ can be any of those described herein. For example, in some embodiments, R¹⁰⁰ can be selected from:

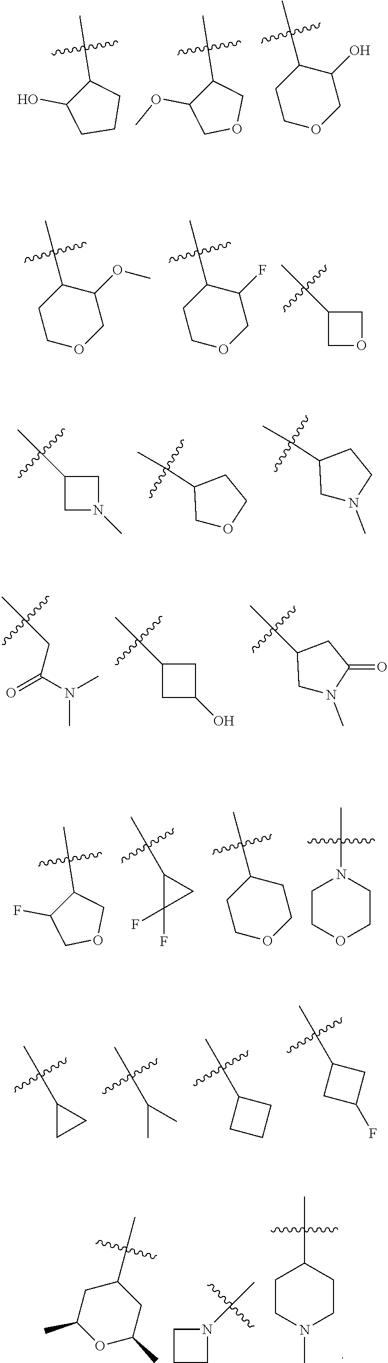

In some embodiments, R¹⁰⁰ can be selected from:

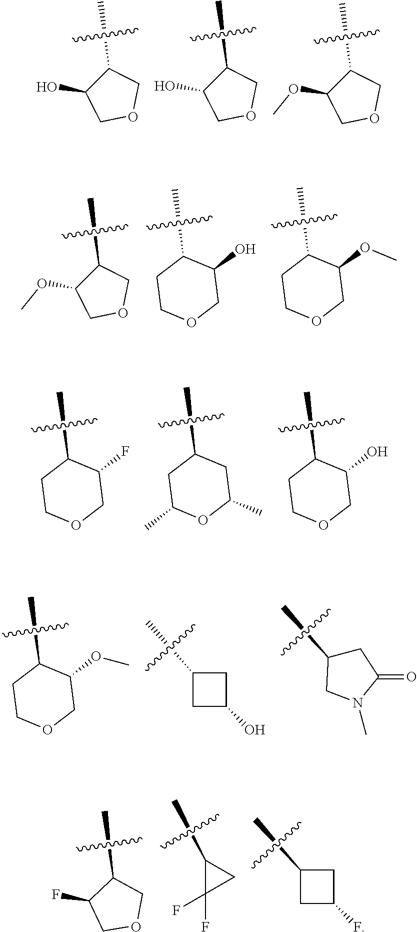

In some embodiments, R¹⁰⁰ can be selected from:

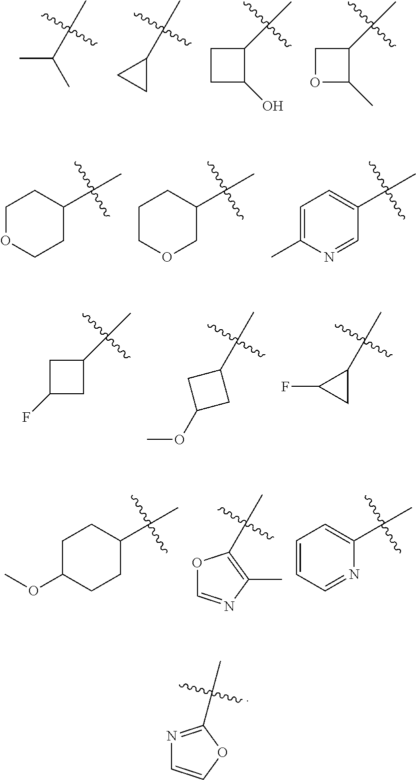

In some embodiments, R¹⁰⁰ can be selected from:

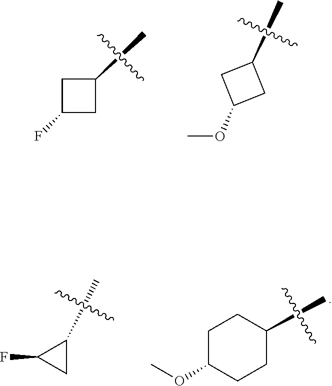

In some embodiments, R¹ in Formula I or II (e.g., I-1, I-2, I-3, I-4, II-1, II-2, II-3 or II-4, or any subformulae thereof) can be an optionally substituted C₃₋₈ carbocyclic ring, such as a C₃₋₆ cycloalkyl. In some embodiments, two optional substituents of the C₃₋₈ carbocyclic ring together with the intervening atoms are joined together to form a ring structure, which can be a spiro, bridged, or fused ring. In some embodiments, two optional substituents, e.g., two substituents on adjacent ring atoms or the same ring carbon, of the C₃₋₈ carbocyclic ring together with the intervening atoms are joined together to form a 5 or 6 membered monocyclic heterocyclic structure with 1 or 2 ring heteroatoms independently selected from O and N, wherein the monocyclic heterocyclic structure is optionally substituted with one or more, typically, 1, 2 or 3 substituents independently selected from oxo, F, C₁₋₄ alkyl optionally substituted with 1-3 F, C₁₋₄ heteroalkyl, hydroxyl, and C₁₋₄ alkoxy. In some embodiments, two optional substituents of the C₃₋₈ carbocyclic ring together with the intervening atoms are joined together to form a 5 or 6 membered heteroaryl structure with 1-3 ring heteroatoms independently selected from S, O and N, wherein the heteroaryl structure is optionally substituted with one or more, typically, 1, 2 or 3 substituents independently selected from oxo, F, C₁₋₄ alkyl optionally substituted with 1-3 F, C₁₋₄ heteroalkyl, hydroxyl, and C₁₋₄ alkoxy. As discussed herein, in such embodiments, both the C₃₋₈ carbocyclic ring and the 5 or 6 membered monocyclic heterocyclic structure or 5 or 6 membered heteroaryl structure can be further optionally substituted.

For example, in some embodiments, R¹ in Formula I or II (e.g., I-1, I-2, I-3, I-4, II-1, II-2, II-3 or II-4, or any subformulae thereof) can be selected from:

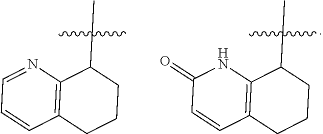 [SMILES: *C1CCCc2cccnc21 ; *C1CCCc2ccc(=O)[nH]c21]

The combination of L¹ and R¹ for the compounds of Formula I or II (e.g., I-1, I-2, I-3, I-4, II-1, II-2, II-3 or II-4, or any subformulae thereof) is not particularly limited, which includes any of those described herein, such as those exemplified, claimed and/or shown in the compounds of Table 1 herein. For example, in some embodiments, when R¹ is an optionally substituted phenyl or optionally substituted 5- or 6-membered heteroaryl or heterocyclyl, L¹ can be NH. In some embodiments, L¹ can be a bond, when R¹ is an optionally substituted heteroaryl, wherein the attaching point of R¹ to the bicyclic heteroaryl core structure in Formula I or II (e.g., I-1, I-2, I-3, I-4, II-1, II-2, II-3 or II-4, or any subformulae thereof) is on a ring having one or more ring heteroatoms.

In some specific embodiments, the compound of Formula I or II (e.g., I-1, I-2, I-3, I-4, II-1, II-2, II-3 or II-4, or any subformulae thereof) can be characterized as having a structure according to Formula I-A or II-A as applicable:

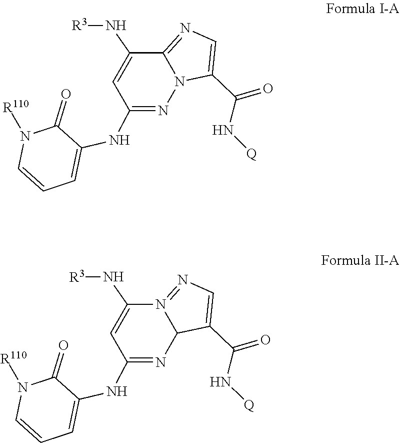

wherein R¹¹⁰ is an optionally substituted C₁₋₆ alkyl or an optionally substituted 3-14 membered ring structure such as an optionally substituted C₃₋₁₀ cycloalkyl, optionally substituted 4-10 membered heterocyclyl, optionally substituted phenyl, or optionally substituted 5-10 membered heteroaryl, wherein R³ and Q include any of those described herein in any combinations. In some embodiments, R¹¹⁰ in Formula I-A or II-A can be any of those defined herein for R¹⁰⁰.

In some embodiments, R¹¹⁰ in Formula I-A or II-A can be an optionally substituted 5 or 6-membered heteroaryl, such as a 5 or 6 membered heteroaryl having 1-4 ring heteroatoms independently selected from nitrogen, oxygen, and sulfur, such as pyridinyl (e.g., 2-, 3-, or 4-pyridinyl), pyrymidinyl, pyridazinyl, pyrazinyl, oxazolyl, isoxazolyl, etc., which is optionally substituted with 1, 2, or 3 substituents independently selected from F, C₁₋₄ alkyl optionally substituted with 1-3 F (e.g., methyl), and C₁₋₄ alkoxy optionally substituted with 1-3 F (e.g., methoxy). For example, in some embodiments, R¹¹⁰ in Formula I-A or II-A can be

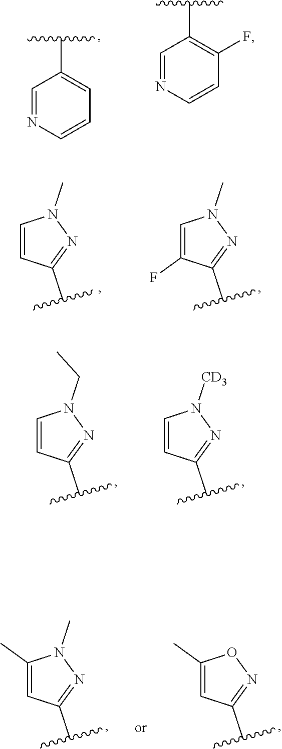

In some embodiments, R¹¹⁰ in Formula I-A or II-A can be an optionally substituted C₃₋₁₀ cycloalkyl, such as optionally substituted cyclopropyl, cyclobutyl, or cyclohexyl, etc. For example, in some embodiments, R¹¹⁰ in Formula I-A or II-A can be a cyclobutyl or cyclohexyl, which is optionally substituted with 1-3 substituents independently selected from F, C₁₋₄ alkyl optionally substituted with 1-3 F (e.g., methyl), and C₁₋₄ alkoxy optionally substituted with 1-3 F (e.g., methoxy). In some embodiments, R¹¹⁰ in Formula I-A or II-A can be selected from

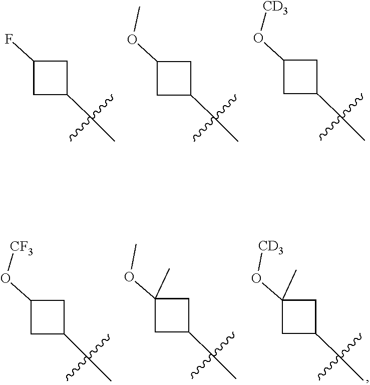

wherein the F, methoxy, CF₃O, or CD₃O can be cis or trans to the pyridone, for example, R¹¹⁰ can be

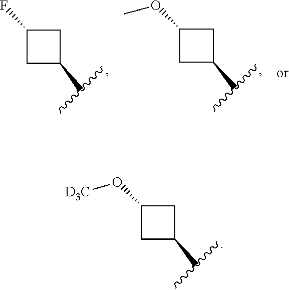

In some embodiments, R¹¹⁰ in Formula I-A or II-A can

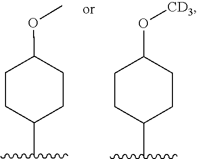

wherein the methoxy or CD₃O can be cis or trans to the pyridone, for example, R¹¹⁰ can be

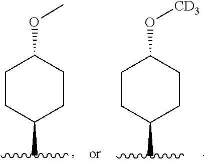

In some embodiments, R¹¹⁰ in Formula I-A or II-A can be an optionally substituted fused, spiro, or bridged bicyclic ring having 6-12 ring members optionally with one or two ring heteroatoms independently selected from O, S, and N. For example, in some embodiments, R¹¹⁰ is a spiro bicyclic structure having one ring oxygen atom, such as

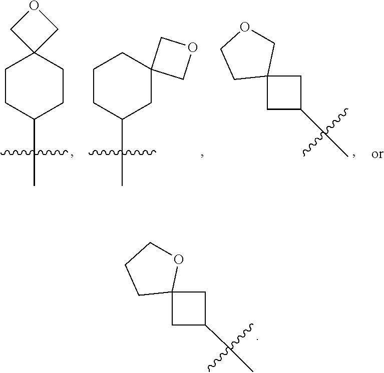

In some embodiments, R¹¹⁰ is a fused bicyclic structure having one ring oxygen atom, such as

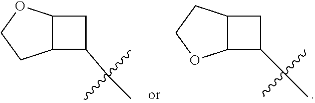 [SMILES: *C1CC2OCCC12 ; *C1CC2CCOC12]

In some embodiments, R¹¹⁰ is a bridged bicyclic structure having one ring oxygen atom, such as

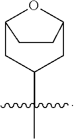 [SMILES: *C1CC2CCC(C1)O2]

The stereochemistry (including relative stereochemistry) of R¹¹⁰ is not particularly limited, for example, in some embodiments, R¹¹⁰ can be

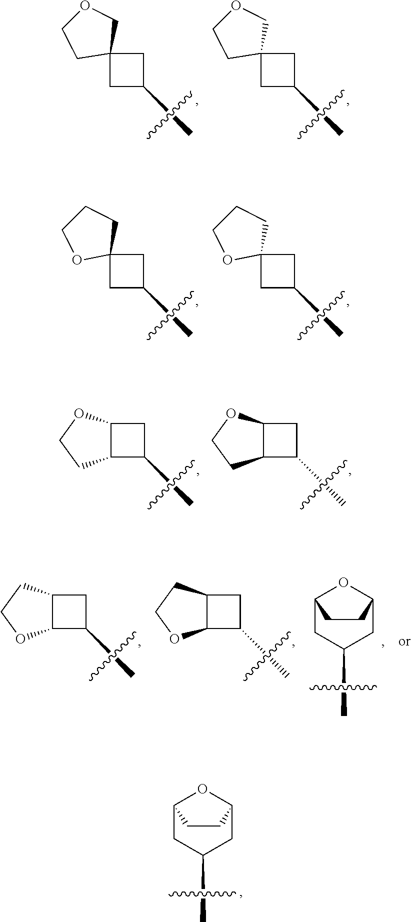

In some embodiments, R¹¹⁰ in Formula I-A or II-A can be an optionally substituted 4-8 membered (e.g., 4-6 membered) saturated monocyclic heterocyclyl containing 1 or 2 ring heteroatoms independently selected from N and O, such as oxetanyl, azetidinyl, tetrahydrofuranyl, tetrahydropyranyl, pyrrolidinyl, piperazinyl, morpholinyl, etc., wherein the heterocyclyl is optionally substituted with one or more, such as 1, 2, or 3 substituents independently selected from oxo, F, C₁₋₄ alkyl optionally substituted with 1-3 F, hydroxyl, and C₁₋₄ alkoxy optionally substituted with 1-3 F (e.g., methoxy). For example, in some embodiments, R¹¹⁰ in Formula I-A or II-A can be

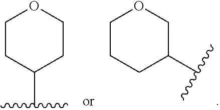 [SMILES: *C1CCOCC1 ; *C1CCCOC1]

In some embodiments, R¹¹⁰ in Formula I-A or II-A can be an optionally substituted phenyl, such as a phenyl which is optionally substituted with 1, 2, or 3 substituents independently selected from F, C₁₋₄ alkyl optionally substituted with 1-3 F (e.g., methyl), and C₁₋₄ alkoxy optionally substituted with 1-3 F (e.g., methoxy).

In some embodiments, R¹¹⁰ in Formula I-A or II-A can be an optionally substituted C₁₋₄ alkyl, such as having a structure of —C₁₋₄ alkylene-(3-8 membered ring), wherein the alkylene and the 3-8 membered ring is optionally substituted. The 3-8 membered ring is not particularly limited and can include for example, a 5 or 6 membered heteroaryl, C₃-6 cycloalkyl, or 4-8 membered heterocyclyl. The C₁₋₄ alkylene can be linear or branched. In some embodiments, the C₁₋₄ alkylene is CH₂. In some embodiments, the 3-8 membered ring is a 4-8 membered saturated heterocyclyl having 1-3 (e.g., 1 or 2) ring heteroatoms independently selected from nitrogen, oxygen, and sulfur, such as oxetanyl, azetidinyl, tetrahydrofuranyl, tetrahydropyranyl, pyrrolidinyl, piperazinyl, morpholinyl, etc., wherein the heterocyclyl is optionally substituted with one or more, such as 1, 2, or 3 substituents independently selected from oxo, F, C₁₋₄ alkyl optionally substituted with 1-3 F, hydroxyl, and C₁₋₄ alkoxy optionally substituted with 1-3 F (e.g., methoxy). In some embodiments, the 3-8 membered ring is a 5 or 6 membered heteroaryl having 1-4 ring heteroatoms independently selected from nitrogen, oxygen, and sulfur, such as pyridinyl (e.g., 2-, 3-, or 4-pyridinyl), pyrimidinyl, pyridazinyl, pyrazinyl, oxazolyl, isoxazolyl, oxadiazolyl, triazolyl, etc., which is optionally substituted with one or more, such as 1, 2, or 3 substituents independently selected from F, C₁₋₄ alkyl optionally substituted with 1-3 F (e.g., methyl), and C₁₋₄ alkoxy optionally substituted with 1-3 F (e.g., methoxy).

In any of the embodiments described herein, unless otherwise specified or contrary from context, in Formula I or II (e.g., I-1, I-2, I-3, I-4, II-1, II-2, II-3 or II-4, or any subformulae thereof), L¹-R¹ can be selected from:

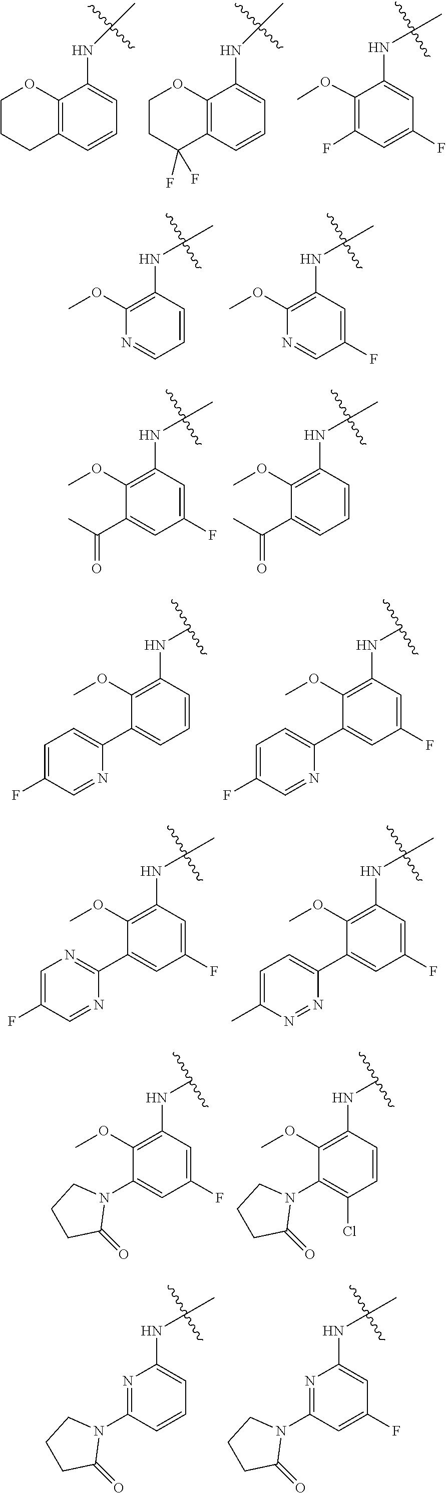

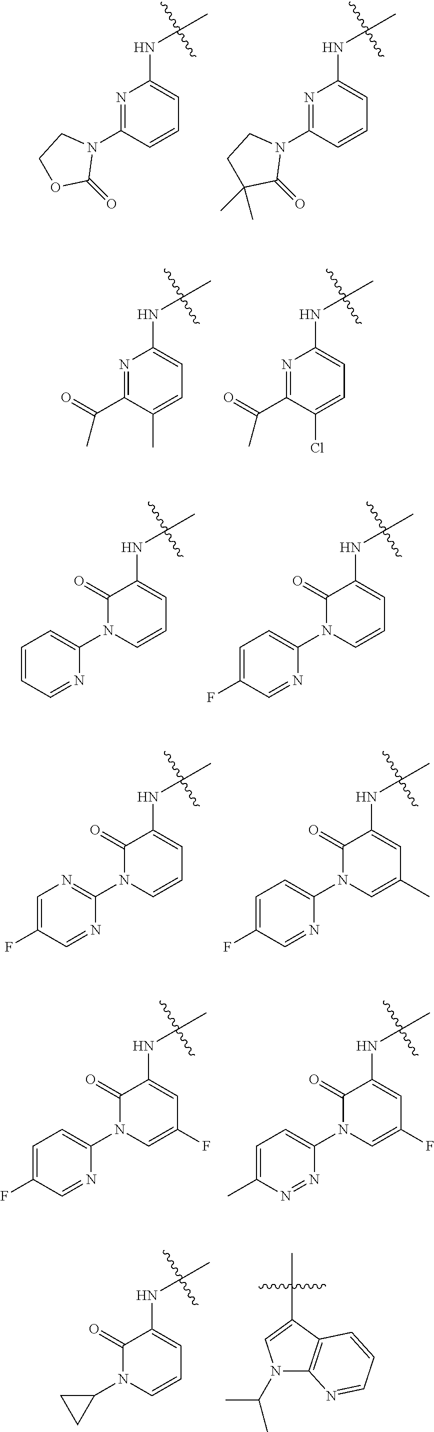

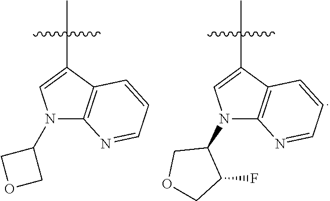

In some embodiments, in Formula I or II (e.g., I-1, I-2, I-3, I-4, II-1, II-2, II-3 or II-4, or any subformulae thereof), L¹-R¹ can be selected from:

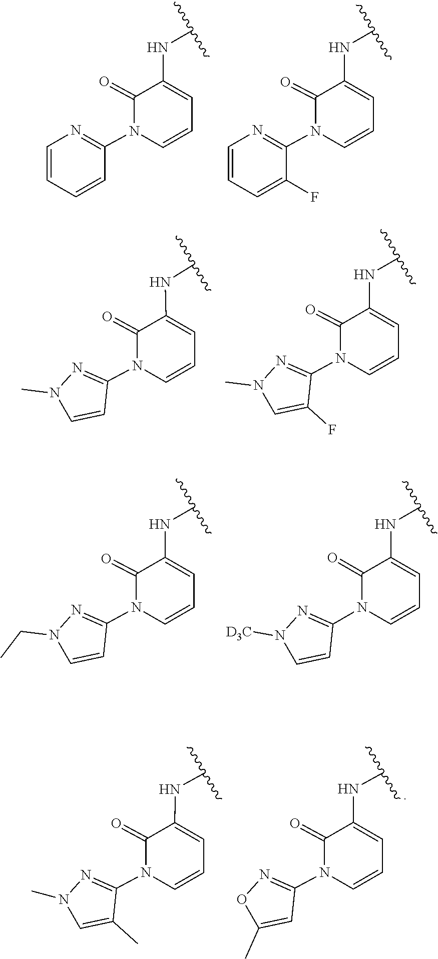

In some embodiments, in Formula I or II (e.g., I-1, I-2, I-3, I-4, II-1, II-2, II-3 or II-4, or any subformulae thereof), L¹-R¹ can be selected from:

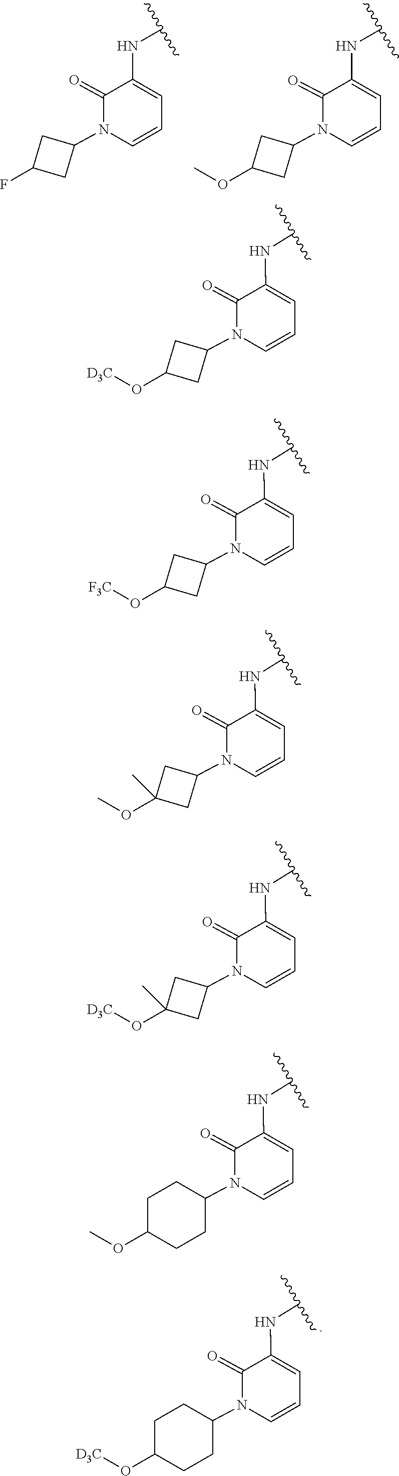

In some embodiments, unless otherwise specified or contrary from context, in Formula I or II (e.g., I-1, I-2, I-3, I-4, II-1, II-2, II-3 or II-4, or any subformulae thereof), L¹-R¹ can be selected from:

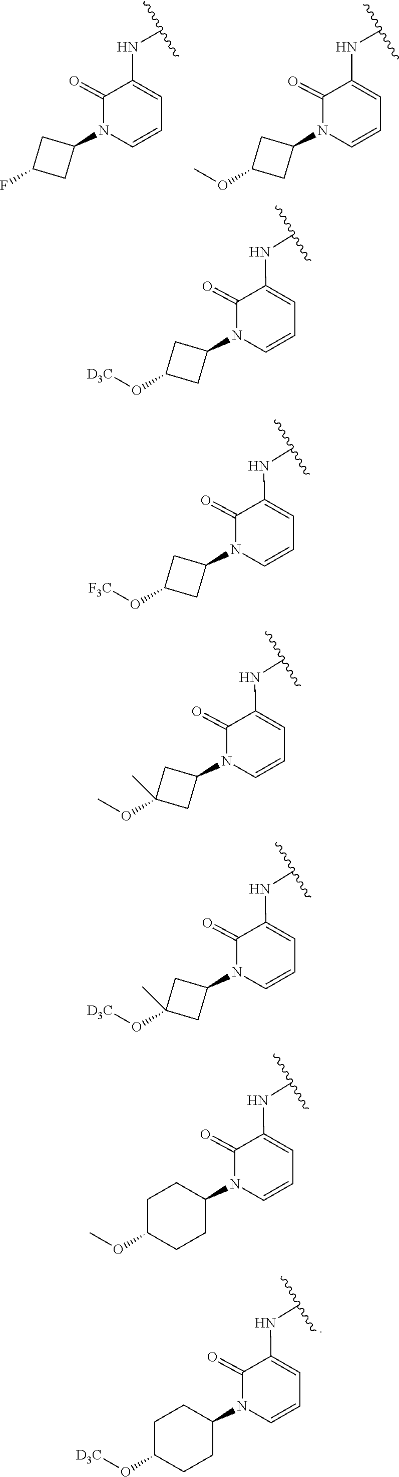

In some embodiments, in Formula I or II (e.g., I-1, I-2, I-3, I-4, II-1, II-2, II-3 or II-4, or any subformulae thereof), L¹-R¹ can be selected from:

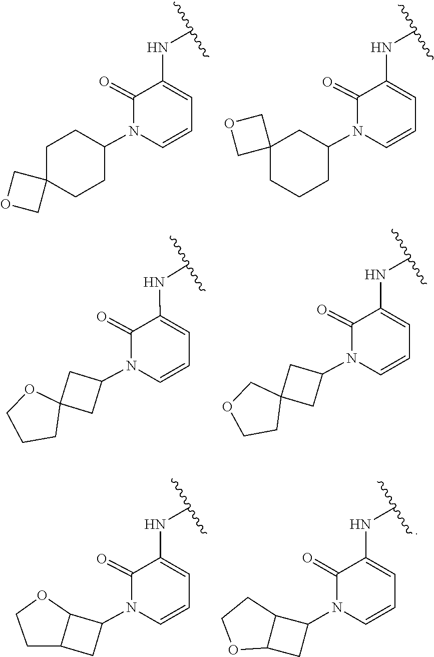

In some embodiments, in Formula I or II (e.g., I-1, I-2, I-3, I-4, II-1, II-2, II-3 or II-4, or any subformulae thereof), L¹-R¹ can be selected from:

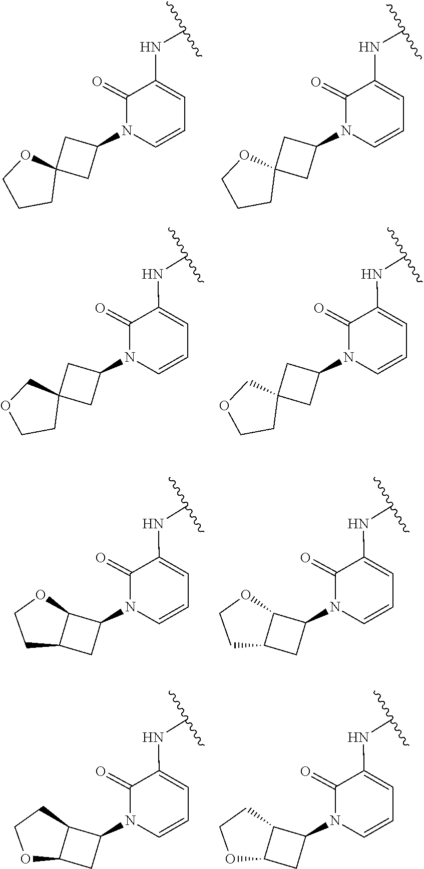

Various groups are suitable for R² in Formula I-1 or II-1. However, in typical embodiments, R² in Formula I-1 or II-1 is hydrogen.

Various groups are suitable for R³ in Formula I or II. However, in typical embodiments, R³ in Formula I or II (e.g., I-1, I-2, I-3, I-4, II-1, II-2, II-3 or II-4, or any subformulae thereof) is hydrogen or a C₁₋₄ alkyl, such as methyl. In some embodiments, R³ in Formula I or II (e.g., I-1, I-2, I-3, I-4, II-1, II-2, II-3 or II-4, or any subformulae thereof) is CD₃.

In some embodiments, as applicable, R² and R³ in Formula I or II (e.g., I-1 or II-1) can also be joined to form a 5-7 membered heterocyclic ring having 1 or 2 ring heteroatoms, or a 5-membered or 6-membered heteroaryl ring having 1-3 ring heteroatoms, wherein at least one ring heteroatom is nitrogen as required by Formula I or II, and any additional ring heteroatom(s) is nitrogen, oxygen, or sulfur, wherein the 5-7 membered heterocyclic ring is optionally substituted with one or two substituents independently selected from oxo, F, and methyl, wherein the 5-membered or 6-membered heteroaryl ring is optionally substituted with one or two substituents independently selected from F, Cl, CN, and methyl.

In some embodiments, the present disclosure also provide a compound selected from the compounds disclosed in Table 1A below, or a pharmaceutically acceptable salt thereof:

TABLE 1A
List of Compounds

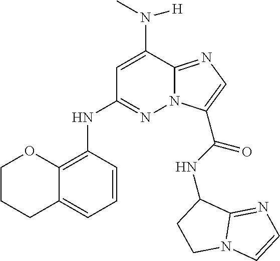 [SMILES: CNc1cc(Nc2cccc3c2OCCC3)nn2c(C(=O)NC3CCn4ccnc43)cnc12]

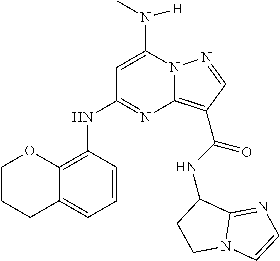 [SMILES: CNc1cc(Nc2cccc3c2OCCC3)nc2c(C(=O)NC3CCn4ccnc43)cnn12]

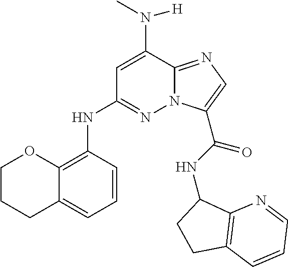 [SMILES: CNc1cc(Nc2cccc3c2OCCC3)nn2c(C(=O)NC3CCc4cccnc43)cnc12]

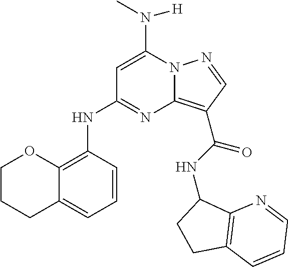 [SMILES: CNc1cc(Nc2cccc3c2OCCC3)nc2c(C(=O)NC3CCc4cccnc43)cnn12]

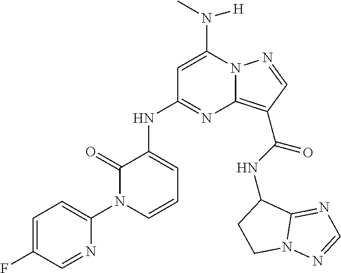 [SMILES: CNc1cc(Nc2cccn(-c3ccc(F)cn3)c2=O)nc2c(C(=O)NC3CCn4ncnc43)cnn12]

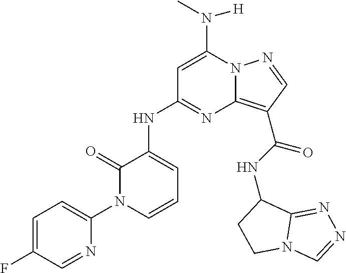 [SMILES: CNc1cc(Nc2cccn(-c3ccc(F)cn3)c2=O)nc2c(C(=O)NC3CCn4cnnc43)cnn12]

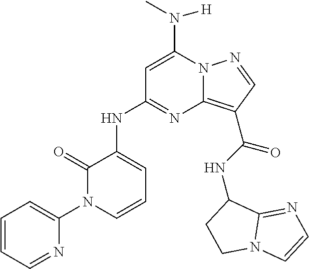 [SMILES: CNc1cc(Nc2cccn(-c3ccccn3)c2=O)nc2c(C(=O)NC3CCn4ccnc43)cnn12]

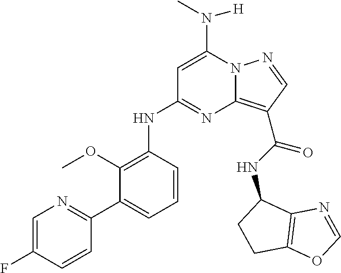 [SMILES: CNc1cc(Nc2cccc(-c3ccc(F)cn3)c2OC)nc2c(C(=O)N[C@@H]3CCc4ocnc43)cnn12]

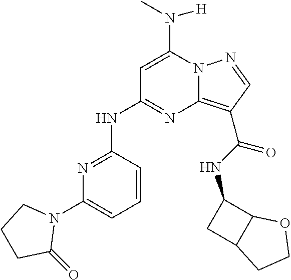 [SMILES: CNc1cc(Nc2cccc(N3CCCC3=O)n2)nc2c(C(=O)N[C@@H]3CC4CCOC43)cnn12]

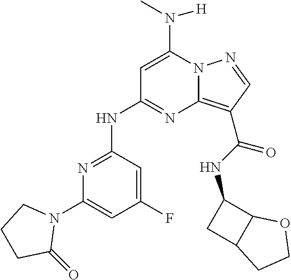 [SMILES: CNc1cc(Nc2cc(F)cc(N3CCCC3=O)n2)nc2c(C(=O)N[C@@H]3CC4CCOC43)cnn12]

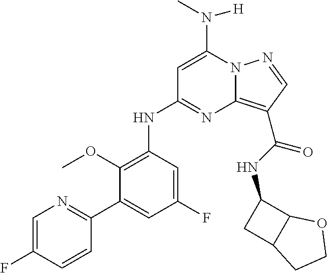 [SMILES: CNc1cc(Nc2cc(F)cc(-c3ccc(F)cn3)c2OC)nc2c(C(=O)N[C@@H]3CC4CCOC43)cnn12]

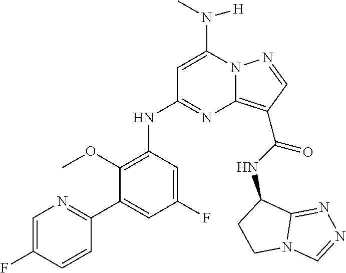 [SMILES: CNc1cc(Nc2cc(F)cc(-c3ccc(F)cn3)c2OC)nc2c(C(=O)N[C@@H]3CCn4cnnc43)cnn12]

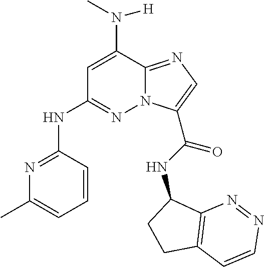 [SMILES: CNc1cc(Nc2cccc(C)n2)nn2c(C(=O)N[C@@H]3CCc4ccnnc43)cnc12]

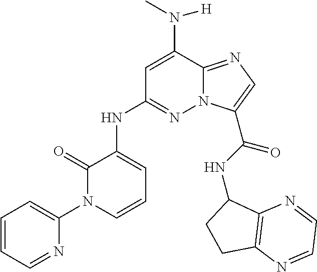 [SMILES: CNc1cc(Nc2cccn(-c3ccccn3)c2=O)nn2c(C(=O)NC3CCc4nccnc43)cnc12]

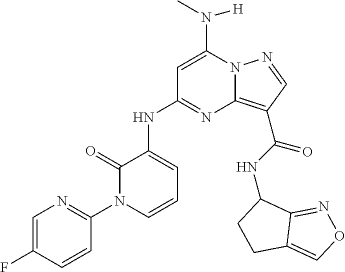 [SMILES: CNc1cc(Nc2cccn(-c3ccc(F)cn3)c2=O)nc2c(C(=O)NC3CCc4conc43)cnn12]

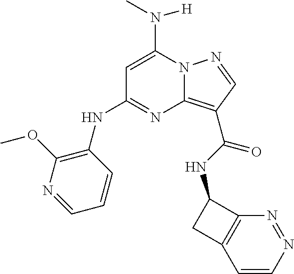 [SMILES: CNc1cc(Nc2cccnc2OC)nc2c(C(=O)N[C@@H]3Cc4ccnnc43)cnn12]

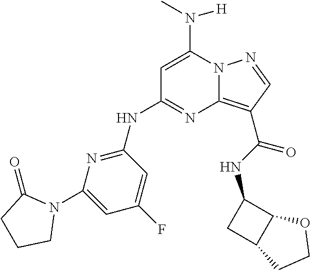 [SMILES: CNc1cc(Nc2cc(F)cc(N3CCCC3=O)n2)nc2c(C(=O)N[C@@H]3C[C@H]4CCO[C@H]43)cnn12]

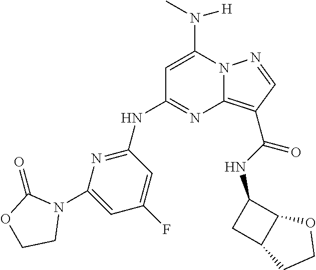 [SMILES: CNc1cc(Nc2cc(F)cc(N3CCOC3=O)n2)nc2c(C(=O)N[C@@H]3C[C@H]4CCO[C@H]43)cnn12]

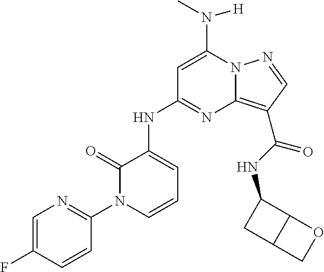 [SMILES: CNc1cc(Nc2cccn(-c3ccc(F)cn3)c2=O)nc2c(C(=O)N[C@@H]3CC4COC43)cnn12]

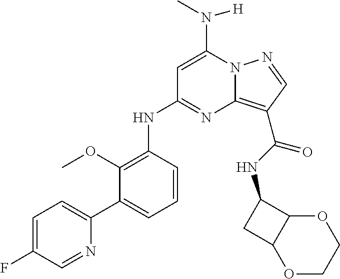 [SMILES: CNc1cc(Nc2cccc(-c3ccc(F)cn3)c2OC)nc2c(C(=O)N[C@@H]3CC4OCCOC43)cnn12]

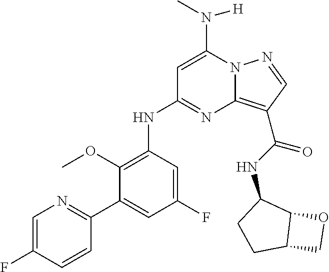 [SMILES: CNc1cc(Nc2cc(F)cc(-c3ccc(F)cn3)c2OC)nc2c(C(=O)N[C@@H]3CC[C@H]4CO[C@H]43)cnn12]

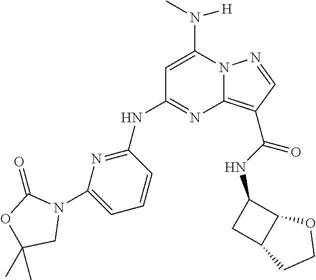 [SMILES: CNc1cc(Nc2cccc(N3CC(C)(C)OC3=O)n2)nc2c(C(=O)N[C@@H]3C[C@H]4CCO[C@H]43)cnn12]

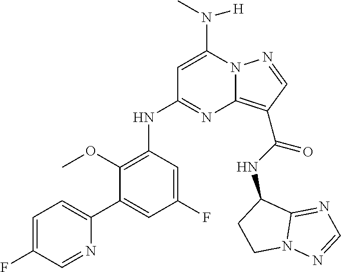 [SMILES: CNc1cc(Nc2cc(F)cc(-c3ccc(F)cn3)c2OC)nc2c(C(=O)N[C@@H]3CCn4ncnc43)cnn12]

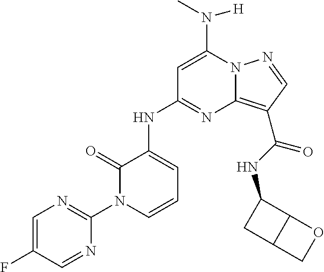 [SMILES: CNc1cc(Nc2cccn(-c3ncc(F)cn3)c2=O)nc2c(C(=O)N[C@@H]3CC4COC43)cnn12]

In some embodiments, the present disclosure also provide a compound selected from the compounds disclosed in Table 1B below, or a pharmaceutically acceptable salt thereof:

TABLE 1B
List of Compounds

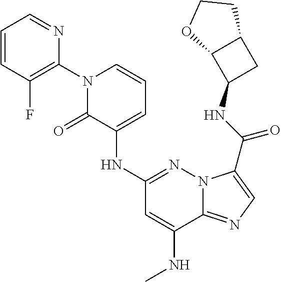 [SMILES: CNc1cc(Nc2cccn(-c3ncccc3F)c2=O)nn2c(C(=O)N[C@@H]3C[C@H]4CCO[C@H]43)cnc12]

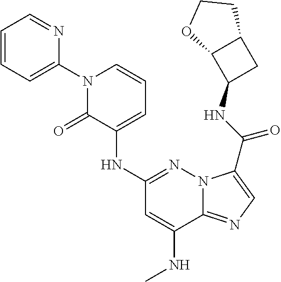 [SMILES: CNc1cc(Nc2cccn(-c3ccccn3)c2=O)nn2c(C(=O)N[C@@H]3C[C@H]4CCO[C@H]43)cnc12]

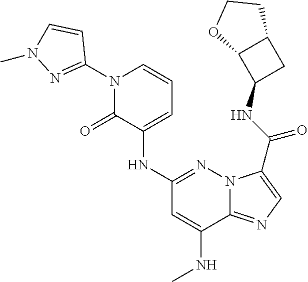 [SMILES: CNc1cc(Nc2cccn(-c3ccn(C)n3)c2=O)nn2c(C(=O)N[C@@H]3C[C@H]4CCO[C@H]43)cnc12]

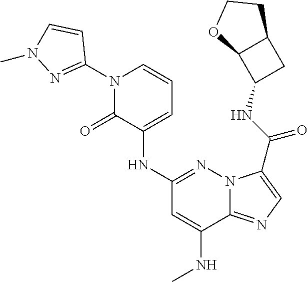 [SMILES: CNc1cc(Nc2cccn(-c3ccn(C)n3)c2=O)nn2c(C(=O)N[C@H]3C[C@@H]4CCO[C@@H]43)cnc12]

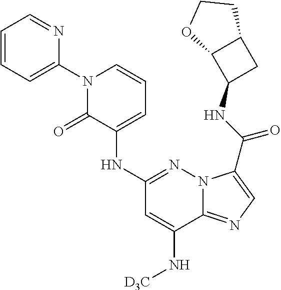 [SMILES: [2H]C([2H])([2H])Nc1cc(Nc2cccn(-c3ccccn3)c2=O)nn2c(C(=O)N[C@@H]3C[C@H]4CCO[C@H]43)cnc12]

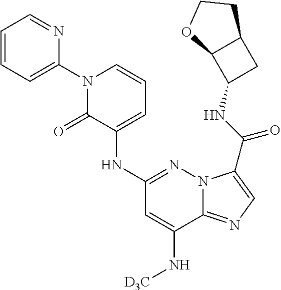 [SMILES: [2H]C([2H])([2H])Nc1cc(Nc2cccn(-c3ccccn3)c2=O)nn2c(C(=O)N[C@H]3C[C@@H]4CCO[C@@H]43)cnc12]

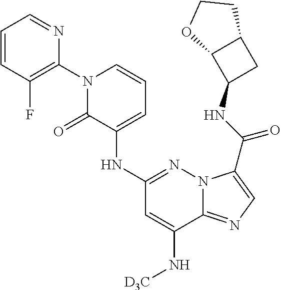 [SMILES: [2H]C([2H])([2H])Nc1cc(Nc2cccn(-c3ncccc3F)c2=O)nn2c(C(=O)N[C@@H]3C[C@H]4CCO[C@H]43)cnc12]

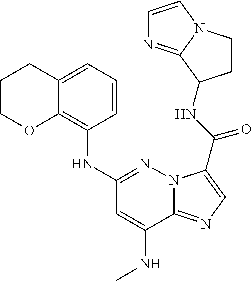 [SMILES: CNc1cc(Nc2cccc3c2OCCC3)nn2c(C(=O)NC3CCn4ccnc43)cnc12]

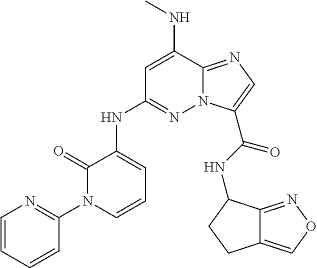 [SMILES: CNc1cc(Nc2cccn(-c3ccccn3)c2=O)nn2c(C(=O)NC3CCc4conc43)cnc12]

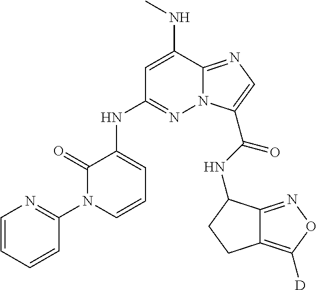 [SMILES: [2H]c1onc2c1CCC2NC(=O)c1cnc2c(NC)cc(Nc3cccn(-c4ccccn4)c3=O)nn12]

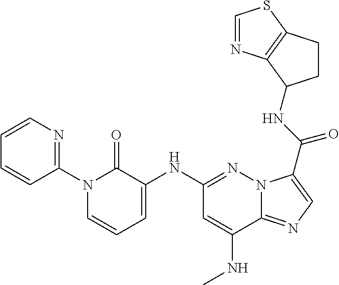 [SMILES: CNc1cc(Nc2cccn(-c3ccccn3)c2=O)nn2c(C(=O)NC3CCc4scnc43)cnc12]

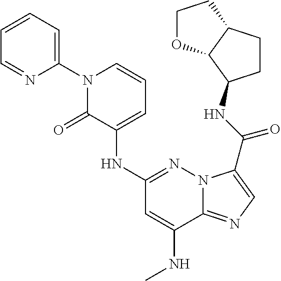 [SMILES: CNc1cc(Nc2cccn(-c3ccccn3)c2=O)nn2c(C(=O)N[C@@H]3CC[C@H]4CCO[C@H]43)cnc12]

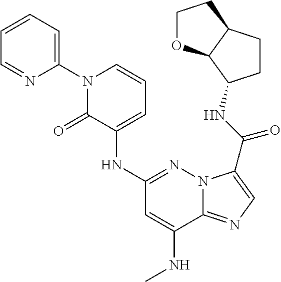 [SMILES: CNc1cc(Nc2cccn(-c3ccccn3)c2=O)nn2c(C(=O)N[C@H]3CC[C@@H]4CCO[C@@H]43)cnc12]

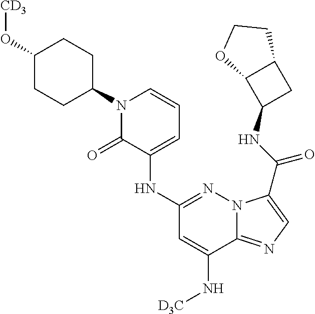 [SMILES: [2H]C([2H])([2H])Nc1cc(Nc2cccn([C@H]3CC[C@H](OC([2H])([2H])[2H])CC3)c2=O)nn2c(C(=O)N[C@@H]3C[C@H]4CCO[C@H]43)cnc12]

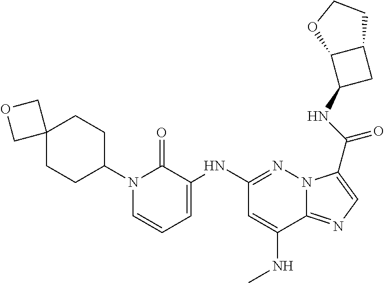 [SMILES: CNc1cc(Nc2cccn(C3CCC4(CC3)COC4)c2=O)nn2c(C(=O)N[C@@H]3C[C@H]4CCO[C@H]43)cnc12]

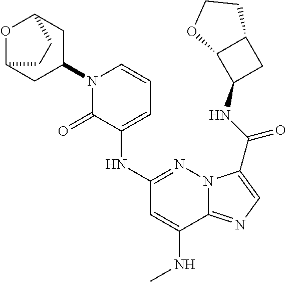 [SMILES: CNc1cc(Nc2cccn([C@@H]3C[C@H]4CC[C@@H](C3)O4)c2=O)nn2c(C(=O)N[C@@H]3C[C@H]4CCO[C@H]43)cnc12]

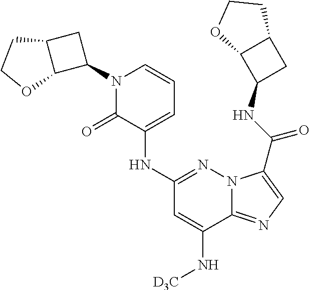 [SMILES: [2H]C([2H])([2H])Nc1cc(Nc2cccn([C@@H]3C[C@H]4CCO[C@H]43)c2=O)nn2c(C(=O)N[C@@H]3C[C@H]4CCO[C@H]43)cnc12]

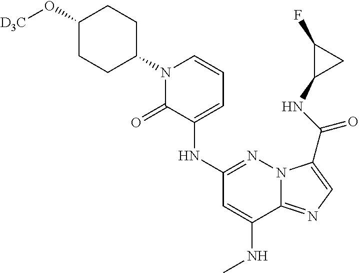 [SMILES: [2H]C([2H])([2H])O[C@H]1CC[C@@H](n2cccc(Nc3cc(NC)c4ncc(C(=O)N[C@@H]5C[C@@H]5F)n4n3)c2=O)CC1]

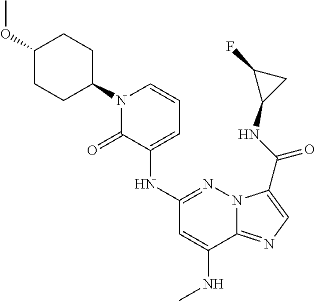 [SMILES: CNc1cc(Nc2cccn([C@H]3CC[C@H](OC)CC3)c2=O)nn2c(C(=O)N[C@@H]3C[C@@H]3F)cnc12]

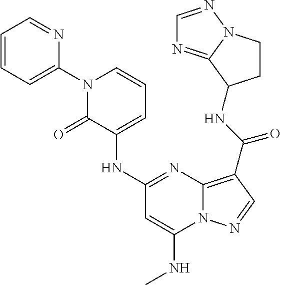 [SMILES: CNc1cc(Nc2cccn(-c3ccccn3)c2=O)nc2c(C(=O)NC3CCn4ncnc43)cnn12]

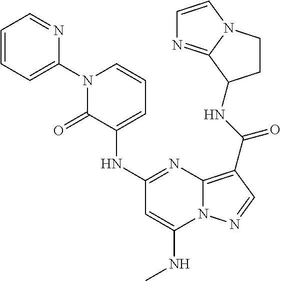 [SMILES: CNc1cc(Nc2cccn(-c3ccccn3)c2=O)nc2c(C(=O)NC3CCn4ccnc43)cnn12]

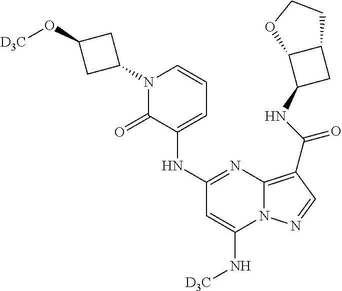 [SMILES: [2H]C([2H])([2H])Nc1cc(Nc2cccn([C@H]3C[C@H](OC([2H])([2H])[2H])C3)c2=O)nc2c(C(=O)N[C@@H]3C[C@H]4CCO[C@H]43)cnn12]

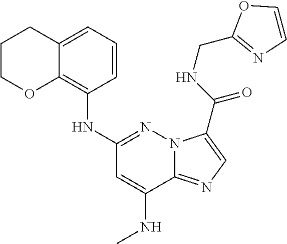 [SMILES: CNc1cc(Nc2cccc3c2OCCC3)nn2c(C(=O)NCc3ncco3)cnc12]

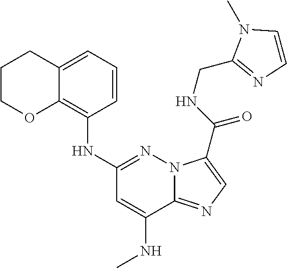 [SMILES: CNc1cc(Nc2cccc3c2OCCC3)nn2c(C(=O)NCc3nccn3C)cnc12]

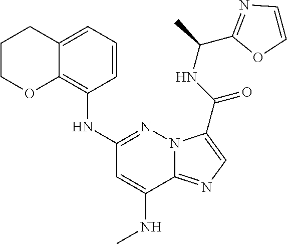 [SMILES: CNc1cc(Nc2cccc3c2OCCC3)nn2c(C(=O)N[C@@H](C)c3ncco3)cnc12]

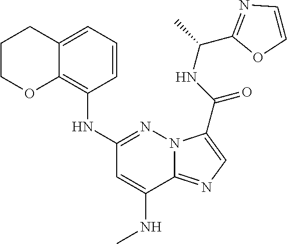 [SMILES: CNc1cc(Nc2cccc3c2OCCC3)nn2c(C(=O)N[C@H](C)c3ncco3)cnc12]

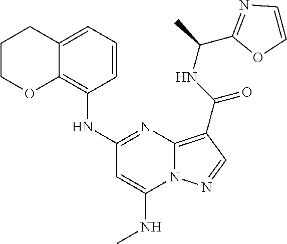 [SMILES: CNc1cc(Nc2cccc3c2OCCC3)nc2c(C(=O)N[C@@H](C)c3ncco3)cnn12]

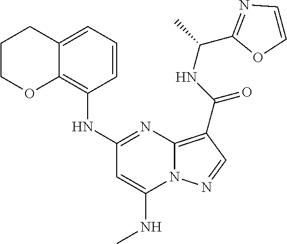 [SMILES: CNc1cc(Nc2cccc3c2OCCC3)nc2c(C(=O)N[C@H](C)c3ncco3)cnn12]

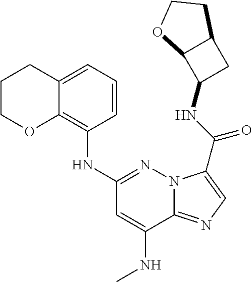

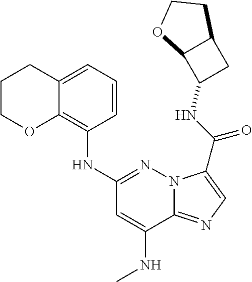

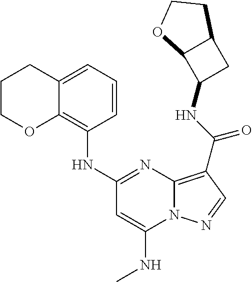

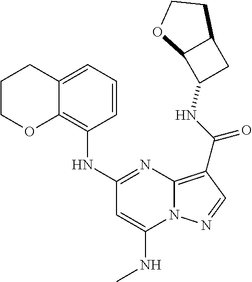

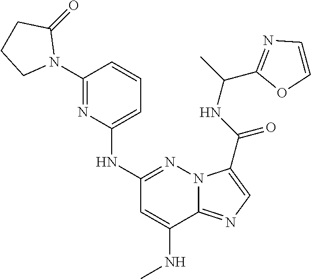 [SMILES: CNc1cc(Nc2cccc(N3CCCC3=O)n2)nn2c(C(=O)NC(C)c3ncco3)cnc12]

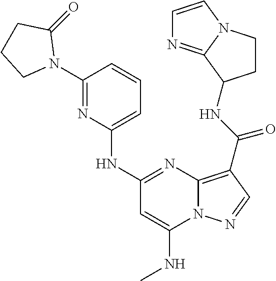 [SMILES: CNc1cc(Nc2cccc(N3CCCC3=O)n2)nc2c(C(=O)NC3CCn4ccnc43)cnn12]

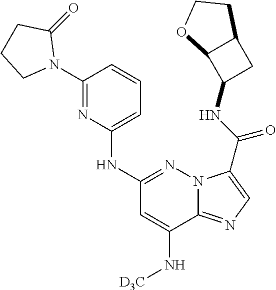

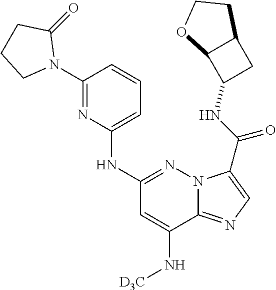

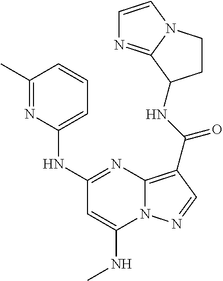 [SMILES: CNc1cc(Nc2cccc(C)n2)nc2c(C(=O)NC3CCn4ccnc43)cnn12]

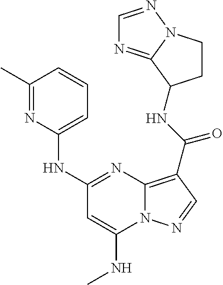 [SMILES: CNc1cc(Nc2cccc(C)n2)nc2c(C(=O)NC3CCn4ncnc43)cnn12]

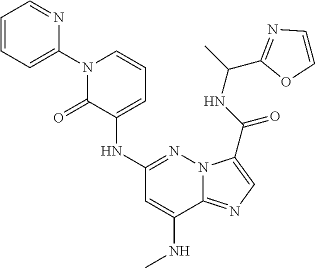 [SMILES: CNc1cc(Nc2cccn(-c3ccccn3)c2=O)nn2c(C(=O)NC(C)c3ncco3)cnc12]

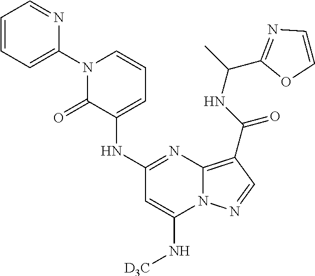 [SMILES: [2H]C([2H])([2H])Nc1cc(Nc2cccn(-c3ccccn3)c2=O)nc2c(C(=O)NC(C)c3ncco3)cnn12]

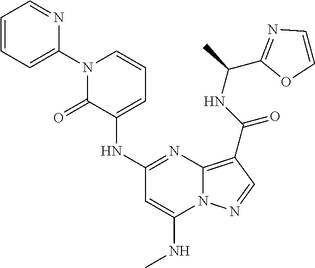 [SMILES: CNc1cc(Nc2cccn(-c3ccccn3)c2=O)nc2c(C(=O)N[C@@H](C)c3ncco3)cnn12]

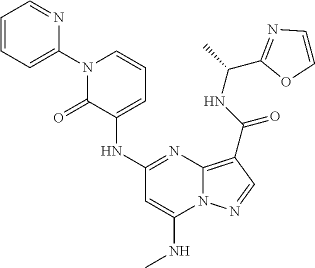 [SMILES: CNc1cc(Nc2cccn(-c3ccccn3)c2=O)nc2c(C(=O)N[C@H](C)c3ncco3)cnn12]

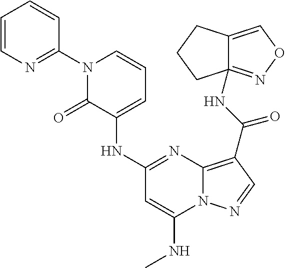

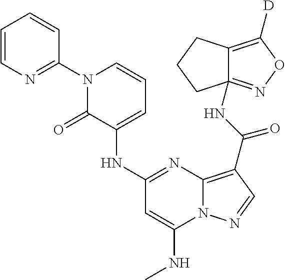

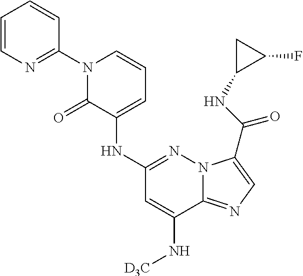 [SMILES: [2H]C([2H])([2H])Nc1cc(Nc2cccn(-c3ccccn3)c2=O)nn2c(C(=O)N[C@@H]3C[C@@H]3F)cnc12]

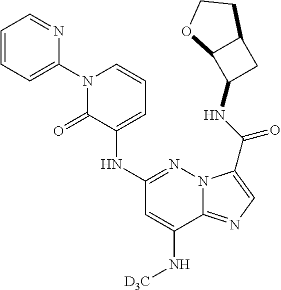

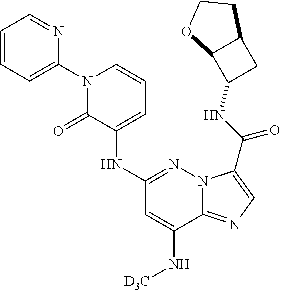

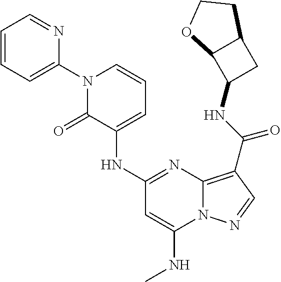

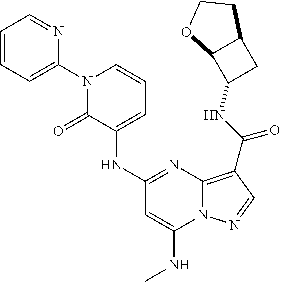

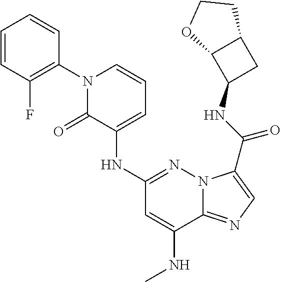 [SMILES: CNc1cc(Nc2cccn(-c3ccccc3F)c2=O)nn2c(C(=O)N[C@@H]3C[C@H]4CCO[C@H]43)cnc12]

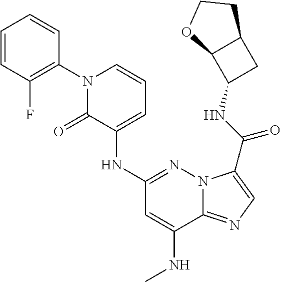 [SMILES: CNc1cc(Nc2cccn(-c3ccccc3F)c2=O)nn2c(C(=O)N[C@H]3C[C@@H]4CCO[C@@H]43)cnc12]

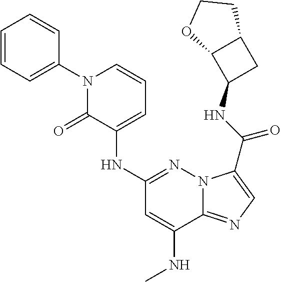 [SMILES: CNc1cc(Nc2cccn(-c3ccccc3)c2=O)nn2c(C(=O)N[C@@H]3C[C@H]4CCO[C@H]43)cnc12]

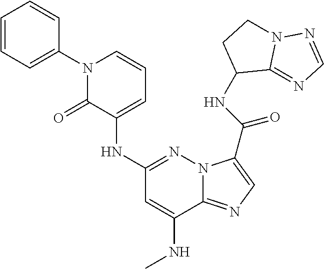 [SMILES: CNc1cc(Nc2cccn(-c3ccccc3)c2=O)nn2c(C(=O)NC3CCn4ncnc43)cnc12]

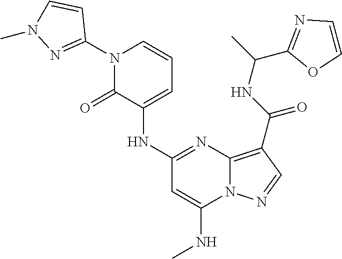 [SMILES: CNc1cc(Nc2cccn(-c3ccn(C)n3)c2=O)nc2c(C(=O)NC(C)c3ncco3)cnn12]

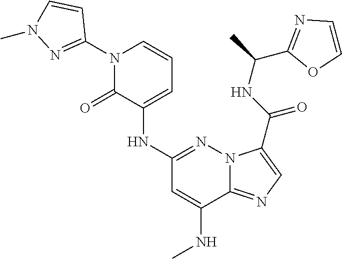 [SMILES: CNc1cc(Nc2cccn(-c3ccn(C)n3)c2=O)nn2c(C(=O)N[C@@H](C)c3ncco3)cnc12]

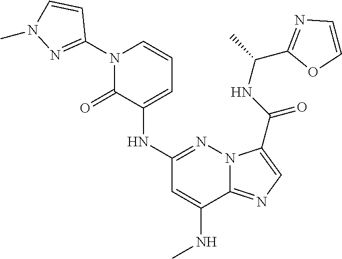 [SMILES: CNc1cc(Nc2cccn(-c3ccn(C)n3)c2=O)nn2c(C(=O)N[C@H](C)c3ncco3)cnc12]

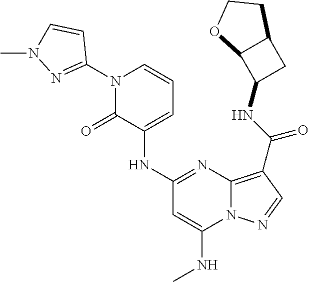

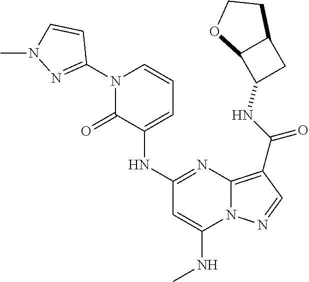

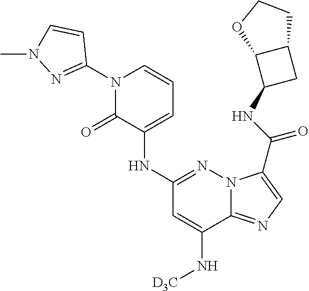 [SMILES: [2H]C([2H])([2H])Nc1cc(Nc2cccn(-c3ccn(C)n3)c2=O)nn2c(C(=O)N[C@@H]3C[C@H]4CCO[C@H]43)cnc12]

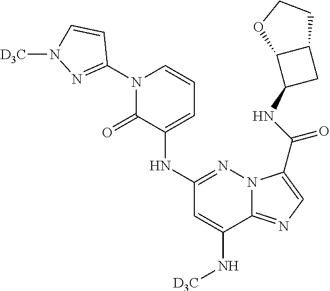 [SMILES: [2H]C([2H])([2H])Nc1cc(Nc2cccn(-c3ccn(C([2H])([2H])[2H])n3)c2=O)nn2c(C(=O)N[C@@H]3C[C@H]4CCO[C@H]43)cnc12]

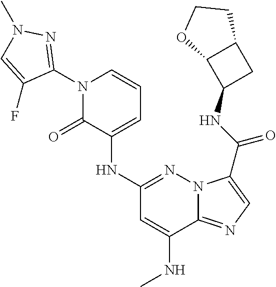 [SMILES: CNc1cc(Nc2cccn(-c3nn(C)cc3F)c2=O)nn2c(C(=O)N[C@@H]3C[C@H]4CCO[C@H]43)cnc12]

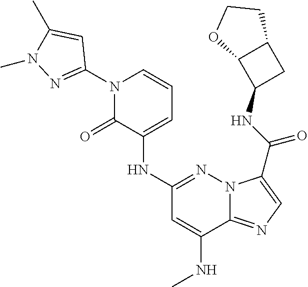 [SMILES: CNc1cc(Nc2cccn(-c3cc(C)n(C)n3)c2=O)nn2c(C(=O)N[C@@H]3C[C@H]4CCO[C@H]43)cnc12]

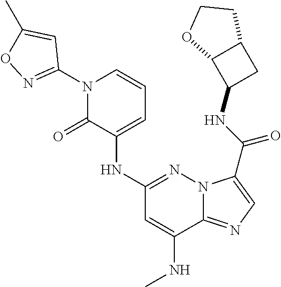

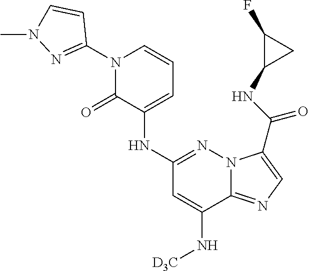 [SMILES: [2H]C([2H])([2H])Nc1cc(Nc2cccn(-c3ccn(C)n3)c2=O)nn2c(C(=O)N[C@@H]3C[C@@H]3F)cnc12]

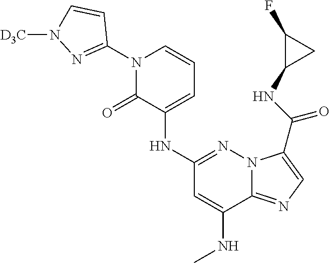 [SMILES: [2H]C([2H])([2H])n1ccc(-n2cccc(Nc3cc(NC)c4ncc(C(=O)N[C@@H]5C[C@@H]5F)n4n3)c2=O)n1]

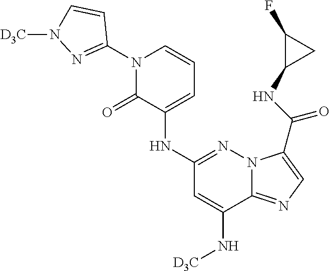 [SMILES: [2H]C([2H])([2H])Nc1cc(Nc2cccn(-c3ccn(C([2H])([2H])[2H])n3)c2=O)nn2c(C(=O)N[C@@H]3C[C@@H]3F)cnc12]

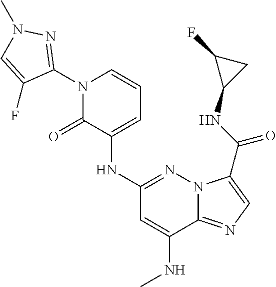 [SMILES: CNc1cc(Nc2cccn(-c3nn(C)cc3F)c2=O)nn2c(C(=O)N[C@@H]3C[C@@H]3F)cnc12]

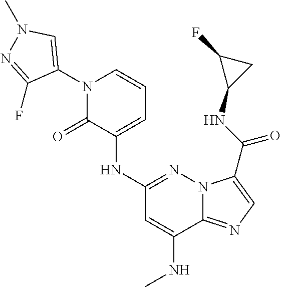 [SMILES: CNc1cc(Nc2cccn(-c3cn(C)nc3F)c2=O)nn2c(C(=O)N[C@@H]3C[C@@H]3F)cnc12]

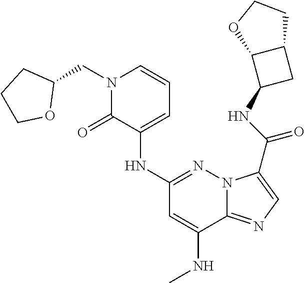 [SMILES: CNc1cc(Nc2cccn(C[C@H]3CCCO3)c2=O)nn2c(C(=O)N[C@@H]3C[C@H]4CCO[C@H]43)cnc12]

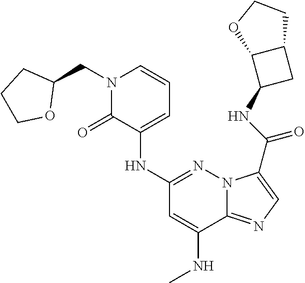 [SMILES: CNc1cc(Nc2cccn(C[C@@H]3CCCO3)c2=O)nn2c(C(=O)N[C@@H]3C[C@H]4CCO[C@H]43)cnc12]

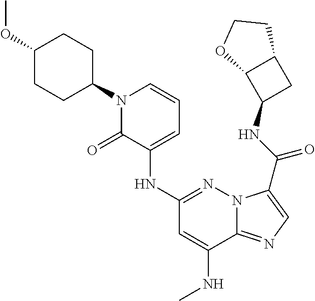 [SMILES: CNc1cc(Nc2cccn([C@H]3CC[C@H](OC)CC3)c2=O)nn2c(C(=O)N[C@@H]3C[C@H]4CCO[C@H]43)cnc12]

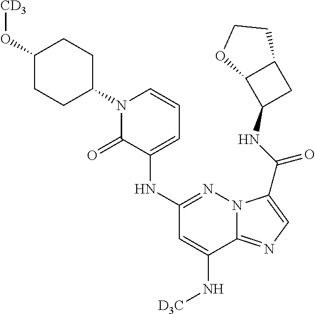 [SMILES: [2H]C([2H])([2H])Nc1cc(Nc2cccn([C@H]3CC[C@@H](OC([2H])([2H])[2H])CC3)c2=O)nn2c(C(=O)N[C@@H]3C[C@H]4CCO[C@H]43)cnc12]

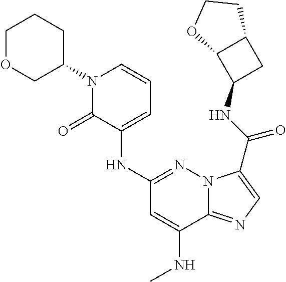 [SMILES: CNc1cc(Nc2cccn([C@H]3CCCOC3)c2=O)nn2c(C(=O)N[C@@H]3C[C@H]4CCO[C@H]43)cnc12]

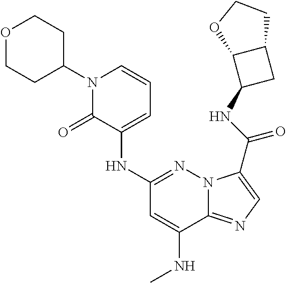 [SMILES: CNc1cc(Nc2cccn(C3CCOCC3)c2=O)nn2c(C(=O)N[C@@H]3C[C@H]4CCO[C@H]43)cnc12]

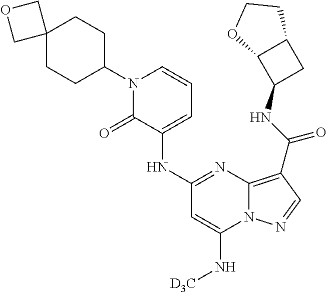 [SMILES: [2H]C([2H])([2H])Nc1cc(Nc2cccn(C3CCC4(CC3)COC4)c2=O)nc2c(C(=O)N[C@@H]3C[C@H]4CCO[C@H]43)cnn12]

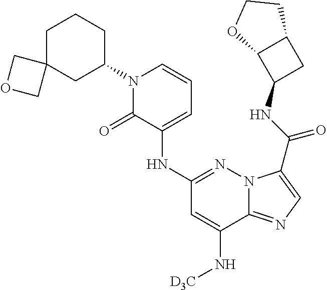 [SMILES: [2H]C([2H])([2H])Nc1cc(Nc2cccn([C@H]3CCCC4(COC4)C3)c2=O)nn2c(C(=O)N[C@@H]3C[C@H]4CCO[C@H]43)cnc12]

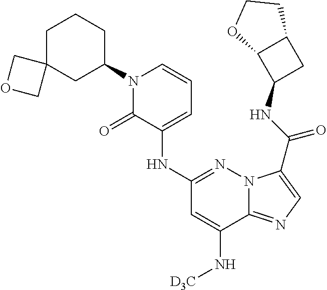 [SMILES: [2H]C([2H])([2H])Nc1cc(Nc2cccn([C@@H]3CCCC4(COC4)C3)c2=O)nn2c(C(=O)N[C@@H]3C[C@H]4CCO[C@H]43)cnc12]

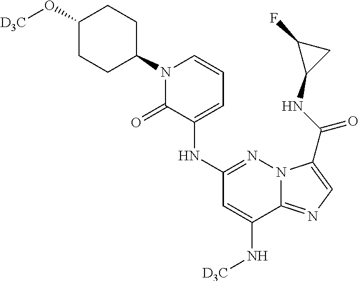 [SMILES: [2H]C([2H])([2H])Nc1cc(Nc2cccn([C@H]3CC[C@H](OC([2H])([2H])[2H])CC3)c2=O)nn2c(C(=O)N[C@@H]3C[C@@H]3F)cnc12]

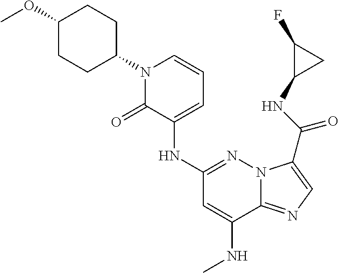 [SMILES: CNc1cc(Nc2cccn([C@H]3CC[C@@H](OC)CC3)c2=O)nn2c(C(=O)N[C@@H]3C[C@@H]3F)cnc12]

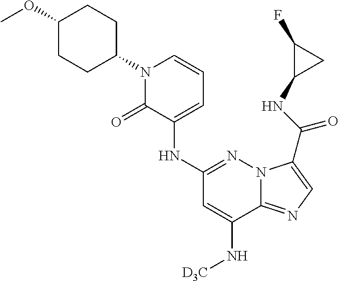 [SMILES: [2H]C([2H])([2H])Nc1cc(Nc2cccn([C@H]3CC[C@@H](OC)CC3)c2=O)nn2c(C(=O)N[C@@H]3C[C@@H]3F)cnc12]

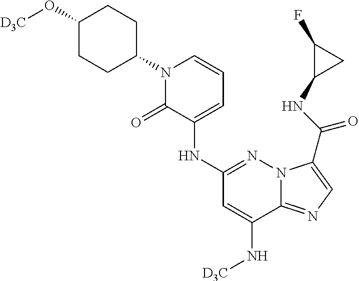 [SMILES: [2H]C([2H])([2H])Nc1cc(Nc2cccn([C@H]3CC[C@@H](OC([2H])([2H])[2H])CC3)c2=O)nn2c(C(=O)N[C@@H]3C[C@@H]3F)cnc12]

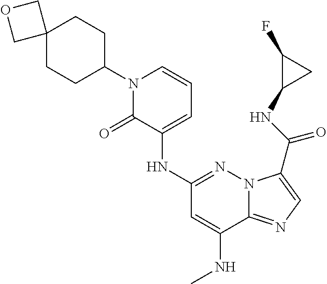 [SMILES: CNc1cc(Nc2cccn(C3CCC4(CC3)COC4)c2=O)nn2c(C(=O)N[C@@H]3C[C@@H]3F)cnc12]

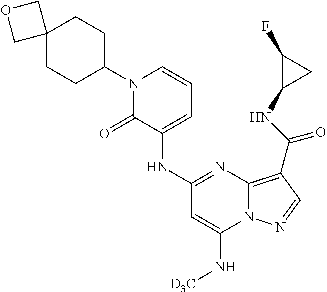 [SMILES: [2H]C([2H])([2H])Nc1cc(Nc2cccn(C3CCC4(CC3)COC4)c2=O)nc2c(C(=O)N[C@@H]3C[C@@H]3F)cnn12]

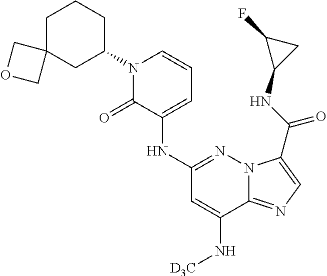 [SMILES: [2H]C([2H])([2H])Nc1cc(Nc2cccn([C@H]3CCCC4(COC4)C3)c2=O)nn2c(C(=O)N[C@@H]3C[C@@H]3F)cnc12]

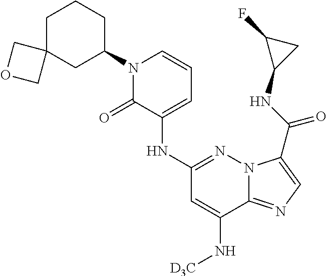 [SMILES: [2H]C([2H])([2H])Nc1cc(Nc2cccn([C@@H]3CCCC4(COC4)C3)c2=O)nn2c(C(=O)N[C@@H]3C[C@@H]3F)cnc12]

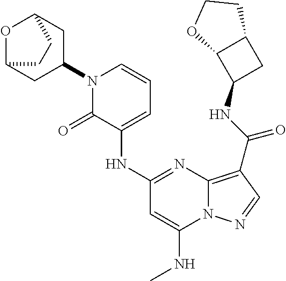 [SMILES: CNc1cc(Nc2cccn([C@@H]3C[C@H]4CC[C@@H](C3)O4)c2=O)nc2c(C(=O)N[C@@H]3C[C@H]4CCO[C@H]43)cnn12]

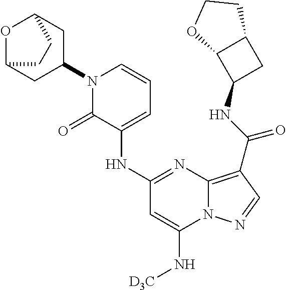 [SMILES: [2H]C([2H])([2H])Nc1cc(Nc2cccn([C@@H]3C[C@H]4CC[C@@H](C3)O4)c2=O)nc2c(C(=O)N[C@@H]3C[C@H]4CCO[C@H]43)cnn12]

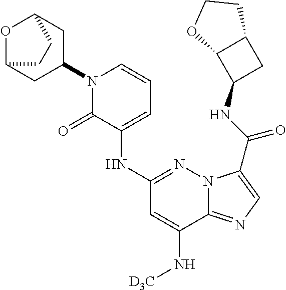 [SMILES: [2H]C([2H])([2H])Nc1cc(Nc2cccn([C@@H]3C[C@H]4CC[C@@H](C3)O4)c2=O)nn2c(C(=O)N[C@@H]3C[C@H]4CCO[C@H]43)cnc12]

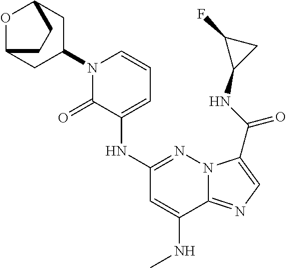 [SMILES: CNc1cc(Nc2cccn([C@H]3C[C@H]4CC[C@@H](C3)O4)c2=O)nn2c(C(=O)N[C@@H]3C[C@@H]3F)cnc12]

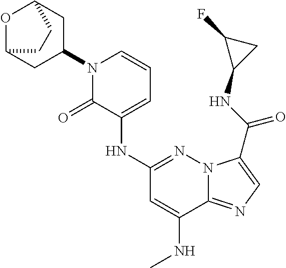 [SMILES: CNc1cc(Nc2cccn([C@@H]3C[C@H]4CC[C@@H](C3)O4)c2=O)nn2c(C(=O)N[C@@H]3C[C@@H]3F)cnc12]

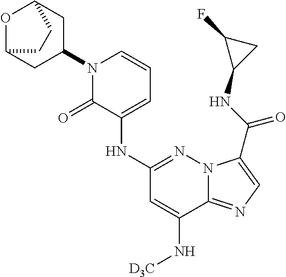 [SMILES: [2H]C([2H])([2H])Nc1cc(Nc2cccn([C@@H]3C[C@H]4CC[C@@H](C3)O4)c2=O)nn2c(C(=O)N[C@@H]3C[C@@H]3F)cnc12]

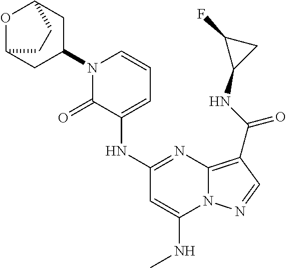 [SMILES: CNc1cc(Nc2cccn([C@@H]3C[C@H]4CC[C@@H](C3)O4)c2=O)nc2c(C(=O)N[C@@H]3C[C@@H]3F)cnn12]

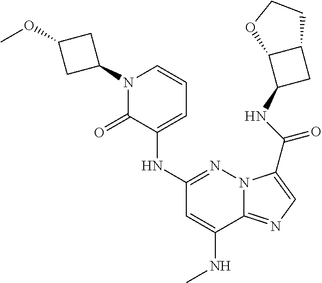 [SMILES: CNc1cc(Nc2cccn([C@H]3C[C@H](OC)C3)c2=O)nn2c(C(=O)N[C@@H]3C[C@H]4CCO[C@H]43)cnc12]

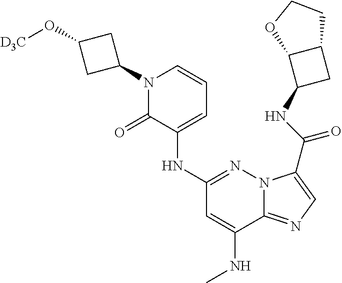 [SMILES: [2H]C([2H])([2H])O[C@H]1C[C@H](n2cccc(Nc3cc(NC)c4ncc(C(=O)N[C@@H]5C[C@H]6CCO[C@H]65)n4n3)c2=O)C1]

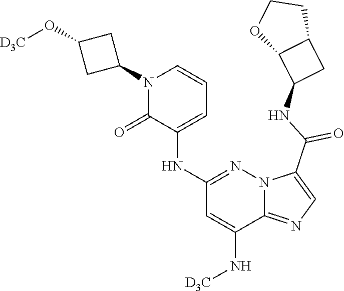 [SMILES: [2H]C([2H])([2H])Nc1cc(Nc2cccn([C@H]3C[C@H](OC([2H])([2H])[2H])C3)c2=O)nn2c(C(=O)N[C@@H]3C[C@H]4CCO[C@H]43)cnc12]

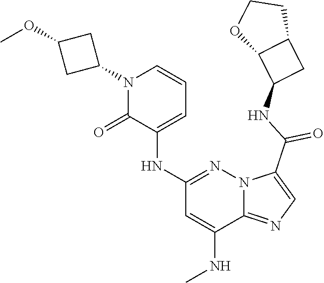 [SMILES: CNc1cc(Nc2cccn([C@H]3C[C@@H](OC)C3)c2=O)nn2c(C(=O)N[C@@H]3C[C@H]4CCO[C@H]43)cnc12]

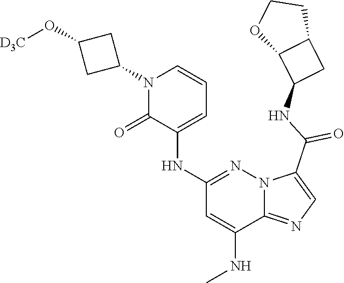 [SMILES: [2H]C([2H])([2H])O[C@H]1C[C@@H](n2cccc(Nc3cc(NC)c4ncc(C(=O)N[C@@H]5C[C@H]6CCO[C@H]65)n4n3)c2=O)C1]

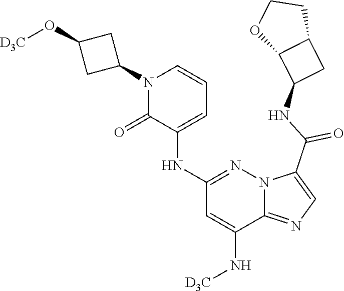 [SMILES: [2H]C([2H])([2H])Nc1cc(Nc2cccn([C@H]3C[C@@H](OC([2H])([2H])[2H])C3)c2=O)nn2c(C(=O)N[C@@H]3C[C@H]4CCO[C@H]43)cnc12]

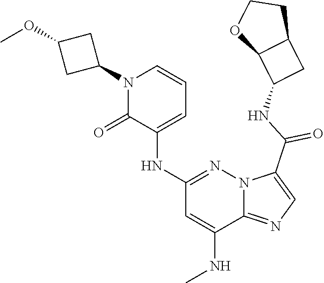 [SMILES: CNc1cc(Nc2cccn([C@H]3C[C@H](OC)C3)c2=O)nn2c(C(=O)N[C@H]3C[C@@H]4CCO[C@@H]43)cnc12]

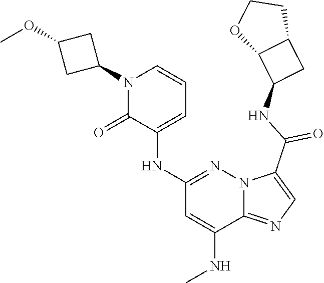 [SMILES: CNc1cc(Nc2cccn([C@H]3C[C@H](OC)C3)c2=O)nn2c(C(=O)N[C@@H]3C[C@H]4CCO[C@H]43)cnc12]

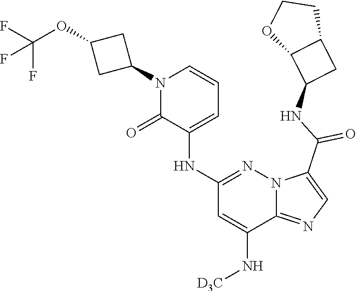 [SMILES: [2H]C([2H])([2H])Nc1cc(Nc2cccn([C@H]3C[C@H](OC(F)(F)F)C3)c2=O)nn2c(C(=O)N[C@@H]3C[C@H]4CCO[C@H]43)cnc12]

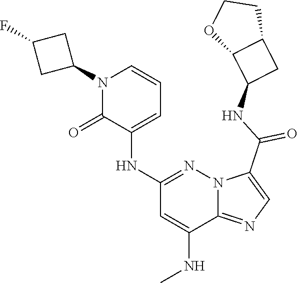 [SMILES: CNc1cc(Nc2cccn([C@H]3C[C@H](F)C3)c2=O)nn2c(C(=O)N[C@@H]3C[C@H]4CCO[C@H]43)cnc12]

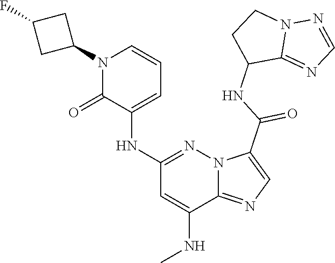 [SMILES: CNc1cc(Nc2cccn([C@H]3C[C@H](F)C3)c2=O)nn2c(C(=O)NC3CCn4ncnc43)cnc12]

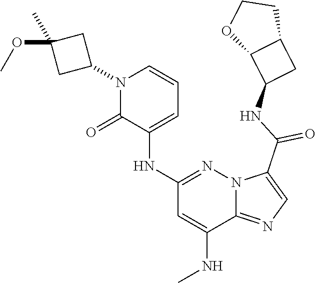 [SMILES: CNc1cc(Nc2cccn([C@H]3C[C@](C)(OC)C3)c2=O)nn2c(C(=O)N[C@@H]3C[C@H]4CCO[C@H]43)cnc12]

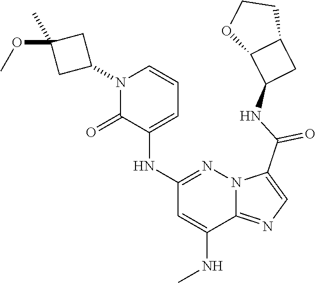 [SMILES: CNc1cc(Nc2cccn([C@H]3C[C@](C)(OC)C3)c2=O)nn2c(C(=O)N[C@@H]3C[C@H]4CCO[C@H]43)cnc12]

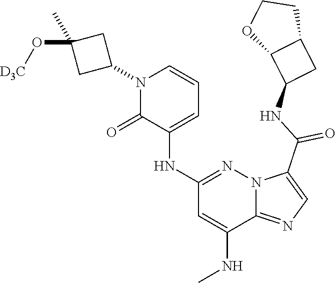 [SMILES: [2H]C([2H])([2H])O[C@]1(C)C[C@@H](n2cccc(Nc3cc(NC)c4ncc(C(=O)N[C@@H]5C[C@H]6CCO[C@H]65)n4n3)c2=O)C1]

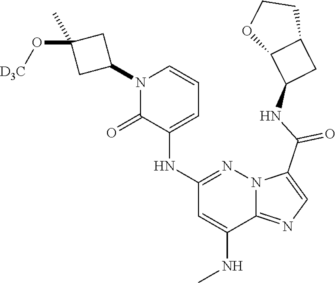 [SMILES: [2H]C([2H])([2H])O[C@]1(C)C[C@H](n2cccc(Nc3cc(NC)c4ncc(C(=O)N[C@@H]5C[C@H]6CCO[C@H]65)n4n3)c2=O)C1]

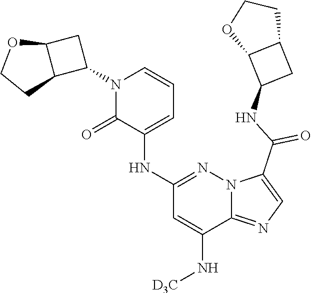 [SMILES: [2H]C([2H])([2H])Nc1cc(Nc2cccn([C@H]3C[C@@H]4OCC[C@@H]43)c2=O)nn2c(C(=O)N[C@@H]3C[C@H]4CCO[C@H]43)cnc12]

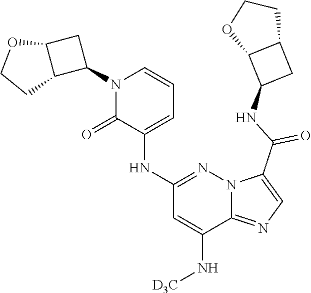 [SMILES: [2H]C([2H])([2H])Nc1cc(Nc2cccn([C@@H]3C[C@H]4OCC[C@@H]34)c2=O)nn2c(C(=O)N[C@@H]3C[C@H]4CCO[C@H]43)cnc12]

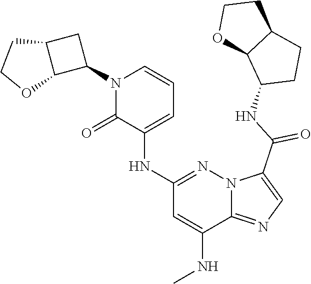 [SMILES: CNc1cc(Nc2cccn([C@@H]3C[C@H]4CCO[C@H]43)c2=O)nn2c(C(=O)N[C@H]3CC[C@@H]4CCO[C@@H]43)cnc12]

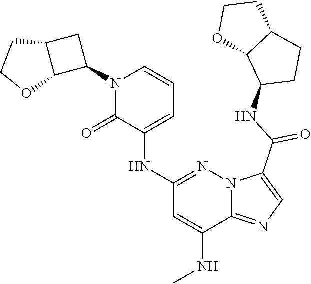 [SMILES: CNc1cc(Nc2cccn([C@@H]3C[C@H]4CCO[C@H]43)c2=O)nn2c(C(=O)N[C@@H]3CC[C@H]4CCO[C@H]43)cnc12]

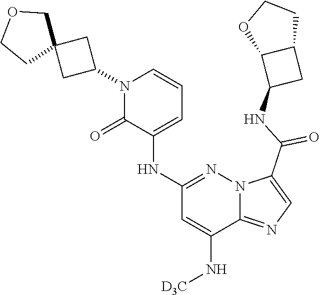

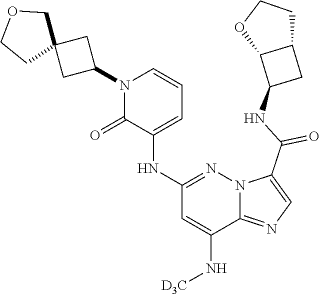

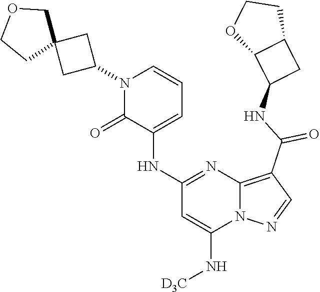

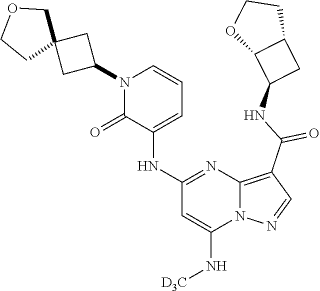

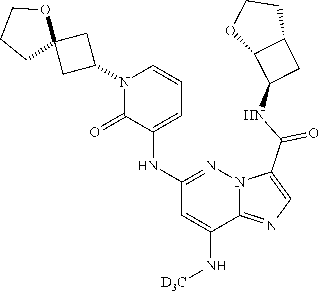

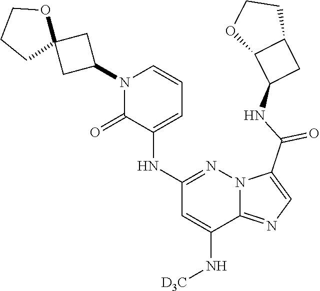

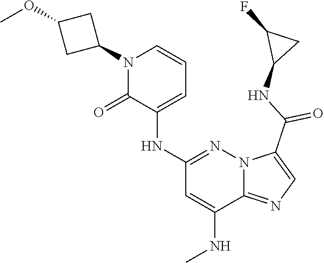 [SMILES: CNc1cc(Nc2cccn([C@H]3C[C@H](OC)C3)c2=O)nn2c(C(=O)N[C@@H]3C[C@@H]3F)cnc12]

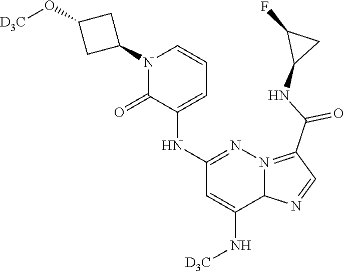

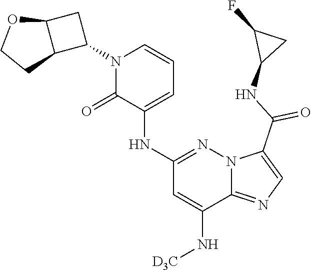 [SMILES: [2H]C([2H])([2H])Nc1cc(Nc2cccn([C@H]3C[C@@H]4OCC[C@@H]43)c2=O)nn2c(C(=O)N[C@@H]3C[C@@H]3F)cnc12]

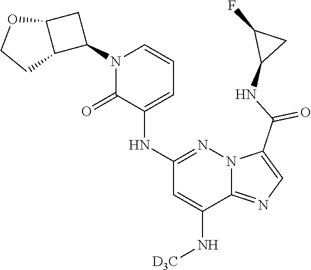 [SMILES: [2H]C([2H])([2H])Nc1cc(Nc2cccn([C@@H]3C[C@H]4OCC[C@@H]34)c2=O)nn2c(C(=O)N[C@@H]3C[C@@H]3F)cnc12]

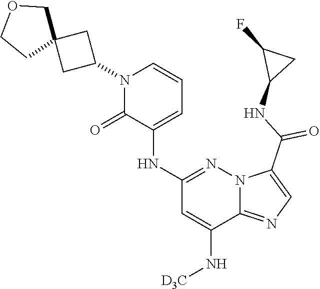

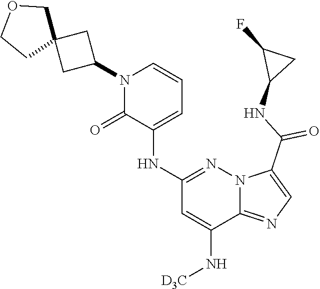

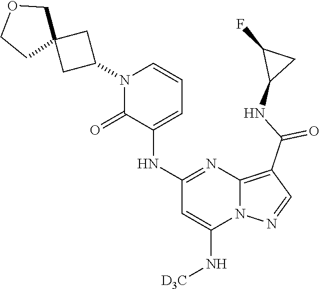

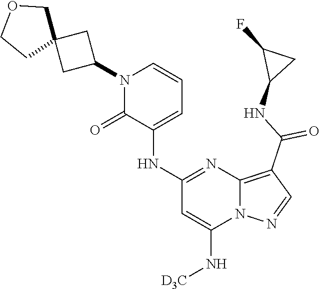

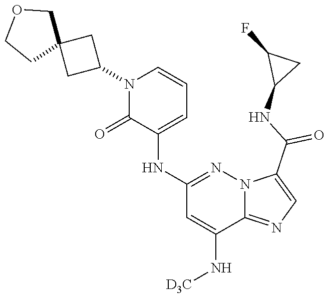

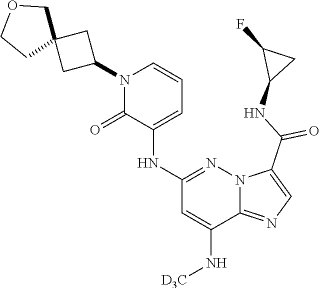

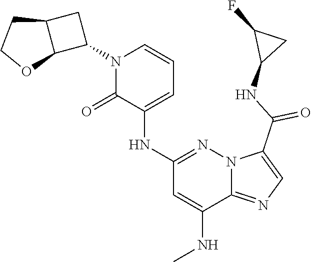 [SMILES: CNc1cc(Nc2cccn([C@H]3C[C@@H]4CCO[C@@H]43)c2=O)nn2c(C(=O)N[C@@H]3C[C@@H]3F)cnc12]

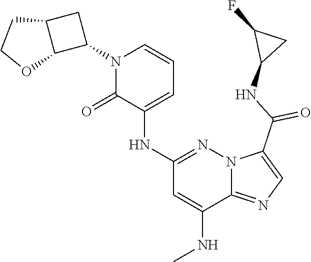 [SMILES: CNc1cc(Nc2cccn([C@H]3C[C@H]4CCO[C@H]43)c2=O)nn2c(C(=O)N[C@@H]3C[C@@H]3F)cnc12]

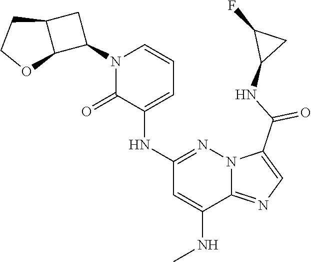 [SMILES: CNc1cc(Nc2cccn([C@@H]3C[C@@H]4CCO[C@@H]43)c2=O)nn2c(C(=O)N[C@@H]3C[C@@H]3F)cnc12]

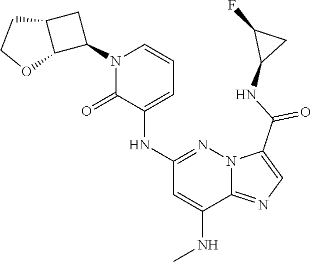 [SMILES: CNc1cc(Nc2cccn([C@@H]3C[C@H]4CCO[C@H]43)c2=O)nn2c(C(=O)N[C@@H]3C[C@@H]3F)cnc12]

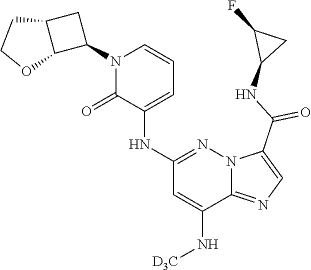 [SMILES: [2H]C([2H])([2H])Nc1cc(Nc2cccn([C@@H]3C[C@H]4CCO[C@H]43)c2=O)nn2c(C(=O)N[C@@H]3C[C@@H]3F)cnc12]

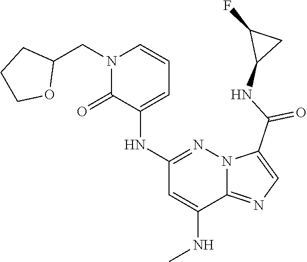 [SMILES: CNc1cc(Nc2cccn(CC3CCCO3)c2=O)nn2c(C(=O)N[C@@H]3C[C@@H]3F)cnc12]

Some compounds of Table 1B contain bold (non-wedged) bond(s) “ [symbol] ” and/or hashed (non-wedged) bond(s) “ [symbol] ” in the structures. As used herein, unless obviously contrary, the use of such non-wedged bolded and hashed bonds in the structures is to show relative stereochemistry, such as cis or trans for two ring substituents. As used herein, unless obviously contrary, the bolded wedged bonds “ [symbol] ” and/or hashed wedged bonds “ [symbol] ” are used in the structures to indicate absolute stereochemistry. For achiral centers, either wedged or non-wedged bonds may be used to indicate relative stereochemistry. As used herein, unless obviously contrary, when the stereochemistry of a chiral center is not specifically drawn, it should be understood that either configurations of the chiral center is intended. Similarly, as used herein, unless obviously contrary, when the relative stereochemistry is not specifically drawn, it should be understood that any relative stereochemistry such as cis or trans is intended.

Compounds of Table 1A and 1B can exist in various stereoisomeric forms, such as individual isomer, an individual enantiomer and/or diastereomer, as applicable, or a mixture of stereoisomers, including racemic mixtures and mixtures enriched in one or more stereoisomers. In some embodiments, when applicable, a compound shown Table 1A or 1B can exist as the as-drawn isomer having greater than 80% ee, greater than 90% ee, greater than 95% ee, greater than 98% ee, greater than 99% ee, or the other enantiomer is non-detectable. In some embodiments, when applicable, a compound shown Table 1A or 1B can exist as the as-drawn isomer having greater than 80% de, greater than 90% de, greater than 95% de, greater than 98% de, greater than 99% de, or the other diastereomer(s) is non-detectable. In some embodiments, when applicable, a compound shown Table 1A or 1B can also exist as a mixture of stereoisomers in any ratio, such as a racemic mixture.

In some embodiments, to the extent applicable, the genus of compounds described herein also excludes any specifically known single compounds prior to this disclosure. In some embodiments, to the extent applicable, any sub-genus of compounds prior to this disclosure that are entirely within a genus of compounds described herein can also be excluded from such genus herein.

The compounds herein can be prepared by those skilled in the art in view of the present disclosure. Representative procedures for preparing compounds of the present disclosure are shown in the Examples section.

For example, using compounds of Formula I as an example, the compounds of the present disclosure can be typically prepared by a series of coupling reactions. As shown in Scheme 1 below, a compound of S-1 can be coupled with an R¹-L¹-donor S-2 to form an intermediate compound S-3. Upon deprotecting the carboxylic acid protecting group Pg², the compound of S-4 can be obtained, which can then be coupled with an amine S-5 to provide the compound of Formula I. The couple sequence can also be reversed, in which the Q-NH group is first introduced, followed by introducing the R¹-L¹-. In scheme 1, Pg¹ is hydrogen or an amine protecting group such as a substituted benzyl group, e.g., paramethoxybenzyl; Pg² is hydrogen or a carboxyl protecting group, such as a C₁₋₄ alkyl, such as methyl, ethyl, etc.; G¹⁰ is hydrogen or a suitable metal or non-metal such as boronic acid or ester; Lg¹ is a leaving group such as a halide, e.g., Cl. The variables R¹, R³, L¹, X, and Q are defined and preferred herein. Compounds of Formula II can be prepared similarly.

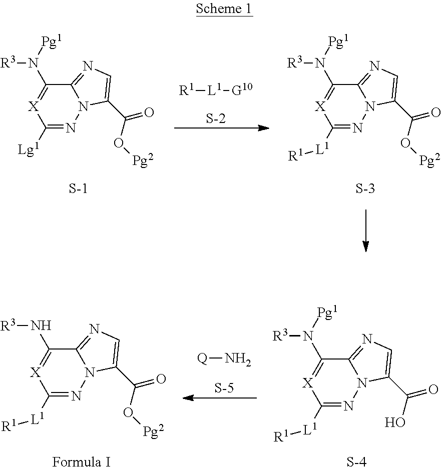

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, 11-2, II-3, II-3-A, II-3-B, II-3-C, II-3-D, II-3-E, 11-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof, e.g., in a therapeutically effective amount. In any of the embodiments described herein, the pharmaceutical composition can comprise a therapeutically effective amount of a compound selected from any of the specific compounds disclosed in Table 1A or 1B herein, or any of Compound Nos. 1-107, or a pharmaceutically acceptable salt thereof.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

Compounds of the present disclosure can be used alone, in combination with each other, or in combination with one or more additional therapeutic agents, e.g., an additional TYK2 inhibitor, an additional anti-inflammatory agent such as an NSAID, etc. These additional therapeutic agents include those known in the art, such as those TYK2 inhibitors and additional agents suitable for combined use with TYK2 inhibitors disclosed for example, in PCT publication Nos. WO2014/074661, WO2015/089143, WO2017/087590, WO2018/067432, WO2018/071794, WO2018/075937, WO2018/093968, WO2019/023468, WO2019/103952, WO2019/183186, WO2020/055636, WO2020/081508, WO2020/086616, WO2020/112937, and WO2020/123225, and U.S. Pat. Nos. 9,505,748, 10,000,480, and 10,294,256, the content of each of which is herein incorporated by reference in its entireties.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, such as psoriasis, psoriatic arthritis, Crohn's disease, ulcerative colitis, inflammatory bowel disease, and/or systemic lupus erythematosus, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment/Use

Compounds of the present disclosure have various utilities. For example, compounds of the present disclosure can be used as therapeutic active substances for the treatment and/or prophylaxis of a TYK2-mediated disease or disorder. As shown in the Examples section, representative compounds of the present disclosure were potent TYK2 inhibitors. Accordingly, some embodiments of the present disclosure are also directed to methods of using one or more compounds of the present disclosure or pharmaceutical compositions herein for treating or preventing a TYK2-mediated disease or disorder in a subject in need thereof, such as for treating psoriasis, psoriatic arthritis, Crohn's disease, ulcerative colitis, inflammatory bowel disease, and/or systemic lupus erythematosus in a subject in need thereof.

In some embodiments, the present disclosure provides a method of inhibiting TYK2 in a subject or biological sample, which comprises contacting the subject or biological sample with an effective amount of the compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or a pharmaceutical composition described herein.

In some embodiments, the present disclosure provides a method of treating or preventing a TYK2-mediated disease or disorder in a subject in need thereof. In some embodiments, the method comprises administering to the subject an effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein. In some embodiments, the TYK2-mediated disease or disorder is associated with type I interferon, IL-10, IL-12, and/or IL-23 signaling. In some embodiments, the TYK2-mediated disease or disorder is associated with IL-12, IL-23 and/or IFNα. In some embodiments, the TYK2-mediated disease or disorder is associated with type I interferon signaling. In some embodiments, the TYK2-mediated disease or disorder is associated with IL-10 signaling. In some embodiments, the TYK2-mediated disease or disorder is associated with IL-12 signaling. In some embodiments, the TYK2-mediated disease or disorder is associated with IL-23 signaling. In some embodiments, the TYK2-mediated disease or disorder is an autoimmune disease or disorder, an inflammatory disease or disorder, a proliferative disease or disorder, an endocrine disease or disorder (e.g., polycystic ovary syndrome, Crouzon's syndrome, or type 1 diabetes), a neurological disease or disorder (e.g., Alzheimer's disease), and/or a disease or disorder associated with transplantation (e.g., transplant rejection or graft versus host disease). In some embodiments, the endocrine disease or disorder is polycystic ovary syndrome, Crouzon's syndrome, or type 1 diabetes. In some embodiments, the neurological disease or disorder is Alzheimer's disease.

In some embodiments, the present disclosure also provides a method of treating or preventing an autoimmune disease or disorder in a subject in need thereof, which comprises administering to the subject an effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, 11-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein. In some embodiments, the autoimmune disease or disorder is selected from type 1 diabetes, ankylosing spondylitis, cutaneous lupus erythematosus, systemic lupus erythematosus, multiple sclerosis, systemic sclerosis, psoriasis, Behçet's disease, POEMS syndrome, Crohn's disease, ulcerative colitis, inflammatory bowel disease, and combinations thereof.

In some embodiments, the present disclosure also provides a method of treating or preventing an inflammatory disease or disorder in a subject in need thereof, which comprises administering to the subject an effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, 11-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein. In some embodiments, the inflammatory disease or disorder is selected from rheumatoid arthritis, asthma, chronic obstructive pulmonary disease, psoriasis, hepatomegaly, Crohn's disease, ulcerative colitis, inflammatory bowel disease, and combinations thereof.

In some embodiments, the present disclosure also provides a method of treating or preventing proliferative disease or disorder, such as a hematological cancer (e.g., leukemia, such as T-cell leukemia, e.g., T-cell acute lymphoblastic leukemia (T-ALL)) in a subject in need thereof, which comprises administering to the subject an effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein. In some embodiments, the proliferative disease or disorder is polycythemia vera, myelofibrosis, or essential thrombocytosis. In some embodiments, the proliferative disease or disorder is a hematological cancer. In some embodiments the proliferative disease or disorder is a leukemia. In some embodiments, the leukemia is a T-cell leukemia. In some embodiments the T-cell leukemia is T-cell acute lymphoblastic leukemia (T-ALL). In some embodiments the proliferative disease or disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and/or R1027H.

In some embodiments, the present disclosure also provides a method of treating or preventing an inflammatory or allergic conditions of the skin in a subject in need thereof, which comprises administering to the subject an effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, 11-2, II-3, II-3-A, II-3-B, II-3-C, II-3-D, II-3-E, 11-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein. In some embodiments, the inflammatory or allergic conditions of the skin is selected from psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, cutaneous lupus erythematosus, systemic lupus erythematosus, pemphigus vulgaris, pemphigus foliaceus, paraneoplastic pemphigus, epidermolysis bullosa acquisita, acne vulgaris, other inflammatory or allergic conditions of the skin, and combinations thereof.

In some particular embodiments, the present disclosure also provides a method of treating psoriasis in a subject in need thereof, which comprises administering to the subject a therapeutically effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein.

In some particular embodiments, the present disclosure also provides a method of treating psoriatic arthritis in a subject in need thereof, which comprises administering to the subject a therapeutically effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein.

In some particular embodiments, the present disclosure also provides a method of treating systemic lupus erythematosus in a subject in need thereof, which comprises administering to the subject a therapeutically effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, 11-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein.

In some particular embodiments, the present disclosure also provides a method of treating Crohn's disease in a subject in need thereof, which comprises administering to the subject a therapeutically effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein.

In some particular embodiments, the present disclosure also provides a method of treating ulcerative colitis in a subject in need thereof, which comprises administering to the subject a therapeutically effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, II-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein.

In some particular embodiments, the present disclosure also provides a method of treating inflammatory bowel disease in a subject in need thereof, which comprises administering to the subject a therapeutically effective amount of a compound of the present disclosure (e.g., a compound of Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, 11-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, or a pharmaceutically acceptable salt thereof) or an effective amount of a pharmaceutical composition described herein.

In some embodiments, the compounds of the present disclosure or pharmaceutical compositions described herein can be used in treating TYK2-mediated diseases or disorders associated with IL-23, IL-12 and/or IFNα, which include, but not limited to, inflammatory diseases such as Crohn's disease, ulcerative colitis, asthma, graft versus host disease, allograft rejection, chronic obstructive pulmonary disease; autoimmune diseases such as Graves' disease, rheumatoid arthritis, systemic lupus erythematosis, cutaneous lupus, lupus nephritis, discoid lupus erythematosus, psoriasis; auto-inflammatory diseases including CAPS, TRAPS, FMF, adult onset stills, systemic onset juvenile idiopathic arthritis, gout, gouty arthritis; metabolic diseases including type 2 diabetes, atherosclerosis, myocardial infarction; destructive bone diseases or disorders such as bone resorption disease, osteoarthritis, osteoporosis, multiple myeloma-related bone disease or disorder; proliferative diseases or disorders such as acute myelogenous leukemia, chronic myelogenous leukemia; angiogenic diseases or disorders such as angiogenic diseases or disorders including solid tumors, ocular neovasculization, and infantile haemangiomas; infectious diseases such as sepsis, septic shock, and Shigellosis; neurodegenerative diseases such as Alzheimer's disease, Parkinson's disease, cerebral ischemias or neurodegenerative disease caused by traumatic injury, oncologic and viral diseases such as metastatic melanoma, Kaposi's sarcoma, multiple myeloma, and HIV infection and CMV retinitis, AIDS, respectively.

In some embodiments, the compounds of the present disclosure or pharmaceutical compositions described herein can be used in treating TYK2-mediated diseases or disorders associated with IL-23, IL-12 and/or IFNα, which include, without limitation, pancreatitis (acute or chronic), asthma, allergies, adult respiratory distress syndrome, chronic obstructive pulmonary disease, glomerulonephritis, rheumatoid arthritis, systemic lupus erythematosis, cutaneous lupus, lupus nephritis, discoid lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, graft vs. host disease, inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic 3-cell disease; diseases characterized by massive neutrophil infiltration; rheumatoid spondylitis, gouty arthritis and other arthritic conditions, cerebral malaria, chronic pulmonary inflammatory disease, silicosis, pulmonary sarcoidosis, bone resorption disease, allograft rejections, fever and myalgias due to infection, cachexia secondary to infection, keloid formation, scar tissue formation, ulcerative colitis, pyresis, influenza, osteoporosis, osteoarthritis, acute myelogenous leukemia, chronic myelogenous leukemia, metastatic melanoma, Kaposi's sarcoma, multiple myeloma, sepsis, septic shock, and Shigellosis; Alzheimer's disease, Parkinson's disease, cerebral ischemias or neurodegenerative disease caused by traumatic injury; angiogenic diseases or disorders including solid tumors, ocular neovasculization, and infantile haemangiomas; viral diseases including acute hepatitis infection (including hepatitis A, hepatitis B and hepatitis C), HIV infection and CMV retinitis, AIDS, ARC or malignancy, and herpes; stroke, myocardial ischemia, ischemia in stroke heart attacks, organ hyposia [should this be hypoxia], vascular hyperplasia, cardiac and renal reperfusion injury, thrombosis, cardiac hypertrophy, thrombin-induced platelet aggregation, endotoxemia and/or toxic shock syndrome, conditions associated with prostaglandin endoperoxidase syndase-2, and pemphigus vulgaris. In some preferred embodiments, the compounds of the present disclosure or pharmaceutical compositions described herein can be used in treating Crohn's disease, ulcerative colitis, allograft rejection, rheumatoid arthritis, psoriasis, ankylosing spondylitis, psoriatic arthritis, and/or pemphigus vulgaris. In some preferred embodiments, the compounds of the present disclosure or pharmaceutical compositions described herein can be used in treating ischemia reperfusion injury, including cerebral ischemia reperfusions injury arising from stroke and/or cardiac ischemia reperfusion injury arising from myocardial infarction. In some preferred embodiments, the compounds of the present disclosure or pharmaceutical compositions described herein can be used in treating multiple myeloma.

Additional diseases or disorders that can be suitably treated with the compounds, compositions, and/or methods of the present disclosure herein include any of those known diseases or disorders mediated by TYK2, some of such are disclosed for example, in PCT publication Nos. WO2014/074661, WO2015/089143, WO2017/087590, WO2018/067432, WO2018/071794, WO2018/075937, WO2018/093968, WO2019/023468, WO2019/103952, WO2019/183186, WO2020/055636, WO2020/081508, WO2020/086616, WO2020/112937, and WO2020/123225, and U.S. Pat. Nos. 9,505,748, 10,000,480, and 10,294,256, the content of each of which is herein incorporated by reference in its entireties.

As used herein, the term “TYK2-mediated” disorders, diseases, and/or conditions as used herein means any disease or other deleterious condition in which TYK2 or a mutant thereof is known to play a role. Such TYK2-mediated diseases or disorders include but are not limited to autoimmune diseases or disorders, inflammatory diseases or disorders, proliferative diseases or disorders, endocrine diseases or disorders, neurological diseases or disorders and diseases or disorders associated with transplantation.

The administering in the methods herein is not limited. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally.

Compounds of the present disclosure can be used as a monotherapy or in a combination therapy. In some embodiments according to the methods described herein, compounds of the present disclosure can be administered as the only active ingredient(s). In some embodiments according to the methods described herein, compounds of the present disclosure can also be co-administered with an additional therapeutic agent, either concurrently or sequentially in any order, to the subject in need thereof.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable groups for the variables in compounds of Formula I or II, or a subformula thereof, as applicable, are independently selected. Non-limiting useful groups for the variables in compounds of Formula I or II, or a subformula thereof, as applicable, include any of the respective groups, individually or in any combination, as shown in the specific compounds described in Table 1A or 1B herein. For example, in some embodiments, suitable groups as R¹ in Formula I or II include any of the R¹ groups shown in specific examples compounds described in Table 1A or 1B herein, without regard to the other variables shown in the specific compounds. In some embodiments, compounds of Formula I or II can include a R¹ group according to any of the R¹ groups shown in the specific compounds described in Table 1A or 1B herein in combination at least one other variable (e.g, L¹) according to the specific compounds described in Table 1A or 1B herein, wherein the R¹ and at least one other variable can derive from the same compound or a different compound. Any of such combinations are contemplated and within the scope of the present disclosure.

The described embodiments of the present disclosure can be combined. Such combination is contemplated and within the scope of the present disclosure. For example, it is contemplated that the definition(s) of any one or more of L¹, R¹, R³, X, and Q of Formula I or II can be combined with the definition of any one or more of the other(s) of L¹, R¹, R³, X, and Q, as applicable, and the resulted compounds from the combination are within the scope of the present disclosure.

The symbol, [symbol] displayed perpendicular to (or otherwise crossing) a bond, indicates the point at which the displayed moiety is attached to the remainder of the molecule. It should be noted that the immediately connected group or groups maybe shown beyond the symbol, [symbol] to indicate direction of attachment, as would be understood by those skilled in the art.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by SFC or HPLC area, or both, or with a non-detectable amount of the other stereoisomer(s). For example, in some embodiments, the compound can exist predominantly as the as-drawn stereoisomer, with an enantiomeric excess (“ee”) of greater than 70%, such as such as having greater than 80% ee, greater than 90% ee, greater than 95% ee, greater than 98% ee, greater than 99% ee, or the other enantiomer is non-detectable. In some embodiments, the compound can exist predominantly as the as-drawn stereoisomer, with an diastereomeric excess (“de”) of greater than 70%, such as such as having greater than 80% de, greater than 90% de, greater than 95% de, greater than 98% de, greater than 99% de, or the other diastereomer(s) is non-detectable. The presence and/or amounts of stereoisomers can be determined by those skilled in the art in view of the present disclosure, including through the use of a chiral HPLC or chiral SFC. As understood by those skilled in the art, when a “*” is shown in the chemical structures herein, unless otherwise contradictory from context, it is to designate that the corresponding chiral center is enantiomerically pure or enriched in either of the configurations or is enantiomerically pure or enriched in the as-dawn configuration, such as with less than 20%, less than 10%, less than 5%, less than 1%, by weight, by HPLC or SFC area, or both, or with a non-detectable amount of the other stereoisomer(s). In cases where two “*” are associated with two non-chiral carbons in chemical structures herein, unless otherwise contradictory from context, it is to designate that one substituent on one of the two non-chiral carbons and one substutuent on the other of the two non-chiral carbons have one of the two possible relative stereochemistry (or enriched in one), such as cis or trans to each other. Also, when no stereochemistry is specifically drawn, and no “*” is used in the chemical structures, unless otherwise contradictory from context, it should be understood that such structures include the corresponding compound in any stereoisomeric forms, including individual isomers substantially free of other isomers and mixtures of various isomers including racemic mixtures.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to Formula I (e.g., I-1, I-1-A, I-1-B, I-1-C, I-1-D, I-1-E, I-2, I-3, I-3-A, I-3-B, I-3-C, I-3-D, I-3-E, I-4, I-4-A, I-4-B, I-4-C, I-4-D, I-4-E, I-4-E1, I-4-E2, I-4-F, or I-A), Formula II (e.g., II-1, II-1-A, II-1-B, II-1-C, II-1-D, II-1-E, II-2, II-3, II-3-A, II-3-B, II-3-C, II-3-D, II-3-E, 11-4, II-4-A, II-4-B, II-4-C, II-4-D, II-4-E, II-4-E1, II-4-E2, II-4-F, or II-A), or any of the specific compounds disclosed in Table 1A or 1B herein, or any of Compound Nos. 1-107, isotopically labeled compound(s) thereof (such as a deuterated analog wherein one or more of the hydrogen atoms is/are substituted with a deuterium atom with an abundance above its natural abundance, e.g., a CD₃ analog when the compound has a CH₃ group), possible regioisomers, possible geometric isomers, possible stereoisomers thereof (including diastereoisomers, enantiomers, and racemic mixtures), tautomers thereof, conformational isomers thereof, pharmaceutically acceptable esters thereof, and/or possible pharmaceutically acceptable salts thereof (e.g., acid addition salt such as HCl salt or base addition salt such as Na salt). To be clear, collectively, compound Nos. 1-107 refer to the compounds in the Examples section labeled with an integer only, such as 1, 2, etc. up to 107, or when applicable, may be additionally followed by labels “a”, “b”, “c”, or “d” for the corresponding stereoisomers. See e.g., Synthetic Examples 1-21 and Table 2 herein. Exemplified synthesis and characterizations of Compound Nos. 1-107 are shown in the Examples section. Detailed exemplified procedures were shown in the synthetic examples, e.g., 1-21. Hydrates and solvates of the compounds of the present disclosure are considered compositions of the present disclosure, wherein the compound(s) is in association with water or solvent, respectively.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl can include one to twelve carbon atoms (i.e., C₁₋₁₂ alkyl) or the number of carbon atoms designated. In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group includes methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁₋₄ heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Carbocyclyl” or “carbocyclic” as used by itself or as part of another group refers to a radical of a non-aromatic cyclic hydrocarbon group having at least 3 carbon atoms, e.g., from 3 to 10 ring carbon atoms (“C₃₋₁₀ carbocyclyl”), and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. For the avoidance of doubt, the carbocyclyl groups herein also include ring systems in which one or more rings are aryl ring(s), provided that the carbocyclyl ring as a whole is not aromatic, and the point of attachment can be on any ring. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl. As used herein, the term “carbocyclylene” as used by itself or as part of another group refers to a divalent radical derived from the carbocyclyl group defined herein.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring. As used herein, the term “cycloalkylene” as used by itself or as part of another group refers to a divalent radical derived from a cycloalkyl group, for example,

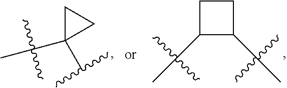 [SMILES: *C1(*)CC1 ; *C1CCC1*]

etc.

“Heterocyclyl” or “heterocyclic” as used by itself or as part of another group refers to a radical of a 3-membered or larger, such as 3- to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a fused, bridged, or spiro bicyclic system (“bicyclic heterocyclyl”), and can be saturated or can be partially unsaturated. Heterocyclyl bicyclic ring systems can include one or more heteroatoms in one or both rings, and the point of attachment can be on any ring. For the avoidance of doubt, the heterocyclyl groups herein include ring systems in which one or more rings are carbocyclic ring defined herein, and the point of attachment can be on any ring. In addition, the heterocyclyl groups herein also include ring systems in which one or more rings are aryl or heteroaryl ring(s), provided that the heterocyclyl ring as a whole is not a heteroaryl ring, and the point of attachment can be on any ring. As used herein, the term “heterocyclylene” as used by itself or as part of another group refers to a divalent radical derived from the heterocyclyl group defined herein. The heterocyclyl or heterocylylene can be optionally linked to the rest of the molecule through a carbon or nitrogen atom.

Exemplary non-limiting heterocyclyl groups include azirdinyl, oxiranyl, thiiranyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, pyrrolyl-2,5-dione, dioxolanyl, oxasulfuranyl, disulfuranyl, oxazolidin-2-one, triazolinyl, oxadiazolinyl, thiadiazolinyl, piperidinyl, tetrahydropyranyl, dihydropyridinyl, thianyl, piperazinyl, morpholinyl, dithianyl, dioxanyl, triazinanyl, azepanyl, oxepanyl, thiepanyl, azocanyl, oxecanyl, thiocanyl, indolinyl, isoindolinyl, dihydrobenzofuranyl, dihydrobenzothienyl, benzoxazolinonyl, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). As used herein, the term “arylene” as used by itself or as part of another group refers to a divalent radical derived from the aryl group defined herein.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-14 membered monocyclic, bicyclic, or tricyclic 4n+2 aromatic ring system (e.g., having 6 or 10 pi electrons shared in a cyclic array) having ring carbon atoms and at least one, preferably, 1-4, ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. In bicyclic heteroaryl groups wherein one ring does not contain a heteroatom (e.g., indolyl, quinolinyl, and the like), the point of attachment can be on either ring, i.e., either the ring bearing a heteroatom (e.g., 2-indolyl) or the ring that does not contain a heteroatom (e.g., 5-indolyl). As used herein, the term “heteroarylene” as used by itself or as part of another group refers to a divalent radical derived from the heteroaryl group defined herein.

Exemplary non-limiting heteroaryl groups include pyrrolyl, furanyl, thiophenyl, imidazolyl, pyrazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, triazinyl, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, purinyl, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocyclyl, aryl, or heteroaryl ring. Substitution can occur on any available carbon, oxygen, or nitrogen atom, and can form a spirocycle. Typically, substitution herein does not result in an O—O, O—N, S—S, S—N(except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, OH, hydroxyalkyl, haloalkyl, O-alkyl, O-haloalkyl, -alkylene-O-alkyl, O-aryl, O-alkylene-aryl, acyl, C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), N(Y₁)(Y₂), -alkylene-N(Y₁)(Y₂), C(O)N(Y₁)(Y₂) and S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to CF₃), O—(C₁-C₈)alkyl groups, OH, S—(C₁-C₈)alkyl groups, —SH, —NH(C₁-C₈)alkyl groups, N((C₁-C₈)alkyl)₂ groups, —NH₂, —C(O)NH₂, C(O)NH(C₁-C₈)alkyl groups, C(O)N((C₁-C₈)alkyl)₂, —NHC(O)H, —NHC(O) (C₁-C₈)alkyl groups, —NHC(O) (C₃-C₈)cycloalkyl groups, N((C₁-C₈)alkyl)C(O)H, N((C₁-C₈)alkyl)C(O)(C₁-C₈)alkyl groups, NHC(O)NH₂, NHC(O)NH(C₁-C₈)alkyl groups, N((C₁-C₈)alkyl)C(O)NH₂ groups, NHC(O)N((C₁-C₈)alkyl)₂ groups, N((C₁-C₈)alkyl)C(O)N((C₁-C₈)alkyl)₂ groups, N((C₁-C₈)alkyl)C(O)NH((C₁-C₈)alkyl), C(O)H, C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH—)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents include, but are not limited to, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkylene, heteroalkyl, heteroalkylene, alkenyl, alkynyl, carbocyclic, carbocyclylene, cycloalkyl, cycloalkylene, alkoxy, cycloalkoxy, heterocyclyl, or heterocyclylene herein can each be independently unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl, arylene, heteroaryl or heteroarylene group herein can each be independently unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from 0, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from tautomerization. The exact ratio of the tautomers depends on several factors, including for example temperature, solvent, and pH. Tautomerizations are known to those skilled in the art. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

The term “effective amount” or “therapeutically effective amount” refers to that amount of a compound or combination of compounds as described herein that is sufficient to effect the intended application including, but not limited to, prophylaxis or treatment of diseases. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells and/or tissues. The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of embodiments herein can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. The abbreviations used in the Examples section should be understood as having their ordinary meanings in the art unless specifically indicated otherwise or obviously contrary from context. The examples are illustrative only and do not limit the claimed invention in any way.

Exemplary embodiments of steps for performing the synthesis of products described herein are described in greater detail infra. Some of the compounds discussed herein can be prepared by separating from the corresponding racemic or diastereomeric mixtures. As would be understood by a person of ordinary skill in the art, the compounds described in the Examples section immediately prior to the chiral separation step, e.g., by supercritical fluid chromatography (SFC), exist in racemic and/or stereoisomeric mixture forms, the bolded but not wedged bonds and/or the hashed but not wedged bonds are used in the chemical structure drawings to indicate relative stereochemistry. It should be understood that the enantiomeric excesses (“ee”) and/or diastereomeric excesses (“de”) reported for these examples are only representative from the exemplified procedures herein and not limiting; those skilled in the art would understand that such enantiomers with a different ee, such as a higher ee, or diastereomers with a different de, such as a higher de, can be obtained in view of the present disclosure.

### Intermediate Synthesis 1. Synthesis of ethyl 6-chloro-8-((4-methoxybenzyl)(methyl)amino)imidazo[1,2-b]pyridazine-3-carboxylate (Intermediate I)

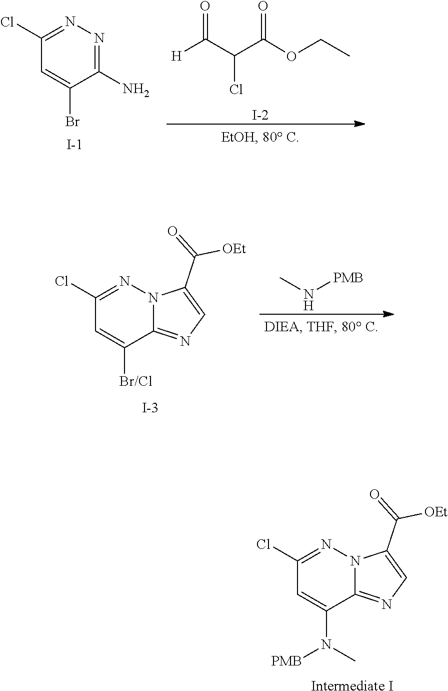

To a solution of 4-bromo-6-chloropyridazin-3-amine (I-1, 2.40 g, 11.5 mmol) in ethyl alcohol (20 mL) was added ethyl 2-chloro-3-oxopropanoate (I-2, 2.77 g, 18.4 mmol) dropwise at room temperature, and the mixture was stirred at 80° C. overnight. The reaction mixture was concentrated under reduced pressure, diluted with water (60 mL) and extracted with dichloromethane (3×50 mL). The combined organic layers were dried over anhydrous sodium sulfate, filtered and concentrated under reduced pressure. The residue was subjected to silica gel column chromatography to afford a mixture of ethyl 8-bromo-6-chloroimidazo[1,2-b]pyridazine-3-carboxylate and ethyl 6,8-dichloroimidazo[1,2-b]pyridazine-3-carboxylate (I-3, 1.90 g, crude, approximately 1:1) as a yellow solid. LC-MS (ESI): m/z 303.9, 260.2 [M+H]⁺.

To a mixture of ethyl 8-bromo-6-chloroimidazo[1,2-b]pyridazine-3-carboxylate and ethyl 6,8-dichloroimidazo[1,2-b]pyridazine-3-carboxylate (I-3, 1.90 g, crude) in tetrahydrofuran (18 mL) were added 1-(4-methoxyphenyl)-N-methylmethanamine (1.42 g, 9.40 mmol) and N,N-diisopropylethylamine (1.87 g, 14.4 mmol). The reaction mixture was stirred at 80° C. for 2 hrs. Then the mixture was cooled to room temperature and concentrated under reduced pressure. The residue was subjected to reverse phase chromatography to afford ethyl 6-chloro-8-((4-methoxybenzyl)(methyl)amino)imidazo[1,2-b]pyridazine-3-carboxylate (Intermediate I, 2.50 g) as a yellow solid. LC-MS (ESI): m/z 375.3 [M+H]⁺.

### Intermediate Synthesis 2. Synthesis of ethyl 6-chloro-8-((4-methoxybenzyl)(methyl-d₃)amino)imidazo[1,2-b]pyridazine-3-carboxylate (Intermediate II)

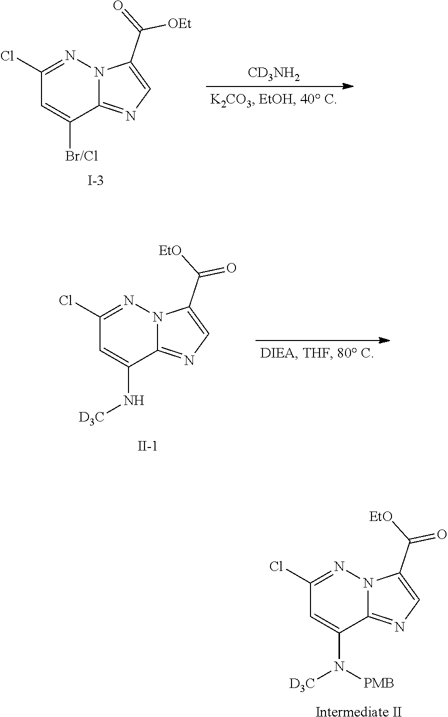

To a mixture of ethyl 8-bromo-6-chloroimidazo[1,2-b]pyridazine-3-carboxylate, ethyl 6,8-dichloroimidazo[1,2-b]pyridazine-3-carboxylate (I-3, 5.00 g, crude) and potassium carbonate (4.00 g, 28.8 mmol) in ethyl alcohol (100 mL) was added methan-d₃-amine hydrochloride (1.42 g, 20.2 mmol) in portions at 0° C. The resulting mixture was stirred at 40° C. overnight. The reaction mixture was concentrated under reduced pressure and poured into ice water. The precipitated solids were collected by filtration, washed with water (3×10 mL), and then dried in vacuum to afford ethyl 6-chloro-8-((methyl-d₃)amino)imidazo[1,2-b]pyridazine-3-carboxylate (II-1, 3.50 g, 70%) as a yellow solid. LC-MS (ESI): m/z 258.1 [M+H]⁺.

To a mixture of ethyl 6-chloro-8-((methyl-d₃)amino)imidazo[1,2-b]pyridazine-3-carboxylate (II-1, 3.50 g, 13.6 mmol) and cesium carbonate (6.64 g, 20.4 mmol) in N,N-dimethylformamide (50 mL) was added 4-methoxybenzyl chloride (3.19 g, 20.4 mmol) in portions at 0° C., and the resulting mixture was stirred at 40° C. for 1 hr. The reaction mixture was then quenched with ice water. The precipitated solids were collected by filtration, washed with water (3×10 mL), and then dried in vacuum to afford ethyl 6-chloro-8-((4-methoxybenzyl)(methyl-d₃)amino)imidazo[1,2-b]pyridazine-3-carboxylate (Intermediate II, 4.90 g, 95%) as a brown solid. LC-MS (ESI): m/z 378.1 [M+H]⁺.

### Intermediate Synthesis 3. Synthesis of ethyl 5-chloro-7-((4-methoxybenzyl)(methyl)amino)pyrazolo[1,5-a]pyrimidine-3-carboxylate (Intermediate III)

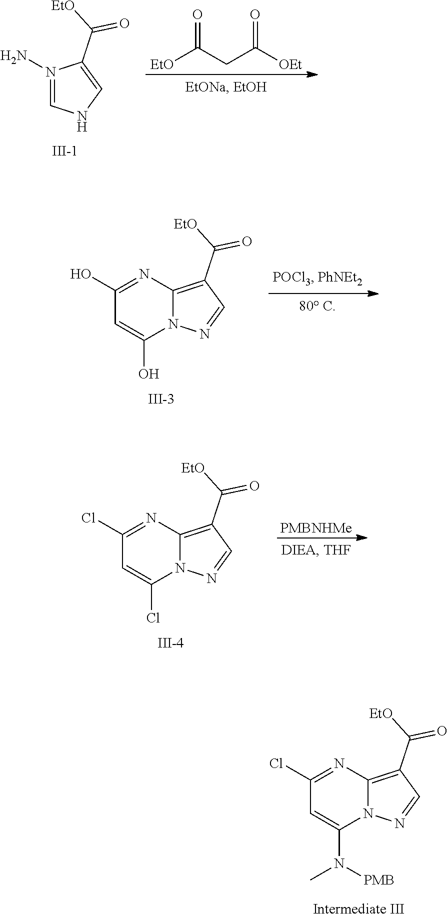

To a mixture of ethyl 3-amino-1H-pyrazole-4-carboxylate (III-1, 5.50 g, 35.4 mmol) and diethyl malonate (III-2, 11.4 g, 70.9 mmol) in ethyl alcohol (100 mL) was added sodium ethoxide (7.24 g, 106 mmol) in portions at 0° C., and the reaction mixture was stirred at 80° C. for 10 hrs. The mixture was concentrated under reduced pressure, diluted with water (200 mL) and acidified to pH˜ 4 with 6 M aq. HCl. Then the mixture was filtered and the filter cake was washed with water (3×10 mL) and dried in vacuum to afford ethyl 5,7-dihydroxypyrazolo[1,5-a]pyrimidine-3-carboxylate (III-3, 6.50 g, crude) as a white solid. LC-MS (ESI): m/z 224.2 [M+H]⁺.

To a mixture of ethyl 5,7-dihydroxypyrazolo[1,5-a]pyrimidine-3-carboxylate (III-3, 2.00 g, crude) and N,N-diethylaniline (2 mL) was added phosphorus oxychloride (9 mL) at 0° C., and the reaction mixture was stirred at 80° C. for 2 hrs. The mixture was cooled to room temperature, poured into ice water and adjusted to pH 7 with saturated solution of sodium bicarbonate. The precipitated solids were collected by filtration, washed with water (3×10 mL), and then dried in vacuum to afford ethyl 5,7-dichloropyrazolo[1,5-a]pyrimidine-3-carboxylate (III-4, 2.30 g, crude) as an off-white solid. LC-MS (ESI): m/z 260.2 [M+H]*.

To a mixture of ethyl 5,7-dichloropyrazolo[1,5-a]pyrimidine-3-carboxylate (III-4, 19.0 g, 73.4 mmol) and N,N-diisopropylethylamine (18.9 g, 147 mmol) in tetrahydrofuran (200 mL) was added 1-(4-methoxyphenyl)-N-methylmethanamine (16.6 g, 110 mmol) at room temperature, and the resulting mixture was stirred at 70° C. overnight. The reaction mixture was concentrated under reduced pressure. The crude product was recrystallized from ethyl acetate/petroleum ether=1/10. The solids were collected by filtration, washed with petroleum ether (3×10 mL), and then dried in vacuum to afford ethyl 5-chloro-7-((4-methoxybenzyl)(methyl)amino)pyrazolo[1,5-a]pyrimidine-3-carboxylate (Intermediate III, 15.0 g, crude) as a yellow solid. LC-MS (ESI): m/z 375.2 [M+H]⁺.

### Intermediate Synthesis 4. Synthesis of ethyl 5-chloro-7-((4-methoxybenzyl)(methyl-d₃)amino)pyrazolo[1,5-a]pyrimidine-3-carboxylate (Intermediate IV)

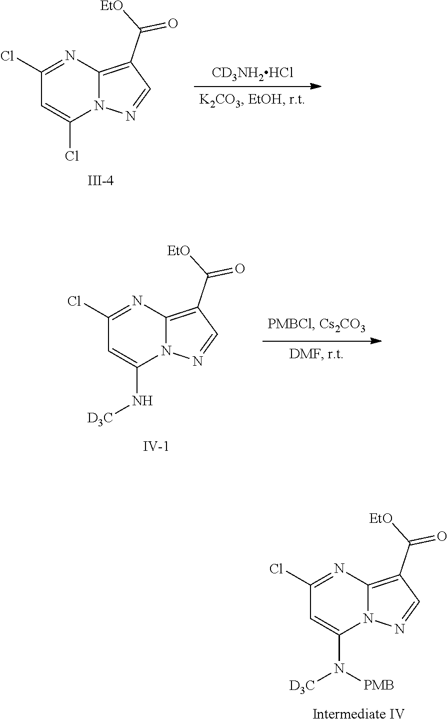

To a solution of ethyl 5,7-dichloropyrazolo[1,5-a]pyrimidine-3-carboxylate (III-4, 2.00 g, 7.69 mmol) in ethyl alcohol (40 mL) were added potassium carbonate (1.59 g, 11.5 mmol) and methyl-d₃-amine monohydrochloride (570 mg, 8.08 mmol) in portions at 0° C., and the resulting mixture was stirred at room temperature overnight. The reaction mixture was quenched with ice water. The precipitated solids were collected by filtration, washed with water (2×10 mL), and then dried in vacuum to afford ethyl 5-chloro-7-((methyl-d₃)amino)pyrazolo[1,5-a]pyrimidine-3-carboxylate (IV-1, 1.90 g, crude) as a white solid. LC-MS (ESI): m/z 258.1 [M+H]⁺.

To a mixture of ethyl 5-chloro-7-((methyl-d₃)amino)pyrazolo[1,5-a]pyrimidine-3-carboxylate (IV-1, 500 mg, crude) and cesium carbonate (1.26 g, 3.88 mmol) in N,N-dimethylformamide (20 mL) was added 4-methoxybenzyl chloride (456 mg, 2.91 mmol) at room temperature. The resulting mixture was stirred at room temperature for 3 hrs. The reaction mixture was then diluted with water at 0° C. and extracted with ethyl acetate (3×20 mL). The combined organic layers were dried over anhydrous sodium sulfate, filtered and concentrated under reduced pressure. The residue was subjected to silica gel column chromatography to afford ethyl 5-chloro-7-((4-methoxybenzyl)(methyl-d₃)amino)pyrazolo[1,5-a]pyrimidine-3-carboxylate (Intermediate IV, 530 mg) as a white solid. LC-MS (ESI): m/z 378.2 [M+H]⁺.

### Intermediate Synthesis 5. Synthesis of (1R,5S,7R)-2-oxabicyclo[3.2.0]heptan-7-amine hydrochloride (Intermediate V)

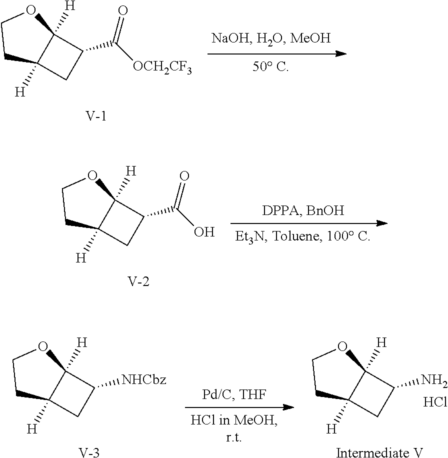

To a solution of 2,2,2-trifluoroethyl (1R,5S,7R)-2-oxabicyclo[3.2.0]heptane-7-carboxylate (V-1, 1.00 g, 4.50 mmol) in methanol (10 mL) and water (5 mL) was added sodium hydroxide (540 mg, 13.0 mmol), and the resulting mixture was stirred at 50° C. for 1 hr under nitrogen atmosphere. The reaction mixture was adjusted to pH 3-4 with 6 M aq. HCl, and then extracted with ethyl acetate (3×20 mL). The organic layer was dried over anhydrous sodium sulfate and concentrated under reduced pressure to afford (1R,5S,7R)-2-oxabicyclo[3.2.0]heptane-7-carboxylic acid (V-2, 650 mg, crude) as a colorless oil. LC-MS (ESI): m/z 143.1 [M+H]⁺. (Compound V-1 was synthesized according to Journal of the American Chemical Society. 2007, 129 (42), 12686-12687.)

To a solution of (1R,5S,7R)-2-oxabicyclo[3.2.0]heptane-7-carboxylic acid (V-2, 400 mg, 2.80 mmol) in toluene (2 mL) were added triethylamine (566 mg, 5.60 mmol) and diphenyl azidophosphate (930 mg, 3.40 mmol), and the resulting mixture was stirred at room temperature for 1 hr under nitrogen atmosphere. Then to the mixture was added 2-methypropan-2-ol (108 mg, 2.80 mmol) and the resulting mixture was stirred at 100° C. for additional 30 mins under nitrogen atmosphere. The reaction mixture was concentrated under reduced pressure. The residue was subjected to reverse phase flash chromatography to afford benzyl ((1R,5S,7R)-2-oxabicyclo[3.2.0]heptan-7-yl)carbamate (V-3, 160 mg, 23%) as a colorless oil. LC-MS (ESI): m/z 248.1 [M+H]⁺.

To a solution of benzyl ((1R,5S,7R)-2-oxabicyclo[3.2.0]heptan-7-yl)carbamate (V-3, 3.60 g, 14.6 mmol) in ethyl acetate (20 mL) was added palladium (10% on carbon, 2.00 g). The mixture was stirred at room temperature for 3 hrs under hydrogen atmosphere (1 atm). The reaction mixture was filtered and then to the filtrate was added HCl (100 mL, 4 M in methanol). The resulting mixture was concentrated under reduced pressure to afford (1R,5S,7R)-2-oxabicyclo[3.2.0]heptan-7-amine hydrochloride (Intermediate V, 2.60 g, crude) as an orange oil. LC-MS (ESI): m/z 114.1 [M+H].

### Synthetic Example 1. Synthesis of N-((1R,5S,7R)-2-oxabicyclo[3.2.0]heptan-7-yl)-6-((3′-fluoro-2-oxo-2H-[1,2′-bipyridin]-3-yl)amino)-8-(methylamino)imidazo[1,2-b]pyridazine-3-carboxamide (1)

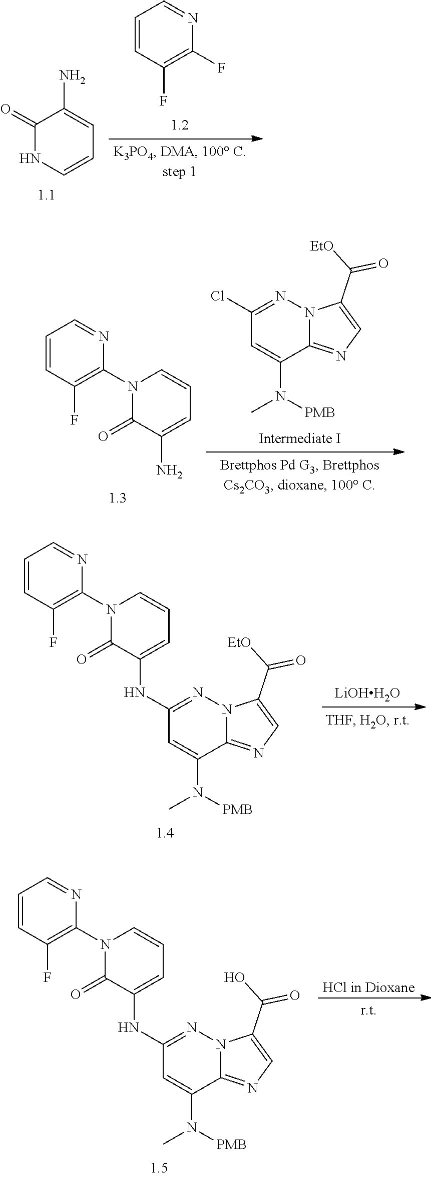

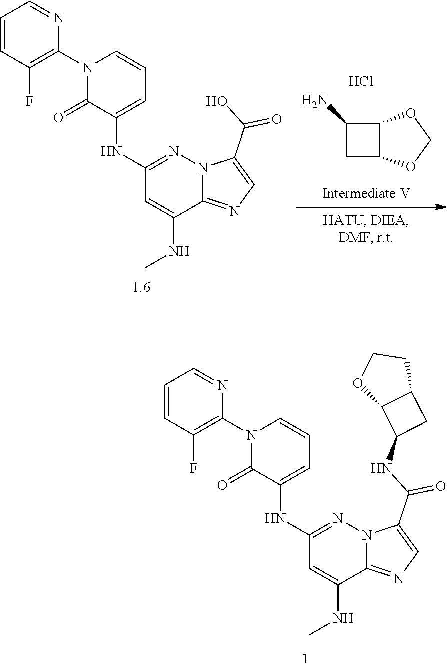 [SMILES: CNc1cc(Nc2cccn(-c3ncccc3F)c2=O)nn2c(C(=O)O)cnc12 ; N[C@@H]1C[C@H]2OCO[C@@H]12 ; Cl ; CNc1cc(Nc2cccn(-c3ncccc3F)c2=O)nn2c(C(=O)N[C@@H]3C[C@H]4CCO[C@H]43)cnc12]

To a solution of 2,3-difluoropyridine (1.2, 1.00 g, 8.69 mmol) in N,N-dimethylacetamide (30 mL) were added 3-aminopyridin-2(1H)-one (1.1, 1.15 g, 10.4 mmol) and potassium phosphate (5.53 g, 26.0 mmol), and the resulting mixture was stirred at 100° C. overnight. The reaction mixture was cooled to room temperature and filtered. The filter cake was washed with dichloromethane (3×100 mL) and the filtrate was washed with water (3×20 mL). Then the organic layer was dried over anhydrous sodium sulfate, filtered and concentrated under reduced pressure. The residue was subjected to silica gel column chromatography to afford 3-amino-3′-fluoro-2H-[1,2′-bipyridin]-2-one (1.3, 900 mg, 50%) as a yellow solid. LC-MS (ESI): m/z 206.1 [M+H]⁺.

### Procedure for Coupling (Procedure A):

To a solution of ethyl 6-chloro-8-((4-methoxybenzyl)(methyl)amino)imidazo[1,2-b]pyridazine-3-carboxylate (Intermediate I, 300 mg, 0.80 mmol) in dioxane (15 mL) were added 3-amino-3′-fluoro-2H-[1,2′-bipyridin]-2-one (1.3, 197 mg, 0.96 mmol), BrettPhos Pd G3 (73.0 mg, 0.08 mmol), BrettPhos (86.0 mg, 0.16 mmol) and cesium carbonate (522 mg, 1.60 mmol), and the resulting mixture was stirred at 100° C. for 2 hrs under nitrogen atmosphere. The mixture was cooled to room temperature and filtered. The filter cake was washed with ethyl acetate (3×10 mL). The filtrate was washed with water (3×10 mL), dried over anhydrous sodium sulfate, filtered and concentrated under reduced pressure. The residue was subjected to silica gel column chromatography to afford ethyl 6-((3′-fluoro-2-oxo-2H-[1,2′-bipyridin]-3-yl)amino)-8-((4-methoxybenzyl)(methyl)amino)imidazo[1,2-b]pyridazine-3-carboxylate (1.4, 320 mg, 73%) as a green oil. LC-MS (ESI): m/z 544.3 [M+H]⁺.

### Procedure for Ester Hydrolysis (Procedure B):

To a suspension of ethyl 6-((3′-fluoro-2-oxo-2H-[1,2′-bipyridin]-3-yl)amino)-8-((4-methoxybenzyl)(methyl)amino)imidazo[1,2-b]pyridazine-3-carboxylate (1.4, 320 mg, 0.59 mmol) in water (6 mL) and tetrahydrofuran (12 mL) was added lithium hydroxide monohydrate (247 mg, 5.89 mmol), and the resulting mixture was stirred at room temperature overnight. The mixture was adjusted to pH 7 with 1 M aq. HCl and then filtered. The filter cake was washed with water (3×5 mL), and then subjected to silica gel column chromatography to afford 6-((3′-fluoro-2-oxo-2H-[1,2′-bipyridin]-3-yl)amino)-8-((4-methoxybenzyl)(methyl)amino)imidazo[1,2-b]pyridazine-3-carboxylic acid (1.5, 166 mg, 54%) as a yellow solid. LC-MS (ESI): m/z 516.3 [M+H]⁺.

Procedure for PMB-Deprotection (procedure C):

A mixture of 6-((3′-fluoro-2-oxo-2H-[1,2′-bipyridin]-3-yl)amino)-8-((4-methoxybenzyl)(methyl)amino)imidazo[1,2-b]pyridazine-3-carboxylic acid (1.5, 156 mg, 0.30 mmol) and hydrochloric acid (3 mL, 4 M in dioxane) was stirred at room temperature for 1 hr. The reaction mixture was concentrated under reduced pressure to afford 6-((3′-fluoro-2-oxo-2H-[1,2′-bipyridin]-3-yl)amino)-8-(methylamino)imidazo[1,2-b]pyridazine-3-carboxylic acid (1.6, 117 mg, crude) as a red solid. LC-MS (ESI): m/z 396.1 [M+H]⁺.

To a mixture of 6-((3′-fluoro-2-oxo-2H-[1,2′-bipyridin]-3-yl)amino)-8-(methylamino)imidazo[1,2-b]pyridazine-3-carboxylic acid (1.6, 78.0 mg, crude), 2-(7-azabenzotriazol-1-yl)-N,N,N′,N′-tetramethyluronium hexafluorophosphate (110 mg, 0.29 mmol) and N,N-diisopropylethylamine (77.0 mg, 0.59 mmol) in N,N-dimethylformamide (2 mL) was added (1R,5S,7R)-2-oxabicyclo[3.2.0]heptan-7-amine hydrochloride (Intermediate V, 35.9 mg, 0.24 mmol), and the resulting mixture was stirred at room temperature overnight. The resulting mixture was diluted with dichloromethane (20 mL) and washed with water (5×15 mL). The organic layer was dried over anhydrous sodium sulfate, filtered and concentrated under reduced pressure. The residue was subjected to silica gel column chromatography to obtain the crude product (50.0 mg), which was further purified by Prep-HPLC to afford N-((1R,5S,7R)-2-oxabicyclo[3.2.0]heptan-7-yl)-6-((3′-fluoro-2-oxo-2H-[1,2′-bipyridin]-3-yl)amino)-8-(methylamino)imidazo[1,2-b]pyridazine-3-carboxamide (1, 42.4 mg, 43%, ee=97%) as a white solid. LC-MS (ESI): m/z 491.1 [M+H]¹H NMR (400 MHz, CDCl₃) δ 8.78 (d, J=6.0 Hz, 1H), 8.44 (d, J=4.4 Hz, 1H), 8.09 (s, 1H), 8.05 (d, J=7.2 Hz, 1H), 7.74 (s, 1H), 7.71-7.69 (m, 1H), 7.56-7.47 (m, 1H), 7.16 (d, J=6.4 Hz, 1H), 6.50 (t, J=7.2 Hz, 1H), 6.12 (q, J=5.2 Hz, 1H), 5.69 (s, 1H), 4.67-4.58 (m, 1H), 4.49-4.39 (m, 1H), 4.24-4.15 (m, 1H), 4.12-4.01 (m, 1H), 3.04 (d, J=5.2 Hz, 4H), 2.20-2.06 (m, 2H), 2.05-1.89 (m, 2H).

### Synthetic Example 2. Synthesis of N-((1R,5S,7R)-2-oxabicyclo[3.2.0]heptan-7-yl)-8-(methylamino)-6-((2-oxo-2H-[1,2′-bipyridin]-3-yl)amino)imidazo[1,2-b]pyridazine-3-carboxamide (2)

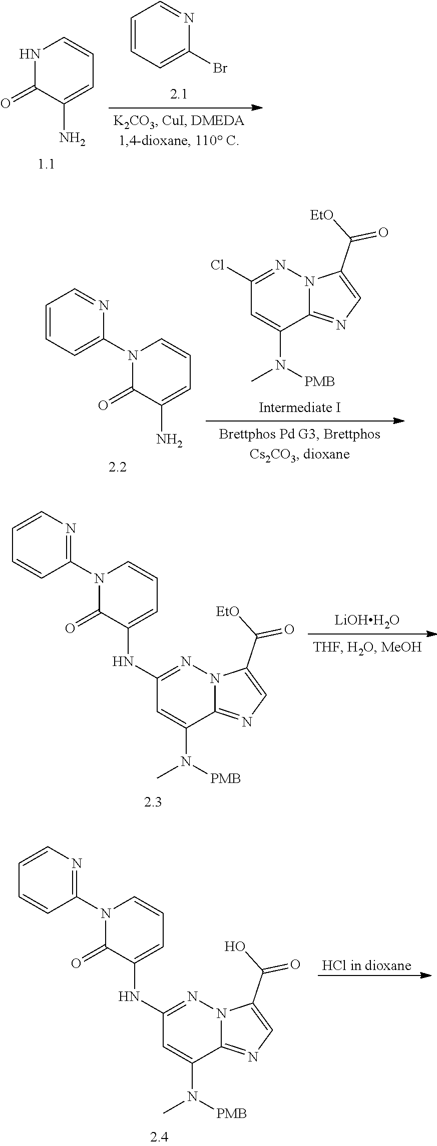

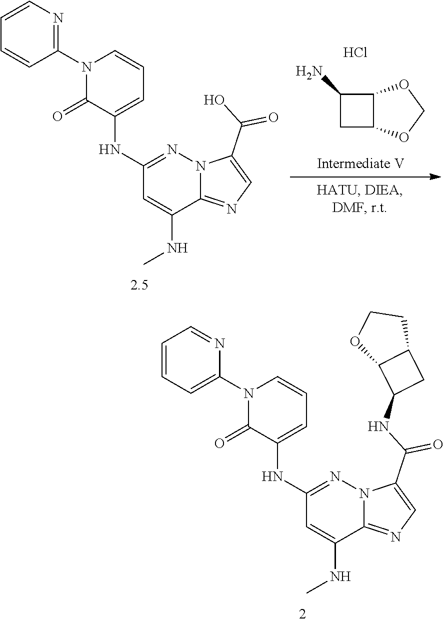 [SMILES: CNc1cc(Nc2cccn(-c3ccccn3)c2=O)nn2c(C(=O)O)cnc12 ; N[C@@H]1C[C@H]2OCO[C@@H]12 ; Cl ; CNc1cc(Nc2cccn(-c3ccccn3)c2=O)nn2c(C(=O)N[C@@H]3C[C@H]4CCO[C@H]43)cnc12]

To a solution of 3-aminopyridin-2(1H)-one (1.1, 3.00 g, 27.2 mmol) in 1,4-dioxane (80 mL) were added 2-bromopyridine (2.1, 5.17 g, 32.7 mmol), N,N-dimethyl-1,2-ethanediamine (960 mg, 10.9 mmol), potassium carbonate (7.53 g, 54.5 mmol) and copper iodide (1.04 g, 5.45 mmol), and the mixture was stirred at 110° C. overnight under nitrogen atmosphere. The mixture was cooled to room temperature and diluted with water (80 mL), and the aqueous layer was extracted with ethyl acetate (3×100 mL). The combined organic layers were dried over anhydrous sodium sulfate, filtered and concentrated under reduced pressure. The residue was subjected to silica gel column chromatography to afford 3-amino-2H-[1,2′-bipyridin]-2-one (2.2, 2.20 g, 43%) as a light red solid. LC-MS (ESI): m/z 188.2 [M+H]⁺.

Compound 2.3 was synthesized from 2.2 similarly to procedure A to afford ethyl 8-((4-methoxybenzyl)(methyl)amino)-6-((2-oxo-2H-[1,2′-bipyridin]-3-yl)amino)imidazo[1,2-b]pyridazine-3-carboxylate (2.3) as a green solid. LC-MS (ESI): m/z 526.15 [M+H]⁺.

Compound 2.4 was synthesized from 2.3 similarly to procedure B to afford 8-((4-methoxybenzyl)(methyl)amino)-6-((2-oxo-2H-[1,2′-bipyridin]-3-yl)amino)imidazo[1,2-b]pyridazine-3-carboxylic acid (2.4) as a white solid. LC-MS (ESI): m/z 498.25 [M+H]⁺.

Compound 2.5 was synthesized from 2.4 similarly to procedure C to afford 8-(methylamino)-6-((2-oxo-2H-[1,2′-bipyridin]-3-yl)amino)imidazo[1,2-b]pyridazine-3-carboxylic acid (2.5) as a white solid. LC-MS (ESI): m/z 378.16 [M+H]⁺.

To a mixture of 8-(methylamino)-6-((2-oxo-2H-[1,2′-bipyridin]-3-yl)amino)imidazo[1,2-b]pyridazine-3-carboxylic acid (2.5, 38.0 mg, crude) and (1R,5S,7R)-2-oxabicyclo[3.2.0]heptan-7-amine hydrochloride (Intermediate V, 15.0 mg, 0.10 mmol, crude) in N,N-dimethylformamide (2 mL) were added 2-(7-azabenzotriazol-1-yl)-N,N,N′,N′-tetramethyluronium hexafluorophosphate (45.0 mg, 0.12 mmol) and N,N-diisopropylethylamine (39.0 mg, 0.30 mmol). The mixture was stirred at room temperature for 1 hr. The mixture was filtered and subjected to Prep-HPLC to afford N-((1R,5S,7R)-2-oxabicyclo[3.2.0]heptan-7-yl)-8-(methylamino)-6-((2-oxo-2H-[1,2′-bipyridin]-3-yl)amino)imidazo[1,2-b]pyridazine-3-carboxamide (2, 16.9 mg, 35%, ee>99%) as an off-white solid. LC-MS (ESI): m/z 473.40 [M+H]⁺. ¹H NMR (300 MHz, CDCl₃) δ 8.79 (d, J=6.9 Hz, 1H), 8.63 (d, J=4.8 Hz, 1H), 8.09 (s, 1H), 8.07-7.98 (m, 1H), 7.98-7.85 (m, 2H), 7.80 (s, 1H), 7.55 (dd, J=7.2, 1.7 Hz, 1H), 7.44-7.34 (m, 1H), 6.48 (t, J=7.2 Hz, 1H), 6.23-6.08 (m, 1H), 5.70 (s, 1H), 4.68-4.59 (m, 1H), 4.49-4.40 (m, 1H), 4.25-4.14 (m, 1H), 4.14-3.99 (m, 1H), 3.05 (d, J=5.1 Hz, 4H), 2.17-1.94 (m, 4H).

### Synthetic Example 3. Synthesis of N-((1R,5S,7R)-2-oxabicyclo[3.2.0]heptan-7-yl)-6-((1-(1-methyl-1H-pyrazol-3-yl)-2-oxo-1,2-dihydropyridin-3-yl)amino)-8-(methylamino)imidazo[1,2-b]pyridazine-3-carboxamide (3a) and N-((1S,5R,7S)-2-oxabicyclo[3.2.0]heptan-7-yl)-6-((1-(1-methyl-1H-pyrazol-3-yl)-2-oxo-1,2-dihydropyridin-3-yl)amino)-8-(methylamino)imidazo[1,2-b]pyridazine-3-carboxamide (3b)

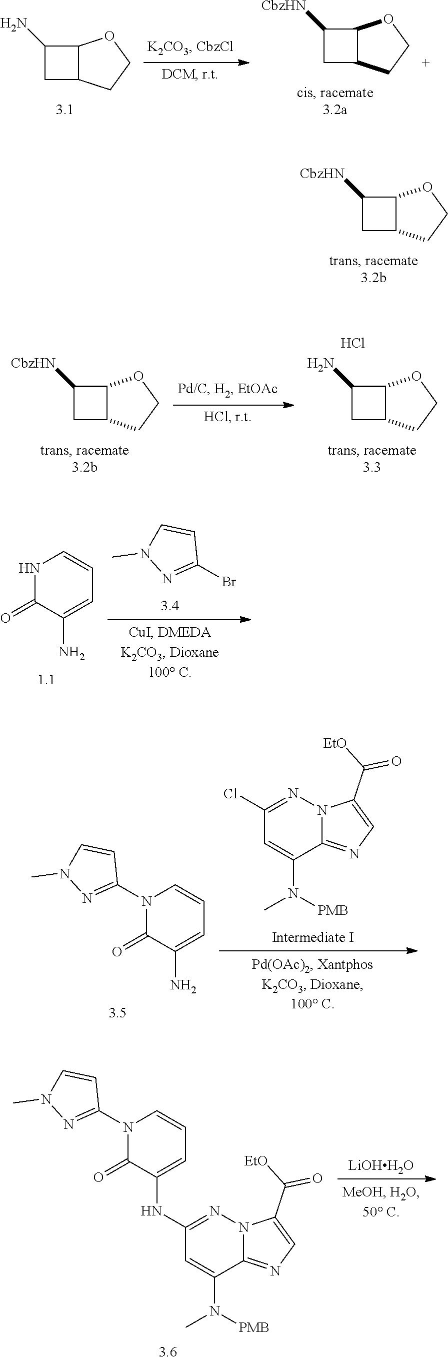

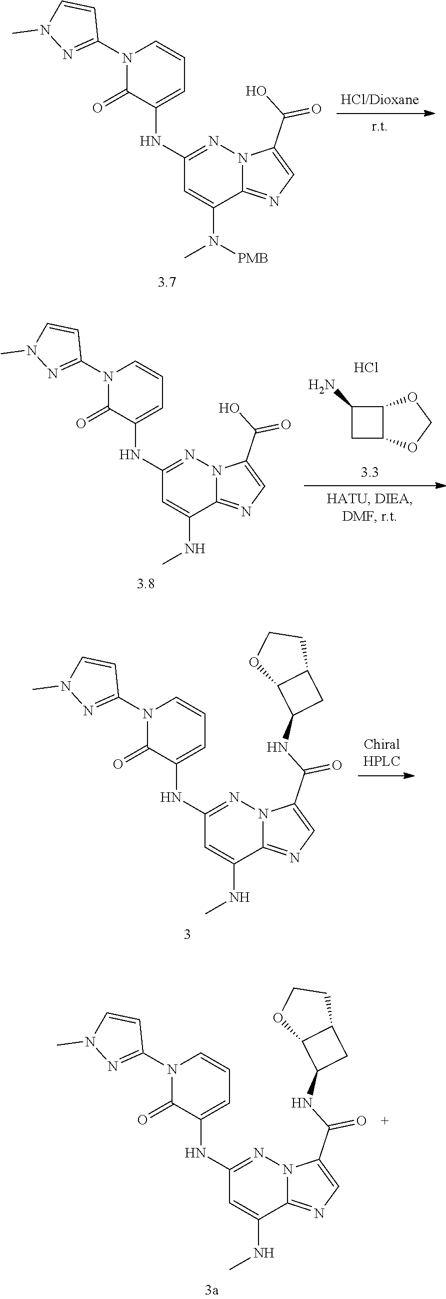

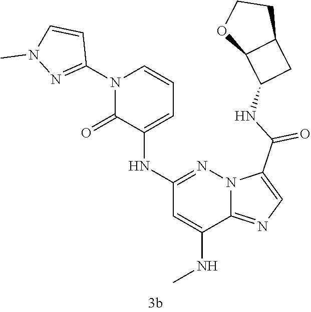 [SMILES: CNc1cc(Nc2cccn(-c3ccn(C)n3)c2=O)nn2c(C(=O)N[C@H]3C[C@@H]4CCO[C@@H]43)cnc12]

To a mixture of 2-oxabicyclo[3.2.0]heptan-7-amine (3.1, 300 mg, 2.65 mmol) and potassium carbonate (733 mg, 5.30 mmol) in dichloromethane (10 mL) was added benzyl carbonochloridate (543 mg, 3.18 mmol), and the resulting mixture was stirred at room temperature overnight. The reaction mixture was diluted with water (20 mL) and extracted with dichloromethane (3×15 mL). The combined organic layers were dried over anhydrous sodium sulfate, filtered and concentrated under reduced pressure. The residue was subjected to silica gel column chromatography to afford trans-benzyl (2-oxabicyclo[3.2.0]heptan-7-yl)carbamate (3.2b, 347 mg, 52%) as a white solid. LC-MS (ESI): m/z 248.05 [M+H]⁺.

To a solution of trans-benzyl (2-oxabicyclo[3.2.0]heptan-7-yl)carbamate (3.2b, 300 mg, 1.21 mmol) in ethyl acetate (12 mL) was added palladium (10% on carbon, 60 mg) and the mixture was degassed and backfilled with hydrogen atmosphere and stirred at room temperature for 1 hr (1 atm). The reaction mixture was filtered and washed with methanol (3×5 mL). The filtrate was treated with hydrochloric acid (20 mL, 6 M in methanol) and stirred at room temperature for additional 30 mins. Then the mixture was concentrated under reduced pressure to afford trans-2-oxabicyclo[3.2.0]heptan-7-amine hydrochloride (3.3, 152 mg, crude) as a colorless oil. LC-MS (ESI): m/z 114.15 [M+H]⁺.

To a mixture of 3-aminopyridin-2(1H)-one (1.1, 1.00 g, 9.08 mmol) and 3-bromo-1-methyl-1H-pyrazole (3.4, 1.61 g, 9.99 mmol) in dioxane (20 mL) were added copper iodide (259 mg, 1.36 mmol), N,N-dimethyl-1,2-ethanediamine (240 mg, 2.72 mmol) and potassium carbonate (2.51 g, 18.1 mmol), and the resulting mixture was stirred at 110° C. for 2 hrs under nitrogen atmosphere. The mixture was diluted with water (30 mL) and extracted with ethyl acetate (3×15 mL). The combined layers were dried over anhydrous sodium sulfate, filtered and concentrated under reduced pressure. The residue was subjected to silica gel column chromatography to afford 3-amino-1-(1-methyl-1H-pyrazol-3-yl)pyridin-2(1H)-one (3.5, 550 mg, 31%) as a brown oil. LC-MS (ESI): m/z 191.1 [M+H]⁺.

Compound 3.6 was synthesized from 3.5 similarly to procedure A to afford ethyl 8-((4-methoxybenzyl)(methyl)amino)-6-((1-(1-methyl-1H-pyrazol-3-yl)-2-oxo-1,2-dihydropyridin-3-yl)amino)imidazo[1,2-b]pyridazine-3-carboxylate (3.6) as a brown solid. LC-MS (ESI): m/z 529.3 [M+H]⁺.

Compound 3.7 was synthesized from 3.6 similarly to procedure B to afford 8-((4-methoxybenzyl)(methyl)amino)-6-((1-(1-methyl-1H-pyrazol-3-yl)-2-oxo-1,2-dihydropyridin-3-yl)amino)imidazo[1,2-b]pyridazine-3-carboxylic acid (3.7) as a grey solid. LC-MS (ESI): m/z 501.2 [M+H]⁺.

Compound 3.8 was synthesized from 3.7 similarly to procedure C to afford 6-((1-(1-methyl-1H-pyrazol-3-yl)-2-oxo-1,2-dihydropyridin-3-yl)amino)-8-(methylamino)imidazo[1,2-b]pyridazine-3-carboxylic acid (3.8) as a brown solid. LC-MS (ESI): m/z 381.1 [M+H⁺].

To a mixture of 6-((1-(1-methyl-1H-pyrazol-3-yl)-2-oxo-1,2-dihydropyridin-3-yl)amino)-8-(methylamino)imidazo[1,2-b]pyridazine-3-carboxylic acid (3.8, 77.0 mg, crude) and trans-2-oxabicyclo[3.2.0]heptan-7-amine hydrochloride (3.3, 36.0 mg, crude) in N,N-dimethylformamide (3 mL) were added N,N-diisopropylethylamine (52.0 mg, 0.40 mmol) and 2-(7-azabenzotriazol-1-yl)-N,N,N′,N′-tetramethyluronium hexafluorophosphate (153 mg, 0.40 mmol), and the resulting mixture was stirred at room temperature overnight. The mixture was filtered and the filtrate was subjected to Prep-HPLC to afford trans-N-(2-oxabicyclo[3.2.0]heptan-7-yl)-6-((1-(1-methyl-1H-pyrazol-3-yl)-2-oxo-1,2-dihydropyridin-3-yl)amino)-8-(methylamino)imidazo[1,2-b]pyridazine-3-carboxamide (3, 34.0 mg). The racemate was separated by chiral HPLC to afford N-((1R,5S,7R)-2-oxabicyclo[3.2.0]heptan-7-yl)-6-((1-(1-methyl-1H-pyrazol-3-yl)-2-oxo-1,2-dihydropyridin-3-yl)amino)-8-(methylamino)imidazo[1,2-b]pyridazine-3-carboxamide (3a, 11.8 mg, ee>99%) and N-((1S,5R,7S)-2-oxabicyclo[3.2.0]heptan-7-yl)-6-((1-(1-methyl-1H-pyrazol-3-yl)-2-oxo-1,2-dihydropyridin-3-yl)amino)-8-(methylamino)imidazo[1,2-b]pyridazine-3-carboxamide (3b, 10.1 mg, ee>95%), both as a white solid.

3a: Chiral HPLC Retention time: 12.05 min. LC-MS (ESI): m/z 476.2 [M+H]⁺. ¹H NMR (400 MHz, CDCl₃) δ 8.72 (d, J=7.4 Hz, 1H), 8.00 (s, 1H), 7.89 (d, J=7.2 Hz, 1H), 7.75 (s, 1H), 7.54 (d, J=7.0 Hz, 1H), 7.36 (s, 1H), 6.77 (s, 1H), 6.41-6.27 (m, 1H), 6.07 (s, 1H), 5.61 (s, 1H), 4.60-4.44 (m, 1H), 4.43-4.28 (m, 1H), 4.18-4.05 (m, 1H), 4.04-3.93 (m, 1H), 3.90 (s, 3H), 3.03-2.85 (m, 4H), 2.15-1.82 (m, 4H).

3b: Chiral HPLC Retention time: 13.85 min. LC-MS (ESI): m/z 476.2 [M+H]⁺. ¹H NMR (400 MHz, CDCl₃) δ 8.72 (s, 1H), 7.99 (d, J=7.5 Hz, 1H), 7.89 (d, J=7.7 Hz, 1H), 7.76 (s, 1H), 7.54 (d, J=7.5 Hz, 1H), 7.36 (s, 1H), 6.77 (s, 1H), 6.37-6.29 (m, 1H), 6.14 (s, 1H), 5.60 (d, J=5.4 Hz, 1H), 4.60-4.46 (m, 1H), 4.44-4.28 (m, 1H), 4.18-4.05 (m, 1H), 4.04-3.72 (m, 4H), 3.08-2.74 (m, 4H), 2.15-1.76 (m, 4H).

Prep-HPLC method: column: xbridge prep C18 OBD column, 19*150 mm, 5 m; mobile phase A: water (10 mmol/L NH₄HCO₃), mobile phase B: CH₃CN; flow rate: 25 mL/min; gradient: 21% B to 40% B in 7 min, 40% B; wave length: 220 nm.

Chiral HPLC Method: column: chiral art cellulose-SB, 2*25 cm, 5 m; mobile phase A: Hex:MtBE=1:1(0.5% 2 M NH₃-MeOH), mobile phase B: MeOH-HPLC; flow rate: 20 mL/min; gradient: 50% B to 50% B in 24 min; wave length: 252/214 nm; sample solvent: DCM-HPLC; injection volume: 0.50 mL. To be clear, the solvent followed by “—HPLC” as used herein should be understood that the solvent is of HPLC grade.

### Synthetic Example 4. Asymmetric synthesis of N-((1R,5S,7R)-2-oxabicyclo[3.2.0]heptan-7-yl)-6-((1-(1-methyl-1H-pyrazol-3-yl)-2-oxo-1,2-dihydropyridin-3-yl)amino)-8-(methylamino)imidazo[1,2-b]pyridazine-3-carboxamide (3a)

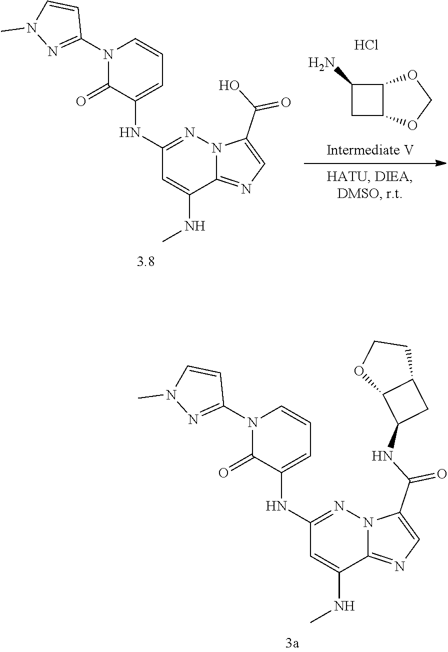 [SMILES: CNc1cc(Nc2cccn(-c3ccn(C)n3)c2=O)nn2c(C(=O)O)cnc12 ; N[C@@H]1C[C@H]2OCO[C@@H]12 ; Cl ; CNc1cc(Nc2cccn(-c3ccn(C)n3)c2=O)nn2c(C(=O)N[C@@H]3C[C@H]4CCO[C@H]43)cnc12]

To a mixture of 6-((1-(1-methyl-1H-pyrazol-3-yl)-2-oxo-1,2-dihydropyridin-3-yl)amino)-8-(methylamino)imidazo[1,2-b]pyridazine-3-carboxylic acid (3.8, 60.0 mg, 0.16 mmol) and (1R,5S,7R)-2-oxabicyclo[3.2.0]heptan-7-amine hydrochloride (Intermediate V, 28.7 mg, crude) in dimethylsulfoxide (2 mL) were added 2-(7-azabenzotriazol-1-yl)-N,N,N′,N′-tetramethyluronium hexafluorophosphate (91.2 mg, 0.24 mmol) and N,N-diisopropylethylamine (62.0 mg, 0.48 mmol), and the resulting mixture was stirred at room temperature for 1 hr. The mixture was filtered and the filtrate was subjected to Prep-HPLC to afford N-((1R,5S,7R)-2-oxabicyclo[3.2.0]heptan-7-yl)-6-((1-(1-methyl-1H-pyrazol-3-yl)-2-oxo-1,2-dihydropyridin-3-yl)amino)-8-(methylamino)imidazo[1,2-b]pyridazine-3-carboxamide (3a, 41.0 mg, ee>99%) as a white solid. LC-MS (ESI): m/z 476.2 [M+H]⁺.

### Synthetic Example 5. Synthesis of N-((1R,5S,7R)-2-oxabicyclo[3.2.0]heptan-7-yl)-8-((methyl-d₃)amino)-6-((2-oxo-2H-[1,2′-bipyridin]-3-yl)amino)imidazo[1,2-b]pyridazine-3-carboxamide (4a) and N-((1S,5R,7S)-2-oxabicyclo[3.2.0]heptan-7-yl)-8-((methyl-d₃)amino)-6-((2-oxo-2H-[1,2′-bipyridin]-3-yl)amino)imidazo[1,2-b]pyridazine-3-carboxamide (4b)

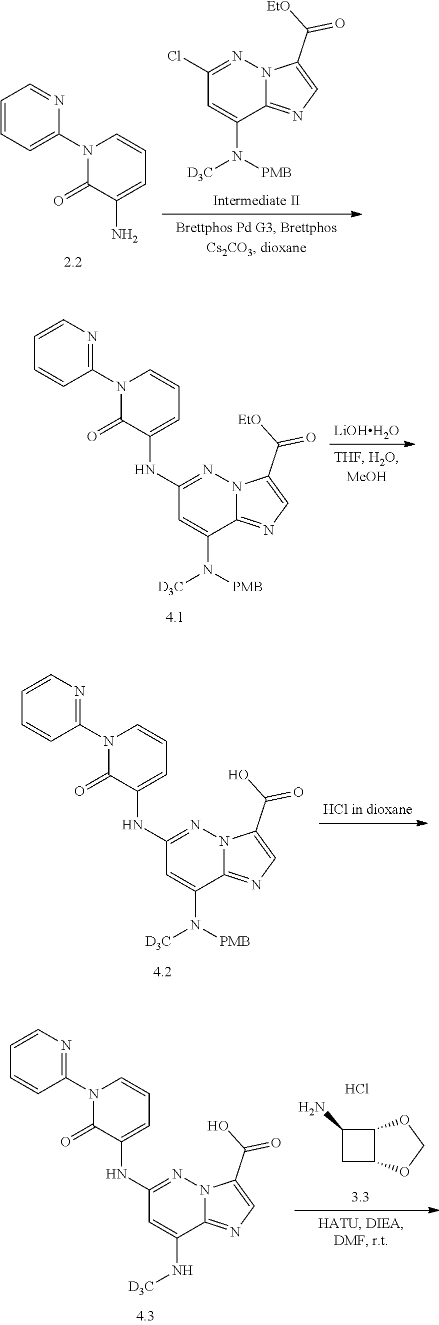

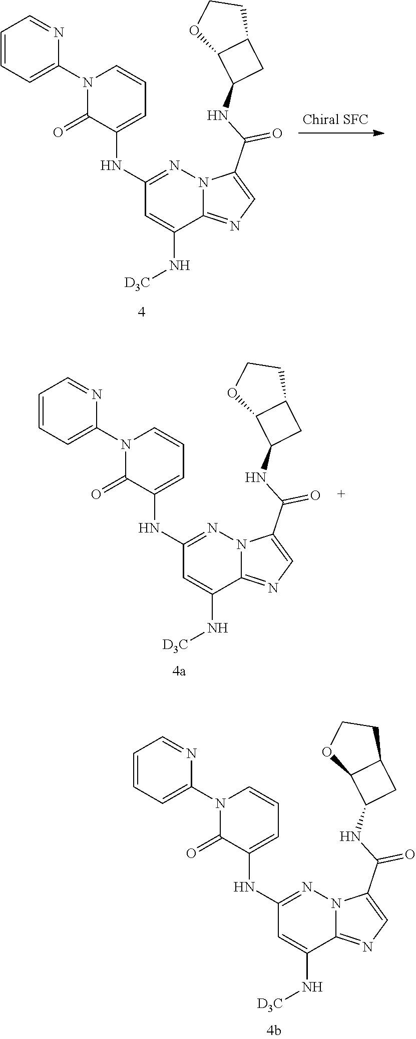 [SMILES: [2H]C([2H])([2H])Nc1cc(Nc2cccn(-c3ccccn3)c2=O)nn2c(C(=O)N[C@@H]3C[C@H]4CCO[C@H]43)cnc12 ; [2H]C([2H])([2H])Nc1cc(Nc2cccn(-c3ccccn3)c2=O)nn2c(C(=O)N[C@@H]3C[C@H]4CCO[C@H]43)cnc12 ; [2H]C([2H])([2H])Nc1cc(Nc2cccn(-c3ccccn3)c2=O)nn2c(C(=O)N[C@H]3C[C@@H]4CCO[C@@H]43)cnc12]

Compound 4.1 was synthesized from Intermediate II similarly to procedure A to afford ethyl 8-((4-methoxybenzyl)(methyl-d₃)amino)-6-((2-oxo-2H-[1,2′-bipyridin]-3-yl)amino)imidazo[1,2-b]pyridazine-3-carboxylate (4.1) as a yellow solid. LC-MS (ESI): m/z 529.2 [M+H]⁺.

Compound 4.2 was synthesized from 4.1 similarly to procedure B to afford 8-((4-methoxybenzyl)(methyl-d₃)amino)-6-((2-oxo-2H-[1,2′-bipyridin]-3-yl)amino)imidazo[1,2-b]pyridazine-3-carboxylic acid (4.2) as a brown solid. LC-MS (ESI): m/z 501.2 [M+H]⁺.

Compound 4.3 was synthesized from 4.2 similarly to procedure C to afford 8-((methyl-d₃)amino)-6-((2-oxo-2H-[1,2′-bipyridin]-3-yl)amino)imidazo[1,2-b]pyridazine-3-carboxylic acid (4.3) as a yellow solid. LC-MS (ESI): m/z 381.1 [M+H]⁺.

To a mixture of 8-((methyl-d₃)amino)-6-((2-oxo-2H-[1,2′-bipyridin]-3-yl)amino)imidazo[1,2-b]pyridazine-3-carboxylic acid (4.3, 60.0 mg, crude), N,N-diisopropylethylamine (60.6 mg, 0.47 mmol) and 2-(7-azabenzotriazol-1-yl)-N,N,N′,N′-tetramethyluronium hexafluorophosphate (91.2 mg, 0.24 mmol) in N,N-dimethylformamide (2.50 mL) was added trans-2-oxabicyclo[3.2.0]heptan-7-amine hydrochloride (3.3, 28.4 mg, crude), and the resulting mixture was stirred at room temperature for 1 hr. The mixture was filtered and the filtrate was subjected to Prep-HPLC to afford trans-N-(2-oxabicyclo[3.2.0]heptan-7-yl)-8-((methyl-d₃)amino)-6-((2-oxo-2H-[1,2′-bipyridin]-3-yl)amino)imidazo[1,2-b]pyridazine-3-carboxamide (4, 43.6 mg) as a white solid. The racemate was separated by chiral SFC to afford N-((1R,5S,7R)-2-oxabicyclo[3.2.0]heptan-7-yl)-8-((methyl-d₃)amino)-6-((2-oxo-2H-[1,2′-bipyridin]-3-yl)amino)imidazo[1,2-b]pyridazine-3-carboxamide (4a, 13.8 mg, ee>99%) and N-((1S,5R,7S)-2-oxabicyclo[3.2.0]heptan-7-yl)-8-((methyl-d₃)amino)-6-((2-oxo-2H-[1,2′-bipyridin]-3-yl)amino)imidazo[1,2-b]pyridazine-3-carboxamide (4b, 17.1 mg, ee>99%), both as a white solid.

4a: SFC Retention time: 9.78 min. LC-MS (ESI): m/z 476.25 [M+H]⁺. ¹H NMR (300 MHz, DMSO-d₆) δ 8.79 (d, J=7.5 Hz, 1H), 8.67 (s, 1H), 8.66-8.60 (m, 1H), 8.16 (dd, J=7.3, 1.8 Hz, 1H), 8.05-8.04 (m, 1H), 7.89-7.82 (m, 2H), 7.58-7.51 (m, 2H), 7.47 (s, 1H), 6.44 (t, J=7.2 Hz, 1H), 6.39 (s, 1H), 4.49 (dd, J=6.6, 3.3 Hz, 1H), 4.36-4.23 (m, 1H), 4.13-4.02 (m, 1H), 4.01-3.88 (m, 1H), 3.01-2.87 (m, 1H), 2.07-1.63 (m, 4H).

4b: SFC Retention time: 14.05 min. LC-MS (ESI): m/z 476.25 [M+H]⁺. ¹H NMR (400 MHz, DMSO-d₆) δ 8.78 (d, J=7.5 Hz, 1H), 8.67-8.61 (m, 2H), 8.15 (dd, J=7.3, 1.7 Hz, 1H), 8.04 (td, J=7.7, 1.9 Hz, 1H), 7.91-7.81 (m, 2H), 7.59-7.50 (m, 2H), 7.44 (s, 1H), 6.44 (t, J=7.2 Hz, 1H), 6.38 (s, 1H), 4.48 (dd, J=6.7, 3.2 Hz, 1H), 4.35-4.24 (m, 1H), 4.07-4.05 (m, 1H), 4.00-3.89 (m, 1H), 2.99-2.69 (m, 1H), 2.07-1.89 (m, 2H), 1.87-1.71 (m, 2H).

Prep-HPLC method: column: ymc-actus triart C18 ExRS, 30*150 mm, 5 m; mobile phase A: water (10 mmol/L NH₄HCO₃), mobile phase B: CH₃CN; flow rate: 60 mL/min; gradient: 25% B to 49% B in 7 min, 49% B; wave length: 254 nm.

SFC Method: column: chiral art amylose-SA, 3*25 cm, 5 m; mobile phase A: CO₂, mobile phase B: MeOH:DCM=4:1; flow rate: 80 mL/min; gradient: isocratic 55% B; column temperature (° C.): 35; back pressure(bar): 100; wave length: 254 nm; sample solvent: MeOH:DCM=1:1; injection volume: 1.60 mL.

### Synthetic Example 6. Asymmetric synthesis of N-((1R,5S,7R)-2-oxabicyclo[3.2.0]heptan-7-yl)-8-((methyl-d₃)amino)-6-((2-oxo-2H-[1,2′-bipyridin]-3-yl)amino)imidazo[1,2-b]pyridazine-3-carboxamide (4a)

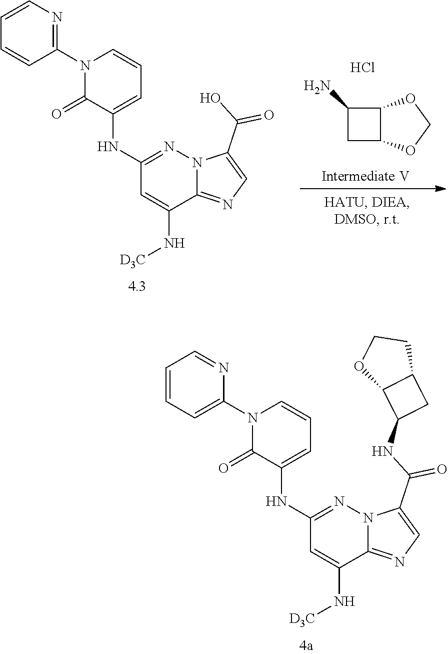 [SMILES: [2H]C([2H])([2H])Nc1cc(Nc2cccn(-c3ccccn3)c2=O)nn2c(C(=O)O)cnc12 ; N[C@@H]1C[C@H]2OCO[C@@H]12 ; Cl ; [2H]C([2H])([2H])Nc1cc(Nc2cccn(-c3ccccn3)c2=O)nn2c(C(=O)N[C@@H]3C[C@H]4CCO[C@H]43)cnc12]

To a mixture of 8-((methyl-d₃)amino)-6-((2-oxo-2H-[1,2′-bipyridin]-3-yl)amino)imidazo[1,2-b]pyridazine-3-carboxylic acid (4.3, 50.0 mg, crude), N,N-diisopropylethylamine (50.4 mg, 0.47 mmol) and 2-(7-azabenzotriazol-1-yl)-N,N,N′,N′-tetramethyluronium hexafluorophosphate (74.1 mg, 0.24 mmol) in dimethylsulfoxide (2.50 mL) was added (1R,5S,7R)-2-oxabicyclo[3.2.0]heptan-7-amine hydrochloride (Intermediate V, 23.3 mg, crude), and the resulting mixture was stirred at room temperature for 1 hr. The mixture was filtered and the filtrate was subjected to Prep-HPLC to afford N-((1R,5S,7R)-2-oxabicyclo[3.2.0]heptan-7-yl)-8-((methyl-d₃)amino)-6-((2-oxo-2H-[1,2′-bipyridin]-3-yl)amino)imidazo[1,2-b]pyridazine-3-carboxamide (4a, 34.0 mg, ee>99%) as a white solid. LC-MS (ESI): m/z 476.2 [M+H]⁺.

### Synthetic Example 7. Synthesis of N-((1R,5S,7R)-2-oxabicyclo[3.2.0]heptan-7-yl)-6-((3′-fluoro-2-oxo-2H-[1,2′-bipyridin]-3-yl)amino)-8-((methyl-d₃)amino)imidazo[1,2-b]pyridazine-3-carboxamide (5)

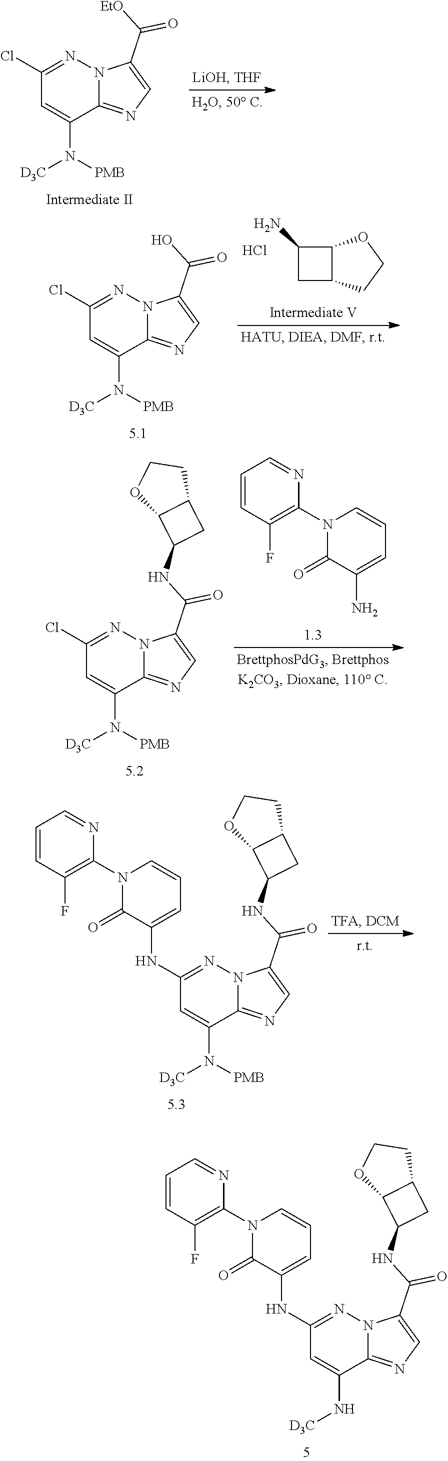

To a suspension of ethyl 6-chloro-8-((4-methoxybenzyl)(methyl-d₃)amino)imidazo[1,2-b]pyridazine-3-carboxylate (Intermediate II, 1.50 g, 3.97 mmol) in a mixed solvent of tetrahydrofuran (3 mL), methanol (3 mL) and water (6 mL) was added lithium hydroxide monohydrate (834 mg, 19.8 mmol), and the resulting mixture was stirred at 50° C. for 1 hr. The mixture was adjusted to pH 6 with 1 M aq. hydrochloric acid. The precipitated solids were collected by filtration, washed with water (2×5 mL), and then dried in vacuum to afford 6-chloro-8-((4-methoxybenzyl)(methyl-d₃)amino)imidazo[1,2-b]pyridazine-3-carboxylic acid (5.1, 1.36 g, 98%) as a yellow solid. LC-MS (ESI): m/z 350.1 [M+H]⁺.

To a mixture of 6-chloro-8-((4-methoxybenzyl)(methyl-d₃)amino)imidazo[1,2-b]pyridazine-3-carboxylic acid (5.1, 500 mg, 1.42 mmol) and (1R,5S,7R)-2-oxabicyclo[3.2.0]heptan-7-amine hydrochloride (Intermediate V, 321 mg, crude) in N,N-dimethylformamide (6 mL) were added N,N-diisopropylethylamine (923 mg, 7.14 mmol) and 2-(7-azabenzotriazol-1-yl)-N,N,N′,N′-tetramethyluronium hexafluorophosphate (1.09 g, 2.86 mmol), and the resulting mixture was stirred at room temperature for 1 hr. The mixture was diluted with dichloromethane (20 mL) and washed with water (3×15 mL). The organic layer was dried over anhydrous sodium sulfate, filtered and concentrated under reduced pressure. The residue was subjected to reverse phase flash chromatography to afford N-((1R,5S,7R)-2-oxabicyclo[3.2.0]heptan-7-yl)-6-chloro-8-((4-methoxybenzyl)(methyl-d₃)amino)imidazo[1,2-b]pyridazine-3-carboxamide (5.2, 410 mg, 64%) as a yellow solid. LC-MS (ESI): m/z 445.2 [M+H]⁺.

To a mixture of N-((1R,5S,7R)-2-oxabicyclo[3.2.0]heptan-7-yl)-6-chloro-8-((4-methoxybenzyl)(methyl-d₃)amino)imidazo[1,2-b]pyridazine-3-carboxamide (5.2, 100 mg, 0.23 mmol) and 3-amino-3′-fluoro-2H-[1,2′-bipyridin]-2-one (1.3, 69.0 mg, 0.34 mmol) in 1,4-dioxane (5 mL) were added BrettPhos Pd G3 (81.0 mg, 0.09 mmol), BrettPhos (48.0 mg, 0.09 mmol) and potassium carbonate (124 mg, 0.90 mmol). The resulting mixture was stirred at 110° C. for 3 hrs under nitrogen atmosphere. Then it was diluted with water (20 mL) and the aqueous layer was extracted with dichloromethane (3×15 mL). The combined organic layers were dried over anhydrous sodium sulfate, filtered and concentrated under reduced pressure. The residue was subjected to Prep-TLC to afford N-((1R,5S,7R)-2-oxabicyclo[3.2.0]heptan-7-yl)-6-((3′-fluoro-2-oxo-2H-[1,2′-bipyridin]-3-yl)amino)-8-((4-methoxybenzyl)(methyl-d₃)amino)imidazo[1,2-b]pyridazine-3-carboxamide (5.3, 137 mg, crude) as a green solid. LC-MS (ESI): m/z 614.20 [M+H]⁺.

To a solution of N-((1R,5S,7R)-2-oxabicyclo[3.2.0]heptan-7-yl)-6-((3′-fluoro-2-oxo-2H-[1,2′-bipyridin]-3-yl)amino)-8-((4-methoxybenzyl)(methyl-d₃)amino)imidazo[1,2-b]pyridazine-3-carboxamide (5.3, 160 mg, crude) in dichloromethane (4 mL) was added trifluoroacetic acid (4 mL), and the resulting mixture was stirred at room temperature for 1 hr. The resulting mixture was concentrated under reduced pressure. The residue was subjected to Prep-HPLC to afford N-((1R,5S,7R)-2-oxabicyclo[3.2.0]heptan-7-yl)-6-((3′-fluoro-2-oxo-2H-[1,2′-bipyridin]-3-yl)amino)-8-((methyl-d₃)amino)imidazo[1,2-b]pyridazine-3-carboxamide (5, 67.3 mg, ee>97%) as a blue solid. LC-MS (ESI): m/z 494.10 [M+H]⁺. ¹H NMR (400 MHz, DMSO-d₆) δ 8.78 (d, J=7.5 Hz, 1H), 8.69 (s, 1H), 8.51 (d, J=4.7 Hz, 1H), 8.19 (dd, J=7.5, 1.7 Hz, 1H), 8.06 (t, J=9.0 Hz, 1H), 7.86 (s, 1H), 7.74-7.72 (m, 1H), 7.47 (s, 1H), 7.40 (dd, J=7.1, 1.7 Hz, 1H), 6.46 (t, J=7.1 Hz, 1H), 6.37 (s, 1H), 4.49 (dd, J=6.8, 3.2 Hz, 1H), 4.34-4.24 (m, 1H), 4.11-4.02 (m, 1H), 4.00-3.90 (m, 1H), 2.98-2.88 (m, 1H), 2.07-1.89 (m, 2H), 1.88-1.67 (m, 2H).

### Synthetic Example 8. Synthesis of 6-(chroman-8-ylamino)-N-(6,7-dihydro-5H-pyrrolo[1,2-a]imidazol-7-yl)-8-(methylamino)imidazo[1,2-b]pyridazine-3-carboxamide (6)

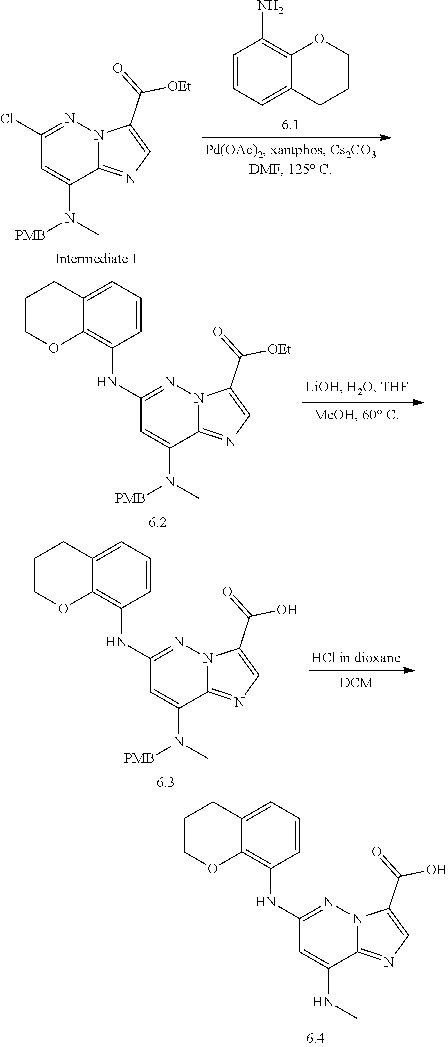

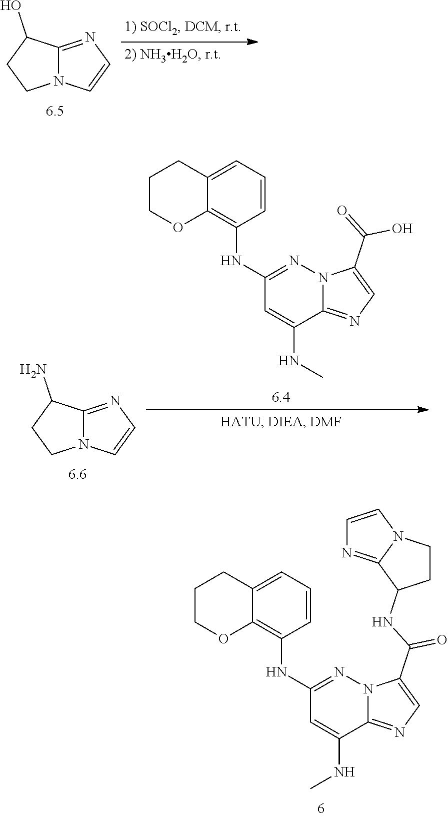 [SMILES: OC1CCn2ccnc21 ; NC1CCn2ccnc21 ; CNc1cc(Nc2cccc3c2OCCC3)nn2c(C(=O)O)cnc12 ; CNc1cc(Nc2cccc3c2OCCC3)nn2c(C(=O)NC3CCn4ccnc43)cnc12]

Compound 6.2 was synthesized from Intermediate I similarly to procedure A to afford ethyl 6-(chroman-8-ylamino)-8-((4-methoxybenzyl)(methyl)amino)imidazo[1,2-b]pyridazine-3-carboxylate (6.2) as a yellow solid. LC-MS (ESI): m/z 488.2 [M+H]⁺.

Compound 6.3 was synthesized from 6.2 similarly to procedure B to afford 6-(chroman-8-ylamino)-8-((4-methoxybenzyl)(methyl)amino)imidazo[1,2-b]pyridazine-3-carboxylic acid (6.3) as a white solid. LC-MS (ESI): m/z 460.2 [M+H]⁺.

Compound 6.4 was synthesized from 6.3 similarly to procedure C to afford 6-(chroman-8-ylamino)-8-(methylamino)imidazo[1,2-b]pyridazine-3-carboxylic acid (6.4) as a white solid. LC-MS (ESI): m/z 340.1 [M+H]⁺.

To a solution of 6,7-dihydro-5H-pyrrolo[1,2-a]imidazol-7-ol (6.5, 200 mg, 1.61 mmol) in dichloromethane (5 mL) was added thionyl chloride (383 mg, 3.22 mmol) at 0° C., and the resulting mixture was stirred at room temperature for 1.5 hrs. Then to the mixture was added ammonium hydroxide (5 mL) dropwise at room temperature and the mixture was stirred at room temperature for additional 1 hr. The reaction mixture was concentrated under reduced pressure and the residue was diluted with dichloromethane/methanol (20 mL, V/V=10/1). Then the mixture was filtered and the filter cake was washed with dichloromethane (3×5 mL). The filtrate was concentrated under reduced pressure to afford 6,7-dihydro-5H-pyrrolo[1,2-a]imidazol-7-amine (6.6, 220 mg, crude) as a yellow oil. LC-MS (ESI): m/z 124.2 [M+H]⁺.

To a mixture of 6-(chroman-8-ylamino)-8-(methylamino)imidazo[1,2-b]pyridazine-3-carboxylic acid (6.4, 50.0 mg, crude) and 6,7-dihydro-5H-pyrrolo[1,2-a]imidazol-7-amine (6.6, 36.0 mg, crude) in N,N-dimethylformamide (3 mL) were added N,N-diisopropylethylamine (57.0 mg, 0.44 mmol) and 2-(7-azabenzotriazol-1-yl)-N,N,N′,N′-tetramethyluronium hexafluorophosphate (112 mg, 0.29 mmol), and the resulting mixture was stirred at room temperature overnight. The mixture was filtered and the filtrate was subjected to Prep-HPLC to afford 6-(chroman-8-ylamino)-N-(6,7-dihydro-5H-pyrrolo[1,2-a]imidazol-7-yl)-8-(methylamino)imidazo[1,2-b]pyridazine-3-carboxamide (6, 6.90 mg) as a white solid. LC-MS (ESI): m/z 445.3 [M+H]⁺. ¹H NMR (300 MHz, DMSO-d₆) δ 9.11 (d, J=9.0 Hz, 1H), 8.17 (s, 1H), 7.85 (s, 1H), 7.42-7.41 (m, 1H), 7.25-7.14 (m, 2H), 6.94 (s, 1H), 6.67 (d, J=7.2 Hz, 1H), 6.13 (t, J=7.8 Hz, 1H), 6.03 (s, 1H), 5.47 (q, J=8.7 Hz, 1H), 4.25-4.06 (m, 2H), 4.06-3.83 (m, 2H), 2.95-2.80 (m, 4H), 2.73-2.61 (m, 2H), 2.05-1.77 (m, 3H).

### Synthetic Example 9. Synthesis of N-(5,6-dihydro-4H-cyclopenta[c]isoxazol-6-yl)-8-(methylamino)-6-((2-oxo-2H-[1,2′-bipyridin]-3-yl)amino)imidazo[1,2-b]pyridazine-3-carboxamide (7)

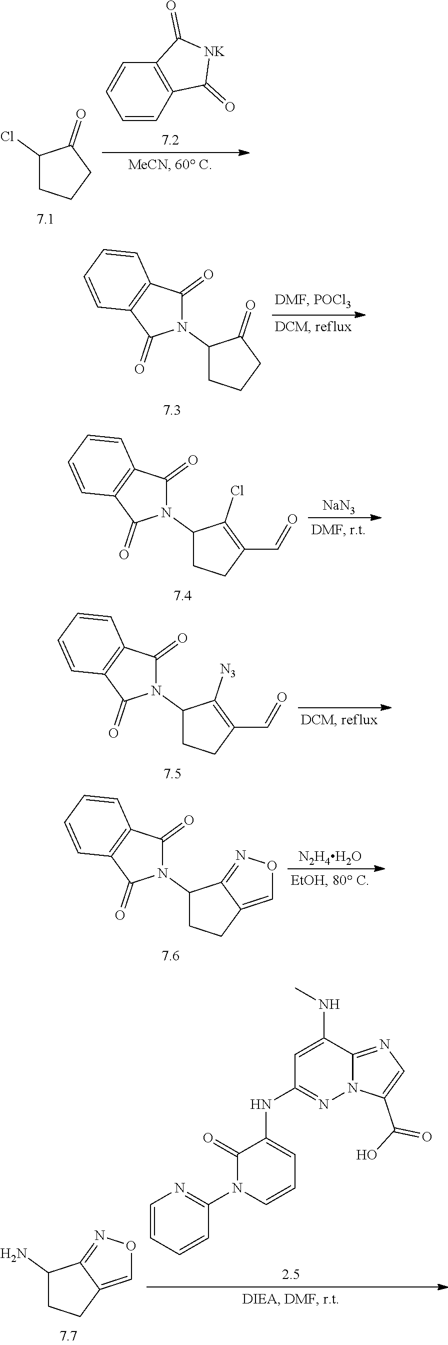

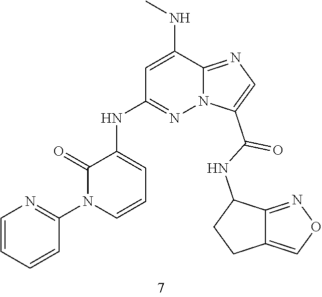 [SMILES: CNc1cc(Nc2cccn(-c3ccccn3)c2=O)nn2c(C(=O)NC3CCc4conc43)cnc12]

To a solution of 2-chlorocyclopentan-1-one (7.1, 4.24 mL, 42.2 mmol) in acetonitrile (50 mL) was added potassium 1,3-dioxoisoindolin-2-ide (7.2, 10.2 g, 54.8 mmol), and the mixture was stirred at 60° C. for 16 hrs. The mixture was concentrated under reduced pressure, diluted with water (50 mL) and then extracted with ethyl acetate (3×50 mL). The combined organic layers were dried over anhydrous sodium sulfate, filtered and concentrated under reduced pressure. The residue was subjected to silica gel column chromatography to afford 2-(2-oxocyclopentyl)isoindoline-1,3-dione (7.3,) as a white solid. ¹H NMR (500 MHz, CDCl₃) δ 7.90 (dd, J=5.5, 3.1 Hz, 2H), 7.86 (dd, J=5.4, 3.1 Hz, 2H), 4.60 (t, J=10.3 Hz, 1H), 2.57-2.48 (m, 2H), 2.44-2.35 (m, 2H), 2.33-2.22 (m, 1H), 2.01-1.87 (m, 1H).

To a solution of 2-(2-oxocyclopentyl)isoindoline-1,3-dione (7.3, 2.40 g, 10.5 mmol) in dichloromethane (40 mL) were added N,N-dimethylformamide (2.40 mL, 31.4 mmol) and phosphoryl chloride (2.00 mL, 20.9 mmol) at 0° C. Then the mixture was stirred at 40° C. for 16 hrs. The reaction mixture was quenched with water (30 mL), adjusted to pH 7 with saturated aq. NaHCO₃ and extracted with ethyl acetate (3×50 mL). The combined organic layers were washed with water (4×30 mL), dried over anhydrous sodium sulfate, filtered and concentrated under reduced pressure. The residue was subjected to silica gel column chromatography to afford 2-chloro-3-(1,3-dioxoisoindolin-2-yl)cyclopent-1-ene-1-carbaldehyde (7.4,) as a white solid. ¹H NMR (500 MHz, CDCl₃) δ 10.08 (s, 1H), 7.91 (dd, J=5.4, 3.0 Hz, 2H), 7.80 (dd, J=5.5, 3.0 Hz, 2H), 5.54 (ddt, J=9.6, 7.2, 2.4 Hz, 1H), 3.02-2.92 (m, 1H), 2.72-2.61 (m, 1H), 2.58-2.47 (m, 1H), 2.39-2.30 (m, 1H).

To a solution of 2-chloro-3-(1,3-dioxoisoindolin-2-yl)cyclopent-1-ene-1-carbaldehyde (7.4, 1.00 g, 3.63 mmol) in N,N-dimethylformamide (10 mL) was added sodium azide (283 mg, 4.35 mmol) at 0° C., and the mixture was stirred at room temperature for 16 hrs. The mixture was diluted with water (20 mL) and extracted with ethyl acetate (3×20 mL). The combined organic layers were washed with water (4×30 mL), dried over anhydrous sodium sulfate, and then filtered and concentrated under reduced pressure to afford 2-azido-3-(1,3-dioxoisoindolin-2-yl)cyclopent-1-ene-1-carbaldehyde (7.5, 1.00 g, crude) as a yellow oil.

A solution of 2-azido-3-(1,3-dioxoisoindolin-2-yl)cyclopent-1-ene-1-carbaldehyde (7.5, 1.00 g, crude) in dichloromethane (2 mL) was stirred at 40° C. for 16 hrs. The mixture was concentrated under reduced pressure. The residue was subjected to silica gel column chromatography to afford 2-(5,6-dihydro-4H-cyclopenta[c]isoxazol-6-yl)isoindoline-1,3-dione (7.6, 200 mg) as a white solid. ¹H NMR (500 MHz, CDCl₃) δ 8.07 (s, 1H), 7.86 (dd, J=5.5, 3.1 Hz, 2H), 7.75 (dd, J=5.5, 3.0 Hz, 2H), 5.84 (dd, J=9.1, 6.2 Hz, 1H), 3.17-3.07 (m, 1H), 3.04-2.92 (m, 1H), 2.89-2.74 (m, 2H).

To a solution of 2-(5,6-dihydro-4H-cyclopenta[c]isoxazol-6-yl)isoindoline-1,3-dione (7.6, 200 mg, 0.78 mmol) in ethanol (2 mL) was added hydrazine hydrate (0.02 mL), and the mixture was stirred at 80° C. for 1 hr. The mixture was concentrated under reduced pressure. The residue was subjected to silica gel column chromatography to afford 5,6-dihydro-4H-cyclopenta[c]isoxazol-6-amine (7.7, 84 mg, 86%). LC-MS (ESI): m/z 125.14 [M+H]⁺.

To a solution of 8-(methylamino)-6-((2-oxo-2H-[1,2′-bipyridin]-3-yl)amino) imidazo[1,2-b]pyridazine-3-carboxylic acid (2.5, 100 mg, crude) and 5,6-dihydro-4H-cyclopenta[c]isoxazol-6-amine (7.7, 38.7 mg, 0.31 mmol) in N,N-dimethylformamide (2 mL) were added 2-(7-azabenzotriazol-1-yl)-N,N,N′,N′-tetramethyluronium hexafluorophosphate (148 mg, 0.39 mmol) and N,N-diisoprethylamine (100 mg, 0.78 mmol), and the mixture was stirred at room temperature for 1 hr. The mixture was filtered and the filtrate was subjected to Prep-HPLC to afford N-(5,6-dihydro-4H-cyclopenta[c]isoxazol-6-yl)-8-(methylamino)-6-((2-oxo-2H-[1,2′-bipyridin]-3-yl)amino)imidazo[1,2-b]pyridazine-3-carboxamide (7, 30 mg) as a white solid. LC-MS (ESI): m/z 483.98 [M+H]⁺. ¹H NMR (500 MHz, DMSO-d₆)=8.96 (d, J=8.5 Hz, 1H), 8.63 (d, J=3.3 Hz, 2H), 8.52 (d, J=1.3 Hz, 1H), 8.03 (td, J=7.8, 1.9 Hz, 1H), 7.94 (s, 1H), 7.89 (dd, J=7.3, 1.8 Hz, 1H), 7.82 (d, J=8.1 Hz, 1H), 7.55-7.48 (m, 2H), 7.43 (dd, J=7.0, 1.7 Hz, 1H), 6.39 (s, 1H), 5.93 (t, J=7.2 Hz, 1H), 5.68 (q, J=8.3 Hz, 1H), 2.93-2.85 (m, 4H), 2.82-2.74 (m, 1H), 2.71-2.65 (m, 1H), 2.37-2.28 (m, 1H).

### Synthetic Example 10. Synthesis of N-(5,6-dihydro-4H-cyclopenta[c]isoxazol-6-yl-3-d)-8-(methylamino)-6-((2-oxo-2H-[1,2′-bipyridin]-3-yl)amino)imidazo[1,2-b]pyridazine-3-carboxamide (8)

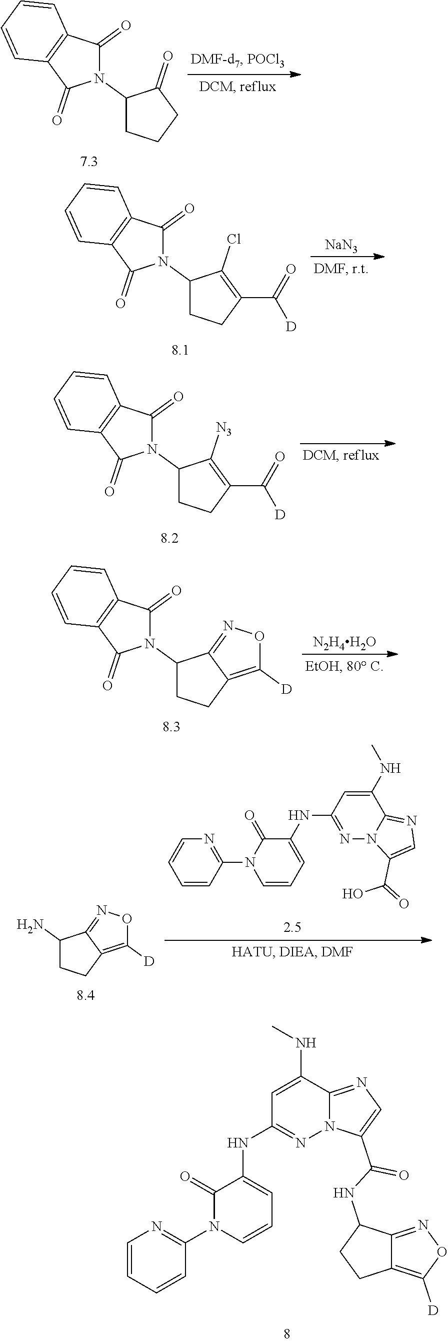

To a solution of 2-(2-oxocyclopentyl)isoindoline-1,3-dione (7.3, 1.00 g, 4.36 mmol) in dichloromethane (5 mL) were added N,N-dimethylformamide-d₇ (0.70 g, 8.72 mmol) and phosphorus oxychloride (1.95 mL, 20.9 mmol) at 0° C., and the mixture was stirred at 40° C. for 16 hrs. The reaction mixture was quenched with water (30 mL), adjusted to pH 7 with saturated aq. NaHCO₃ and extracted with ethyl acetate (3×50 mL). The combined organic layers were washed with water (4×30 mL), dried over anhydrous sodium sulfate, filtered and concentrated under reduced pressure. The residue was subjected to silica gel column chromatography to afford 2-chloro-3-(1,3-dioxoisoindolin-2-yl)cyclopent-1-ene-1-carbaldehyde-d as a white solid. ¹H NMR (500 MHz, CDCl₃) δ 7.91 (dd, J=5.5, 3.1 Hz, 2H), 7.79 (dd, J=5.5, 3.0 Hz, 2H), 5.58-5.49 (m, 1H), 3.02-2.92 (m, 1H), 2.73-2.61 (m, 1H), 2.58-2.47 (m, 1H), 2.41-2.29 (m, 1H).

To a solution of 2-chloro-3-(1,3-dioxoisoindolin-2-yl)cyclopent-1-ene-1-carbaldehyde-d (8.1, 0.70 g, 2.53 mmol) in N,N-dimethylformamide (5 mL) was added sodium azide (197 mg, 3.04 mmol) at 0° C., and the mixture was stirred at room temperature for 16 hrs. The mixture was diluted with water (20 mL) and extracted with ethyl acetate (3×15 mL). The combined organic layers were washed with water (4×15 mL), dried over anhydrous sodium sulfate, and then filtered and concentrated under reduced pressure to afford 2-azido-3-(1,3-dioxoisoindolin-2-yl)cyclopent-1-ene-1-carbaldehyde-d (8.2, 700 mg, crude) as a yellow oil.

A solution of 2-azido-3-(1,3-dioxoisoindolin-2-yl)cyclopent-1-ene-1-carbaldehyde-d (8.2, 700 mg, 2.47 mmol) in dichloromethane (10 mL) was stirred at 40° C. for 16 hrs. The mixture was concentrated under reduced pressure. The residue was subjected to silica gel column chromatography to afford 2-(5,6-dihydro-4H-cyclopenta[c]isoxazol-6-yl-3-d)isoindoline-1,3-dione (8.3, 200 mg, 32%) as a white solid.

To a solution of 2-(5,6-dihydro-4H-cyclopenta[c]isoxazol-6-yl-3-d)isoindoline-1,3-dione (8.3, 150 mg, 0.58 mmol) in ethanol (2 mL) was added hydrazine hydrate (50 uL). The mixture was stirred at 80° C. for 1 hr. The mixture was concentrated under reduced pressure. The residue was subjected to silica gel column chromatography to afford 5,6-dihydro-4H-cyclopenta[c]isoxazol-3-d-6-amine (8.4, 61.0 mg, 84%). LC-MS (ESI): m/z 126.21 [M+H]⁺.

To a solution of 8-(methylamino)-6-((2-oxo-2H-[1,2′-bipyridin]-3-yl)amino) imidazo[1,2-b]pyridazine-3-carboxylic acid (2.5, 80.0 mg, crude) and 5,6-dihydro-4H-cyclopenta[c]isoxazol-3-d-6-amine (8.4, 31.2 mg, 0.25 mmol) in N,N-dimethylformamide (2 mL) were added 2-(7-azabenzotriazol-1-yl)-N,N,N′,N′-tetramethyluronium hexafluorophosphate (118 mg, 0.31 mmol) and N,N-diisoprethylamine (80.0 mg, 0.62 mmol), and the mixture was stirred at room temperature for 1 hr. The mixture was filtered and the filtrate was subjected to Prep-HPLC to afford N-(5,6-dihydro-4H-cyclopenta[c]isoxazol-6-yl-3-d)-8-(methylamino)-6-((2-oxo-2H-[1,2′-bipyridin]-3-yl)amino)imidazo[1,2-b]pyridazine-3-carboxamide (8, 37.0 mg) as a white solid. LC-MS (ESI): m/z 484.98 [M+H]⁺. ¹H NMR (500 MHz, DMSO-d₆) δ=8.96 (d, J=8.5 Hz, 1H), 8.63 (d, J=3.4 Hz, 2H), 8.03 (td, J=7.8, 1.9 Hz, 1H), 7.94 (s, 1H), 7.89 (dd, J=7.4, 1.8 Hz, 1H), 7.82 (d, J=8.1 Hz, 1H), 7.56-7.48 (m, 2H), 7.43 (dd, J=7.1, 1.8 Hz, 1H), 6.39 (s, 1H), 5.93 (t, J=7.2 Hz, 1H), 5.68 (q, J=8.3 Hz, 1H), 2.95-2.83 (m, 4H), 2.82-2.75 (m, 1H), 2.72-2.64 (m, 1H), 2.34-2.31 (m, 1H).

### Synthetic Example 11. Synthesis of N-(5,6-dihydro-4H-cyclopenta[d]thiazol-4-yl)-8-(methylamino)-6-((2-oxo-2H-[1,2′-bipyridin]-3-yl)amino)imidazo[1,2-b]pyridazine-3-carboxamide (9)

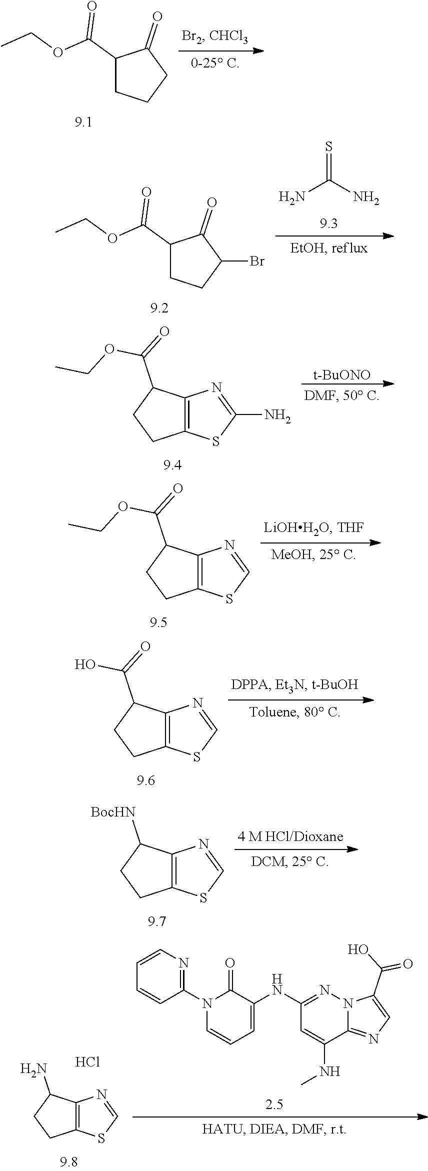

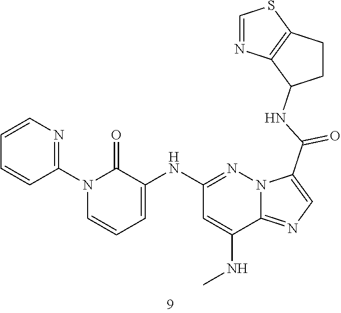 [SMILES: CNc1cc(Nc2cccn(-c3ccccn3)c2=O)nn2c(C(=O)NC3CCc4scnc43)cnc12]

To a solution of ethyl 2-oxocyclopentane-1-carboxylate (9.1, 15.0 g, 96.0 mmol) in chloroform (100 mL) was added bromine (96.0 mmol, 4.95 mL) dropwise at 0° C. over 15 mins, then the mixture was stirred at 25° C. for additional 1 hr. The mixture was quenched with saturated aq. NaHCO₃ (200 mL) at 0° C. The organic layer was washed with brine (100 mL), dried over anhydrous sodium sulfate, and then filtered and concentrated under reduced pressure to afford ethyl 3-bromo-2-oxocyclopentane-1-carboxylate (9.2, 21.5 g, crude) as a brown oil.

A mixture of ethyl 3-bromo-2-oxocyclopentane-1-carboxylate (9.2, 22 g, crude) and thiourea (9.3, 7.12 g, 93.6 mmol) in ethyl alcohol (100 mL) was stirred at 25° C. for 1 hr, then the mixture was heated to 80° C. for 19 hrs. The mixture was cooled to room temperature and concentrated under reduced pressure. The residue was diluted with dichloromethane (200 mL) and washed with saturated aq. NaHCO₃ (300 mL). The organic layer was dried over anhydrous sodium sulfate, filtered and concentrated under reduced pressure. The residue was subjected to silica gel column chromatography to afford ethyl 2-amino-5,6-dihydro-4H-cyclopenta[d]thiazole-4-carboxylate (9.4, 3.20 g) as a white solid. LC-MS (ESI): m/z 213.0 [M+H]⁺. ¹H NMR (400 MHz, DMSO-d₆) δ 5.17 (br s, 2H), 4.22-4.17 (m, 2H), 3.81-3.77 (m, 1H), 2.92-2.91 (m, 1H), 2.78-2.76 (m, 1H), 2.68-2.66 (m, 2H), 1.26 (t, J=7.2 Hz, 3H).

To a solution of ethyl 2-amino-5,6-dihydro-4H-cyclopenta[d]thiazole-4-carboxylate (9.4, 2.30 g, 10.8 mmol) in N,N-dimethylformamide (15 mL) was added tert-butyl nitrite (11.92 mmol, 1.42 mL) dropwise over 5 mins, and the mixture was stirred at 50° C. for 1 hr. The mixture was diluted with water (30 mL) and extracted with ethyl acetate (3×30 mL). The combined organic layers were concentrated under reduced pressure. The residue was subjected to silica gel column chromatography to afford ethyl 5,6-dihydro-4H-cyclopenta[d]thiazole-4-carboxylate (9.5, 510 mg, 23%) as a yellow oil. 1H NMR (400 MHz, CDCl₃) δ 8.83 (s, 1H), 4.26-4.20 (m, 2H), 4.04-4.02 (m, 1H), 3.08-3.06 (m, 1H), 2.88-2.83 (m, 3H), 1.29 (t, J=7.2 Hz, 3H).

To a suspension of ethyl 5,6-dihydro-4H-cyclopenta[d]thiazole-4-carboxylate (9.5, 510 mg, 2.59 mmol) in a mixed solvent of water (1 mL), methanol (2 mL) and tetrahydrofuran (2 mL) was added lithium hydroxide monohydrate (217 mg, 5.17 mmol), and the mixture was stirred at 25° C. for 20 hrs. The mixture was diluted with water (15 mL) and ethyl acetate (10 mL). The aqueous layer was adjusted to pH˜4 with 2 M aq. HCl and extracted with ethyl acetate (3×15 mL). The combined organic layers were dried over anhydrous sodium sulfate and concentrated under reduced pressure to afford 5,6-dihydro-4H-cyclopenta[d]thiazole-4-carboxylic acid (9.6, 385 mg, 88% yield) as a brown solid. LC-MS (ESI): m/z 170.0 [M+H]⁺. ¹H NMR (400 MHz, DMSO-d₆) δ 12.4 (br s, 1H), 8.93 (s, 1H), 3.89-3.87 (m, 1H), 2.95-2.64 (m, 4H)

To a mixture of 5,6-dihydro-4H-cyclopenta[d]thiazole-4-carboxylic acid (9.6, 360 mg, 2.13 mmol) and triethylamine (2.13 mmol, 296 uL) in toluene (20 mL) were added and tertiary butanol (21.2 mmol, 2.03 mL) and diphenyl azidophosphate (2.13 mmol, 461 uL), and the mixture was stirred at 25° C. for 1 hr. Then the mixture was heated to 80° C. and stirred for 19 hrs. The mixture was concentrated under reduced pressure. The residue was subjected to silica gel column chromatography to afford tert-butyl (5,6-dihydro-4H-cyclopenta[d]thiazol-4-yl)carbamate (9.7, 122 mg, 23% yield) as a white solid. LC-MS (ESI): m/z 241.1 [M+H]⁺. ¹H NMR (400 MHz, CDCl₃) δ 8.70 (s, 1H), 5.11 (s, 1H), 4.91 (s, 1H), 3.04-3.01 (m, 2H), 2.89-2.88 (m, 1H), 2.38-2.35 (m, 1H), 1.46 (s, 9H)

To a solution of tert-butyl (5,6-dihydro-4H-cyclopenta[d]thiazol-4-yl)carbamate (9.7, 120 mg, 499 umol) in dichloromethane (2 mL) was added hydrochloric acid (2 mL, 4 M in dioxane), then the mixture was stirred at 25° C. for 1 hr. The mixture was concentrated under reduced pressure to afford 5,6-dihydro-4H-cyclopenta[d]thiazol-4-amine hydrochloride (9.8, 67.6 mg, crude) as a white solid. LC-MS (ESI): m/z 141.1 [M+H]⁺. ¹H NMR (400 MHz, DMSO-d₆) δ 9.09 (s, 1H), 8.56 (br s, 2H), 4.61 (br s, 1H), 3.10-3.05 (m, 1H), 2.95-2.86 (m, 2H), 2.44-2.42 (m, 1H).

To a solution of 8-(methylamino)-6-((2-oxo-2H-[1,2′-bipyridin]-3-yl)amino) imidazo[1,2-b]pyridazine-3-carboxylic acid (2.5, 100 mg, crude) and 5,6-dihydro-4H-cyclopenta[d]thiazol-4-amine hydrochloride (9.8, 43.4 mg, crude) in N,N-dimethylformamide (2 mL) were added N,N-diisopropylethylamine (100 mg, 0.78 mmol) and 2-(7-azabenzotriazol-1-yl)-N,N,N′,N-tetramethyluronium hexafluorophosphate (148 mg, 0.39 mmol), and the mixture was stirred at room temperature for 1 hr. The reaction mixture was filtered and the filtrate was subjected to Prep-HPLC to afford N-(5,6-dihydro-4H-cyclopenta[d]thiazol-4-yl)-8-(methylamino)-6-((2-oxo-2H-[1,2′-bipyridin]-3-yl)amino)imidazo[1,2-b]pyridazine-3-carboxamide (9, 46.0 mg) as a white solid. LC-MS (ESI): m/z 500.25 [M+H]⁺. ¹H NMR (500 MHz, DMSO-d₆) δ=8.96 (s, 1H), 8.85 (d, J=8.6 Hz, 1H), 8.63 (dd, J=5.0, 1.9 Hz, 1H), 8.59 (s, 1H), 8.03 (td, J=7.8, 1.9 Hz, 1H), 7.93 (s, 1H), 7.80 (d, J=8.1 Hz, 1H), 7.73 (dd, J=7.3, 1.8 Hz, 1H), 7.55-7.47 (m, 2H), 7.38 (dd, J=7.1, 1.7 Hz, 1H), 6.38 (s, 1H), 5.74 (t, J=7.2 Hz, 1H), 5.62-5.55 (m, 1H), 3.11-3.03 (m, 2H), 2.96-2.88 (m, 1H), 2.86 (d, J=4.9 Hz, 3H), 2.36-2.27 (m, 1H).

### Synthetic Example 12. Synthesis of N-(hexahydro-2H-cyclopenta[b]furan-6-yl)-8-(methylamino)-6-((2-oxo-2H-[1,2′-bipyridin]-3-yl)amino)imidazo[1,2-b]pyridazine-3-carboxamide (10a) and N-(hexahydro-2H-cyclopenta[b]furan-6-yl)-8-(methylamino)-6-((2-oxo-2H-[1,2′-bipyridin]-3-yl)amino)imidazo[1,2-b]pyridazine-3-carboxamide (10b)

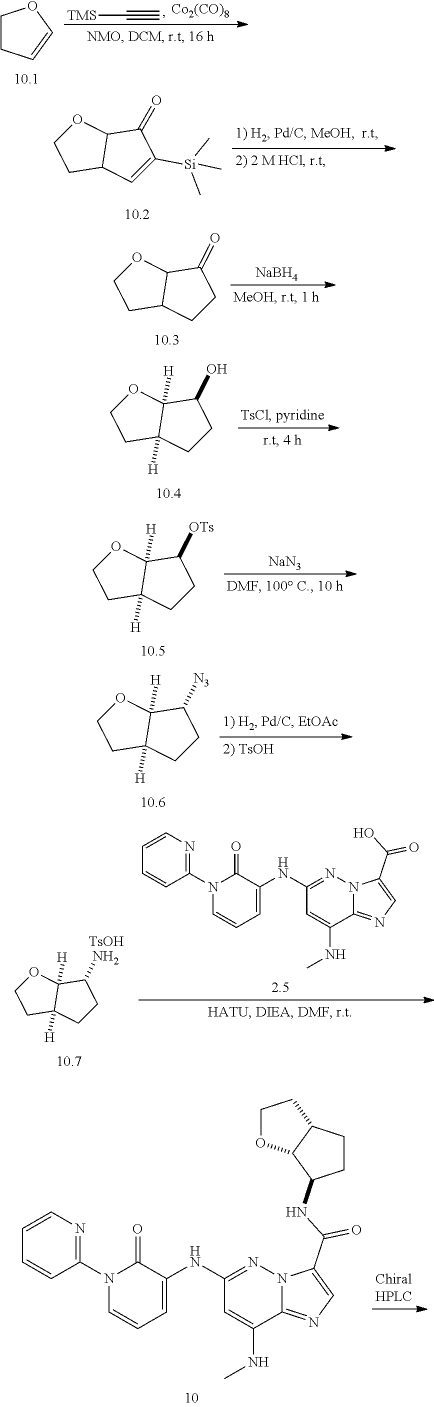

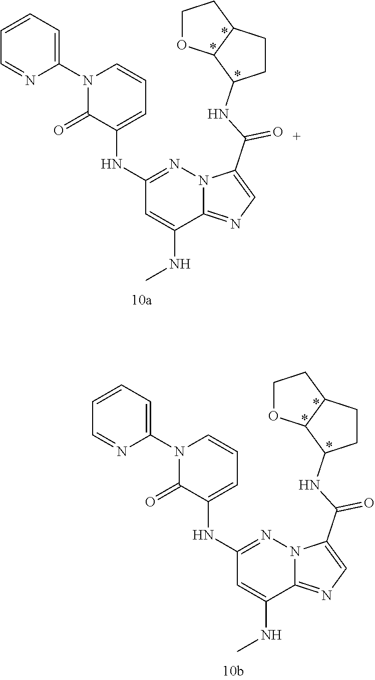 [SMILES: CNc1cc(Nc2cccn(-c3ccccn3)c2=O)nn2c(C(=O)NC3CCC4CCOC43)cnc12 ; CNc1cc(Nc2cccn(-c3ccccn3)c2=O)nn2c(C(=O)NC3CCC4CCOC43)cnc12]

To a solution of (3.83 g, 11.2 mmol) in dichloromethane (120 mL) was added ethynyltrimethylsilane (1.10 g, 11.2 mmol) dropwise, and the mixture was stirred at room temperature for 1 hr. Then to the mixture were added 2,3-dihydrofuran (10.1, 3.92 g, 56.0 mmol) and 4-methylmorpholin-4-ium-4-olate (7.87 g, 67.2 mmol) at 0° C. The resulting mixture was warmed to room temperature and stirred for additional 16 hrs. The mixture was concentrated under reduced pressure. The residue was subjected to silica gel column chromatography to afford 5-(trimethylsilyl)-2,3,3a,6a-tetrahydro-6H-cyclopenta[b]furan-6-one (10.2, 1.30 g, 6.62 mmol, 59%) as a colorless oil. ¹H NMR (500 MHz, CDCl₃) δ 7.62 (d, J=2.7 Hz, 1H), 4.28 (d, J=5.6 Hz, 1H), 3.95 (ddd, J=9.3, 7.5, 2.0 Hz, 1H), 3.50-3.41 (m, 2H), 2.12-2.05 (m, 1H), 1.83-1.75 (m, 1H), 0.18 (s, 9H).

A mixture of 5-(trimethylsilyl)-2,3,3a,6a-tetrahydro-6H-cyclopenta[b]furan-6-one (10.2, 1.30 g, 6.62 mmol) and palladium (10% on carbon, 1.41 g) in methanol (20 mL) was degassed and backfilled with hydrogen three times. The mixture was stirred at room temperature for 1 hr under hydrogen atmosphere (1 atm). Then the mixture was filtered and to the filtrate was added 2 M aq. HCl (5 mL), and the resulting mixture was stirred at room temperature for additional 3 hrs. The mixture was concentrated to remove methanol, diluted with water (20 mL) and then extracted with ethyl acetate (3×15 mL). The combined organic layers were concentrated under reduced pressure. The residue was subjected to silica gel column chromatography to afford hexahydro-6H-cyclopenta[b]furan-6-one (10.3, 400 mg, 48%) as a colorless oil. ¹H NMR (500 MHz, CDCl₃) δ 4.10 (d, J=7.6 Hz, 1H), 3.97-3.81 (m, 2H), 3.04-2.92 (m, 1H), 2.45-2.25 (m, 2H), 2.24-2.11 (m, 2H), 1.82-1.69 (m, 2H).

To a solution of hexahydro-6H-cyclopenta[b]furan-6-one (10.3, 350 mg, 2.77 mmol) in methanol (5 mL) was added sodium borohydride (46.9 mg, 1.39 mmol) at 0° C., and the mixture was stirred at room temperature for 1 hr. The reaction mixture was quenched with 2 M aq. HCl (1 mL), diluted with water (20 mL) and extracted with ethyl acetate (3×15 mL). The combined organic layers were dried over anhydrous sodium sulfate and concentrated under reduced pressure. The residue was subjected to silica gel column chromatography to afford cis-hexahydro-2H-cyclopenta[b]furan-6-ol (10.4, 200 mg, 56%) as a colorless oil. ¹HNMR (500 MHz, CDCl₃) δ 4.24 (dd, J=7.1, 5.2 Hz, 1H), 3.96 (dt, J=7.8, 5.4 Hz, 1H), 3.90-3.78 (m, 2H), 2.73-2.63 (m, 1H), 2.17-2.10 (m, 1H), 1.83-1.77 (m, 1H), 1.72-1.66 (m, 1H), 1.62-1.55 (m, 2H), 1.48-1.42 (m, 1H).

To a solution of cis-hexahydro-2H-cyclopenta[b]furan-6-ol (10.4, 350 mg, 2.73 mmol) in pyridine (3 mL) was added 4-methylbenzenesulfonyl chloride (781 mg, 4.10 mmol), and the mixture was stirred at room temperature for 4 hrs. The mixture was diluted with water (20 mL) and extracted with ethyl acetate (3×15 mL). The combined organic layers were dried over anhydrous sodium sulfate and concentrated under reduced pressure. The residue was subjected to silica gel column chromatography to afford cis-hexahydro-2H-cyclopenta[b]furan-6-yl 4-methylbenzenesulfonate (10.5, 360 mg, 47%) as a white solid. ¹H NMR (500 MHz, CDCl₃) δ 7.84 (d, J=8.3 Hz, 2H), 7.32 (d, J=8.0 Hz, 2H), 4.63 (ddd, J=9.5, 6.4, 4.6 Hz, 1H), 4.18 (dd, J=6.4, 4.6 Hz, 1H), 3.93-3.81 (m, 1H), 3.78-3.68 (m, 1H), 2.67-2.58 (m, 1H), 2.44 (s, 3H), 2.17-2.06 (m, 1H), 1.91-1.76 (m, 2H), 1.75-1.65 (m, 1H), 1.58-1.54 (m, 1H), 1.47-1.38 (m, 1H).

A solution of cis-hexahydro-2H-cyclopenta[b]furan-6-yl 4-methylbenzenesulfonate (10.5, 360 mg, 1.28 mmol) and sodium azide (166 mg, 2.55 mmol) in N,N-dimethylformamide (2 mL) was stirred at 100° C. for 10 hrs. The mixture was diluted with water (15 mL) and extracted with ethyl acetate (2×10 mL). The combined organic layers were washed with water (3×10 mL), dried over anhydrous sodium sulfate and filtered. The filtrate contained trans-6-azidohexahydro-2H-cyclopenta[b]furan (10.6, 15 mL, a solution in ethyl acetate), which was used in next step directly.

To a solution of trans-6-azidohexahydro-2H-cyclopenta[b]furan (10.6, 15 ml, a solution in ethyl acetate) was added palladium (10% on carbon, 271 mg). The reaction mixture was degassed and backfilled with hydrogen three times, an then stirred at room temperature for 2 hrs under hydrogen atmosphere (1 atm). The reaction mixture was filtered and 4-methylbenzene-1-sulfonic acid (219 mg, 1.27 mmol) was added to the filtrate. The resulting mixture was stirred at room temperature for 1 hr and then concentrated under reduced pressure to afford trans-hexahydro-2H-cyclopenta[b]furan-6-amine 4-methylbenzenesulfonate (10.7, 380 mg) as a white solid.

To a solution of 8-(methylamino)-6-((2-oxo-2H-[1,2′-bipyridin]-3-yl)amino) imidazo[1,2-b]pyridazine-3-carboxylic acid (2.5, 100 mg, crude) and trans-hexahydro-2H-cyclopenta[b]furan-6-amine 4-methylbenzenesulfonate (10.7, 92.7 mg, 0.31 mmol) in N,N-dimethylformamide (2 mL) were added N,N-diisopropylethylamine (100 mg, 0.78 mmol) and 2-(7-azabenzotriazol-1-yl)-N,N,N′,N′-tetramethyluronium hexafluorophosphate (148 mg, 0.39 mmol), and the mixture was stirred at room temperature for 1 hr. The reaction mixture was filtered and the filtrate was subjected to Prep-HPLC to afford trans-N-(hexahydro-2H-cyclopenta[b]furan-6-yl)-8-(methylamino)-6-((2-oxo-2H-[1,2′-bipyridin]-3-yl)amino)imidazo[1,2-b]pyridazine-3-carboxamide (10, 36.0 mg) as a white solid. The racemic mixture was further separated by chiral HPLC to afford N-(hexahydro-2H-cyclopenta[b]furan-6-yl)-8-(methylamino)-6-((2-oxo-2H-[1,2′-bipyridin]-3-yl)amino)imidazo[1,2-b]pyridazine-3-carboxamide (10a, 11.8 mg, ee>99%) and N-(hexahydro-2H-cyclopenta[b]furan-6-yl)-8-(methylamino)-6-((2-oxo-2H-[1,2′-bipyridin]-3-yl)amino)imidazo[1,2-b]pyridazine-3-carboxamide (10b, 12.6 mg, ee>97%), both as a white solid.

10a: Chiral HPLC Retention time: 26.38 min. LC-MS (ESI): m/z 487.21 [M+H]⁺. ¹H NMR (400 MHz, CDCl₃) δ 8.63 (d, J=4.8 Hz, 1H), 8.44 (d, J=7.1 Hz, 1H), 8.18 (s, 1H), 7.97-7.90 (m, 4H), 7.64-7.61 (m, 1H), 7.43-7.41 (m, 1H), 6.46 (t, J=7.2 Hz, 1H), 5.91 (s, 1H), 4.41-4.39 (m, 1H), 4.39-4.37 (m, 1H), 3.84-3.91 (m, 2H), 3.08 (d, J=4.8 Hz, 3H), 2.79-2.73 (m, 1H), 2.31-2.26 (m, 1H), 2.05-2.01 (m, 2H), 1.58-1.56 (m, 2H), 1.57-1.33 (m, 1H), 1.33-1.26 (m, 1H).

10b: Chiral HPLC Retention time: 32.61 min. LC-MS (ESI): m/z 487.21 [M+H]⁺. ¹H NMR (400 MHz, CDCl₃) δ 8.63 (d, J=4.8 Hz, 1H), 8.44 (d, J=7.1 Hz, 1H), 8.18 (s, 1H), 7.97-7.90 (m, 4H), 7.64-7.61 (m, 1H), 7.43-7.41 (m, 1H), 6.46 (t, J=7.2 Hz, 1H), 5.91 (s, 1H), 4.41-4.39 (m, 1H), 4.39-4.37 (m, 1H), 3.84-3.91 (m, 2H), 3.08 (d, J=4.8 Hz, 3H), 2.79-2.73 (m, 1H), 2.31-2.26 (m, 1H), 2.05-2.01 (m, 2H), 1.58-1.56 (m, 2H), 1.57-1.33 (m, 1H), 1.33-1.26 (m, 1H).

Chiral HPLC Method: column: chiral art cellulose-SB, 2*25 cm, 5 m; mobile phase A: MtBE (10 mM NH₃-MeOH), mobile phase B: EtOH-HPLC; flow rate: 20 mL/min; gradient: 25% B to 25% B in 40 min; wave length: 214/250 nm; sample solvent: MeOH:DCM=1:1-HPLC; injection volume: 0.30 mL.

### Synthetic Example 13. Synthesis of N-((1R,5S,7R)-2-oxabicyclo[3.2.0]heptan-7-yl)-6-((1-((1r,4R)-4-(methoxy-d₃)cyclohexyl)-2-oxo-1,2-dihydropyridin-3-yl)amino)-8-((methyl-d₃)amino)imidazo[1,2-b]pyridazine-3-carboxamide (11)

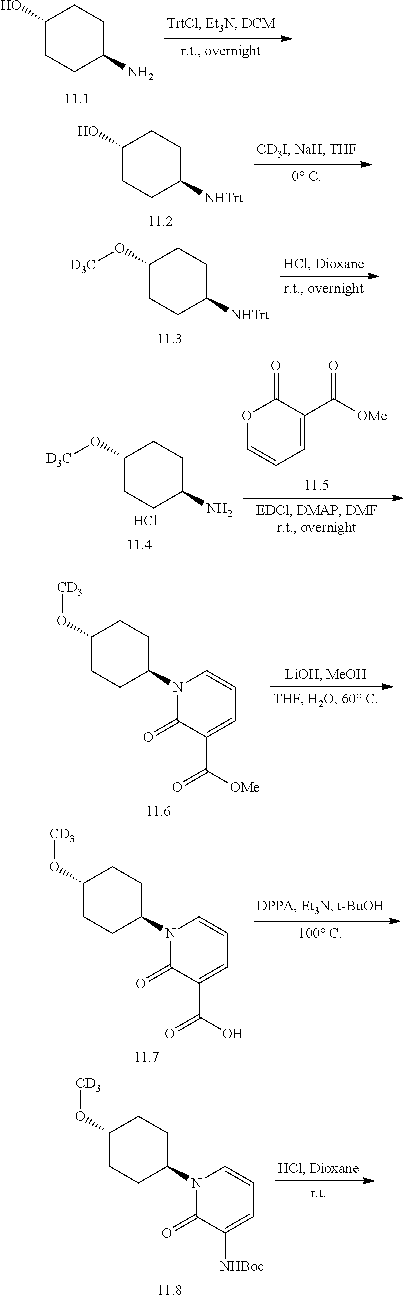

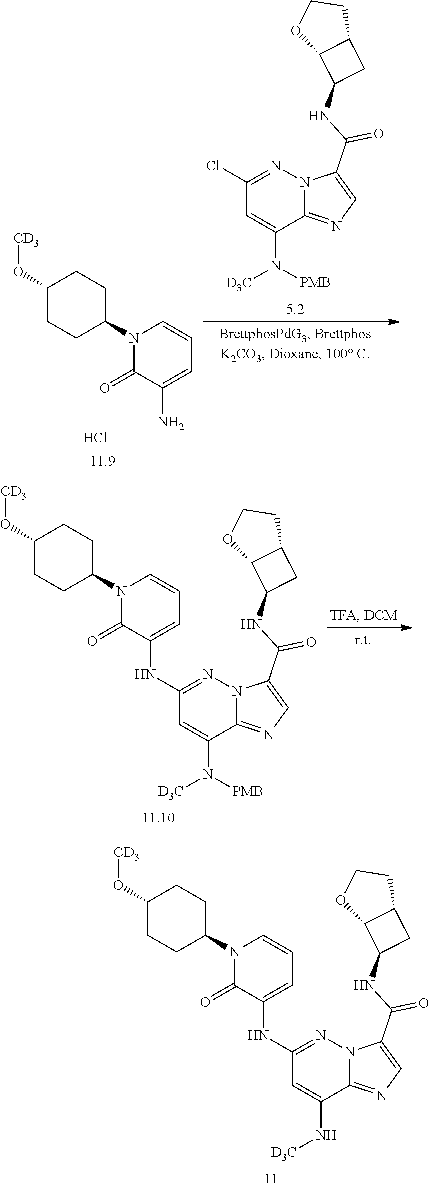

To a solution of (1r,4r)-4-aminocyclohexan-1-ol (11.1, 3.00 g, 26.0 mmol) in dichloromethane (100 mL) were added triphenylmethyl chloride (7.26 g, 26.0 mmol) and triethylamine (7.24 mL, 52.0 mmol), and the mixture was stirred at room temperature overnight under nitrogen atmosphere. The resulting mixture washed with brine (3×50 mL), dried over anhydrous sodium sulfate and concentrated under reduced pressure. The residue was subjected to silica gel column chromatography to afford (1r,4r)-4-(tritylamino)cyclohexan-1-ol (11.2, 7.00 g, 75%) as a white solid. LC-MS (ESI): m/z 358.40 [M+H]⁺.

To a solution of (1r,4r)-4-(tritylamino)cyclohexan-1-ol (11.2, 3.00 g, 8.39 mmol) in tetrahydrofuran (20 mL) was added sodium hydride (1.34 g, 33.6 mmol, 60% in oil) at 0° C., and the mixture was stirred at 0° C. for 30 mins under nitrogen atmosphere. Then to the mixture was added iodomethane-d₃ (1.60 g, 11.0 mmol) dropwise and the resulting mixture was stirred at 0° C. for 4 hrs under nitrogen atmosphere. The reaction mixture was quenched with ice water (50 mL) and extracted with ethyl acetate (3×50 mL). The combined organic layers were washed with brine (3×20 mL), dried over anhydrous sodium sulfate, filtered and concentrated under reduced pressure. The residue was subjected to silica gel column chromatography to afford (1r,4r)-4-(methoxy-d₃)—N-tritylcyclohexan-1-amine (11.3, 2.98 g, 95%) as a yellow solid. LC-MS (ESI): m/z 375.50 [M+H]⁺.

A mixture of (1r,4r)-4-(methoxy-d₃)—N-tritylcyclohexan-1-amine (11.3, 3.00 g, 8.01 mmol) and hydrochloric acid (4 M in 1,4-dioxane, 60 mL) was stirred at room temperature overnight. The resulting mixture was concentrated under reduced pressure. The residue was triturated with petroleum ether/ethyl acetate=20/1 (50 mL). The precipitated solids were collected by filtration, washed with petroleum ether (3×15 mL) and dried in vacuum to afford (1r,4r)-4-(methoxy-d₃)cyclohexan-1-amine hydrochloride (11.4, 1.27 g, 95%) as a yellow solid. LC-MS (ESI): m/z 133.30 [M+H]⁺.

### Procedure D:

To a mixture of (1r,4r)-4-(methoxy-d₃)cyclohexan-1-amine hydrochloride (11.4, 1.12 g, 6.64 mmol) and methyl 2-oxo-2H-pyran-3-carboxylate (11.5, 1.02 g, 6.64 mmol) in N,N-dimethylformamide (15 mL) were added N,N-diisopropylethylamine (2.57 g, 19.9 mmol), and the mixture was stirred at room temperature for 1 hr. Then to the above mixture were added 1-ethyl-3-(3-dimethylaminopropyl)carbodiimide hydrochloride (1.91 g, 9.96 mmol) and 4-dimethylaminopyridine (0.20 g, 1.66 mmol), and the resulting mixture was stirred at room temperature overnight. The reaction mixture was filtered and the filtrate was subjected to reverse phase flash chromatography to afford methyl 1-((1r,4r)-4-(methoxy-d₃)cyclohexyl)-2-oxo-1,2-dihydropyridine-3-carboxylate (11.6, 820 mg, 43%) as a yellow solid. LC-MS (ESI): m/z 269.2 [M+H]⁺.

### Procedure E:

To a suspension of methyl 1-((1r,4r)-4-(methoxy-d₃)cyclohexyl)-2-oxo-1,2-dihydropyridine-3-carboxylate (11.6, 820 mg, 3.06 mmol) in a mixed solvent of water (8 mL), methanol (8 mL) and tetrahydrofuran (16.0 mL) was added lithium hydroxide monohydrate (1.28 g, 30.6 mmol), and the resulting mixture was stirred at 60° C. for 3 hrs. The mixture was cooled to room temperature and adjusted to pH 3-4 with 1 M aq. HCl. The mixture was filtered and the filtrate was concentrated under pressure. The residue was subjected to reverse phase flash chromatography to afford 1-((1r,4r)-4-(methoxy-d₃)cyclohexyl)-2-oxo-1,2-dihydropyridine-3-carboxylic acid (11.7, 400 mg, 49%) as a brown oil. LC-MS (ESI): m/z 255.30 [M+H]⁺.

### Procedure F:

To a mixture of 1-((1r,4r)-4-(methoxy-d₃)cyclohexyl)-2-oxo-1,2-dihydropyridine-3-carboxylic acid (11.7, 270 mg, 1.06 mmol) and triethylamine (429 mg, 4.25 mmol) in tertiary butanol (15 mL) was added diphenyl azidophosphate (876 mg, 3.19 mmol), and the resulting mixture was stirred at 100° C. for 6 hrs. The mixture was cooled to room temperature, quenched with water (30 mL) and extracted with ethyl acetate (3×20 mL). The combined organic layers were washed with brine (3×20 mL), dried over anhydrous sodium sulfate, and then filtered and concentrated under reduced pressure. The residue was subjected to silica gel column chromatography to afford tert-butyl (1-((1r,4r)-4-(methoxy-d₃)cyclohexyl)-2-oxo-1,2-dihydropyridin-3-yl)carbamate (11.8, 80.0 mg, 22%) as a yellow solid. LC-MS (ESI): m/z 326.30 [M+H]⁺.

### Procedure G:

A mixture of tert-butyl (1-((1r,4r)-4-(methoxy-d₃)cyclohexyl)-2-oxo-1,2-dihydropyridin-3-yl)carbamate (11.8, 80.0 mg, 0.25 mmol) and hydrochloric acid (10 mL, 4 M in 1,4-dioxane) was stirred at room temperature for 2 hrs. The resulting mixture was concentrated under reduced pressure to afford 3-amino-1-((1r,4r)-4-(methoxy-d₃)cyclohexyl)pyridin-2(1H)-one hydrochloride (11.9, 77.0 mg, crude) as a yellow solid. LC-MS (ESI): m/z 226.20 [M+H]⁺.

To a stirring mixture of N-((1R,5S,7R)-2-oxabicyclo[3.2.0]heptan-7-yl)-6-chloro-8-((4-methoxybenzyl)(methyl-d₃)amino)imidazo[1,2-b]pyridazine-3-carboxamide (5.2, 50.0 mg, 0.11 mmol) and 3-amino-1-((1r,4r)-4-(methoxy-d₃)cyclohexyl)pyridin-2(1H)-one hydrochloride (11.9, 35.0 mg, 0.13 mmol) in 1,4-dioxane (3 mL) were added BrettPhos Pd G3 (20.0 mg, 0.02 mmol), BrettPhos (24.0 mg, 0.05 mmol) and potassium carbonate (47.0 mg, 0.34 mmol). The resulting mixture was stirred at 100° C. for 2 hrs under nitrogen atmosphere. The mixture was cooled to room temperature, filtered and the filter cake was washed with dichloromethane (3×10 mL), then the filtrate was concentrated under reduced pressure. The residue was subjected to Prep-TLC to afford N-((1R,5S,7R)-2-oxabicyclo[3.2.0]heptan-7-yl)-6-((1-((1r,4R)-4-(methoxy-d₃)cyclohexyl)-2-oxo-1,2-dihydropyridin-3-yl)amino)-8-((4-methoxybenzyl)(methyl-d₃)amino)imidazo[1,2-b]pyridazine-3-carboxamide (11.10, 66.0 mg, 94%) as a green solid. LC-MS (ESI): m/z 634.3 [M+H]⁺.

To a solution of N-((1R,5S,7R)-2-oxabicyclo[3.2.0]heptan-7-yl)-6-((1-((1r,4R)-4-(methoxy-d₃)cyclohexyl)-2-oxo-1,2-dihydropyridin-3-yl)amino)-8-((4-methoxybenzyl)(methyl-d₃)amino)imidazo[1,2-b]pyridazine-3-carboxamide (11.10, 56.0 mg, 0.09 mmol) in dichloromethane (3 mL) was added trifluoroacetic acid (3 mL), and the resulting mixture was stirred at room temperature for 2 hrs. The reaction mixture was concentrated under reduced pressure and the residue was subjected to Prep-HPLC to afford N-((1R,5S,7R)-2-oxabicyclo[3.2.0]heptan-7-yl)-6-((1-((1r,4R)-4-(methoxy-d₃)cyclohexyl)-2-oxo-1,2-dihydropyridin-3-yl)amino)-8-((methyl-d₃)amino)imidazo[1,2-b]pyridazine-3-carboxamide (11, 21.9 mg, 48%) as a white solid. LC-MS (ESI): m/z 514.6 [M+H]⁺. ¹H NMR (400 MHz, DMSO-d₆) δ 8.74 (d, J=7.6 Hz, 1H), 8.50 (s, 1H), 8.00 (dd, J=7.2, 1.6 Hz, 1H), 7.84 (s, 1H), 7.42 (s, 1H), 7.38 (dd, J=7.2, 1.6 Hz, 1H), 6.40-6.24 (m, 2H), 4.85-4.71 (m, 1H), 4.45 (dd, J=7.2, 3.2 Hz, 1H), 4.31-4.20 (m, 1H), 4.11-3.99 (m, 1H), 3.99-3.89 (m, 1H), 3.28-3.16 (m, 1H), 2.96-2.85 (m, 1H), 2.14 (d, J=12.0 Hz, 2H), 2.04-1.68 (m, 8H), 1.41-1.19 (m, 2H).

### Synthetic Example 14. Synthesis of N-((1R,5S,7R)-2-oxabicyclo[3.2.0]heptan-7-yl)-8-(methylamino)-6-((2-oxo-1-(2-oxaspiro[3.5]nonan-7-yl)-1,2-dihydropyridin-3-yl)amino)imidazo[1,2-b]pyridazine-3-carboxamide (12)

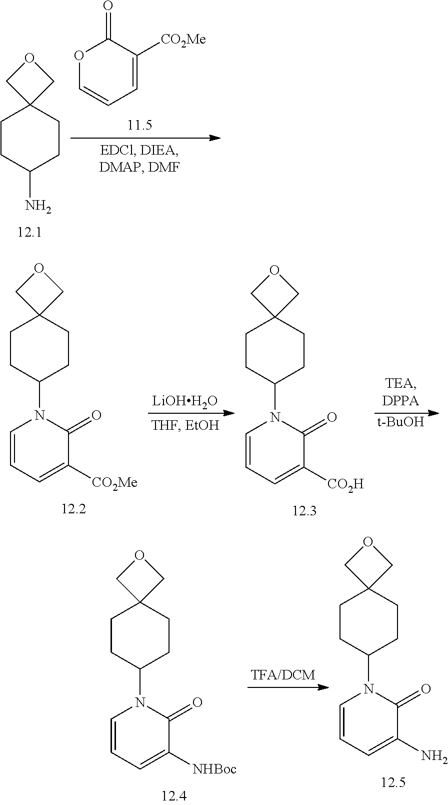

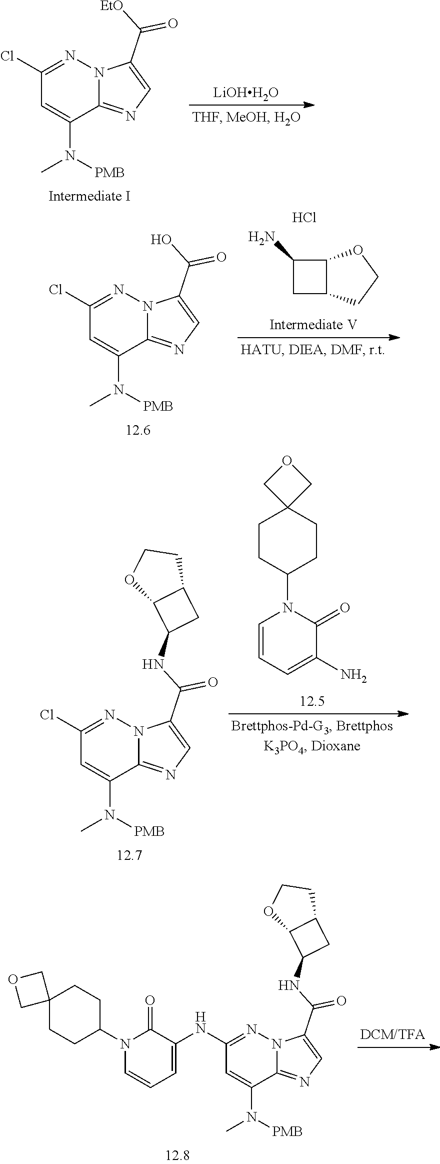

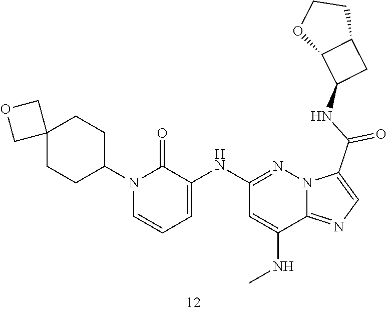 [SMILES: CNc1cc(Nc2cccn(C3CCC4(CC3)COC4)c2=O)nn2c(C(=O)N[C@@H]3C[C@H]4CCO[C@H]43)cnc12]

Compound 12.2 was synthesized from 12.1 similarly to procedure D to afford methyl 2-oxo-1-(2-oxaspiro[3.5]nonan-7-yl)-1,2-dihydropyridine-3-carboxylate (12.2).

Compound 12.3 was synthesized from 12.2 similarly to procedure E to afford 2-oxo-1-(2-oxaspiro[3.5]nonan-7-yl)-1,2-dihydropyridine-3-carboxylic acid (12.3).

Compound 12.4 was synthesized from 12.3 similarly to procedure F to afford tert-butyl (2-oxo-1-(2-oxaspiro[3.5]nonan-7-yl)-1,2-dihydropyridin-3-yl)carbamate (12.4).

Compound 12.5 was synthesized from 12.4 similarly to procedure G to afford 3-amino-1-(2-oxaspiro[3.5]nonan-7-yl)pyridin-2(1H)-one (12.5).

To a solution of ethyl 6-chloro-8-((4-methoxybenzyl)(methyl)amino)imidazo[1,2-b]pyridazine-3-carboxylate (Intermediate I, 3.74 g, 10.0 mmol) in a mixed solvent of ethyl alcohol (10 mL), tetrahydrofuran (10 mL) and water (5 mL) was added lithium hydroxide monohydrate (4.20 g, 100 mmol), and the mixture was stirred at 60° C. for 3 hrs. The reaction mixture was cooled to room temperature and adjusted to pH 6 with 4 M aq. HCl. The precipitated solids were collected by filtration and washed with water (3×10 mL), and then dried in vacuum to afford 6-chloro-8-((4-methoxybenzyl)(methyl)amino) imidazo[1,2-b]pyridazine-3-carboxylic acid (12.6, 3.40 g, 98%). MS (ESI) m/z: 347.1 [M+H]⁺.

To a solution of 6-chloro-8-((4-methoxybenzyl)(methyl)amino) imidazo[1,2-b]pyridazine-3-carboxylic acid (12.6, 3.40 g, 9.83 mmol) in N,N-dimethylformamide (20 mL) were added (1R,5S,7R)-2-oxabicyclo[3.2.0]heptan-7-amine hydrochloride (Intermediate V, 1.76 g, 11.8 mmol), 2-(7-azabenzotriazol-1-yl)-N,N,N′,N′-tetramethyluronium hexafluorophosphate (5.61 g, 14.7 mmol) and N,N-diisopropylethylamine (4.9 mL, 29.50 mmol), and the mixture was stirred for 3 hrs. The reaction mixture was diluted with ethyl acetate (100 mL) and washed with brine (4×50 mL). The organic layer was dried over anhydrous sodium sulfate, filtered and concentrated under reduced pressure. The residue was subjected to silica gel column chromatography to afford N-((1R,5S,7R)-2-oxabicyclo[3.2.0]heptan-7-yl)-6-chloro-8-((4-methoxybenzyl)(methyl)amino)imidazo[1,2-b]pyridazine-3-carboxamide (12.7, 4.06 g, 93%).

To a mixture of N-((1R,5S,7R)-2-oxabicyclo[3.2.0]heptan-7-yl)-6-chloro-8-((4-methoxybenzyl)(methyl)amino)imidazo[1,2-b]pyridazine-3-carboxamide (12.7, 220 mg, 0.30 mmol) and 3-amino-1-(2-oxaspiro[3.5]nonan-7-yl)pyridin-2(1H)-one (12.5, 78.0 mg, 0.33 mmol) in dioxane (5 mL) were added Brettphos Pd G3 (23.7 mg, 0.03 mmol), Brettphos (32.2 mg, 0.06 mmol) and potassium phosphate (203 mg, 0.96 mmol), and the resulting mixture was stirred at 120° C. overnight. The reaction mixture was cooled to room temperature, diluted with water (20 mL) and extracted with ethyl acetate (3×15 mL). The combined organic layers were washed with brine (2×10 mL), dried over anhydrous sodium sulfate, filtrated and concentrated under reduced pressure. The residue was subjected to silica gel column chromatography to afford N-((1R,5S,7R)-2-oxabicyclo[3.2.0]heptan-7-yl)-8-((4-methoxybenzyl)(methyl)amino)-6-((2-oxo-1-(2-oxaspiro[3.5]nonan-7-yl)-1,2-dihydropyridin-3-yl)amino)imidazo[1,2-b]pyridazine-3-carboxamide (12.8, 172 mg, 89%) as a white solid. LC-MS (ESI): m/z 640.8 [M+H]⁺.

To a solution of N-((1R,5S,7R)-2-oxabicyclo[3.2.0]heptan-7-yl)-8-((4-methoxybenzyl)(methyl)amino)-6-((2-oxo-1-(2-oxaspiro[3.5]nonan-7-yl)-1,2-dihydropyridin-3-yl)amino)imidazo[1,2-b]pyridazine-3-carboxamide (12.8, 172 mg, 0.27 mmol) in dichloromethane (2 mL) was added trifluoroacetic acid (2 mL), and the mixture was stirred at room temperature for 3 hrs. The reaction mixture was adjusted to pH 7 with saturated aq. NaHCO₃. The reaction mixture was diluted with water (20 mL) and extracted with dichloromethane (3×15 mL). The combined organic layers were dried over anhydrous sodium sulfate, filtrated and concentrated under reduced pressure. The residue was subjected to Prep-HPLC to afford N-((1R,5S,7R)-2-oxabicyclo[3.2.0]heptan-7-yl)-8-(methylamino)-6-((2-oxo-1-(2-oxaspiro[3.5]nonan-7-yl)-1,2-dihydropyridin-3-yl)amino)imidazo[1,2-b]pyridazine-3-carboxamide (12, 15.0 mg, 18%) as a white solid. LC-MS (ESI): m/z 520.3 [M+H]⁺. ¹H NMR (500 MHz, CDCl₃) δ 8.72 (d, J=6.4 Hz, 1H), 8.09 (s, 1H), 7.89 (d, J=6.1 Hz, 1H), 7.83 (s, 1H), 6.95 (d, J=5.8 Hz, 1H), 6.37 (t, J=7.2 Hz, 1H), 5.74 (s, 1H), 4.92-4.83 (m, 1H), 4.59 (dd, J=6.5, 3.0 Hz, 1H), 4.53 (s, 2H), 4.42 (s, 2H), 4.41-4.37 (m, 1H), 4.22-4.15 (m, 1H), 4.08-4.02 (m, 1H), 3.05 (d, J=5.1 Hz, 3H), 3.03-2.97 (m, 1H), 2.41-2.33 (m, 2H), 2.17-2.09 (m, 1H), 2.09-2.00 (m, 1H), 2.00-1.91 (m, 3H), 1.81-1.68 (m, 3H), 1.61-1.53 (m, 2H).

### Synthetic Example 15. Synthesis of 6-((1-(cis-8-oxabicyclo[3.2.1]octan-3-yl)-2-oxo-1,2-dihydropyridin-3-yl)amino)-N-((1R,5S,7R)-2-oxabicyclo[3.2.0]heptan-7-yl)-8-(methylamino)imidazo[1,2-b]pyridazine-3-carboxamide (13)

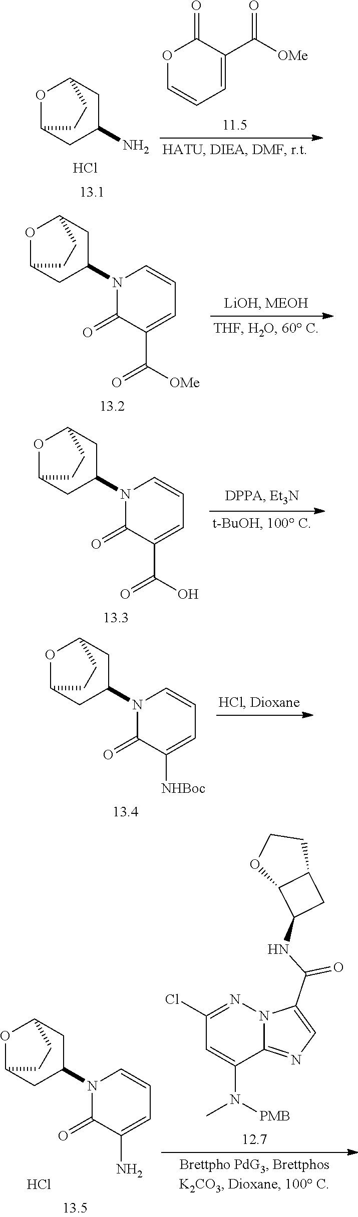

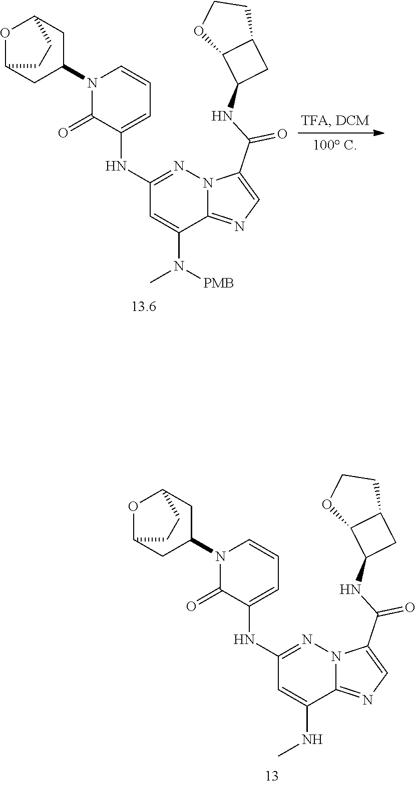

Compound 13.2 was synthesized from 13.1 similarly to procedure D to afford methyl 1-(cis-8-oxabicyclo[3.2.1]octan-3-yl)-2-oxo-1,2-dihydropyridine-3-carboxylate (13.2) as a brown oil. LC-MS (ESI): m/z 264.25 [M+H]⁺.

Compound 13.3 was synthesized from 13.2 similarly to procedure E to afford 1-(cis-8-oxabicyclo[3.2.1]octan-3-yl)-2-oxo-1,2-dihydropyridine-3-carboxylic acid (13.3) as an off-white solid. LC-MS (ESI): m/z 250.05 [M+H]⁺.

Compound 13.4 was synthesized from 13.3 similarly to procedure F to afford tert-butyl (1-(cis-8-oxabicyclo[3.2.1]octan-3-yl)-2-oxo-1,2-dihydropyridin-3-yl)carbamate (13.4) as a white solid. LC-MS (ESI): m/z 321.15 [M+H]⁺.

Compound 13.5 was synthesized from 13.4 similarly to procedure G to afford 3-amino-1-(cis-8-oxabicyclo[3.2.1]octan-3-yl)pyridin-2(1H)-one hydrochloride (13.5) as a yellow solid. LC-MS (ESI): m/z 221.05 [M+H]⁺.

A mixture of N-((1R,5S,7R)-2-oxabicyclo[3.2.0]heptan-7-yl)-6-chloro-8-((4-methoxybenzyl)(methyl)amino)imidazo[1,2-b]pyridazine-3-carboxamide (12.7, 50.0 mg, 0.11 mmol), 3-amino-1-(cis-8-oxabicyclo[3.2.1]octan-3-yl)pyridin-2(1H)-one hydrochloride (13.5, 44.0 mg, 0.17 mmol), BrettPhos (24.0 mg, 0.04 mmol), BrettPhos Pd G3 (20.0 mg, 0.02 mmol) and potassium carbonate (47.0 mg, 0.34 mmol) in dioxane (3 mL) was stirred at 100° C. for 2 hrs under nitrogen atmosphere. The resulting mixture was concentrated under reduced pressure and redissolved with dichloromethane (20 mL). The organic layer was washed with water (2×10 mL), dried over anhydrous sodium sulfate, and then filtrated and concentrated under reduced pressure. The residue was subjected to Prep-TLC (dichloromethane/methanol=15/1) to afford 6-((1-(cis-8-oxabicyclo[3.2.1]octan-3-yl)-2-oxo-1,2-dihydropyridin-3-yl)amino)-N-((1R,5S,7R)-2-oxabicyclo[3.2.0]heptan-7-yl)-8-((4-methoxybenzyl)(methyl)amino)imidazo[1,2-b]pyridazine-3-carboxamide (13.6, 60.0 mg, 87%) as a light green solid. LC-MS (ESI): m/z 626.40 [M+H]⁺.

A solution of 6-((1-(cis-8-oxabicyclo[3.2.1]octan-3-yl)-2-oxo-1,2-dihydropyridin-3-yl)amino)-N-((1R,5S,7R)-2-oxabicyclo[3.2.0]heptan-7-yl)-8-((4-methoxybenzyl)(methyl)amino)imidazo[1,2-b]pyridazine-3-carboxamide (13.6, 55.0 mg, 0.09 mmol) in dichloromethane (2 mL) was added trifluoroacetic acid (2 mL), and the mixture was stirred at room temperature for 1 hr. The resulting mixture was concentrated under reduced pressure and the residue was subjected to Prep-HPLC to afford 6-((1-(cis-8-oxabicyclo[3.2.1]octan-3-yl)-2-oxo-1,2-dihydropyridin-3-yl)amino)-N-((1R,5S,7R)-2-oxabicyclo[3.2.0]heptan-7-yl)-8-(methylamino)imidazo[1,2-b]pyridazine-3-carboxamide (13, 36.9 mg, 83%) as a white solid. LC-MS (ESI): m/z 506.24 [M+H]⁺. ¹H NMR (300 MHz, CDCl₃) δ 8.78 (d, J=6.8 Hz, 1H), 8.08 (s, 1H), 7.93 (d, J=7.1 Hz, 1H), 7.83 (s, 1H), 6.99-6.97 (m, 1H), 6.38 (t, J=7.2 Hz, 1H), 5.99-5.97 (m, 1H), 5.69 (s, 1H), 5.11-5.05 (m, 1H), 4.61-4.60 (m, 3H), 4.42 (m, 1H), 4.24-4.06 (m, 2H), 3.05-2.90 (m, 4H), 2.60-2.51 (m, 2H), 2.19-1.95 (m, 5H), 1.86-1.76 (m, 5H).

### Synthetic Example 16. Synthesis of 6-((1-((1R,5S,7R)-2-oxabicyclo[3.2.0]heptan-7-yl)-2-oxo-1,2-dihydropyridin-3-yl)amino)-N-((1R,5S,7R)-2-oxabicyclo[3.2.0]heptan-7-yl)-8-((methyl-d₃)amino)imidazo[1,2-b]pyridazine-3-carboxamide (14)

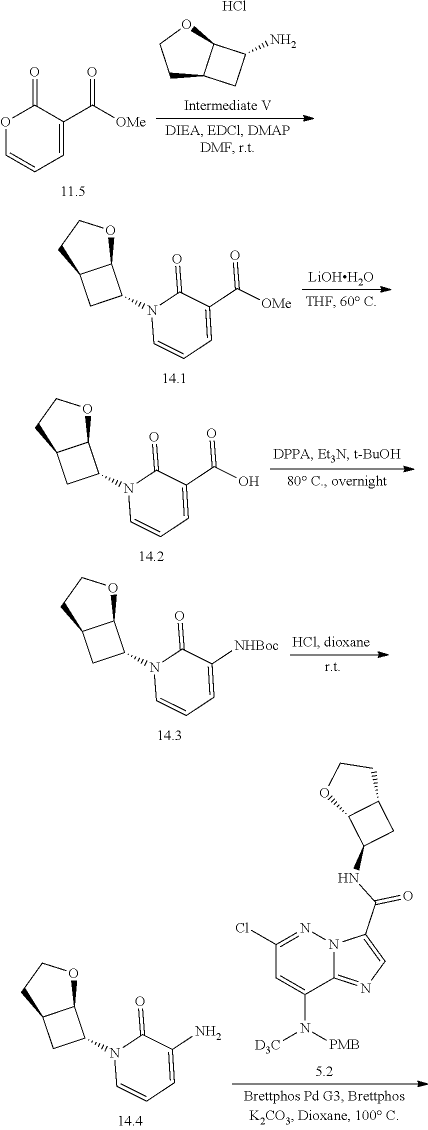

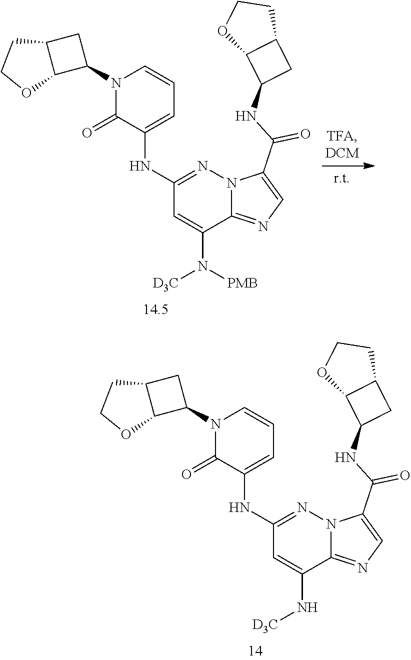

Compound 14.1 was synthesized from 11.5 similarly to procedure D to afford methyl 1-((1R,5S,7R)-2-oxabicyclo[3.2.0]heptan-7-yl)-2-oxo-1,2-dihydropyridine-3-carboxylate (14.1) as a yellow solid. LC-MS (ESI): m/z 250.1 [M+H]⁺.

Compound 14.2 was synthesized from 14.1 similarly to procedure E to afford 1-((1R,5S,7R)-2-oxabicyclo[3.2.0]heptan-7-yl)-2-oxo-1,2-dihydropyridine-3-carboxylic acid (14.2) as a yellow solid. LC-MS (ESI): m/z 236.1 [M+H]⁺.

Compound 14.3 was synthesized from 14.2 similarly to procedure F to afford tert-butyl (1-((1R,5S,7R)-2-oxabicyclo[3.2.0]heptan-7-yl)-2-oxo-1,2-dihydropyridin-3-yl)carbamate (14.3) as a brown oil. LC-MS (ESI): m/z 307.2 [M+H]⁺.

Compound 14.4 was synthesized from 14.3 similarly to procedure G to afford 3-amino-1-((1R,5S,7R)-2-oxabicyclo[3.2.0]heptan-7-yl)pyridin-2(1H)-one (14.4) as a white solid. LC-MS (ESI): m/z 207.1 [M+H]⁺.

To a mixture of N-((1R,5S,7R)-2-oxabicyclo[3.2.0]heptan-7-yl)-6-chloro-8-((4-methoxybenzyl)(methyl-d₃)amino)imidazo[1,2-b]pyridazine-3-carboxamide (5.2, 133 mg, 0.30 mmol) and 3-amino-1-((1R,5S,7R)-2-oxabicyclo[3.2.0]heptan-7-yl)pyridin-2(1H)-one (14.4, 61.0 mg, 0.30 mmol) in dioxane (10 mL) were added BrettPhos Pd G3 (54.0 mg, 0.06 mmol), BrettPhos (32.0 mg, 0.06 mmol) and potassium carbonate (165 mg, 1.20 mmol), and the resulting mixture was stirred at 100° C. for 1 hr under nitrogen atmosphere. The resulting mixture was concentrated under reduced pressure and redissolved with dichloromethane (20 mL). The organic layer was washed with water (2×10 mL), dried over anhydrous sodium sulfate, and then filtrated and concentrated under reduced pressure. The residue was subjected to Prep-TLC (dichloromethane/methanol=10/1) to afford 6-((1-((1R,5S,7R)-2-oxabicyclo[3.2.0]heptan-7-yl)-2-oxo-1,2-dihydropyridin-3-yl)amino)-N-((1R,5S,7R)-2-oxabicyclo[3.2.0]heptan-7-yl)-8-((4-methoxybenzyl)(methyl-d₃)amino)imidazo[1,2-b]pyridazine-3-carboxamide (14.5, 101 mg, 55%) as a blue solid. LC-MS (ESI): m/z 615.3 [M+H]⁺.

A mixture of 6-((1-((1R,5S,7R)-2-oxabicyclo[3.2.0]heptan-7-yl)-2-oxo-1,2-dihydropyridin-3-yl)amino)-N-((1R,5S,7R)-2-oxabicyclo[3.2.0]heptan-7-yl)-8-((4-methoxybenzyl)(methyl-d₃)amino)imidazo[1,2-b]pyridazine-3-carboxamide (14.5, 100 mg, 0.16 mmol) and trifluoroacetic acid (2 mL) in dichloromethane (2 mL) was stirred at room temperature for 1 hr. The resulting mixture was concentrated under reduced pressure and the residue was subjected to Prep-HPLC to afford 6-((1-((1R,5S,7R)-2-oxabicyclo[3.2.0]heptan-7-yl)-2-oxo-1,2-dihydropyridin-3-yl)amino)-N-((1R,5S,7R)-2-oxabicyclo[3.2.0]heptan-7-yl)-8-((methyl-d₃)amino)imidazo[1,2-b]pyridazine-3-carboxamide (14, 39.0 mg, 48%) as a blue solid. LC-MS (ESI): m/z 495.25 [M+H]⁺. ¹H NMR (300 MHz, CDCl₃) δ 8.80 (d, J=6.5 Hz, 1H), 8.08 (s, 1H), 7.94 (d, J=5.9 Hz, 1H), 7.78 (s, 1H), 7.03 (d, J=7.2 Hz, 1H), 6.40 (t, J=7.1 Hz, 1H), 5.99 (s, 1H), 5.68 (s, 1H), 4.99 (d, J=7.0 Hz, 2H), 4.61 (s, 1H), 4.44 (s, 1H), 4.25-4.09 (m, 4H), 3.21-3.10 (m, 1H), 3.02-3.01 (m, 1H), 2.55-2.50 (m, 1H), 2.14-1.96 (m, 6H), 1.91-1.83 (m, 1H).

### Synthetic Example 17. Synthesis of N-((1R,2S)-2-fluorocyclopropyl)-6-((1-((1s,4s)-4-(methoxy-d₃)cyclohexyl)-2-oxo-1,2-dihydropyridin-3-yl)amino)-8-(methylamino)imidazo[1,2-b]pyridazine-3-carboxamide hydrochloride (15)

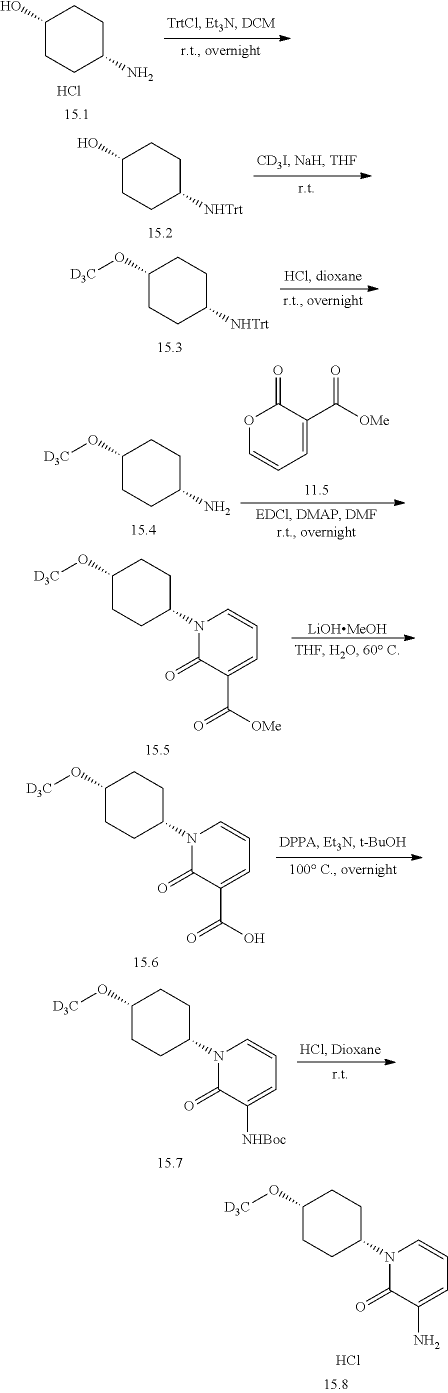

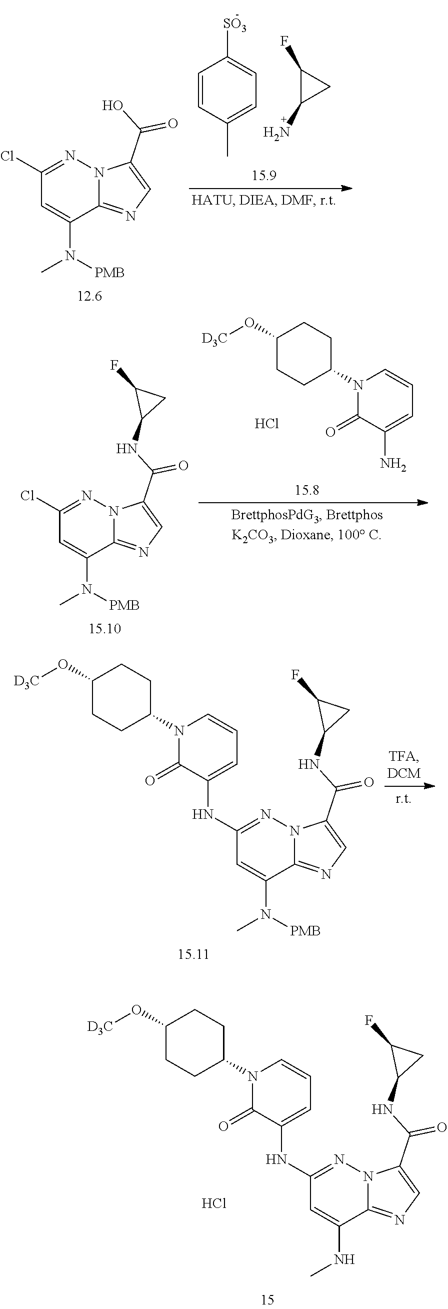

To a mixture of (1s,4s)-4-aminocyclohexan-1-ol hydrochloride (15.1, 5.00 g, 33.0 mmol) and triphenylmethyl chloride (10.0 g, 36.3 mmol) in dichloromethane (100 mL) was added triethylamine (10.0 g, 99.0 mmol), and the resulting mixture was stirred at room temperature overnight. The mixture was concentrated under reduced pressure. The residue was subjected to silica gel column chromatography to afford (1s,4s)-4-(tritylamino)cyclohexan-1-ol (15.2, 10.0 g, 84%) as a white solid. LC-MS (ESI): m/z 358.28 [M+H]⁺.

To a solution of (1s,4s)-4-(tritylamino)cyclohexan-1-ol (15.2, 2.20 g, 6.15 mmol) in tetrahydrofuran (50 mL) was added sodium hydride (0.74 g, 1.85 mmol, 60% in oil) at 0° C., and the mixture was stirred at 0° C. for 15 mins. Then to the mixture was added iodomethane-d₃ (1.34 g, 9.23 mmol) and the resulting mixture was warmed to room temperature and stirred for 4 hrs. The reaction mixture was quenched with ice water (50 mL) and extracted with ethyl acetate (3×30 mL). The combined organic layers were dried over anhydrous sodium sulfate, filtrated and concentrated under reduced pressure. The residue was subjected to silica gel column chromatography to afford (1s,4s)-4-(methoxy-d₃)—N-tritylcyclohexan-1-amine (15.3, 1.30 g, 56%) as a white solid. LC-MS (ESI): m/z 375.26 [M+H]⁺.

A mixture of (1s,4s)-4-(methoxy-d₃)—N-tritylcyclohexan-1-amine (15.3, 1.2 g, 3.20 mmol) and hydrochloric acid (20 mL, 4 M in dioxane) was stirred at room temperature overnight. The resulting mixture was concentrated under reduced pressure. The residue was triturated with petroleum ether/ethyl acetate=10/1 (20 mL). The precipitated solids were collected by filtration and washed with petroleum ether (3×10 mL), and then dried in vacuum to afford (1s,4s)-4-(methoxy-d₃)cyclohexan-1-amine hydrochloride (15.4, 650 mg, crude) as a white solid. LC-MS (ESI): m/z 133.22 [M+H]⁺.

Compound 15.5 was synthesized from 15.4 similarly to procedure D to afford methyl 1-((1s,4s)-4-(methoxy-d₃)cyclohexyl)-2-oxo-1,2-dihydropyridine-3-carboxylate (15.5) as a brown oil. LC-MS (ESI): m/z 269.17 [M+H]⁺.

Compound 15.6 was synthesized from 15.5 similarly to procedure E to afford 1-((1s,4s)-4-(methoxy-d₃)cyclohexyl)-2-oxo-1,2-dihydropyridine-3-carboxylic acid (15.6) as a yellow oil. LC-MS (ESI): m/z 255.13 [M+H]⁺.

Compound 15.7 was synthesized from 15.6 similarly to procedure F to afford tert-butyl (1-((1s,4s)-4-(methoxy-d₃)cyclohexyl)-2-oxo-1,2-dihydropyridin-3-yl)carbamate (15.7) as a grey solid. LC-MS (ESI): m/z 326.27 [M+H]⁺.

Compound 15.8 was synthesized from 15.7 similarly to procedure G to afford 3-amino-1-((1s,4s)-4-(methoxy-d₃)cyclohexyl)pyridin-2(1H)-one hydrochloride (15.8) as a red solid. LC-MS (ESI): m/z 226.20 [M+H].

To a mixture of 6-chloro-8-((4-methoxybenzyl)(methyl)amino)imidazo[1,2-b]pyridazine-3-carboxylic acid (12.6, 750 mg, 2.16 mmol) and (1R,2S)-2-fluorocyclopropan-1-aminium 4-methylbenzenesulfonate (15.9, 802 mg, 3.24 mmol) in N,N-dimethylformamide (10 mL) were added 2-(7-azabenzotriazol-1-yl)-N,N,N′,N′-tetramethyluronium hexafluorophosphate (1.64 g, 4.33 mmol) and N,N-diisopropylethylamine (1.40 g, 10.8 mmol), and the resulting mixture was stirred at room temperature for 1 hr. The reaction mixture was filtered and the filtrate was subjected to reverse phase flash chromatography to afford 6-chloro-N-((1R,2S)-2-fluorocyclopropyl)-8-((4-methoxybenzyl)(methyl)amino)imidazo[1,2-b]pyridazine-3-carboxamide (15.10, 660 mg, 75%) as a yellow solid. LC-MS (ESI): m/z 404.1 [M+H]⁺.

To a mixture of 6-chloro-N-((1R,2S)-2-fluorocyclopropyl)-8-((4-methoxybenzyl)(methyl)amino)imidazo[1,2-b]pyridazine-3-carboxamide (15.10, 200 mg, 0.50 mmol) and 3-amino-1-((1s,4s)-4-(methoxy-d₃)cyclohexyl)pyridin-2(1H)-one hydrochloride (15.8, 194 mg, 0.74 mmol) in dioxane (10 mL) were added BrettPhos Pd G3 (179 mg, 0.20 mmol), BrettPhos (106 mg, 0.20 mmol), potassium carbonate (137 mg, 1.00 mmol), and the resulting mixture was stirred at 100° C. for 3 hrs under nitrogen atmosphere. The reaction mixture was cooled to room temperature and diluted water (20 mL), then the mixture was extracted with dichloromethane (3×15 mL). The combined organic layers were washed with brine (3×10 mL), dried over anhydrous sodium sulfate, and then filtrated and concentrated under reduced pressure. The residue was subjected to silica gel column chromatography to afford N-((1R,2S)-2-fluorocyclopropyl)-6-((1-((1s,4s)-4-(methoxy-d₃)cyclohexyl)-2-oxo-1,2-dihydropyridin-3-yl)amino)-8-((4-methoxybenzyl)(methyl)amino)imidazo[1,2-b]pyridazine-3-carboxamide (15.11, 263 mg, 89%) as a yellow solid. LC-MS (ESI): m/z 593.40 [M+H]⁺.

To a solution of N-((1R,2S)-2-fluorocyclopropyl)-6-((1-((1s,4s)-4-(methoxy-d₃)cyclohexyl)-2-oxo-1,2-dihydropyridin-3-yl)amino)-8-((4-methoxybenzyl)(methyl)amino)imidazo[1,2-b]pyridazine-3-carboxamide (15.11, 263 mg, 0.44 mmol) in dichloromethane (4 mL) was added trifluoroacetic acid (4 mL), and the resulting mixture was stirred at room temperature for 1 hr. The mixture was concentrated under reduced pressure and the residue was subjected to Prep-HPLC to afford the product as a free base. Then it was treated with hydrochloric acid (20 mL, 6 M in methanol). The resulting mixture was concentrated under reduced pressure and dried in vacuum to afford N-((1R,2S)-2-fluorocyclopropyl)-6-((1-((1s,4s)-4-(methoxy-d₃)cyclohexyl)-2-oxo-1,2-dihydropyridin-3-yl)amino)-8-(methylamino)imidazo[1,2-b]pyridazine-3-carboxamide hydrochloride (15, 105 mg, 46%) as a yellow solid. LC-MS (ESI): m/z 473.20 [M+H]⁺. ¹H NMR (400 MHz, CDCl₃) δ 9.22 (d, J=5.4 Hz, 1H), 8.55 (d, J=4.4 Hz, 1H), 8.22 (s, 1H), 8.01 (s, 1H), 7.70 (dd, J=7.2, 1.6 Hz, 1H), 7.17 (dd, J=7.2, 1.6 Hz, 1H), 6.32 (t, J=7.2 Hz, 1H), 5.96 (s, 1H), 5.04-4.94 (m, 1H), 4.92-4.67 (m, 1H), 3.54 (t, J=2.9 Hz, 1H), 3.17-3.10 (m, 1H), 3.08 (d, J=4.8 Hz, 3H), 2.19-2.11 (m, 2H), 1.97-1.86 (m, 2H), 1.74 (d, J=11.9 Hz, 2H), 1.64 (t, J=13.4 Hz, 2H), 1.38-1.27 (m, 1H), 1.17-1.05 (m, 1H).

### Synthetic Example 18. Synthesis of N-((1R,2S)-2-fluorocyclopropyl)-6-((1-((1r,4r)-4-methoxycyclohexyl)-2-oxo-1,2-dihydropyridin-3-yl)amino)-8-(methylamino)imidazo[1,2-b]pyridazine-3-carboxamide (16)

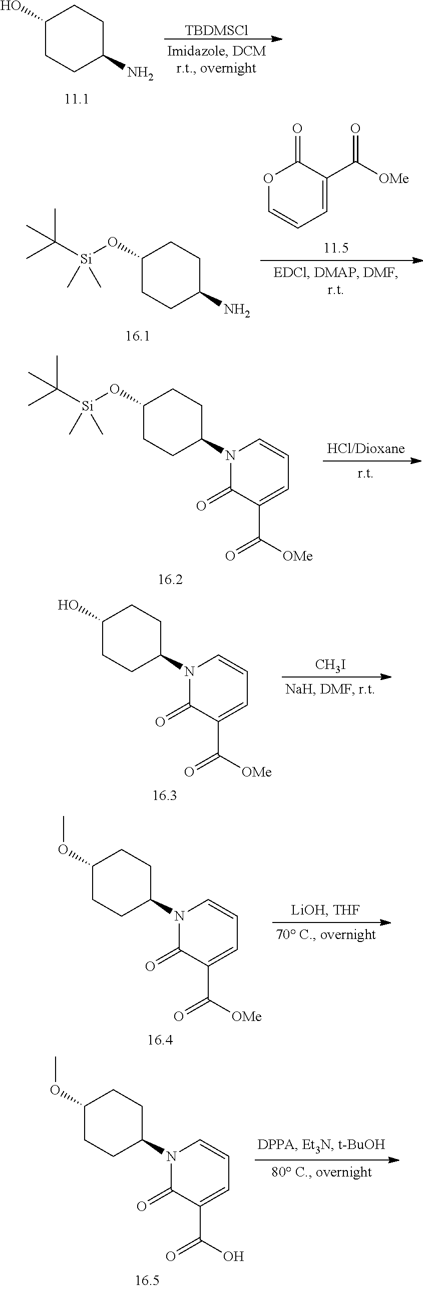

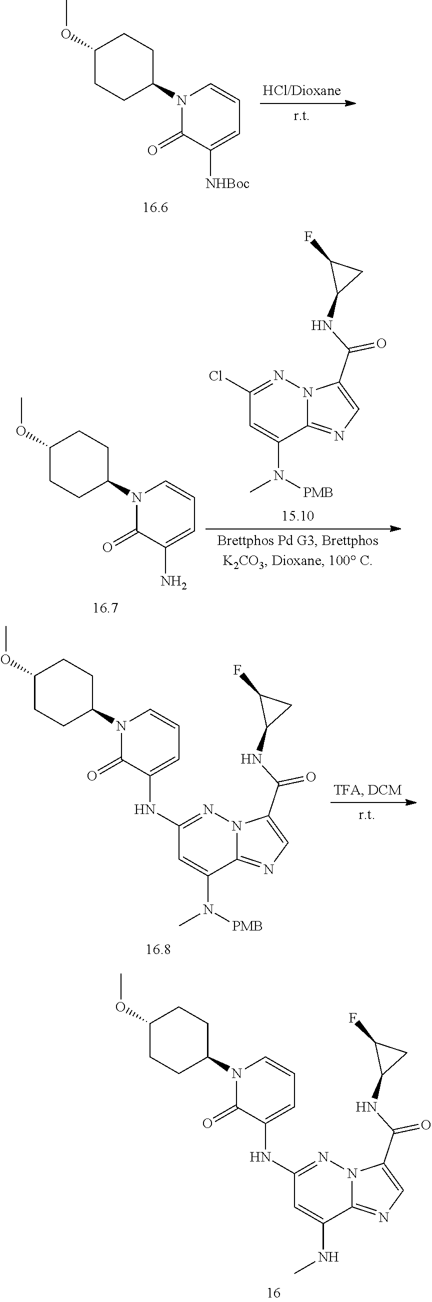

To a mixture of (1r,4r)-4-aminocyclohexan-1-ol (11.1, 10.0 g, 86.8 mmol) and t-butyldimethylchlorosilane (19.6 g, 130 mmol) in dichloromethane (100 mL) was added imidazole (17.7 g, 260 mmol), and the reaction mixture was stirred at room temperature overnight. The mixture was washed with brine (3×50 mL), dried over anhydrous sodium sulfate, and then filtrated and concentrated under reduced pressure. The residue was subjected to silica gel column chromatography to afford (1r,4r)-4-((tert-butyldimethylsilyl)oxy)cyclohexan-1-amine (16.1, 17.0 g, 85%) as a brown oil. LC-MS (ESI): m/z 230.2 [M+H]⁺.

Compound 16.2 was synthesized from 16.1 similarly to procedure D to afford methyl 1-((1r,4r)-4-((tert-butyldimethylsilyl)oxy)cyclohexyl)-2-oxo-1,2-dihydropyridine-3-carboxylate (16.2) as a brown oil. LC-MS (ESI): m/z 366.2 [M+H]⁺.

A mixture of methyl 1-((1r,4r)-4-((tert-butyldimethylsilyl)oxy)cyclohexyl)-2-oxo-1,2-dihydropyridine-3-carboxylate (16.2, 5.00 g, 13.7 mmol) and hydrochloric acid (10 mL, 4 M in dioxane) was stirred at room temperature for 1 hr. The mixture was concentrated under reduced pressure and the residue was subjected to reverse phase flash chromatography to afford methyl 1-((1r,4r)-4-hydroxycyclohexyl)-2-oxo-1,2-dihydropyridine-3-carboxylate (16.3, 2.30 g, 67%) as a brown solid. LC-MS (ESI): m/z 252.1 [M+H]⁺.

A mixture of methyl 1-((1r,4r)-4-hydroxycyclohexyl)-2-oxo-1,2-dihydropyridine-3-carboxylate (16.3, 2.00 g, 7.96 mmol) in N,N-dimethylformamide (20 mL) was added sodium hydride (480 mg, 11.9 mmol, 60% in mineral oil) at 0° C., and the mixture was stirred at 0° C. for 30 mins. Then to the mixture was added iodomethane (1.13 g, 7.96 mmol) and it was stirred at room temperature for 16 hrs. The reaction mixture was quenched with ice water (30 mL) and extracted with ethyl acetate (3×15 mL). The combined organic layers were dried over anhydrous sodium sulfate, and then filtrated and concentrated under reduced pressure. The residue was subjected to reverse phase flash chromatography to afford methyl 1-((1r,4r)-4-methoxycyclohexyl)-2-oxo-1,2-dihydropyridine-3-carboxylate (16.4, 300 mg, 14%) as a red solid. LC-MS (ESI): m/z 266.1 [M+H]⁺.

Compound 16.5 was synthesized from 16.4 similarly to procedure E to afford 1-((1r,4r)-4-methoxycyclohexyl)-2-oxo-1,2-dihydropyridine-3-carboxylic acid (16.5) as a brown solid. LC-MS (ESI): m/z 252.1 [M+H]⁺.

Compound 16.6 was synthesized from 16.5 similarly to procedure F to afford tert-butyl (1-((1r,4r)-4-methoxycyclohexyl)-2-oxo-1,2-dihydropyridin-3-yl)carbamate (16.6) as a white solid. LC-MS (ESI): m/z 323.2 [M+H]⁺.

Compound 16.7 was synthesized from 16.6 similarly to procedure G to afford 3-amino-1-((1r,4r)-4-methoxycyclohexyl)pyridin-2(1H)-one hydrochloride (16.7) as a brown oil. LC-MS (ESI): m/z 223.1 [M+H]⁺.

To a mixture of 6-chloro-N-((1R,2S)-2-fluorocyclopropyl)-8-((4-methoxybenzyl)(methyl)amino)imidazo[1,2-b]pyridazine-3-carboxamide (15.10, 150 mg, 0.37 mmol) and 3-amino-1-((1r,4r)-4-methoxycyclohexyl)pyridin-2(1H)-one hydrochloride (16.7, 99 mg, 0.44 mmol) in dioxane (5 mL) were added BrettPhos Pd G₃ (67 mg, 0.07 mmol), BrettPhos (40 mg, 0.07 mmol) and potassium carbonate (154 mg, 1.11 mmol), and the reaction mixture was stirred at 100° C. for 3 hrs under nitrogen atmosphere. The reaction mixture was cooled to room temperature and diluted water (20 mL), then the mixture was extracted with dichloromethane (3×15 mL). The combined organic layers were washed with brine (3×10 mL), dried over anhydrous sodium sulfate, and then filtrated and concentrated under reduced pressure. The residue was subjected to silica gel column chromatography to afford N-((1R,2S)-2-fluorocyclopropyl)-8-((4-methoxybenzyl)(methyl)amino)-6-((1-((1r,4r)-4-methoxycyclohexyl)-2-oxo-1,2-dihydropyridin-3-yl)amino)imidazo[1,2-b]pyridazine-3-carboxamide (16.8, 200 mg, 91%) as a brown oil. LC-MS (ESI): m/z 590.3 [M+H]⁺.

A solution of N-((1R,2S)-2-fluorocyclopropyl)-8-((4-methoxybenzyl)(methyl)amino)-6-((1-((1r,4r)-4-methoxycyclohexyl)-2-oxo-1,2-dihydropyridin-3-yl)amino)imidazo[1,2-b]pyridazine-3-carboxamide (16.8, 100 mg, 0.15 mmol) in dichloromethane (4 mL) was added trifluoroacetic acid (4 mL), and the reaction mixture was stirred at room temperature for 1 hr. The mixture was concentrated under reduced pressure and the residue was subjected to Prep-HPLC to afford N-((1R,2S)-2-fluorocyclopropyl)-6-((1-((1r,4r)-4-methoxycyclohexyl)-2-oxo-1,2-dihydropyridin-3-yl)amino)-8-(methylamino)imidazo[1,2-b]pyridazine-3-carboxamide (16, 43.4 mg, 61%) as a white solid. LC-MS (ESI): m/z 470.2 [M+H]⁺. ¹H NMR (400 MHz, DMSO-d₆) δ 8.62 (d, J=4.0 Hz, 1H), 8.48 (s, 1H), 7.90 (s, 1H), 7.84 (d, J=6.4 Hz, 1H), 7.48 (s, 1H), 7.35 (d, J=7.2, 1H), 6.35 (s, 1H), 6.29 (t, J=7.2 Hz, 1H), 4.95-4.77 (m, 2H), 3.27-3.25 (m, 4H), 2.98-2.96 (m, 1H), 2.87 (d, J=4.8 Hz, 3H), 2.14 (d, J=12.2 Hz, 2H), 1.81-1.76 (m, 4H), 1.42-1.13 (m, 3H), 0.99-0.95 (m, 1H).

### Synthetic Example 19. Synthesis of N-(6,7-dihydro-5H-pyrrolo[1,2-b][1,2,4]triazol-7-yl)-7-(methylamino)-5-((2-oxo-2H-[1,2′-bipyridin]-3-yl)amino)pyrazolo[1,5-a]pyrimidine-3-carboxamide (17)

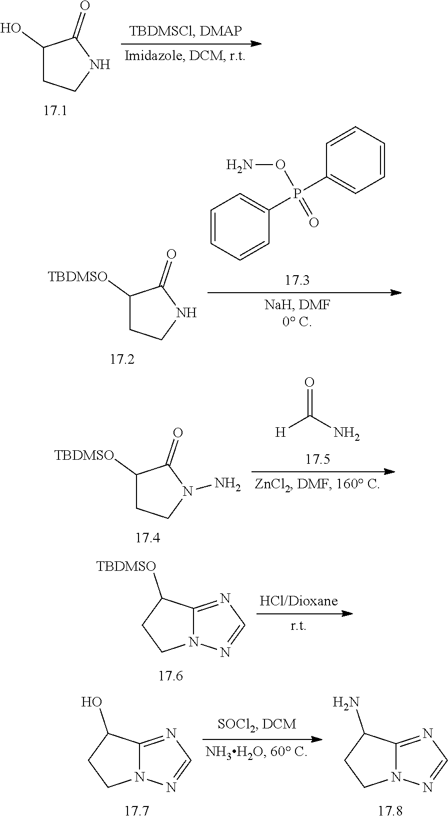

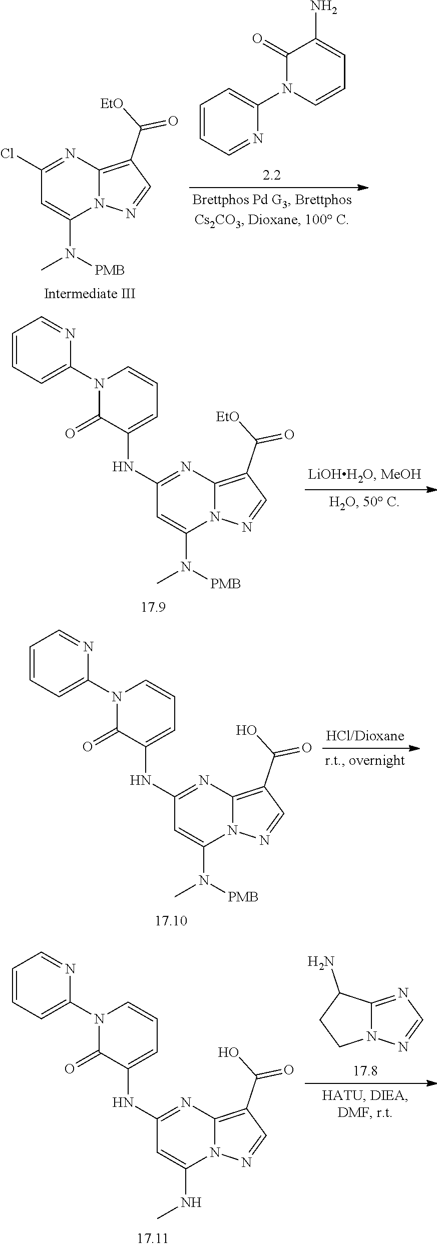

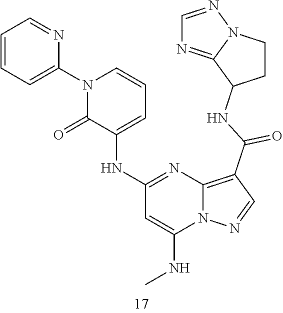 [SMILES: CNc1cc(Nc2cccn(-c3ccccn3)c2=O)nc2c(C(=O)NC3CCn4ncnc43)cnn12]

To a solution of 3-hydroxypyrrolidin-2-one (17.1, 5.00 g, 49.5 mmol) in dichloromethane (100 mL) were added t-butyldimethylchlorosilane (8.91 g, 59.4 mmol), 4-dimethylaminopyridine (200 mg, 1.60 mmol) and imidazole (6.73 g, 99.0 mmol), and the reaction mixture was stirred at room temperature for 12 hrs. The mixture was washed with brine (3×30 mL), dried over anhydrous sodium sulfate, and then filtrated and concentrated under reduced pressure. The residue was subjected to reverse phase flash chromatography to afford 3-((tert-butyldimethylsilyl)oxy)pyrrolidin-2-one (17.2, 9.07 g, 85%) as a white solid. LC-MS (ESI): m/z 215.2 [M+H]⁺.

To a solution of 3-((tert-butyldimethylsilyl)oxy)pyrrolidin-2-one (17.2, 10.0 g, 46.4 mmol) in N,N-dimethylformamide (150 mL) was added sodium hydride (2.78 g, 69.6 mmol, 60% in mineral oil) at 0° C., and the mixture was stirred at 0° C. for 30 mins. Then to the above mixture was added (aminooxy)diphenylphosphine oxide (17.3, 16.2 g, 69.6 mmol) and the resulting mixture was stirred at 0° C. for 1 h. The reaction mixture was quenched with ice water (150 ml) and extracted with ethyl acetate (3×50 mL). The combined organic layers were dried over anhydrous sodium sulfate, and then filtrated and concentrated under reduced pressure. The residue was subjected to reverse phase flash chromatography to afford 1-amino-3-((tert-butyldimethylsilyl)oxy)pyrrolidin-2-one (17.4, 4.50 g, 42%) as a white solid. LC-MS (ESI): m/z 231.1 [M+H]⁺.

To a mixture of 1-amino-3-((tert-butyldimethylsilyl)oxy)pyrrolidin-2-one (17.4, 2.00 g, 8.68 mmol) and zinc chloride (360 mg, 2.60 mmol) in N,N-dimethylformamide (20 mL) was added formamide (17.5, 1.96 g, 43.4 mmol), and the mixture was stirred at 160° C. for 16 hrs under nitrogen atmosphere. The mixture was cooled to room temperature, quenched with water (50 mL) dropwise and extracted with ethyl acetate (3×50 mL). The combined organic layers were washed with brine (4×30 mL), dried over anhydrous sodium sulfate, and then filtrated and concentrated under reduced pressure. The residue was subjected to reverse phase flash chromatography to afford 7-((tert-butyldimethylsilyl)oxy)-6,7-dihydro-5H-pyrrolo[1,2-b][1,2,4]triazole (17.6, 540 mg, 26%) as a white solid. LC-MS (ESI): m/z 240.2 [M+H]⁺.

A mixture of 7-((tert-butyldimethylsilyl)oxy)-6,7-dihydro-5H-pyrrolo[1,2-b][1,2,4]triazole (17.6, 480 mg, 2.00 mmol) and hydrochloric acid (10 mL, 4 M in dioxane) was stirred at room temperature for 1 hr. The resulting mixture was concentrated under reduced pressure to afford 6,7-dihydro-5H-pyrrolo[1,2-b][1,2,4]triazol-7-ol (17.7, 350 mg, crude) as a white solid. LC-MS (ESI): m/z 126.1 [M+H]⁺.

To a mixture of 6,7-dihydro-5H-pyrrolo[1,2-b][1,2,4]triazol-7-ol (17.7, 350 mg, crude) in dichloromethane (15 mL) was added thionyl chloride (666 mg, 5.60 mmol) at 0° C., and the mixture was stirred at room temperature for 2 hrs under nitrogen atmosphere. Then to the mixture was added ammonium hydroxide (35 mL) and the resulting mixture was stirred at 60° C. overnight. The reaction mixture was concentrated under reduced pressure and the residue was subjected to reverse phase flash chromatography to afford 6,7-dihydro-5H-pyrrolo[1,2-b][1,2,4]triazol-7-amine (17.8, 180 mg) as a brown oil. LC-MS (ESI): m/z 125.0 [M+H]⁺.

Compound 17.9 was synthesized from Intermediate III similarly to procedure A to afford ethyl 7-((4-methoxybenzyl)(methyl)amino)-5-((2-oxo-2H-[1,2′-bipyridin]-3-yl)amino)pyrazolo[1,5-a]pyrimidine-3-carboxylate (17.9) as a red solid. LC-MS (ESI): m/z 526.2 [M+H]⁺.

Compound 17.10 was synthesized from 17.9 similarly to procedure B to afford 7-((4-methoxybenzyl)(methyl)amino)-5-((2-oxo-2H-[1,2′-bipyridin]-3-yl)amino)pyrazolo[1,5-a]pyrimidine-3-carboxylic acid (17.10) as a brown solid. LC-MS (ESI): m/z 498.3 [M+H]⁺.

Compound 17.11 was synthesized from 17.10 similarly to procedure C to afford 7-(methylamino)-5-((2-oxo-2H-[1,2′-bipyridin]-3-yl)amino)pyrazolo[1,5-a]pyrimidine-3-carboxylic acid (17.11) as a brown solid. LC-MS (ESI): m/z 378.3 [M+H]⁺.

To a mixture of 7-(methylamino)-5-((2-oxo-2H-[1,2′-bipyridin]-3-yl)amino)pyrazolo[1,5-a]pyrimidine-3-carboxylic acid (17.11, 150 mg, crude) and (benzotriazollyloxy)tris(dimethylamino)phosphonium hexafluophosphate (352 mg, 0.79 mmol) in N,N-dimethylformamide (2 mL) were added N,N-diisopropylethylamine (154 mg, 1.19 mmol) and 6,7-dihydro-5H-pyrrolo[1,2-b][1,2,4]triazol-7-amine (17.8, 74.0 mg, 0.60 mmol), and the reaction mixture was stirred at room temperature for 3 hrs. The reaction was diluted with water (20 mL) and extracted with dichloromethane (3×15 mL). The combined organic layers were washed with brine (3×15 mL), dried over anhydrous sodium sulfate, and then filtrated and concentrated under reduced pressure. The residue was subjected to Prep-HPLC to afford N-(6,7-dihydro-5H-pyrrolo[1,2-b][1,2,4]triazol-7-yl)-7-(methylamino)-5-((2-oxo-2H-[1,2′-bipyridin]-3-yl)amino)pyrazolo[1,5-a]pyrimidine-3-carboxamide (17, 11.9 mg) as an off-white solid. LC-MS (ESI): m/z 484.15 [M+H]⁺. ¹H NMR (300 MHz, CDCl₃): δ 8.62 (s, 1H), 8.35-8.29 (m, 2H), 8.17-8.11 (m, 2H), 8.02-8.00 (m, 1H), 7.96-7.84 (m, 2H), 7.50-7.48 (m, 1H), 7.39-7.28 (m, 1H), 6.38 (s, 1H), 6.00 (s, 1H), 5.80-5.63 (m, 1H), 5.49 (s, 1H), 4.54-4.33 (m, 1H), 4.30-4.14 (m, 1H), 3.54-3.33 (m, 1H), 3.07 (m, 3H), 2.86-2.61 (m, 1H).

### Synthetic Example 20. Synthesis of N-(6,7-dihydro-5H-pyrrolo[1,2-a]imidazol-7-yl)-7-(methylamino)-5-((2-oxo-2H-[1,2′-bipyridin]-3-yl)amino)pyrazolo[1,5-a]pyrimidine-3-carboxamide (18)

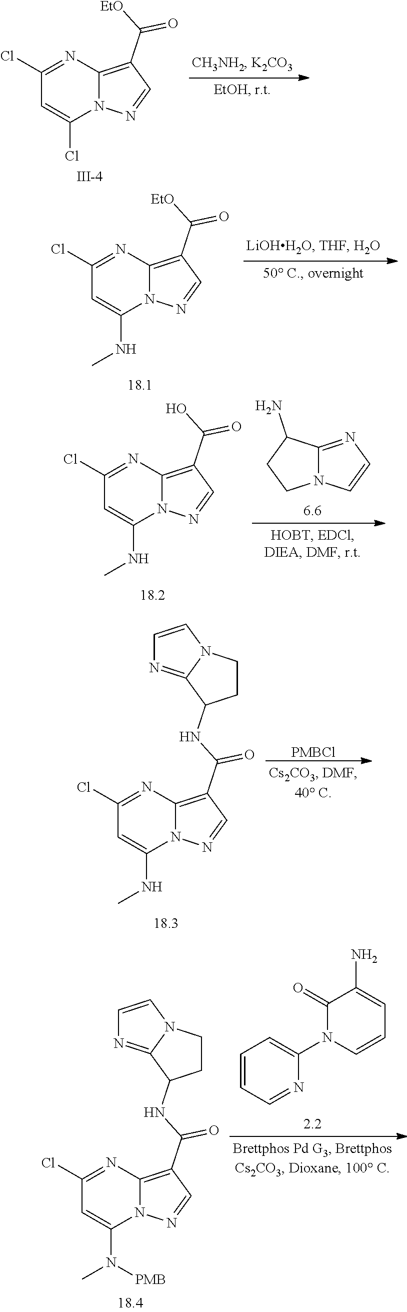

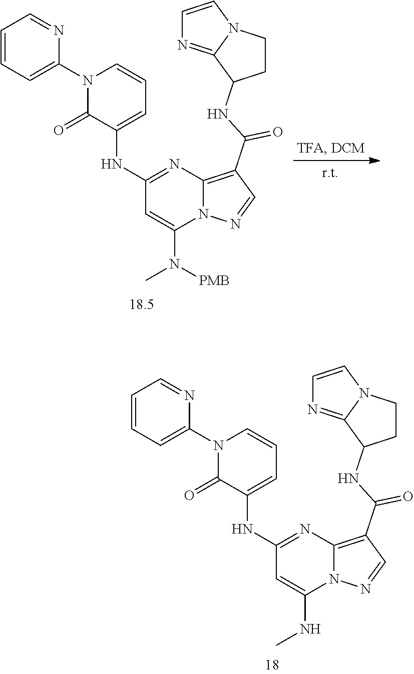

To a mixture of ethyl 5,7-dichloropyrazolo[1,5-a]pyrimidine-3-carboxylate (III-4, 1.50 g, 5.90 mmol) and potassium carbonate (810 mg, 5.90 mmol) in ethyl alcohol (15 mL) was added methylamine (3.50 mL, 7.10 mmol, 2.0 M in tetrahydrofuran), and the resulting mixture was stirred at room temperature for 2 hrs. Then the mixture was concentrated under reduced pressure and diluted with water (30 mL). The precipitated solids were collected by filtration and washed with diethyl ether (3×10 ml), and then dried in vacuum to afford ethyl 5-chloro-7-(methylamino)pyrazolo[1,5-a]pyrimidine-3-carboxylate (18.1, 1.20 g, crude) as an off-white solid.

To a suspension of ethyl 5-chloro-7-(methylamino)pyrazolo[1,5-a]pyrimidine-3-carboxylate (18.1, 4.00 g, 7.85 mmol) in tetrahydrofuran (20 mL) and water (20 mL) was added lithium hydroxide monohydrate (1.65 g, 39.3 mmol), and the resulting mixture was stirred at 50° C. overnight. Then the mixture was adjusted to pH 4 with 2 M aq. HCl and concentrated under reduced pressure. The residue was diluted with water (50 mL) and the precipitated solids were collected by filtration and washed with water (3×10 mL), and then dried in vacuum to afford 5-chloro-7-(methylamino)pyrazolo[1,5-a]pyrimidine-3-carboxylic acid (18.2, 4.00 g, crude) as a light brown solid. LC-MS (ESI): m/z 227.1 [M+H]⁺.

To a solution of 5-chloro-7-(methylamino)pyrazolo[1,5-a]pyrimidine-3-carboxylic acid (18.2, 2.50 g, crude) and 6,7-dihydro-5H-pyrrolo[1,2-a]imidazol-7-amine (6.6, 2.72 g, 22.0 mmol) in N,N-dimethylformamide (20 mL) were added 1-hydroxybenzotriazole (2.98 g, 22.0 mmol), 1-ethyl-3-(3-dimethylaminopropyl)carbodiimide hydrochloride (4.23 g, 22.0 mmol) and N,N-diisopropylethylamine (4.28 g, 33.0 mmol), and the resulting mixture was stirred at room temperature for 3 hrs. The reaction was diluted with water (50 mL) and extracted with dichloromethane (3×30 mL). The combined organic layers were washed with brine (4×20 mL), dried over anhydrous sodium sulfate, and then filtrated and concentrated under reduced pressure. The residue was subjected to silica gel column chromatography to afford 5-chloro-N-(6,7-dihydro-5H-pyrrolo[1,2-a]imidazol-7-yl)-7-(methylamino)pyrazolo[1,5-a]pyrimidine-3-carboxamide (18.3, 920 mg, 25%) as an off-white solid. LC-MS (ESI): m/z 332.1 [M+H]⁺.

To a mixture of 5-chloro-N-(6,7-dihydro-5H-pyrrolo[1,2-a]imidazol-7-yl)-7-(methylamino)pyrazolo[1,5-a]pyrimidine-3-carboxamide (18.3, 570 mg, 1.72 mmol) and 4-methoxybenzyl chloride (403 mg, 2.58 mmol) in N,N-dimethylformamide (10 mL) was added cesium carbonate (840 mg, 2.58 mmol) in portions, and the resulting mixture was stirred at 40° C. for 3 hrs. The reaction was diluted with water (20 mL) and extracted with dichloromethane (3×15 mL). The combined organic layers were washed with brine (4×15 mL), dried over anhydrous sodium sulfate, and then filtrated and concentrated under reduced pressure. The residue was subjected to silica gel column chromatography to afford 5-chloro-N-(6,7-dihydro-5H-pyrrolo[1,2-a]imidazol-7-yl)-7-((4-methoxybenzyl)(methyl)amino)pyrazolo[1,5-a]pyrimidine-3-carboxamide (18.4, 675 mg, 86%) as a yellow solid. LC-MS (ESI): m/z 452.3 [M+H]⁺.

To a mixture of 5-chloro-N-(6,7-dihydro-5H-pyrrolo[1,2-a]imidazol-7-yl)-7-((4-methoxybenzyl)(methyl)amino)pyrazolo[1,5-a]pyrimidine-3-carboxamide (18.4, 100 mg, 0.22 mmol) and 3-amino-2H-[1,2′-bipyridin]-2-one (2.2, 83.0 mg, 0.44 mmol) in dioxane (10 mL) were added BrettPhos (12.0 mg, 0.02 mmol), BrettPhos Pd G3 (20.0 mg, 0.02 mmol) and cesium carbonate (144 mg, 0.44 mmol), and the mixture was stirred at 100° C. for 16 hrs. The reaction mixture was cooled to room temperature and filtered. The filtrate was concentrated under reduced pressure. The residue was subjected to silica gel column chromatography to afford N-(6,7-dihydro-5H-pyrrolo[1,2-a]imidazol-7-yl)-7-((4-methoxybenzyl)(methyl)amino)-5-((2-oxo-2H-[1,2′-bipyridin]-3-yl)amino)pyrazolo[1,5-a]pyrimidine-3-carboxamide (18.5, 70.0 mg, 52%) as a yellow solid. LC-MS (ESI): m/z 603.3 [M+H]⁺.

To the solution of N-(6,7-dihydro-5H-pyrrolo[1,2-a]imidazol-7-yl)-7-((4-methoxybenzyl)(methyl)amino)-5-((2-oxo-2H-[1,2′-bipyridin]-3-yl)amino)pyrazolo[1,5-a]pyrimidine-3-carboxamide (18.5, 70.0 mg, 0.12 mmol) in dichloromethane (2 mL) was added trifluoroacetic acid (2 mL), and the mixture was stirred at room temperature for 1 hr. The mixture was concentrated under reduced pressure. The residue was subjected to Prep-HPLC to afford N-(6,7-dihydro-5H-pyrrolo[1,2-a]imidazol-7-yl)-7-(methylamino)-5-((2-oxo-2H-[1,2′-bipyridin]-3-yl)amino)pyrazolo[1,5-a]pyrimidine-3-carboxamide (18, 34.3 mg, 61%) as a white solid. LC-MS (ESI): m/z 483.1 [M+H]⁺. ¹H NMR (400 MHz, CD₃₀D) δ 8.63-8.57 (m, 1H), 8.26 (s, 1H), 8.09-7.97 (m, 2H), 7.81-7.74 (m, 1H), 7.55-7.48 (m, 1H), 7.37 (dd, J=7.0, 1.8 Hz, 1H), 7.20 (s, 1H), 7.12 (s, 1H), 5.87 (s, 1H), 5.82 (t, J=7.3 Hz, 1H), 5.65 (t, J=7.7 Hz, 1H), 4.32-4.22 (m, 1H), 4.12-4.01 (m, 1H), 3.26-3.16 (m, 1H), 3.03 (s, 3H), 2.54-2.41 (m, 1H).

### Synthetic Example 21. Synthesis of N-((1R,5S,7R)-2-oxabicyclo[3.2.0]heptan-7-yl)-5-((1-((1r,3r)-3-(methoxy-d₃)cyclobutyl)-2-oxo-1,2-dihydropyridin-3-yl)amino)-7-((methyl-d₃)amino)pyrazolo[1,5-a]pyrimidine-3-carboxamide (19)

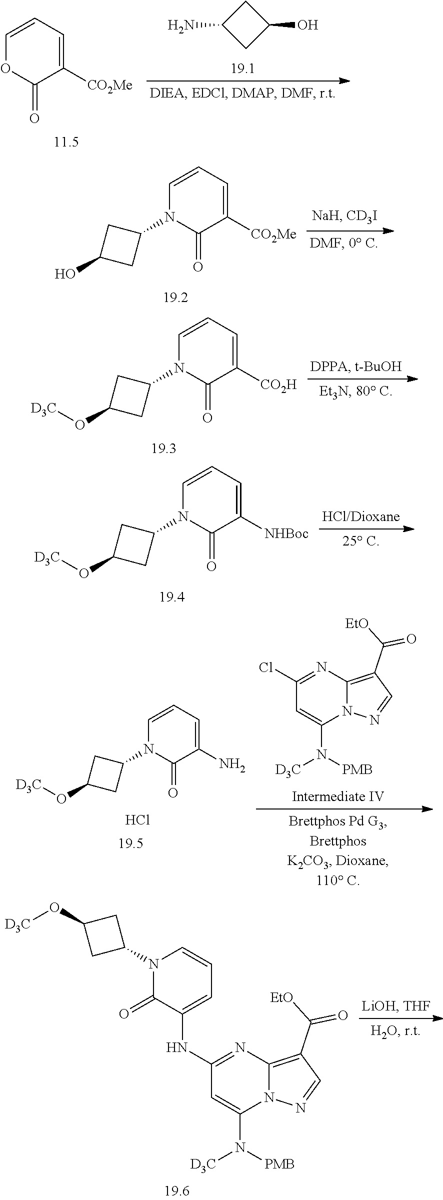

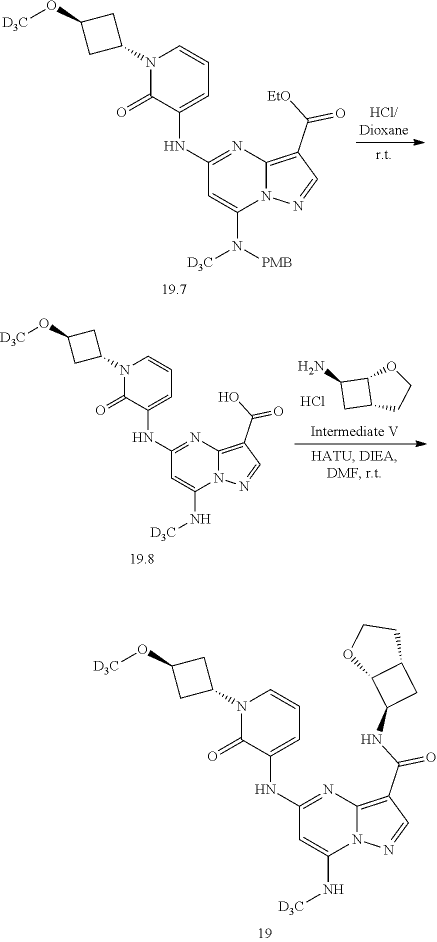

Compound 19.2 was synthesized from 11.5 similarly to procedure D to afford methyl 1-((1r,3r)-3-hydroxycyclobutyl)-2-oxo-1,2-dihydropyridine-3-carboxylate (19.2) as a yellow oil. LC-MS (ESI): m/z 224.1 [M+H]⁺.

To a solution of methyl 1-((1r,3r)-3-hydroxycyclobutyl)-2-oxo-1,2-dihydropyridine-3-carboxylate (19.2, 3.40 g, 15.3 mmol) in N,N-dimethylformamide (50 mL) was added sodium hydride (2.44 g, 61.0 mmol, 60% in oil) at 0° C., and the mixture was stirred at 0° C. for 30 mins. Then to the above mixture was added iodomethane-d₃ (3.31 g, 22.9 mmol) and the resulting mixture was stirred at 0° C. for additional 2 hrs. The reaction mixture was quenched with ice water (80 mL), adjusted to pH 4 with 6 M aq. HCl and extracted with ethyl acetate (3×30 mL). The combined organic layers were washed with brine (4×30 mL), dried over anhydrous sodium sulfate, and then filtrated and concentrated under reduced pressure. The residue was subjected to silica gel column chromatography to afford 1-((1r,3r)-3-(methoxy-d₃)cyclobutyl)-2-oxo-1,2-dihydropyridine-3-carboxylic acid (19.3, 1.70 g, 46%) as a brown solid. LC-MS (ESI): m/z 227.40 [M+H]⁺.

Compound 19.4 was synthesized from 19.3 similarly to procedure F to afford tert-butyl (1-((1r,3r)-3-(methoxy-d₃)cyclobutyl)-2-oxo-1,2-dihydropyridin-3-yl)carbamate (19.4) as an orange oil. LC-MS (ESI): m/z 298.40 [M+H]⁺.

Compound 19.5 was synthesized from 19.4 similarly to procedure G to afford 3-amino-1-((1r,3r)-3-(methoxy-d₃)cyclobutyl)pyridin-2(1H)-one hydrochloride (19.5) as a yellow solid. LC-MS (ESI): m/z 198.35 [M+H]⁺.

Compound 19.6 was synthesized from 19.5 similarly to procedure A to afford ethyl 5-((1-((1r,3r)-3-(methoxy-d₃)cyclobutyl)-2-oxo-1,2-dihydropyridin-3-yl)amino)-7-((4-methoxybenzyl)(methyl-d₃)amino)pyrazolo[1,5-a]pyrimidine-3-carboxylate (19.6) as a green oil. LC-MS (ESI): m/z 539.4 [M+H]⁺.

Compound 19.7 was synthesized from 19.6 similarly to procedure B to afford 5-((1-((1r,3r)-3-(methoxy-d₃)cyclobutyl)-2-oxo-1,2-dihydropyridin-3-yl)amino)-7-((4-methoxybenzyl)(methyl-d₃)amino)pyrazolo[1,5-a]pyrimidine-3-carboxylic acid (19.7) as a yellow solid. LC-MS (ESI): m/z 511.6 [M+H]⁺.

Compound 19.8 was synthesized from 19.7 similarly to procedure C to afford 5-((1-((1r,3r)-3-(methoxy-d₃)cyclobutyl)-2-oxo-1,2-dihydropyridin-3-yl)amino)-7-((methyl-d₃)amino)pyrazolo[1,5-a]pyrimidine-3-carboxylic acid (19.8) as a reddish brown solid. LC-MS (ESI): m/z 391.1 [M+H]⁺.

To a mixture of 5-((1-((1r,3r)-3-(methoxy-d₃)cyclobutyl)-2-oxo-1,2-dihydropyridin-3-yl)amino)-7-((methyl-d₃)amino)pyrazolo[1,5-a]pyrimidine-3-carboxylic acid (19.8, 50.0 mg, 0.12 mmol) and (1R,5S,7R)-2-oxabicyclo[3.2.0]heptan-7-amine hydrochloride (Intermediate V, 22.0 mg, crude) in N,N-dimethylformamide (2 mL) were added 2-(7-azabenzotriazol-1-yl)-N,N,N′,N′-tetramethyluronium hexafluorophosphate (98.0 mg, 0.25 mmol) and N,N-diisopropylethylamine (50 mg, 0.38 mmol), and the resulting mixture was stirred at room temperature for 1 hr. The mixture was filtered and the filtrate was subjected to Prep-HPLC to afford N-((1R,5S,7R)-2-oxabicyclo[3.2.0]heptan-7-yl)-5-((1-((1r,3r)-3-(methoxy-d₃)cyclobutyl)-2-oxo-1,2-dihydropyridin-3-yl)amino)-7-((methyl-d₃)amino)pyrazolo[1,5-a]pyrimidine-3-carboxamide (19, 41.7 mg, 66%) as a white solid. LC-MS (ESI): m/z 486.2 [M+H]⁺. ¹H NMR (400 MHz, DMSO-d₆) δ 8.87 (s, 1H), 8.25 (d, J=7.0 Hz, 1H), 8.18 (s, 1H), 8.01 (d, J=7.6 Hz, 1H), 7.84 (s, 1H), 7.50 (d, J=7.0 Hz, 1H), 6.32 (t, J=7.2 Hz, 1H), 6.17 (s, 1H), 5.33-5.22 (m, 1H), 4.38 (dd, J=6.8, 3.0 Hz, 1H), 4.28-4.19 (m, 1H), 4.10-4.01 (m, 2H), 3.97-3.89 (m, 1H), 2.94 (s, 1H), 2.57-2.52 (m, 1H), 2.45-2.43 (m, 3H), 2.06-1.97 (m, 1H), 1.91-1.78 (t, 2H), 1.76-1.68 (i, 1H).

Compounds of the present disclosure can be synthesized by those skilled in the art in view of the present disclosure. Further exemplary compounds were synthesized by following similar procedures/methods described herein in the Examples section. Exemplary characterizations, such as mass spectrum, ¹H NMR, ee/de value and separation conditions as applicable for additional exemplary compounds are provided in Table 2, below.

TABLE 2
Exemplary Characterization of Additional Compounds of the Present disclosure

| Com | | |
|---|---|---|
| pound | | LC-MS; ¹H NMR (ppm), ee/de value; separation |
| No. | Structure | conditions |
| 20 | 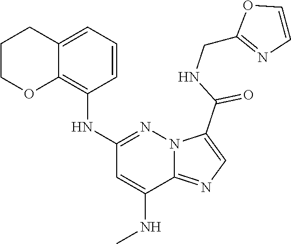 [SMILES: CNc1cc(Nc2cccc3c2OCCC3)nn2c(C(=O)NCc3ncco3)cnc12] | LC-MS (ESI): m/z 420.15 [M + H]⁺; ¹H NMR (300 MHz, DMSO-d₆) δ 9.49 (t, J = 6.2 Hz, 1H), 8.24 (s, 1H), 8.05 (s, 1H), 7.85 (s, 1H), 7.52- 7.40 (m, 2H), 7.17 (s, 1H), 6.72 (d, J = 7.8 Hz, 1H), 6.61 (t, J = 7.8 Hz, 1H), 6.09 (s, 1H), 4.61 (d, J = 6.3 Hz, 2H), 4.22 (t, J = 4.8 Hz, 2H), 2.87 (d, J = 4.8 Hz, 3H), 2.75 (t, J = 6.3 Hz, 2H), 2.02-1.86 (m, 2H). |
| 21 | 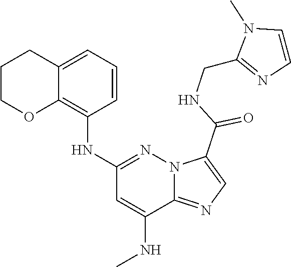 [SMILES: CNc1cc(Nc2cccc3c2OCCC3)nn2c(C(=O)NCc3nccn3C)cnc12] | LC-MS (ESI): m/z 433.3 [M + H]⁺; ¹H NMR (400 MHz, DMSO-d₆) δ 9.30 (t, J = 6.0 Hz, 1H), 8.18 (s, 1H), 7.83 (s, 1H), 7.52 (d, J = 7.2 Hz, 1H), 7.45-7.37 (m, 1H), 7.06 (s, 1H), 6.77 (s, 1H), 6.73-6.60 (m, 2H), 6.11 (s, 1H), 4.53 (d, J = 6.0 Hz, 2H), 4.23 (t, J = 4.8 Hz, 1H), 3.62 (s, 3H), 2.85 (d, J = 4.8 Hz, 3H), 2.75 (t, J = 6.4 Hz, 2H), 2.00- 1.89 (m, 2H). |
| 22a | 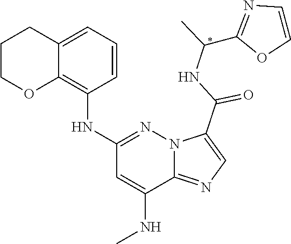 [SMILES: CNc1cc(Nc2cccc3c2OCCC3)nn2c(C(=O)NC(C)c3ncco3)cnc12] Enantiomer 1 (earlier eluting enantiomer) | LC-MS (ESI): m/z 434.15 [M + H]⁺; ¹H NMR (300 MHz, DMSO-d₆) δ 9.20 (d, J = 8.7 Hz, 1H), 8.21 (s, 1H), 8.05 (s, 1H), 7.84 (s, 1H), 7.53-7.37 (m, 2H), 7.16 (s, 1H), 6.73 (d, J = 7.5 Hz, 1H), 6.56 (t, J = 7.8 Hz, 1H), 6.10 (s, 1H), 5.44- 5.28 (m, 1H), 4.21 (t, J = 3.9 Hz, 2H), 2.87 (d, J = 4.8 Hz, 3H), 2.74 (t, J = 6.3 Hz, 2H), 2.00-1.84 (m, 2H), 1.38 (d, J = 7.2 Hz, 3H). ee: 99%; Retention time: 5.846 min (column: chiral art cellulose-SB, 2 × 25 cm, 5 μm; mobile phase A: Hex:DCM = 5:1 (0.1% DEA), mobile phase B: IPA; flow rate: 20 mL/min; gradient: 30% B to 30% B in 9.5 min; wave length: 254/210 nm). |
| 22b | 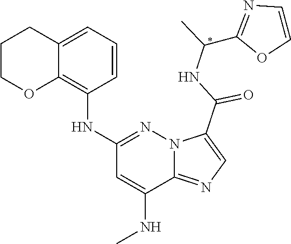 [SMILES: CNc1cc(Nc2cccc3c2OCCC3)nn2c(C(=O)NC(C)c3ncco3)cnc12] Enantiomer 2 (later eluting enantiomer) | LC-MS (ESI): m/z 434.15 [M + H]⁺; ¹H NMR (300 MHz, DMSO-d₆) δ 9.20 (d, J= 8.7 Hz, 1H), 8.21 (s, 1H), 8.05 (s, 1H), 7.84 (s, 1H), 7.51-7.38 (m, 2H), 7.16 (s, 1H), 6.73 (d, J = 7.5 Hz, 1H), 6.56 (t, J = 7.8 Hz, 1H), 6.10 (s, 1H), 5.44- 5.28 (m, 1H), 4.27-4.16 (m, 2H), 2.87 (d, J = 4.8 Hz, 3H), 2.74 (t, J = 6.3 Hz, 2H), 2.01-1.86 (m, 2H), 1.38 (d, J = 7.2 Hz, 3H). ee: 99%; Retention time: 7.075 min (column: chiral art cellulose-SB, 2 × 25 cm, 5 μm; mobile phase A: HEx:DCM = 5:1 (0.1% DEA), mobile phase B: IPA; flow rate: 20 mL/min; gradient: 30% B to 30% B in 9.5 min; wave length: 254/210 nm). |
| 23a | 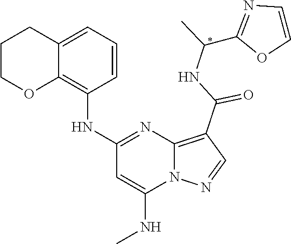 [SMILES: CNc1cc(Nc2cccc3c2OCCC3)nc2c(C(=O)NC(C)c3ncco3)cnn12] Enantiomer 1 (earlier eluting enantiomer) | LC-MS (ESI): m/z 434.2 [M + H]⁺; ¹H NMR (400 MHz, DMSO-d₆) δ 8.66 (s, 1H), 8.34 (d, J = 8.8 Hz, 1H), 8.15 (s, 1H), 8.02 (s, 1H), 7.86- 7.80 (m, 1H), 7.71-7.69 (m, 1H), 7.14 (s, 1H), 6.77 (d, J = 6.4 Hz, 1H), 6.65 (t, J = 7.6 Hz, 1H), 5.89 (s, 1H), 5.35-5.31 (m, 1H), 4.23-4.21 (m, 2H), 2.90 (d, J = 4.8 Hz, 3H), 2.75 (t, J = 6.0 Hz, 2H), 1.97- 1.91 (m, 2H), 1.43 (d, J = 6.8 Hz, 3H). ee: 100%; Retention time: 8.75 min (column: chiral art cellulose-SB, 2 × 25 cm, 5 μm; mobile phase A: Hex:DCM = 5:1 (0.5% 2.0M NH₃—MeOH)--HPLC, mobile phase B: IPA--HPLC; flow rate: 20 mL/min; gradient: 20% B to 20% B in 16 min; wave length: 220/254 nm). |
| 23b | 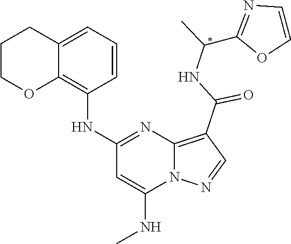 [SMILES: CNc1cc(Nc2cccc3c2OCCC3)nc2c(C(=O)NC(C)c3ncco3)cnn12] Enantiomer 2 (later eluting enantiomer) | LC-MS (ESI): m/z 434.2 [M + H]⁺; ¹H NMR (400 MHz, DMSO-d₆) δ 8.65 (s, 1H), 8.33 (d, J = 8.4 Hz, 1H), 8.15 (s, 1H), 8.02 (s, 1H), 7.82 (d, J = 4.8 Hz, 1H), 7.70 (d, J = 7.6 Hz, 1H), 7.14 (s, 1H), 6.77 (d, J = 7.2 Hz, 1H), 6.65 (t, J = 7.6 Hz, 1H), 5.89 (s, 1H), 5.33 (t, J = 7.6 Hz, 1H), 4.22 (s, 2H), 2.90 (d, J = 4.4 Hz, 3H), 2.75 (t, J = 6.0 Hz, 2H), 1.94 (s, 2H), 1.43 (d, J = 7.2 Hz, 3H). ee: 99%; Retention time: 12.00 min (column: chiral art cellulose-SB, 2 × 25 cm, 5 μm; mobile phase A: Hex:DCM = 5:1 (0.5% 2.0M NH₃—MeOH)--HPLC, mobile phase B: IPA-HPLC; flow rate: 20 mL/min; gradient: 20% B to 20% B in 16 min; wave length: 220/254 nm). |
| 24 | 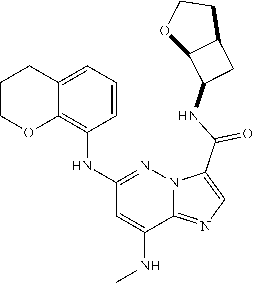 Racemic mixture | LC-MS (ESI): m/z 435.10 [M + H]⁺; ¹H NMR (400 MHz, DMSO-d₆) δ 8.56 (d, J = 7.2 Hz, 1H), 8.15 (s, 1H), 7.77 (s, 1H), 7.44-7.33 (m, 2H), 6.87-6.76 (m, 2H), 5.98 (s, 1H), 4.45-4.35 (m, 2H), 4.23-4.14 (m, 2H), 3.78 (t, J = 8.0 Hz, 1H), 3.19-3.11 (m, 1H), 2.86 (d, J = 4.8 Hz, 3H), 2.80-2.71 (m, 3H), 2.45-2.39 (m, 1H), 1.96-1.89 (m, 2H), 1.59-1.50 (m, 1H), 1.44-1.38 (m, 1H), 1.28-1.21 (m, 1H). |
| 25 | 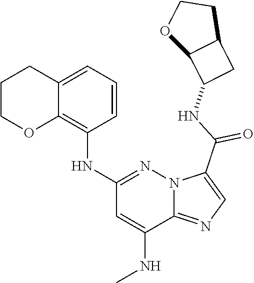 Racemic mixture | LC-MS (ESI): m/z 435.15 [M + H]⁺; ¹H NMR (400 MHz, DMSO-d₆) δ 8.98 (d, J = 7.2 Hz, 1H), 8.25 (s, 1H), 7.81 (s, 1H), 7.50 (t, J = 4.8 Hz, 1H), 7.46-7.36 (m, 1H), 6.86 (d, J = 8.0 Hz, 2H), 6.04 (s, 1H), 4.26-4.15 (m, 4H), 4.07-3.98 (m, 1H), 3.92-3.83 (m, 1H), 2.86 (d, J = 2.8 Hz, 3H), 2.79 (t, J = 6.0 Hz, 3H), 1.99-1.91 (m, 2H), 1.90-1.76 (m, 2H), 1.73-1.57 (m, 2H). |
| 26 | 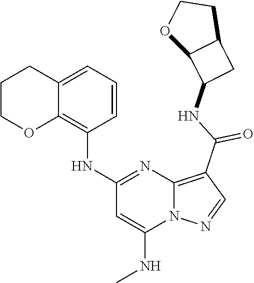 Racemic mixture | LC-MS (ESI): m/z 435.10 [M + H]⁺; ¹H NMR (300 MHz, DMSO-d₆) δ 8.58 (s, 1H), 8.09 (s, 1H), 7.78-7.75 (m, 2H), 7.57-7.54 (m, 1H), 6.85-6.77 (m, 2H), 5.78 (s, 1H), 4.42-4.38 (m, 2H), 4.23-4.19 (m, 2H), 3.81-3.76 (m, 1H), 3.31- 3.24 (m, 1H), 2.90 (d, J = 4.8 Hz, 3H), 2.77-2.72 (m, 3H), 2.51-2.50 (m, 1H), 1.96-1.93 (m, 2H), 1.58-1.51 (m, 2H), 1.36-1.34 (m, 1H). |
| 27 | 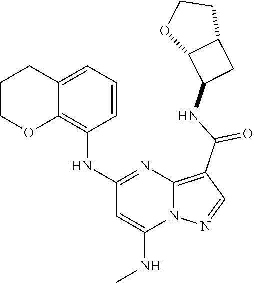 Racemic mixture | LC-MS (ESI): m/z 435.10 [M + H]⁺; ¹H NMR (300 MHz, DMSO-d₆) δ 8.67 (s, 1H), 8.17 (d, J = 7.8 Hz, 1H), 8.10 (s, 1H), 7.80-7.78 (m, 1H), 7.71-7.68 (m, 1H), 6.85-6.83 (m, 2H), 5.82 (s, 1H), 4.25-4.17 (m, 4H), 4.03-4.00 (m, 1H), 3.91- 3.87 (m, 1H), 2.90 (s, 3H), 2.81-2.77 (m, 3H), 1.98- 1.66 (m, 6H). |
| 28 | 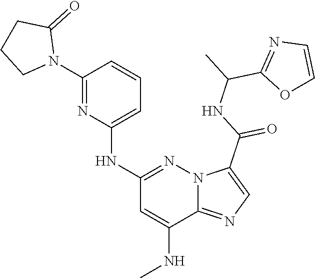 [SMILES: CNc1cc(Nc2cccc(N3CCCC3=O)n2)nn2c(C(=O)NC(C)c3ncco3)cnc12] Racemic mixture | LC-MS (ESI): m/z 462.2 [M + H]⁺; ¹H NMR (300 MHz, DMSO-d₆) δ 9.60 (s, 1H), 9.20 (d, J = 8.4 Hz, 1H), 8.06 (s, 1H), 7.93 (s, 1H), 7.85 (d, J = 8.1 Hz, 1H), 7.70-7.56 (m, 2H), 7.19 (d, J = 9.0 Hz, 2H), 6.63 (s, 1H), 5.47-5.37 (m, 1H), 4.06 (t, J = 7.2 Hz, 2H), 2.92 (d, J = 4.8 Hz, 3H), 2.73- 2.59 (m, 2H), 2.10-2.00 (m, 2H), 1.60 (d, J = 6.9 Hz, 3H). |
| 29 | 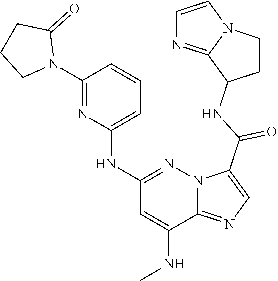 [SMILES: CNc1cc(Nc2cccc(N3CCCC3=O)n2)nn2c(C(=O)NC3CCn4ccnc43)cnc12] Racemic mixture | LC-MS (ESI): m/z 473.2 [M + H]⁺; ¹H NMR (300 MHz, DMSO-d₆) δ 9.84 (s, 1H), 8.17 (d, J = 8.5 Hz, 1H), 8.07 (d, J = 5.5 Hz, 1H), 7.83 (d, J = 8.0 Hz, 1H), 7.39 (t, J = 8.0 Hz, 1H), 7.21 (d, J = 7.4 Hz, 2H), 6.98 (s, 1H), 6.60 (s, 1H), 5.46 (q, J = 7.8 Hz, 1H), 4.19-4.04 (m, 3H), 4.02-3.92 (m, 1H), 3.10-3.01 (m, 1H), 2.96 (d, J = 4.6 Hz, 3H), 2.58 (t, J = 8.0 Hz, 3H), 2.37-2.33 (m, 1H), 2.06- 2.05 (m, 2H). |
| 30 | 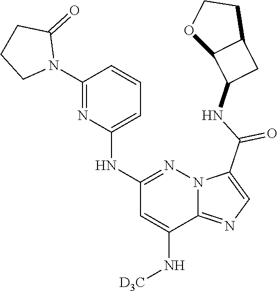 Racemic mixture | LC-MS (ESI): m/z 466.0 [M + H]⁺; ¹H NMR (300 MHz, DMSO-d₆) δ 9.43 (s, 1H), 8.71 (d, J = 7.2 Hz, 1H), 7.93-7.83 (m, 2H), 7.73 (t, J = 8.1 Hz, 1H), 7.60 (s, 1H), 7.18 (d, J = 7.8 Hz, 1H), 6.53 (s, 1H), 4.57-4.41 (m, 2H), 4.04 (t, J = 7.1 Hz, 2H), 3.96-3.84 (m, 1H), 3.69-3.63 (m, 1H), 2.91- 2.80 (m, 1H), 2.67-2.54 (m, 3H), 2.11-1.97 (m, 2H), 1.68-1.54 (m, 3H). |
| 31 | 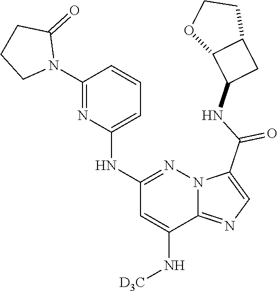 Racemic mixture | LC-MS (ESI): m/z 466.0 [M + H]⁺; ¹H NMR (300 MHz, DMSO-d₆) δ 9.62 (s, 1H), 9.03 (d, J = 7.2 Hz, 1H), 7.96-7.88 (m, 2H), 7.75 (t, J = 8.1 Hz, 1H), 7.60 (s, 1H), 7.18 (d, J = 7.8 Hz, 1H), 6.72 (s, 1H), 4.55 (dd, J = 6.9, 3.1 Hz, 1H), 4.29- 4.17 (m, 1H), 4.07 (t, J = 6.9 Hz, 3H), 3.97-3.89 (m, 1H), 3.01-2.89 (m, 1H), 2.65-2.55 (m, 2H), 2.14-1.91 (m, 4H), 1.90-1.66 (m, 2H). |
| 32 | 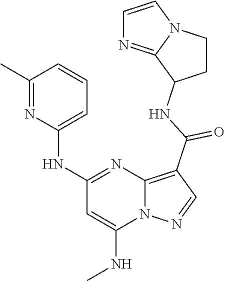 [SMILES: CNc1cc(Nc2cccc(C)n2)nc2c(C(=O)NC3CCn4ccnc43)cnn12] Racemic mixture | LC-MS (ESI): m/z 404.15 [M + H]⁺; ¹H NMR (400 MHz, CDCl₃ ) δ 8.38 (s, 1H), 8.28 (s, 2H), 7.33-7.27 (m, 1H), 7.24-7.15 (m, 2H), 6.95 (s, 1H), 6.72 (d, J = 7.2 Hz, 1H), 6.57 (s, 1H), 6.25 (d, J = 4.4 Hz, 1H), 5.49 (s, 1H), 4.19-4.14 (m, 1H), 3.95 (m, 1H), 3.24-3.22 (m, 1H), 3.08 (d, J = 5.2 Hz, 3H), 2.67-2.51 (m, 1H), 2.46 (s, 3H). |
| 33 | 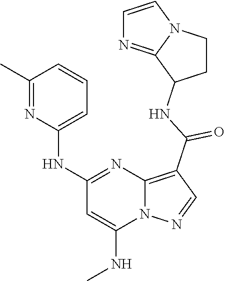 [SMILES: CNc1cc(Nc2cccc(C)n2)nc2c(C(=O)NC3CCn4ccnc43)cnn12] Racemic mixture | LC-MS (ESI): m/z 405.2 [M + H]⁺; ¹H NMR (400 MHz, DMSO-d₆) δ 9.92 (s, 1H), 8.34 (d, J = 8.4 Hz, 1H), 8.25 (s, 1H), 8.15-8.00 (m, 2H), 7.39 (d, J = 8.4 Hz, 1H), 7.24 (t, J = 8.0 Hz, 1H), 6.82 (d, J = 7.2 Hz, 1H), 6.47 (s, 1H), 5.62-5.49 (m, 1H), 4.32-4.28 (m, 1H), 4.16-4.09 (m, 1H), 3.19-3.16 (m, 1H), 2.93 (d, J = 4.8 Hz, 3H), 2.50-2.49 (m, 1H), 2.41 (s, 3H). |
| 34 | 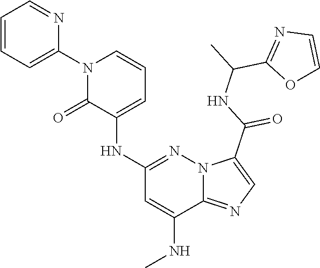 [SMILES: CNc1cc(Nc2cccn(-c3ccccn3)c2=O)nn2c(C(=O)NC(C)c3ncco3)cnc12] Racemic mixture | LC-MS (ESI): m/z 472.3 [M + H]⁺; ¹H NMR (400 MHz, CDCl₃) δ 9.07 (d, J = 8.7 Hz, 1H), 8.68-8.54 (m, 1H), 8.12 (s, 1H), 7.98 (dd, J = 7.3, 1.7 Hz, 1H), 7.96-7.86 (m, 2H), 7.81 (s, 1H), 7.63 (d, J = 0.9 Hz, 1H), 7.48 (dd, J = 7.2, 1.8 Hz, 1H), 7.43-7.32 (m, 1H), 7.08 (s, 1H), 6.29 (t, J = 7.2 Hz, 1H), 6.20 (s, 1H), 5.83-5.63 (m, 2H), 3.01 (d, J = 5.2 Hz, 3H), 1.72 (d, J = 7.2 Hz, 3H). |
| 35 | 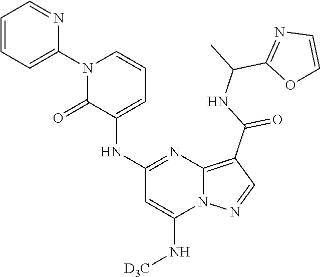 [SMILES: [2H]C([2H])([2H])Nc1cc(Nc2cccn(-c3ccccn3)c2=O)nc2c(C(=O)NC(C)c3ncco3)cnn12] Racemic mixture | LC-MS (ESI): m/z 475.3 [M + H]⁺; ¹H NMR (400 MHz, DMSO-d₆) δ 9.01 (s, 1H), 8.64 (d, J = 4.0 Hz, 1H), 8.31 (d, J = 6.8 Hz, 1H), 8.23 (d, J = 9.2 Hz, 2H), 8.12-8.01 (m, 2H), 7.94 (s, 1H), 7.85-7.83 (m ,1H), 7.59-7.49 (m, 2H), 7.19 (s, 1H), 6.34-6.24 (m, 2H), 5.44 (t, J = 9.0 Hz, 1H), 1.58 (d, J = 6.8 Hz, 3H). |
| 36a | 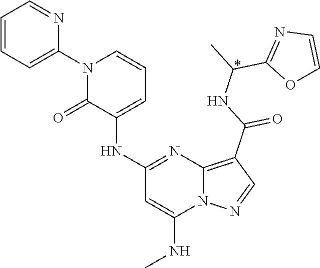 [SMILES: CNc1cc(Nc2cccn(-c3ccccn3)c2=O)nc2c(C(=O)NC(C)c3ncco3)cnn12] Enantiomer 1 (earlier eluting enantiomer) | LC-MS (ESI): m/z 472.18 [M + H]⁺; ¹H NMR (400 MHz, DMSO-d₆) δ 9.02 (s, 1H), 8.64- 8.63 (m, 1H), 8.31-8.29 (m, 1H), 8.24-8.22 (m, 2H), 8.07-8.02 (m, 2H), 7.98-7.95 (m, 1H), 7.85- 7.83 (m, 1H), 7.55-7.52 (m, 2H), 7.18 (s, 1H), 6.3- 6.27 (m, 2H), 5.45-5.41 (m, 1H), 2.91 (d, J = 4.8 Hz, 3H), 1.59 (d, J = 7.2 Hz, 3H). ee: 99%; Retention time: 12.19 min (column: chiral art cellulose-SC, 2*25 cm, 5 μm; mobile phase A: MTBE (10 mM NH₃-MeOH), mobile phase B: MeOH--HPLC; flow rate: 18 mL/min; gradient: 50% B to 50% B in 18 min; wave length: 268/210 nm). |
| 36b | 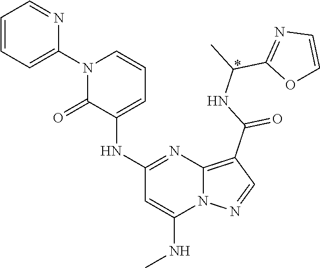 [SMILES: CNc1cc(Nc2cccn(-c3ccccn3)c2=O)nc2c(C(=O)NC(C)c3ncco3)cnn12] Enantiomer 2 (later eluting enantiomer) | LC-MS (ESI): m/z 472.18 [M + H]⁺; ¹H NMR (400 MHz, DMSO-d₆) δ 9.02 (s, 1H), 8.64- 8.63 (m, 1H), 8.31-8.29 (m, 1H), 8.24-8.22 (m, 2H), 8.07-8.02 (m, 2H), 7.98-7.95 (m, 1H), 7.85- 7.83 (m, 1H), 7.55-7.52 (m, 2H), 7.18 (s, 1H), 6.30- 6.27 (m, 2H), 5.47-5.40 (m, 1H), 2.91 (d, J = 4.8 Hz, 3H), 1.59 (d, J = 7.2 Hz, 3H). ee: 99%; Retention time: 15.53 min (column: chiral art cellulose-SC, 2*25 cm, 5 μm; mobile phase A: MTBE (10 mM NH₃—MeOH), mobile phase B: MeOH--HPLC; flow rate: 18 mL/min; gradient: 50% B to 50% B in 18 min; wave length: 268/210 nm). |
| 37 | 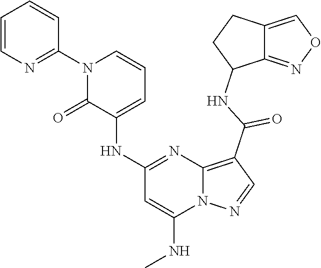 [SMILES: CNc1cc(Nc2cccn(-c3ccccn3)c2=O)nc2c(C(=O)NC3CCc4conc43)cnn12] Racemic mixture | LC-MS (ESI): m/z 484.29 [M + H]⁺; ¹H NMR (500 MHz, DMSO-d₆) δ = 8.97 (s, 1H), 8.63 (dd, J = 4.7, 1.9 Hz, 1H), 8.55 (t, J = 1.3 Hz, 1H), 8.28 (s, 1H), 8.13 (d, J = 8.5 Hz, 1H), 8.09 (dd, J = 7.4, 1.8 Hz, 1H), 8.03 (td, J = 7.7, 1.9 Hz, 1H), 7.96 (q, J = 4.8 Hz, 1H), 7.84-7.78 (m, 1H), 7.56- 7.50 (m, 1H), 7.47 (dd, J = 7.0, 1.7 Hz, 1H), 6.24 (s, 1H), 5.93 (t, J = 7.2 Hz, 1H), 5.63 (q, J = 8.2 Hz, 1H), 3.0-2.83 (m, 4H), 2.82-2.73 (m, 1H), 2.71- 2.63 (m, 1H), 2.32-2.22 (m, 1H). |
| 38 | 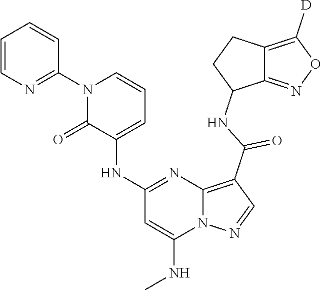 [SMILES: [2H]c1onc2c1CCC2NC(=O)c1cnn2c(NC)cc(Nc3cccn(-c4ccccn4)c3=O)nc12] Racemic mixture | LC-MS (ESI): m/z 485.19 [M + H]⁺; ¹H NMR (500 MHz, DMSO-d₆) δ = 8.97 (s, 1H), 8.63 (dd, J = 5.1, 1.8 Hz, 1H), 8.28 (s, 1H), 8.13 (d, J = 8.5 Hz, 1H), 8.09 (dd, J = 7.4, 1.8 Hz, 1H), 8.03 (td, J = 7.8, 1.9 Hz, 1H), 7.96 (q, J = 4.8 Hz, 1H), 7.81 (d, J = 8.0 Hz, 1H), 7.52 (ddd, J = 7.5, 4.8, 1.0 Hz, 1H), 7.47 (dd, J = 7.0, 1.8 Hz, 1H), 6.24 (s, 1H), 5.93 (t, J = 7.2 Hz, 1H), 5.63 (q, J = 8.2 Hz, 1H), 2.96-2.85 (m, 4H), 2.78 (ddd, J = 15.6, 8.7, 2.8 Hz, 1H), 2.72-2.62 (m, 1H), 2.33-2.21 (m, 1H). |
| 39 | 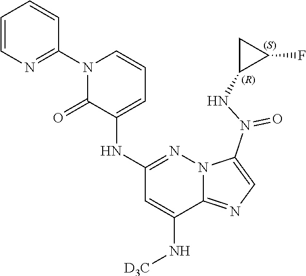 | LC-MS (ESI): m/z 438.29 [M + H]⁺; ¹H NMR (500 MHz, DMSO-d₆) δ = 8.63 (d, J = 5.4 Hz, 3H), 8.04 (td, J = 7.7, 1.9 Hz, 1H), 7.99 (dd, J = 7.3, 1.7 Hz, 1H), 7.91 (s, 1H), 7.84 (d, J = 8.1 Hz, 1H), 7.57-7.50 (m, 2H), 7.47 (s, 1H), 6.43-6.37 (m, 2H), 4.96-4.79 (m, 1H), 3.02-2.97 (m, 1H), 1.28-1.20 (m, 1H), 1.04-0.93 (m, 1H). |
| 40 | 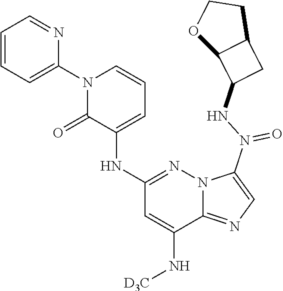 Racemic mixture | LC-MS (ESI): m/z 476.20 [M + H]⁺; ¹H NMR (400 MHz, DMSO-d₆) δ 8.64 (dd, J = 4.8, 1.2 Hz, 1H), 8.57 (s, 1H), 8.46 (d, J = 7.6 Hz, 1H), 8.11-7.96 (m, 2H), 7.93-7.78 (m, 2H), 7.59-7.50 (m, 2H), 7.47 (s, 1H), 6.42 (t, J = 7.2 Hz, 1H), 6.37 (s, 1H), 4.61-4.54 (m, 1H), 4.53-4.44 (m, 1H), 4.00-3.93 (m, 1H), 3.75 (m, 1H), 2.93-2.80 (m, 1H), 2.72-2.58 (m, 1H), 1.78-1.50 (m, 3H). |
| 41 | 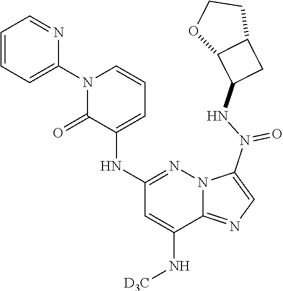 Racemic mixture | LC-MS (ESI): m/z 476.25 [M + H]⁺; ¹H NMR (400 MHz, DMSO-d₆) δ 8.79 (d, J = 7.6 Hz, 1H), 8.71-8.59 (m, 2H), 8.15 (dd, J = 7.2, 1.6 Hz, 1H), 8.05 (m, 1H), 7.92-7.81 (m, 2H), 7.59- 7.49 (m, 2H), 7.44 (s, 1H), 6.44 (t, J = 7.2 Hz, 1H), 6.38 (s, 1H), 4.48 (dd, J = 6.4, 3.2 Hz, 1H), 4.35- 4.26 (m, 1H), 4.07-4.04 (m, 1H), 3.96-3.94 (m, 1H), 3.00-2.88 (m, 1H), 2.08-1.90 (m, 2H), 1.89-1.78 (m, 1H), 1.77-1.68 (m, 1H). |
| 42 | 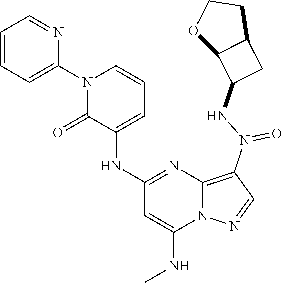 Racemic mixture | LC-MS (ESI): m/z 473.0 [M + H]⁺; ¹H-NMR (400 MHz, DMSO-d₆): δ 8.91 (s, 1H), 8.64 (d, J = 3.6 Hz, 1H), 8.39 (dd, J = 7.2, 1.6 Hz, 1H), 8.20 (s, 1H), 8.06-8.02 (m, 1H), 7.91-7.90 (m, 1H), 7.85 (d, J = 8.0 Hz, 1H), 7.68 (d, J = 7.6 Hz, 1H), 7.57-7.52 (m, 2H), 6.45 (t, J = 7.2 Hz, 1H), 6.25 (s, 1H), 4.62-4.58 (m, 1H), 4.45-4.42 (m, 1H), 4.03-3.99 (m, 1H), 3.84-3.82 (m, 1H), 2.92-2.90 (m, 4H), 2.67-2.64 (m, 1H), 1.71-1.69 (m, 2H), 1.54-1.51 (m, 1H). |
| 43 | 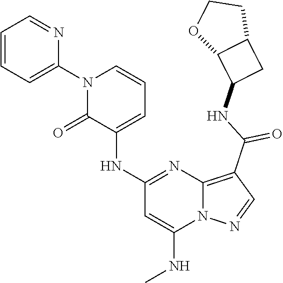 Racemic mixture | LC-MS (ESI): m/z 473.0 [M + H]⁺; ¹H-NMR (400 MHz, CD₃OD): δ 8.62 (d, J = 3.6 Hz, 1H), 8.40 (d, J = 5.6 Hz, 1H), 8.20 (s, 1H), 8.05- 8.03 (m, 1H), 7.84 (d, J = 8.0 Hz, 1H), 7.58-7.49 (m, 2H), 6.53 (t, J = 7.2 Hz, 1H), 5.86 (s, 1H), 4.57- 4.49 (m, 1H),4.38-4.32 (m, 1H), 4.16-4.15 (m, 1H), 4.12-4.01 (m, 1H), 3.68-3.44 (m, 1H), 3.11- 3.03 (m, 4H), 2.20-1.90 (m, 3H), 1.85-1.77 (m, 1H). |
| 44 | 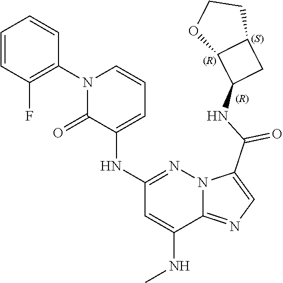 | LC-MS (ESI): m/z 490.2 [M + H]⁺; ¹H NMR (400 MHz, CDCl₃) δ 8.77 (d, J = 6.8 Hz, 1H), 8.09 (s, 1H), 8.03 (d, J = 7.4, 1H), 7.79 (s, 1H), 7.56-7.46 (m, 1H), 7.42-7.39 (m, 1H), 7.36-7.28 (m, 2H), 7.00-6.91 (m, 1H), 6.44 (t, J = 7.2 Hz, 1H), 6.18 (s, 1H), 5.68 (s, 1H), 4.65-4.63 (m, 1H), 4.45-4.44 (m, 1H), 4.20-4.17 (m, 1H), 4.10-4.07 (m, 1H), 3.03-3.02 (m, 4H), 2.21-1.92 (m, 2H), 1.98-1.80 (m, 1H), 1.79-1.76 (m, 1H). ee: 99%; Retention time: 12.97 min (column: chiral art amylose-SA, 3*25 cm, 5 μm; mobile phase A: CO₂, mobile phase B: EtOH:DCM = 3:1--HPLC; flow rate: 70 mL/min; gradient; isocratic 50% B; column temperature (° C.): 35; back pressure(bar): 100; wave length: 254 nm). |
| 45 | 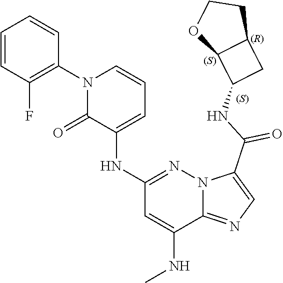 | LC-MS (ESI): m/z 490.2 [M + H]⁺; ¹H NMR (400 MHz, CDCl₃) δ 8.77 (d, J = 6.5 Hz, 1H), 8.08 (s, 1H), 8.03 (dd, J = 7.4, 1.7 Hz, 1H), 7.78 (s, 1H), 7.53-7.45 (m, 1H), 7.42-7.40 (m, 1H), 7.36-7.27 (m, 2H), 6.99-6.92 (m, 1H), 6.44 (t, J = 7.2 Hz, 1H), 6.01 (d, J = 5.5 Hz, 1H), 5.67 (s, 1H), 4.65 (dd, J = 6.8, 3.4 Hz, 1H), 4.45 (d, J = 8.0 Hz, 1H), 4.20-4.18 (m, 1H), 4.08-4.06 (m, 1H), 3.04-3.02 (m, 4H), 2.22-2.02 (m, 2H), 2.02-1.91 (m, 1H), 1.83-1.76 (m, 1H). ee: 99%; Retention time: 17.80 min (column: chiral art amylose-SA, 3*25 cm, 5 μm; mobile phase A: CO₂, mobile phase B: EtOH:DCM = 3:1--HPLC; flow rate: 70 mL/min; gradient: isocratic 50% B; column temperature (° C.): 35; back pressure (bar): 100; wave length: 254 nm). |
| 46 | 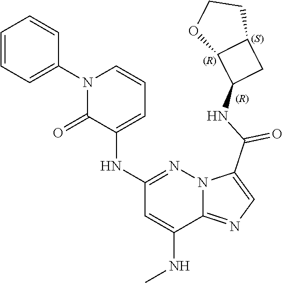 | LC-MS (ESI): m/z 472.2 [M + H]⁺; ¹H NMR (400 MHz, DMSO-d₆) δ 8.78 (d, J = 7.6 Hz, 1H), 8.61 (s, 1H), 8.14 (dd, J = 7.3, 1.8 Hz, 1H), 7.86 (s, 1H), 7.62-7.52 (m, 2H), 7.53-7.43 (m, 4H), 7.31 (dd, J = 6.9, 1.8 Hz, 1H), 6.43-6.35 (m, 2H), 4.50 (dd, J = 6.7, 3.2 Hz, 1H), 4.35-4.26 (m, 1H), 4.07-4.05 (m, 1H), 3.95-3.93 (m, 1H), 2.99- 2.91 (m, 1H), 2.87 (d, J = 4.8 Hz, 3H), 2.11-1.90 (m, 2H), 1.87-1.67 (m, 2H). |
| 47 | 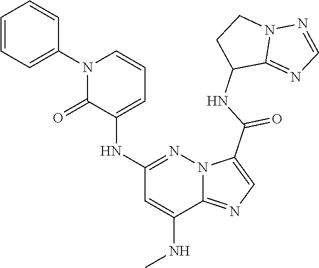 [SMILES: CNc1cc(Nc2cccn(-c3ccccc3)c2=O)nn2c(C(=O)NC3CCn4ncnc43)cnc12] Racemic mixture | LC-MS (ESI): m/z 483.24 [M + H]⁺; ¹H NMR (500 MHz, DMSO-d₆) δ 9.09 (d, J = 8.4 Hz, 1H), 8.58 (s, 1H), 8.03 (s, 1H), 7.93 (s, 1H), 7.91 (dd, J = 7.4, 1.8 Hz, 1H), 7.59-7.53 (m, 2H), 7.52-7.42 (m, 4H), 7.19 (dd, J = 6.9, 1.8 Hz, 1H), 6.37 (s, 1H), 5.93 (t, J = 7.2 Hz, 1H), 5.62 (td, J = 8.6, 6.2 Hz, 1H), 4.31 (td, J = 9.8, 3.5 Hz, 1H), 4.15 (dt, J = 10.5, 7.6 Hz, 1H), 3.25-3.15 (m, 1H), 2.87 (d, J = 4.9 Hz, 3H), 2.66-2.55 (m, 1H). |
| 48 | 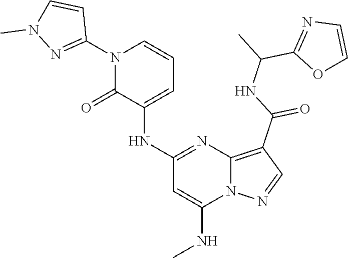 [SMILES: CNc1cc(Nc2cccn(-c3ccn(C)n3)c2=O)nc2c(C(=O)NC(C)c3ncco3)cnn12] Racemic mixture | LC-MS (ESI): m/z 475.0 [M + H]⁺; ¹H NMR (300 MHz, DMSO-d₆) δ 9.04 (s, 1H), 8.30- 8.17 (m, 3H), 8.07 (s, 1H), 7.97 (d, J = 5.1 Hz, 1H), 7.83 (d, J = 2.4 Hz, 1H), 7.63 (dd, J = 7.2, 1.5 Hz, 1H), 7.19 (s, 1H), 6.74 (d, J = 2.4 Hz, 1H), 6.31- 6.20 (m, 2H), 5.51-5.36 (m, 1H), 3.90 (s, 3H), 2.92 (d, J = 4.5 Hz, 3H), 1.58 (d, J = 6.9 Hz, 3H). |
| 49a | 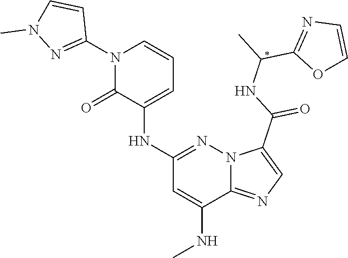 [SMILES: CNc1cc(Nc2cccn(-c3ccn(C)n3)c2=O)nn2c(C(=O)NC(C)c3ncco3)cnc12] Enantiomer 1 (earlier eluting enantiomer) | LC-MS (ESI): m/z 475.2 [M + H]⁺; ¹H NMR (300 MHz, DMSO-d₆) δ 9.04 (d, J = 8.4 Hz, 1H), 8.70 (s, 1H), 8.08 (s, 1H), 8.03-7.89 (m, 2H), 7.82 (d, J = 2.3 Hz, 1H), 7.62-7.47 (m, 2H), 7.18 (s, 1H), 6.74 (d, J = 2.3 Hz, 1H), 6.43 (s, 1H), 6.18 (t, J = 7.2 Hz, 1H), 5.53-5.38 (m, 1H), 3.89 (s, 3H), 2.87 (d, J = 4.8 Hz, 3H), 1.58 (d, J = 7.1 Hz, 3H). ee: 99%; Retention time: 13.10 min (column: chiral art cellulose-SB, 3*25 cm, 5 μm; mobile phase A: CO₂, mobile phase B: IPA:DCM = 2:1 (0.1% 2M NH₃—MeOH); flow rate: 70 mL/min; gradient: isocratic 45% B; column temperature (° C.): 35; back presssure (bar): 100; wave length: 254 nm). |
| 49b | 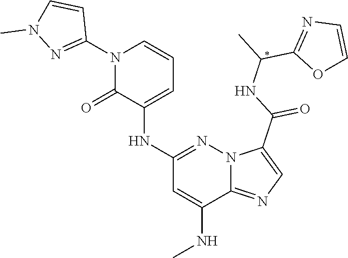 [SMILES: CNc1cc(Nc2cccn(-c3ccn(C)n3)c2=O)nn2c(C(=O)NC(C)c3ncco3)cnc12] Enantiomer 2 (later eluting enantiomer) | LC-MS (ESI): m/z 475.2 [M + H]⁺; ¹H NMR (300 MHz, DMSO-d₆) δ 9.04 (d, J = 8.4 Hz, 1H), 8.70 (s, 1H), 8.08 (s, 1H), 8.03-7.89 (m, 2H), 7.82 (d, J = 2.3 Hz, 1H), 7.62-7.47 (m, 2H), 7.18 (s, 1H), 6.74 (d, J = 2.3 Hz, 1H), 6.43 (s, 1H), 6.18 (t, J = 7.2 Hz, 1H), 5.53-5.38 (m, 1H), 3.89 (s, 3H), 2.87 (d, J = 4.8 Hz, 3H), 1.58 (d, J = 7.1 Hz, 3H). ee: 99%; Retention time: 17.22 min (column: chiral art cellulose-SB, 3*25 cm, 5 μm; mobile phase A: CO₂, mobile phase B: IPA:DCM = 2:1 (0.1% 2M NH₃—MeOH); flow rate: 70 mL/min; gradient: isocratic 45% B; column temperature (° C.): 35; back pressure (bar): 100; wave length: 254 nm). |
| 50 | 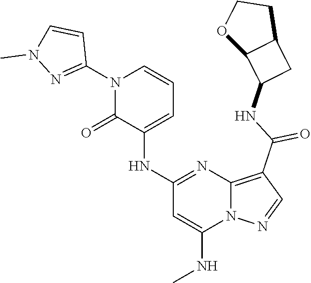 Racemic mixture | LC-MS (ESI): m/z 475.95 [M + H]⁺; ¹H NMR (300 MHz, CDCl₃) δ 8.56 (d, J = 6.9 Hz, 1H), 8.37 (s, 1H), 8.26 (s, 1H), 7.83 (s, 1H), 7.61 (d, J = 6.7 Hz, 1H), 7.45 (d, J = 2.1 Hz, 1H), 6.88 (d, J = 2.1 Hz, 1H), 6.50 (t, J = 7.2 Hz, 1H), 6.29 (s, 1H), 5.50 (s, 1H), 4.86-4.77 (m, 1H), 4.68-4.55 (m, 1H), 4.21-4.11 (m, 1H), 4.09-3.98 (m, 1H), 3.96 (s, 3H), 3.08 (d, J = 5.4 Hz, 3H), 3.01-2.87 (m, 1H), 2.89-2.76 (m, 1H), 2.01-1.55 (m, 3H). |
| 51 | 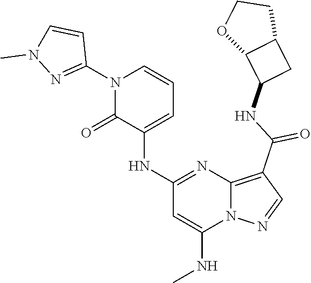 Racemic mixture | LC-MS (ESI): m/z 475.95 [M + H]⁺; ¹H NMR (300 MHz, CDCl₃) δ 8.37 (s, 2H), 8.19 (s, 1H), 8.03 (d, J = 6.6 Hz, 1H), 7.65 (d, J = 6.6 Hz, 1H), 7.45 (s, 1H), 6.86 (s, 1H), 6.40 (t, J = 7.2 Hz, 1H), 6.33 (s, 1H), 5.48 (s, 1H), 4.63 (dd, J = 6.3, 3.0 Hz, 1H), 4.51-4.39 (m, 1H), 4.27-4.17 (m, 1H), 4.14-4.03 (m, 1H), 3.96 (s, 3H), 3.17-2.95 (m, 4H), 2.23-1.89 (m, 4H). |
| 52 | 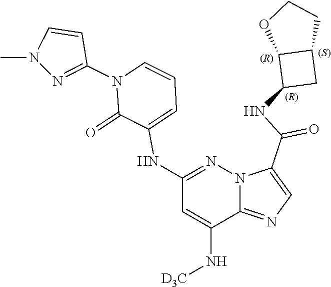 | LC-MS (ESI): m/z 479.27 [M + H]⁺; ¹H NMR (500 MHz, DMSO-d₆) δ 8.78 (d, J = 7.6 Hz, 1H), 8.68 (s, 1H), 8.10 (dd, J = 7.3, 1.8 Hz, 1H), 7.92-7.77 (m, 2H), 7.63 (dd, J = 7.1, 1.7 Hz, 1H), 7.45 (s, 1H), 6.75 (d, J = 2.2 Hz, 1H), 6.44-6.35 (m, 2H), 4.46 (dd, J = 6.8, 3.2 Hz, 1H), 4.34-4.23 (m, 1H), 4.11-4.03 (m, 1H), 3.98-3.92 (m, 1H), 3.90 (s, 3H), 2.96-2.89 (m, 1H), 2.05-1.89 (m, 2H), 1.88-1.77 (m, 1H), 1.76-1.69 (m, 1H). |
| 53 | 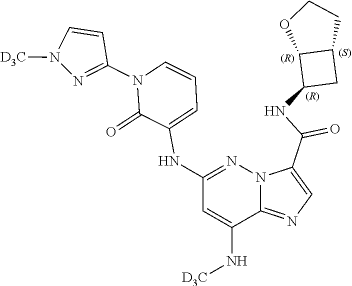 | LC-MS (ESI): m/z 482.3 [M + H]⁺; ¹H NMR (500 MHz, DMSO-d₆) δ 8.78 (d, J = 7.5 Hz, 1H), 8.68 (s, 1H), 8.10 (dd, J = 7.3, 1.8 Hz, 1H), 7.86 (s, 1H), 7.83 (d, J = 2.4 Hz, 1H), 7.63 (dd, J = 7.0, 1.7 Hz, 1H), 7.45 (s, 1H), 6.75 (d, J = 2.3 Hz, 1H), 6.43-6.36 (m, 2H), 4.46 (dd, J = 6.7, 3.2 Hz, 1H), 4.34-4.25 (m, 1H), 4.07 (td, J = 8.5, 7.9, 2.3 Hz, 1H), 3.98-3.91 (m, 1H), 2.96-2.90 (m, 1H), 1.99-1.90 (m, 2H), 1.88-1.78 (m, 1H), 1.76- 1.69 (m, 1H). |
| 54 | 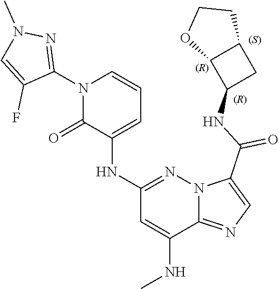 | LC-MS (ESI): m/z 494.20 [M + H]⁺; ¹H NMR (400 MHz, DMSO-d₆) δ 8.76 (d, J = 7.6 Hz, 1H), 8.69 (s, 1H), 8.15 (d, J = 7.4, 1H), 8.04 (d, J = 4.4 Hz, 1H), 7.85 (s, 1H), 7.48 (d, J = 4.8 Hz, 1H), 7.29 (d, J = 6.9 Hz, 1H), 6.42-6.36 (m, 2H), 4.49-4.45 (m, 1H), 4.30-4.28 (m, 1H), 4.07 (t, J = 7.3 Hz, 1H), 3.95 (dd, J = 9.6, 6.3 Hz, 1H), 3.85 (s, 3H), 2.93-2.92 (m, 1H), 2.86 (d, J = 4.9 Hz, 3H), 2.02-1.92 (m, 2H), 1.81-1.70 (m, 2H). |
| 55 | 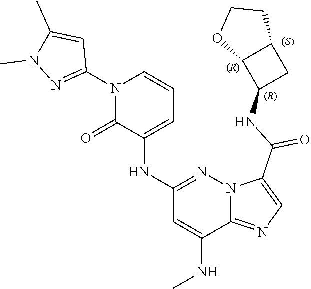 | LC-MS (ESI): m/z 490.3 [M + H]⁺; ¹H NMR (400 MHz, DMSO-d₆) δ 8.76 (d, J = 7.6 Hz, 1H), 8.69 (s, 1H), 8.15 (d, J = 7.4, 1H), 8.04 (d, J = 4.4 Hz, 1H), 7.85 (s, 1H), 7.48 (d, J = 4.8 Hz, 1H), 7.29 (d, J = 6.9 Hz, 1H), 6.42-6.36 (m, 2H), 4.49-4.45 (m, 1H), 4.30-4.28 (m, 1H), 4.07 (t, J = 7.3 Hz, 1H), 3.95 (dd, J = 9.6, 6.3 Hz, 1H), 3.85 (s, 3H), 2.93-2.92 (m, 1H), 2.86 (d, J = 4.9 Hz, 3H), 2.02-1.92 (m, 2H), 1.81-1.70 (m, 2H). |
| 56 | 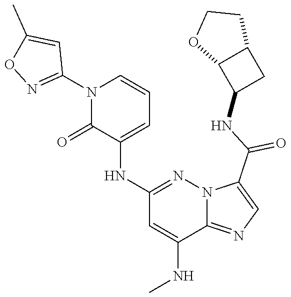 Racemic mixture | LC-MS (ESI): m/z 477.2 [M + H]⁺; ¹H NMR (300 MHz, DMSO-d₆) δ 8.74-8.72 (m, 2H), 8.17 (d, J = 7.2 Hz, 1H), 7.87 (s, 1H), 7.60- 7.47 (m, 2H), 6.89 (s, 1H), 6.52-6.38 (m, 2H), 4.48- 4.46 (m, 1H), 4.28-4.20 (m, 1H), 4.12-3.87 (m, 2H), 2.88-2.73 (m, 4H), 2.53-2.52 (m, 3H), 2.11- 1.93 (m, 2H), 1.90-1.73 (m, 2H). |
| 57 | 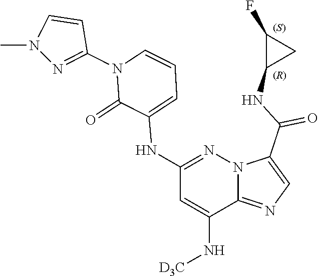 | LC-MS (ESI): m/z 441.24 [M + H]⁺; ¹H NMR (500 MHz, DMSO-d₆) δ 8.65 (s, 1H), 8.64 (d, J = 4.2 Hz, 1H), 7.94 (dd, J = 7.3, 1.7 Hz, 1H), 7.91 (s, 1H), 7.83 (d, J = 2.3 Hz, 1H), 7.62 (dd, J = 7.1, 1.7 Hz, 1H), 7.47 (s, 1H), 6.75 (d, J = 2.3 Hz, 1H), 6.40 (s, 1H), 6.37 (t, J = 7.2 Hz, 1H), 4.98- 4.78 (m, 1H), 3.90 (s, 3H), 3.02-2.96 (m, 1H), 1.27- 1.20 (m, 1H), 1.07-0.90 (m, 1H). |
| 58 | 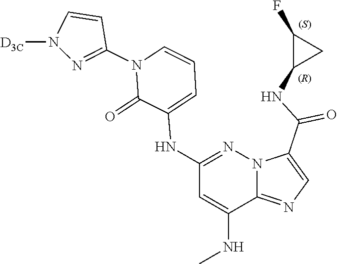 | LC-MS (ESI): m/z 441.2 [M + H]⁺; ¹H NMR (500 MHz, CDCl₃) δ 8.64 (s, 1H), 8.12 (s, 1H), 7.85 (dd, J = 7.4, 1.7 Hz, 1H), 7.79 (s, 1H), 7.58 (dd, J = 7.2, 1.8 Hz, 1H), 7.43 (d, J = 2.3 Hz, 1H), 6.84 (d, J = 2.3 Hz, 1H), 6.37 (t, J = 7.2 Hz, 1H), 5.94 (s, 1H), 5.67 (s, 1H), 4.90-4.68 (m, 1H), 3.22-3.12 (m, 1H), 3.03 (d, J = 5.2 Hz, 3H), 1.33- 1.26 (m, 1H), 1.17-1.05 (m, 1H). |
| 59 | 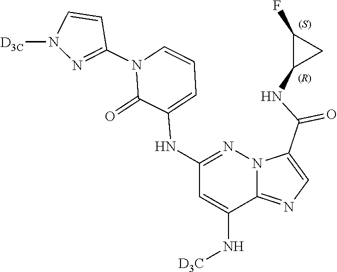 | LC-MS (ESI): m/z 444.2 [M + H]⁺; ¹H NMR (500 MHz, CDCl₃) δ 8.67 (s, 1H), 8.15 (s, 1H), 7.87 (dd, J = 7.0, 1.7 Hz, 1H), 7.81 (s, 1H), 7.61 (dd, J = 7.1, 1.7 Hz, 1H), 7.46 (d, J = 2.3 Hz, 1H), 6.87 (d, J = 2.3 Hz, 1H), 6.39 (t, J = 7.2 Hz, 1H), 5.95 (s, 1H), 5.70 (s, 1H), 4.90-4.72 (m, 1H), 3.27-3.12 (m, 1H), 1.35-1.29 (m, 1H), 1.18- 1.09 (m, 1H). |
| 60 | 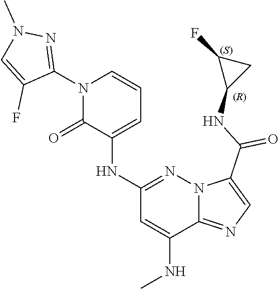 | LC-MS (ESI): m/z 456.30 [M + H]⁺; ¹H NMR (400 MHz, DMSO-d₆) δ 8.81-8.53 (m, 2H), 8.04-8.01 (m, 2H), 7.91 (s, 1H), 7.51 (d, J = 4.8 Hz, 1H), 7.27 (d, J = 5.6 Hz, 1H), 6.46-6.21 (m, 2H), 4.87 (d, J = 63.6 Hz, 1H), 3.85 (s, 3H), 2.86 (d, J = 4.8 Hz, 3H), 1.28-1.24 (m, 1H), 1.02- 0.91 (m, 1H). |
| 61 | 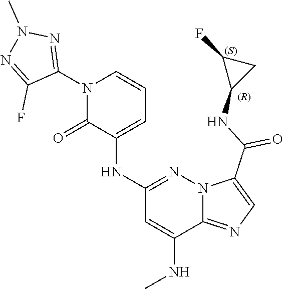 | LC-MS (ESI): m/z 456.0 [M + H]⁺; ¹H NMR (400 MHz, DMSO-d₆) δ 8.63-8.61 (m, 2H), 8.11 (m, 1H), 7.96-7.94 (m, 1H), 7.90 (s, 1H), 7.50-7.48 (m, 1H), 7.29-7.27 (m, 1H), 6.39 (s, 1H), 6.36-6.32 (m, 1H), 4.96-4.77 (m, 1H), 3.80 (s, 3H), 2.99-2.96 (m, 1H), 2.86 (s, 3H), 1.26-1.21 (m, 1H), 1.02-0.92 (m, 1H). |
| 62a | 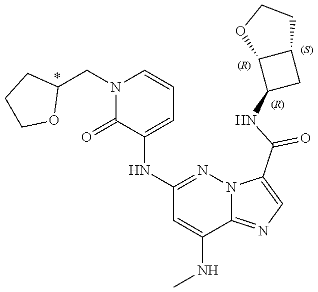 Diastereoisomer 1 (earlier eluting diastereoisomer) | LC-MS (ESI): m/z 480.55 [M + H]⁺; ¹H NMR (400 MHz, CDCl₃) δ 8.75 (d, J = 6.7 Hz, 1H), 8.10-7.98 (m, 1H), 7.90 (d, J = 7.3 Hz, 1H), 7.75 (s, 1H), 7.15-7.01 (m, 1H), 6.44-6.24 (m, 2H), 5.71-5.60 (m, 1H), 4.64-4.54 (m, 1H), 4.49- 4.34 (m, 2H), 4.34-4.12 (m, 2H), 4.12-3.98 (m, 1H), 3.95-3.81 (m, 2H), 3.82-3.71 (m, 1H), 3.01- 3.00 (m, 4H), 2.15-2.01 (m, 3H), 2.00-1.83 (m, 3H), 1.83-1.70 (m, 1H), 1.70-1.54 (m, 1H). de: 99%; Retention time: 16.0 min (column: lux 5 um cellulose-4, 2.12*25 cm, 5 μm; mobile phase A: water (0.05% DEA), mobile phase B: ACN; flow rate: 20 mL/min; gradient: 80% B to 80% B in 20 min; wave length: 262/196 nm). |
| 62b | 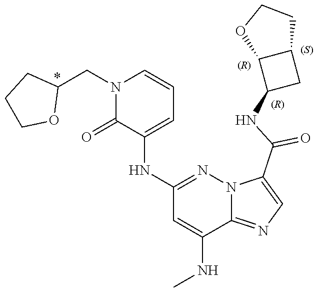 Diastereoisomer 2 (later eluting diastereoisomer) | LC-MS (ESI): m/z 480.50 [M + H]⁺; ¹H NMR (400 MHz, CDCl₃) δ 8.76 (d, J = 6.7 Hz, 1H), 8.05 (s, 1H), 7.95-7.86 (m, 1H), 7.74 (s, 1H), 7.13-7.06 (m, 1H), 6.37-6.17 (m, 2H), 5.66 (s, 1H), 4.62-4.56 (m, 1H), 4.46-4.36 (m, 2H), 4.31- 4.14 (m, 2H), 4.11-3.98 (m, 1H), 3.91-3.82 (m, 2H), 3.82-3.73 (m, 1H), 3.02-3.00 (m, 4H), 2.16- 2.06 (m, 3H), 1.97-1.81 (m, 3H), 1.83-1.72 (m, 1H), 1.69-1.56 (m, 1H). de: 95%; Retention time: 17.8 min (column: lux 5 um cellulose-4, 2.12*25 cm, 5 μm; mobile phase A: water (0.05% DEA), mobile phase B: ACN; flow rate: 20 mL/min; gradient: 80% B to 80% B in 20 min; wave length: 262/196 nm). |
| 63 | 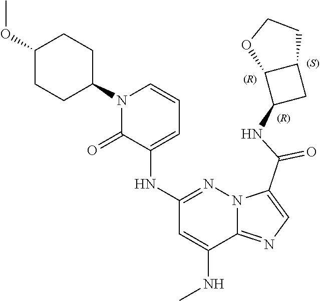 | LC-MS (ESI): m/z 508.3 [M + H]⁺; ¹H NMR (300 MHz, DMSO-d₆) δ 8.74 (d, J = 7.2 Hz, 1H), 8.51 (s, 1H), 8.01 (d, J = 6.6 Hz, 1H), 7.84 (s, 1H), 7.46 (d, J = 4.2 Hz, 1H), 7.38 (d, J = 6.3 Hz, 1H), 6.44-6.25 (m, 2H), 4.80 (s, 1H), 4.45 (s, 1H), 4.28-4.26 (m, 1H), 4.09-4.06 (m, 1H), 3.99-3.94 (m, 1H), 3.32-3.28 (m, 4H), 2.88-2.86 (m, 4H), 2.17-2.13 (m, 2H), 2.00-1.81 (m, 8H), 1.34-1.31 (m, 2H). |
| 64 | 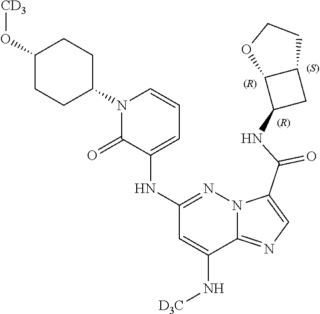 | LC-MS (ESI): m/z 514.4 [M + H]⁺; ¹H NMR (300 MHz, DMSO-d₆) δ 8.76 (d, J = 7.8 Hz, 1H), 8.52 (s, 1H), 8.01 (d, J = 8.0 Hz, 1H), 7.84 (s, 1H), 7.44 (s, 1H), 7.32 (d, J = 6.6 Hz, 1H), 6.41- 6.26 (m, 2H), 4.90-475 (m, 1H), 4.48-4.41 (m, 1H), 4.35-4.20 (m, 1H), 4.12-4.04 (m, 1H), 4.00- 3.84 (m, 1H), 3.51-3.44 (m, 1H), 2.99-2.83 (m, 1H), 2.14-1.66 (m, 8H), 1.66-1.50 (m, 4H). |
| 65 | 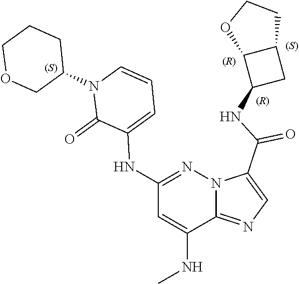 | LC-MS (ESI): m/z 480.2 [M + H]⁺; ¹H NMR (500 MHz, CDCl₃) δ 8.78 (d, J = 6.5 Hz, 1H), 8.09 (s, 1H), 7.96 (dd, J = 7.2, 1.6 Hz, 1H), 7.80 (s, 1H), 6.39 (t, J = 7.2 Hz, 1H), 5.89 (d, J = 5.3 Hz, 1H), 5.70 (s, 1H), 5.09-5.00 (m, 1H), 4.66- 4.60 (m, 1H), 4.48-4.41 (m, 1H), 4.26-4.19 (m, 1H), 4.11-4.02 (m, 2H), 3.95-3.87 (m, 1H), 3.75 (dd, J = 11.5, 7.6 Hz, 1H), 3.71-3.63 (m, 1H), 3.09- 2.99 (m, 4H), 2.19-1.97 (m, 5H), 1.88-1.77 (m, 3H). |
| 66 | 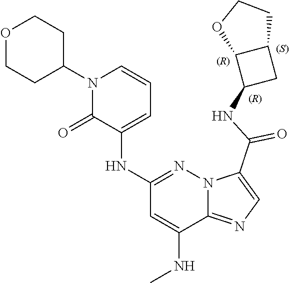 | LC-MS (ESI): m/z 480.2 [M + H]⁺; ¹H NMR (500 MHz, CDCl₃) δ 8.74 (d, J = 6.3 Hz, 1H), 8.08 (s, 1H), 7.93 (d, J = 6.0 Hz, 1H), 7.80 (s, 1H), 7.04 (d, J = 5.8 Hz, 1H), 6.41 (t, J = 7.2 Hz, 1H), 6.34 (s, 1H), 5.72 (s, 1H), 5.27-5.18 (m, 1H), 4.60 (dd, J = 6.5, 3.3 Hz, 1H), 4.45-4.38 (m, 1H), 4.22-4.17 (m, 1H), 4.17-4.12 (m, 2H), 4.08- 4.02 (m, 1H), 3.68-3.60 (m, 2H), 3.05 (d, J = 5.1 Hz, 3H), 3.02-2.96 (m, 1H), 2.17-2.11 (m, 1H), 2.10-2.01 (m, 1H), 2.01-1.88 (m, 5H), 1.82- 1.75 (m, 1H). |
| 67 | 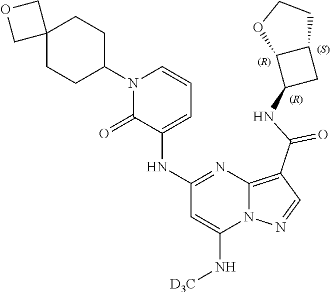 | LC-MS (ESI): m/z 523.3 [M + H]⁺; ¹H NMR (00 MHz, CDCl₃) δ 8.36-8.30 (m, 2H), 8.05 (s, 1H), 7.99 (d, J = 6.8 Hz, 1H), 6.94 (dd, J = 7.1, 1.7 Hz, 1H), 6.34 (t, J = 7.2 Hz, 1H), 6.24 (s, 1H), 5.44 (s, 1H), 4.93-4.82 (m, 1H), 4.58 (dd, J = 6.8, 3.3 Hz, 1H), 4.53 (s, 2H), 4.43-4.34 (m, 3H), 4.23-4.15 (m, 1H), 4.10-4.01 (m, 1H), 3.04- 2.96 (m, 1H), 2.36 (d, J = 13.3 Hz, 2H), 2.13 (td, J = 8.9, 4.4 Hz, 1H), 2.08-2.00 (m, 1H), 1.98-1.88 (m, 3H), 1.79-1.68 (m, 3H), 1.57-1.43 (m, 2H). |
| 68a | 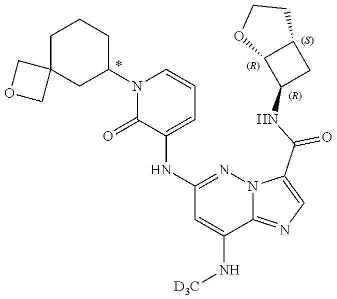 Diastereoisomer 1 (earlier eluting diastereoisomer) | LC-MS (ESI): m/z 523.30 [M + H]⁺; ¹H NMR (400 MHz, DMSO-d₆) δ 8.75 (d, J = 7.5 Hz, 1H), 8.57 (s, 1H), 8.03 (d, J = 7.3 Hz, 1H), 7.85 (s, 1H), 7.45-7.36 (m, 2H), 6.38-6.29 (m, 2H), 4.67 (t, J = 11.4 Hz, 1H), 4.53-4.41 (m, 3H), 4.33- 4.22 (m, 3H), 4.11-4.02 (m, 1H), 4.00-3.90 (m, 1H), 2.97-2.87 (m, 1H), 2.24 (d, J = 12.1 Hz, 1H), 2.11 (d, J = 9.0 Hz, 1H), 2.05-1.90 (m, 3H), 1.83- 1.62 (m, 5H), 1.48-1.34 (m, 2H). de: 100%; Retention time: 7.50 min (column: chiral art cellulose-SB, 2*25 cm 5 μm; mobile phase A: MtBE (10 mM NH₃—MeOH), mobile phase B: EtOH--HPLC; flow rate: 20 mL/min; gradient: 50% B to 50% B in 12 min; wave length: 264/250 nm). |
| 68b | 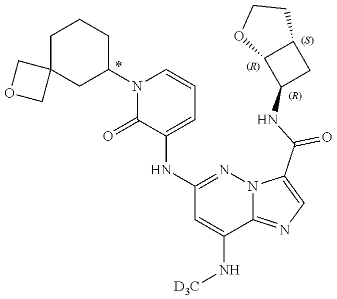 Diastereoisomer 2 (later eluting diastereoisomer) | LC-MS (ESI): m/z 523.30 [M + H]⁺; ¹H NMR (400 MHz, DMSO-d₆) δ 8.76 (d, J = 7.4 Hz, 1H), 8.57 (s, 1H), 8.03 (dd, J = 7.3, 1.6 Hz, 1H), 7.85 (s, 1H), 7.45-7.36 (m, 2H), 6.38-6.30 (m, 2H), 4.73-4.62 (m, 1H), 4.51-4.40 (m, 3H), 4.31- 4.24 (m, 3H), 4.07-4.05 (m, 1H), 3.95-3.93 (m, 1H), 2.98-2.87 (m, 1H), 2.24 (d, J = 12.0 Hz, 1H), 2.11 (d, J = 9.5 Hz, 1H), 2.07-1.60 (m, 8H), 1.49- 1.37 (m, 2H). de: 99%; Retention time: 9.47 min (column: chiral art cellulose-SB, 2*25 cm, 5 μm; mobile phase A: MtBE (10 mM NH₃—MeOH), mobile phase B: EtOH--HPLC; flow rate: 20 mL/min; gradient: 50% B to 50% B in 12 min; wave length: 264/250 nm). |
| 69 | 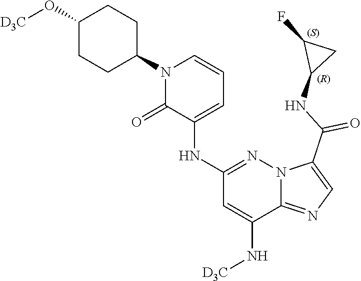 | LC-MS (ESI): m/z 476.6 [M + H]⁺; ¹H NMR (300 MHz, CDCl₃) δ 8.64 (d, J = 4.2 Hz, 1H), 8.13 (s, 1H), 7.86-7.73 (m, 2H), 6.94 (d, J = 6.2 Hz, 1H), 6.34 (t, J = 7.2 Hz, 1H), 6.00 (s, 1H), 5.69 (s, 1H), 5.04-4.62 (m, 2H), 3.29-3.13 (m, 2H), 2.27 (d, J = 12.3 Hz, 2H), 2.06 (d, J = 11.7 Hz, 2H), 1.61-1.41 (m, 4H), 1.35-1.23 (m, 1H), 1.19- 1.03 (m, 1H). |
| 70 | 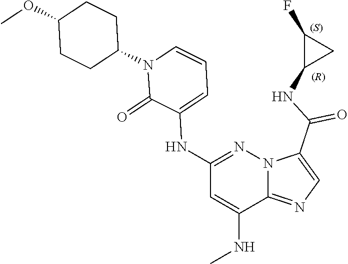 | LC-MS (ESI): m/z 470.15 [M + H]⁺; ¹H NMR (300 MHz, DMSO-d₆) δ 8.75-8.60 (m, 2H), 8.17 (s, 1H), 7.96-7.75 (m, 2H), 7.32 (dd, J = 7.1, 1.7 Hz, 1H), 6.54 (s, 1H), 6.29 (t, J = 7.2 Hz, 1H), 5.02-4.73 (m, 2H), 3.47 (s, 1H), 3.27 (s, 3H), 3.04-2.93 (m, 1H), 2.90 (s, 3H), 2.06-2.02 (m, 2H), 1.88-1.81 (m, 2H), 1.63-1.47 (m, 4H), 1.34- 1.16 (m, 1H), 1.05-0.88 (m, 1H). |
| 71 | 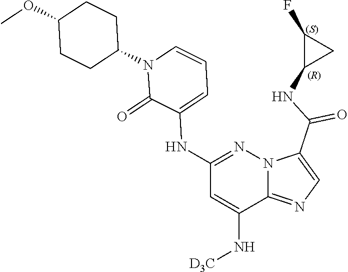 | LC-MS (ESI): m/z 473.15 [M + H]⁺; ¹H NMR (400 MHz, CDCl₃) δ 9.20 (s, 1H), 8.56 (d, J = 4.3 Hz, 1H), 8.22 (s, 1H), 8.01 (s, 1H), 7.70 (d, J = 7.2 Hz, 1H), 7.17 (d, J = 7.1 Hz, 1H), 6.32 (t, J = 7.1 Hz, 1H), 5.96 (s, 1H), 5.00-4.68 (m, 2H), 3.54 (s, 1H), 3.36 (s, 3H), 3.19-3.07 (m, 1H), 2.16 (d, J = 14.0 Hz, 2H), 1.91 (q, J = 12.6 Hz, 2H), 1.75- 1.72 (m, 2H), 1.64 (t, J = 13.7 Hz, 2H), 1.39-1.25 (m, 1H), 1.16-1.05 (m, 1H). |
| 72 | 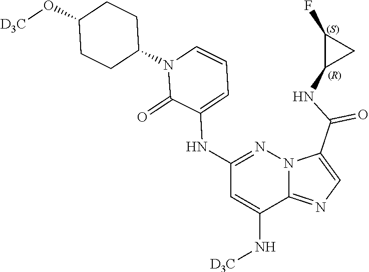 | LC-MS (ESI): m/z 476.20 [M + H]⁺; ¹H NMR (400 MHz, DMSO-d₆) δ 8.62 (d, J = 4.3 Hz, 1H), 8.50 (s, 1H), 7.93-7.91 (m, 1H), 7.84 (dd, J = 7.5, 1.5 Hz, 1H), 7.46 (s, 1H), 7.29 (dd, J = 7.1, 1.7 Hz, 1H), 6.37 (s, 1H), 6.28 (t, J = 7.2 Hz, 1H), 4.97-4.73 (m, 2H), 3.47 (s, 1H), 3.02-2.90 (m, 1H), 2.04-2.02 (m, 2H), 1.94-1.80 (m, 2H), 1.64- 1.50 (m, 4H), 1.28-1.18 (m, 1H), 0.99-0.86 (m, 1H). |
| 73 | 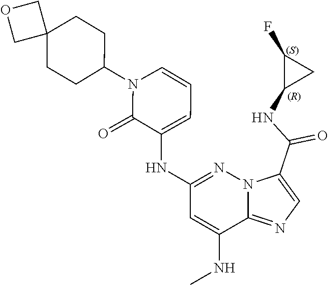 | LC-MS (ESI): m/z 482.2 [M + H]⁺; ¹H NMR (500 MHz, CDCl₃) δ 8.59 (d, J = 4.2 Hz, 1H), 8.13 (s, 1H), 7.78 (d, J = 7.1 Hz, 2H), 6.91 (dd, J = 7.2, 1.6 Hz, 1H), 6.40-6.27 (m, 2H), 5.70 (s, 1H), 4.93-4.67 (m, 2H), 4.53 (s, 2H), 4.42 (s, 2H), 3.14 (dd, J = 9.2, 5.0 Hz, 1H), 3.04 (d, J = 5.1 Hz, 3H), 2.36 (d, J = 13.4 Hz, 2H), 2.00-1.91 (m, 2H), 1.78-1.68 (m, 2H), 1.59-1.49 (m, 2H), 1.35- 1.21 (m, 1H), 1.14-1.02 (m, 1H). |
| 74 | 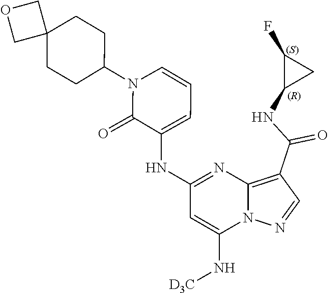 | LC-MS (ESI): m/z 485.2 [M + H]⁺; ¹H NMR (500 MHz, CDCl₃) δ 8.38 (s, 1H), 8.24 (d, J = 7.1 Hz, 1H), 8.07 (s, 1H), 7.82 (d, J = 4.7 Hz, 1H), 6.91 (dd, J = 7.1, 1.7 Hz, 1H), 6.34 (t, J = 7.2 Hz, 1H), 6.21 (s, 1H), 5.43 (s, 1H), 4.93-4.69 (m, 2H), 4.53 (s, 2H), 4.42 (s, 2H), 3.20-3.08 (m, 1H), 2.36 (d, J = 13.0 Hz, 2H), 1.94 (d, J = 12.5 Hz, 2H), 1.76-1.67 (m, 2H), 1.51 (s, 2H), 1.33-1.26 (m, 1H), 1.09-0.97 (m, 1H). |
| 75a | 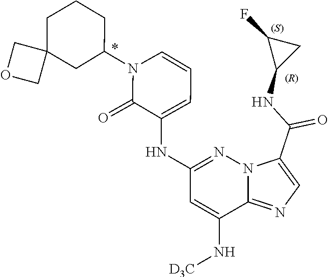 Diastereoisomer 1 (earlier eluting diastereoisomer) | LC-MS (ESI): m/z 485.25 [M + H]⁺; ¹H NMR (400 MHz, Methanol-d₄) δ 7.97 (s, 1H), 7.87 (dd, J = 7.4, 1.6 Hz, 1H), 7.33 (dd, J = 7.1, 1.7 Hz, 1H), 6.44 (t, J = 7.2 Hz, 1H), 6.08 (s, 1H), 4.91- 4.87 (m, 0.5H), 4.82-4.76 (m, 1H), 4.75-4.70 (m, 0.5H), 4.68 (d, J = 5.9 Hz, 1H), 4.56 (dd, J = 5.9, 1.2 Hz, 1H), 4.45-4.37 (m, 2H), 3.04-2.97 (m, 1H), 2.37 (d, J = 12.1 Hz, 1H), 2.22 (d, J = 9.3 Hz, 1H), 1.94-1.79 (m, 3H), 1.74-1.44 (m, 1H), 1.51 (d, J = 9.0 Hz, 2H), 1.34-1.21 (m, 1H), 1.08- 0.94 (m, 1H). de: 100%; Retention time: 7.0 min (column: chiral art cellulose-SB, 3*25 cm, 5 μm; mobile phase A: MtBE (10 mM NH₃—MeOH), mobile phase B: EtOH-- HPLC; flow rate: 40 mL/min; gradient: 50% B to 50% B in 10 min; wave length: 262/206 nm). |
| 75b | 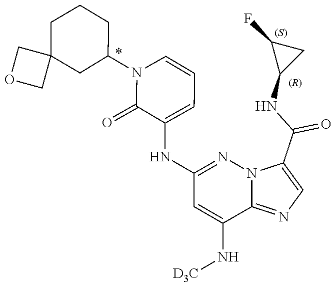 Diastereoisomer 2 (later eluting diastereoisomer) | LC-MS (ESI): m/z 485.25 [M + H]⁺; ¹H NMR (400 MHz, Methanol-d₄) δ 7.98 (s, 1H), 7.91 (dd, J = 7.4, 1.6 Hz, 1H), 7.35 (dd, J = 7.1, 1.7 Hz, 1H), 6.46 (t, J = 7.2 Hz, 1H), 6.11 (s, 1H), 4.91- 4.87 (m, 0.5H), 4.82-4.77 (m, 1H), 4.75-4.70 (m, 0.5H), 4.68 (d, J = 5.7 Hz, 1H), 4.56 (d, J = 5.7 Hz, 1H), 4.45-4.37 (m, 2H), 3.05-2.96 (m, 1H), 2.37 (d, J = 12.3 Hz, 1H), 2.22 (d, J = 9.1 Hz, 1H), 1.95-1.79 (m, 3H), 1.73-1.62 (m, 1H), 1.55- 1.47 (m, 2H), 1.34-1.20 (m, 1H), 1.07-0.94 (m, 1H). de: 99%; Retention time: 9.0 min (column: chiral art cellulose-SB, 3*25 cm, 5 μm; mobile phase A: MtBE (10 mM NH₃—MeOH), mobile phase B: EtOH-- HPLC; flow rate: 40 mL/min; gradient: 50% B to 50% B in 10 min; wave length: 262/206 nm). |
| 76 | 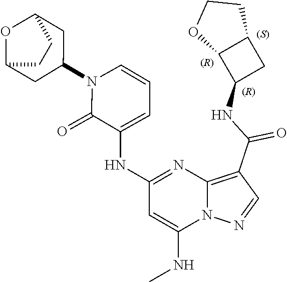 | LC-MS (ESI): m/z 506.20 [M + H]⁺; ¹H NMR (400 MHz, DMSO-d₆) δ 8.89 (s, 1H), 8.24 (d, J = 7.2 Hz, 1H), 8.18 (s, 1H), 7.99 (d, J = 7.6 Hz, 1H), 7.88 (d, J = 5.1 Hz, 1H), 7.49 (d, J = 7.1 Hz, 1H), 6.29 (t, J = 7.2 Hz, 1H), 6.20 (s, 1H), 5.03 (t, J = 9.6 Hz, 1H), 4.47 (d, J = 8.6 Hz, 2H), 4.38 (dd, J = 6.3, 3.1 Hz, 1H), 4.23 (d, J = 8.1 Hz, 1H), 4.06 (t, J = 8.3 Hz, 1H), 3.94 (dd, J = 9.7, 6.6 Hz, 1H), 2.90 (d, J = 4.9 Hz, 4H), 2.32 (q, J = 10.4, 8.4 Hz, 2H), 2.07-1.96 (m, 1H), 1.92-1.80 (m, 6H), 1.65 (t, J = 12.2 Hz, 3H). |
| 77 | 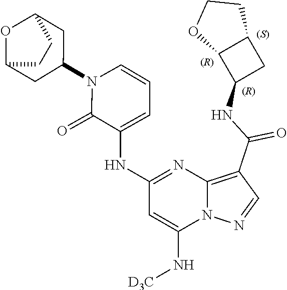 | LC-MS (ESI): m/z 509.20 [M + H]⁺; ¹H NMR (400 MHz, DMSO-d₆) δ 8.90 (s, 1H), 8.25 (dd, J = 7.3, 1.7 Hz, 1H), 8.18 (s, 1H), 8.00 (d, J = 7.6 Hz, 1H), 7.87 (s, 1H), 7.51 (dd, J = 7.0, 1.8 Hz, 1H), 6.30 (t, J = 7.2 Hz, 1H), 6.21 (s, 1H), 5.09- 4.94 (m, 1H), 4.53-4.42 (m, 2H), 4.39 (dd, J = 6.7, 3.1 Hz, 1H), 4.29-4.20 (m, 1H), 4.10-4.03 (m, 1H), 3.97-3.90 (m, 1H), 3.03-2.88 (m, 1H), 2.39- 2.28 (m, 2H), 2.07-1.97 (m, 1H), 1.91-1.77 (m, 6H), 1.66-1.62 (m, 3H). |
| 78 | 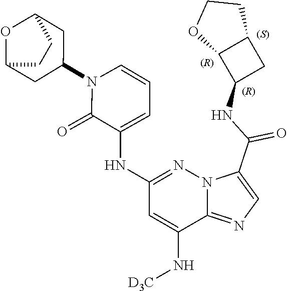 | LC-MS (ESI): m/z 509.20 [M + H]⁺; ¹H NMR (400 MHz, DMSO-d₆) δ 8.75 (d, J = 7.4 Hz, 1H), 8.54 (s, 1H), 8.01 (d, J = 7.2 Hz, 1H), 7.84 (s, 1H), 7.45-7.41 (m, 2H), 6.37 (s, 1H), 6.32 (t, J = 7.1 Hz, 1H), 5.09-4.92 (m, 1H), 4.50-4.41 (m, 3H), 4.33-4.23 (m, 1H), 4.07-4.06 (m, 1H), 3.97- 3.93 (m, 1H), 2.95-2.84 (m, 1H), 2.40-2.27 (m, 2H), 1.95-1.77 (m, 7H), 1.76-1.53 (m, 3H). |
| 79a | 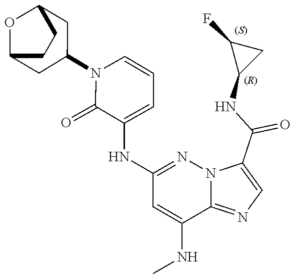 Diastereoisomer 1 (earlier eluting diastereoisomer) | LC-MS (ESI): m/z 468.20 [M + H]⁺; ¹H NMR (300 MHz, DMSO-d₆) δ 8.61 (d, J = 4.2 Hz, 1H), 8.52 (s, 1H), 7.89 (s, 1H), 7.83 (dd, J = 7.4, 1.6 Hz, 1H), 7.49 (d, J = 5.1 Hz, 1H), 7.38 (dd, J = 7.2, 1.7 Hz, 1H), 6.34 (s, 1H), 6.28 (t, J = 7.2 Hz, 1H), 5.35-5.30 (m, 1H), 5.01-4.72 (m, 1H), 4.49 (s, 2H), 3.03-2.92 (m, 1H), 2.86 (d, J = 4.8 Hz, 3H), 2.04-1.82 (m, 6H), 1.75-1.70 (m, 2H), 1.31- 1.15 (m, 1H), 1.04-0.87 (m, 1H). de: 100%; Retention time: 6.68 min (column: ymc-actus triart C18 ExRS, 30%150 mm, 5 μm; mobile phase A: water (10 mmol/L NH₄HCO₃), mobile phase B: ACN; flow rate: 60 mL/min; gradient: 27% B to 38% B in 10 min, 38% B; wave length: 254 nm). |
| 79b | 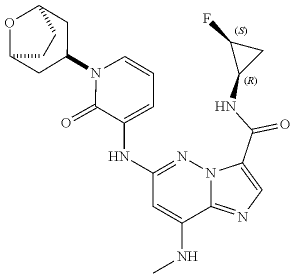 Diastereoisomer 2 (later eluting diastereoisomer) | LC-MS (ESI): m/z 468.20 [M + H]⁺; ¹H NMR (300 MHz, DMSO-d₆) δ 8.61 (d, J = 4.1 Hz, 1H), 8.53 (s, 1H), 7.89 (s, 1H), 7.88-7.83 (m, 1H), 7.52-7.42 (m, 2H), 6.38 (s, 1H), 6.28 (t, J = 7.1 Hz, 1H), 5.07-4.72 (m, 2H), 4.49 (s, 2H), 2.97 (dd, J = 9.0, 4.6 Hz, 1H), 2.86 (d, J = 4.8 Hz, 3H), 2.39-2.25 (m, 2H), 1.97-1.80 (m, 4H), 1.66 (t, J = 12.1 Hz, 2H), 1.31-1.14 (m, 1H), 1.03-0.86 (m, 1H). de: 100%; Retention time: 7.98 min (column: ymc-actus triart C18 ExRS, 30*150 mm, 5 μm; mobile phase A: water (10 mmol/L NH₄HCO₃), mobile phase B: ACN; flow rate: 60 mL/min; gradient: 27% B to 38% B in 10 min, 38% B; wave length: 254 nm). |
| 80 | 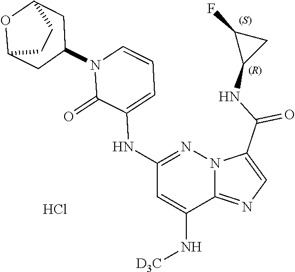 | LC-MS (ESI): m/z 507.20 [M + H]⁺; ¹H NMR (400 MHz, Methanol-d₄) δ 8.42 (d, J = 1.2 Hz, 1H), 7.94 (d, J = 7.3 Hz, 1H), 7.48 (d, J = 7.0 Hz, 1H), 6.56 (d, J = 1.3 Hz, 1H), 6.47 (t, J = 7.2 Hz, 1H), 5.25-5.12 (m, 1H), 4.76-4.69 (m, 1H), 4.55 (d, J = 7.5 Hz, 2H), 3.00 (dt, J = 10.4, 5.4 Hz, 1H), 2.48-2.45 (m, 2H), 2.06-2.04 (m, 2H), 1.90 (t, J = 6.6 Hz, 2H), 1.72-1.70 (m, 2H), 1.30-1.25 (m, 1H), 1.07-0.94 (m, 1H). |
| 81 | 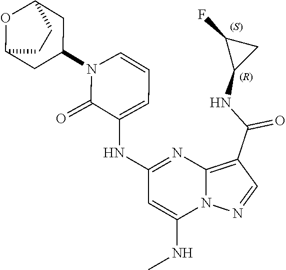 | LC-MS (ESI): m/z 468.15 [M + H]⁺; ¹H NMR (300 MHz, CDCl₃) δ 8.41 (s, 1H), 8.25 (d, J = 7.3 Hz, 1H), 8.11 (s, 1H), 7.84 (d, J = 4.6 Hz, 1H), 6.98 (dd, J = 7.1, 1.7 Hz, 1H), 6.36 (t, J = 7.2 Hz, 1H), 6.27-6.17 (m, 1H), 5.45 (s, 1H), 5.13- 5.01 (m, 1H), 4.93-4.70 (m, 1H), 4.63-4.55 (m, 2H), 3.23-3.13 (m, 1H), 3.09 (d, J = 5.2 Hz, 3H), 2.55-2.52 (m, 2H), 2.17-2.08 (m, 2H), 1.89-1.80 (m, 2H), 1.76-1.72 (m, 2H), 1.35-1.21 (m, 1H), 1.13-0.97 (m, 1H). |
| 82 | 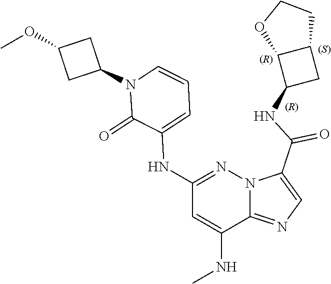 | LC-MS (ESI): m/z 480.2 [M + H]⁺; ¹H NMR (400 MHz, CDCl₃) δ 8.76 (d, J = 4.8 Hz, 1H), 8.01 (s, 1H), 7.91 (d, J = 1.2 Hz 1H), 7.80 (s, 1H), 7.08 (d, J = 5.6 Hz, 1H), 6.40 (t, J = 7.2 Hz, 2H), 5.70 (s, 1H), 5.35-5.38 (m, 1H), 4.59-4.62 (m, 1H), 4.39-4.21 (m, 1H), 4.21-4.05 (m, 3H), 3.34 (s, 3H), 3.05-3.03 (m, 4H), 2.71-2.60 (m, 2H), 2.66-2.54 (m, 2H), 2.10-1.90 (m, 1H), 1.96- 1.94 (m, 1H), 1.93-1.70 (m, 1H), 1.80-1.76 (m, 1H). |
| 83 | 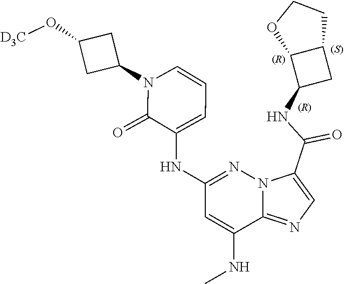 | LC-MS (ESI): m/z 483.2 [M + H]⁺; ¹H NMR (400 MHz, DMSO-d₆) δ 8.75 (d, J = 7.4 Hz, 1H), 8.55 (s, 1H), 8.03 (dd, J = 7.3, 1.7 Hz, 1H), 7.84 (s, 1H), 7.49-7.41 (m, 2H), 6.37-6.29 (m, 2H), 5.33-5.24 (m, 1H), 4.45 (dd, J = 6.8, 3.2 Hz, 1H), 4.32-4.23 (m, 1H), 4.10-4.02 (m, 2H), 3.98- 3.89 (m, 1H), 2.94-2.90 (m, 1H), 2.86 (d, J = 4.8 Hz, 3H), 2.58-2.52 (m, 2H), 2.49-2.41 (m, 2H), 2.03-1.71 (m, 4H). |
| 84 | 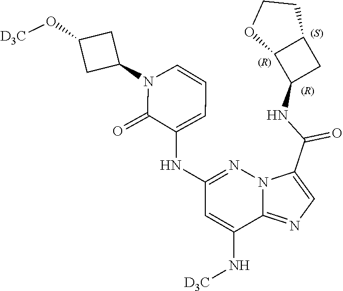 | LC-MS (ESI): m/z 486.25 [M + H]⁺; ¹H NMR (400 MHz, DMSO-d₆) δ 8.75 (d, J = 7.6 Hz, 1H), 8.55 (s, 1H), 8.03 (dd, J = 7.2, 1.6 Hz, 1H), 7.84 (s, 1H), 7.47 (dd, J = 7.2, 1.6 Hz, 1H), 7.42 (s, 1H), 6.37-6.29 (m, 2H), 5.36-5.20 (m, 1H), 4.45 (dd, J = 6.8, 3.2 Hz, 1H), 4.32-4.21 (m, 1H), 4.12- 4.01 (m, 2H), 4.00-3.90 (m, 1H), 2.97-2.85 (m, 1H), 2.57-2.52 (m, 2H), 2.49-2.42 (m, 2H), 2.06- 1.67 (m, 4H). |
| 85 | 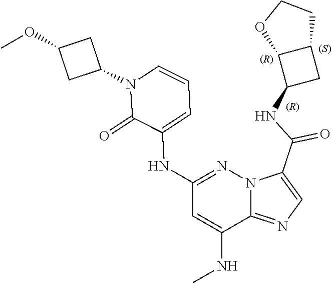 | LC-MS (EIS): m/z 480.20 [M + H]⁺; ¹H NMR (400 MHz, DMSO-d₆) δ 8.76 (d, J = 7.5 Hz, 1H), 8.53 (s, 1H), 8.02 (d, J = 7.5 Hz, 1H), 7.84 (s, 1H), 7.46-7.33 (m, 2H), 6.42-6.29 (m, 2H), 4.77-4.59 (m, 1H), 4.44 (dd, J = 6.9, 3.2 Hz, 1H), 4.34-4.22 (m, 1H), 4.11-4.03 (m, 1H), 3.99- 3.89 (m, 1H), 3.84-3.74 (m, 1H), 3.20 (s, 3H), 2.98- 2.73 (m, 6H), 2.17-2.06 (m, 2H), 2.03-1.94 (m, 1H), 1.93-1.86 (m, 1H), 1.85-1.78 (m, 1H), 1.77- 1.67 (m, 1H). |
| 86 | 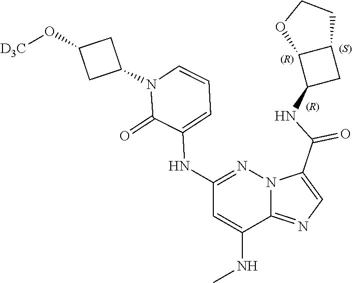 | LC-MS (ESI): m/z 483.51 [M + H]⁺; ¹H NMR (400 MHz, DMSO-d₆) δ 8.77 (d, J = 7.6 Hz, 1H), 8.54 (s, 1H), 8.02 (dd, J = 7.3, 1.7 Hz, 1H), 7.84 (s, 1H), 7.54-7.31 (m, 2H), 6.43-6.25 (m, 2H), 4.71-4.61 (m, 1H), 4.43 (dd, J = 6.7, 3.2 Hz, 1H), 4.32-4.22 (m, 1H), 4.12-4.03 (m, 1H), 3.95 (dd, J = 9.5, 6.3 Hz, 1H), 3.82-3.76 (m, 1H), 2.96- 2.74 (m, 6H), 2.16-1.69 (m, 6H). |
| 87 | 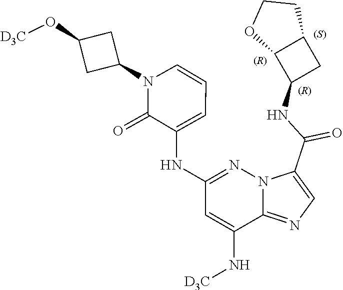 | LC-MS (ESI): m/z 486.2 [M + H]⁺; ¹H NMR (400 MHz, DMSO-d₆) δ 8.75 (d, J = 7.5 Hz, 1H), 8.54 (s, 1H), 8.03 (dd, J = 7.3, 1.6 Hz, 1H), 7.84 (s, 1H), 7.45-7.34 (m, 2H), 6.37-6.30 (m, 2H), 4.75-4.63 (m, 1H), 4.44 (dd, J = 6.8, 3.2 Hz, 1H), 4.28 (d, J = 8.1 Hz, 1H), 4.10-4.03 (m, 1H), 4.00-3.89 (m, 1H), 3.83-3.74 (m, 1H), 2.92 (s, 1H), 2.85-2.76 (m, 2H), 2.16-2.06 (m, 2H), 2.05- 1.67 (m, 4H). |
| 88a | 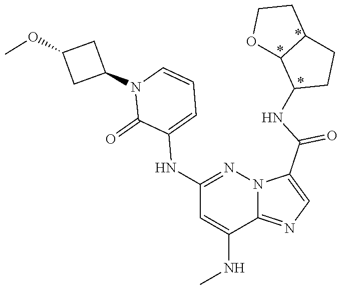 [SMILES: CNc1cc(Nc2cccn([C@H]3C[C@H](OC)C3)c2=O)nn2c(C(=O)NC3CCC4CCOC43)cnc12] Enantiomer 1 (earlier eluting enantiomer), relative stereochemistry of the 5,5-fused bicyclic structure is the same as that in Compound 10 | LC-MS (ESI): m/z 494.30 [M + H]⁺; ¹H NMR (400 MHz, CDCl₃) δ 8.45 (d, J = 7.0 Hz, 1H), 8.12 (s, 1H), 7.89-7.81 (m, 2H), 7.07 (dd, J = 7.1, 1.7 Hz, 1H), 6.35 (t, J = 7.2 Hz, 1H), 5.75 (s, 1H), 5.37 (p, J = 8.2 Hz, 1H), 4.42-4.26 (m, 2H), 4.16-4.09 (m, 1H), 3.93-3.78 (m, 2H), 3.34 (s, 3H), 3.04 (d, J = 5.0 Hz, 3H), 2.78-2.64 (m, 3H), 2.61-2.50 (m, 2H), 2.26 (dq, J = 12.0, 6.0 Hz, 1H), 2.07-1.95 (m, 2H), 1.68-1.54 (m, 2H), 1.53-1.40 (m, 1H). ee: 100%; Retention time: 13.94 min (column: chiral art cellulose-SB, 2*25 cm, 5 μm; mobile phase A: MtBE (10 mM NH₃—MeOH), mobile phase B: EtOH-- HPLC; flow rate: 20 mL/min; gradient: 20% B to 20% B in 18 min; wave length: 262.338 nm). |
| 88b | 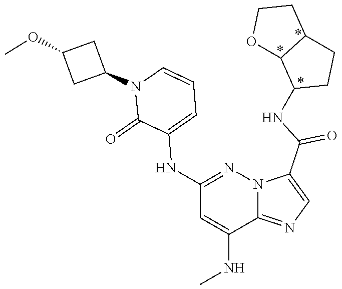 [SMILES: CNc1cc(Nc2cccn([C@H]3C[C@H](OC)C3)c2=O)nn2c(C(=O)NC3CCC4CCOC43)cnc12] Enantiomer 2 (later eluting enantiomer), relative stereochemistry of the 5,5-fused bicyclic structure is the same as that in Compound 10 | LC-MS (ESI): m/z 494.30 [M + H]⁺; ¹H NMR (400 MHz, CDCl₃) δ 8.94 (s, 1H), 8.41 (d, J = 7.0 Hz, 1H), 8.17 (s, 1H), 7.96 (s, 1H), 7.81 (dd, J = 7.4, 1.6 Hz, 1H), 7.12 (dd, J = 7.1, 1.6 Hz, 1H), 6.37 (t, J = 7.1 Hz, 1H), 5.90 (s, 1H), 5.36 (p, J = 8.1 Hz, 1H), 4.37 (dd, J = 7.9, 4.4 Hz, 1H), 4.32-4.23 (m, 1H), 4.17-4.10 (m, 1H), 3.94-3.80 (m, 2H), 3.34 (s, 3H), 3.07 (d, J = 4.7 Hz, 3H), 2.79-2.64 (m, 3H), 2.61-2.50 (m, 2H), 2.27 (dq, J = 11.5, 6.0 Hz, 1H), 2.08-1.95 (m, 2H), 1.53-1.38 (m, 3H). ee: 95%; Retention time: 15.122 min (column: chiral art cellulose-SB, 2*25 cm, 5 μm; mobile phase A: MtBE (10 mM NH₃—MeOH), mobile phase B: EtOH-- HPLC; flow rate: 20 mL/min; gradient: 20% B to 20% B in 18 min; wave length: 262.338 nm). |
| 89 | 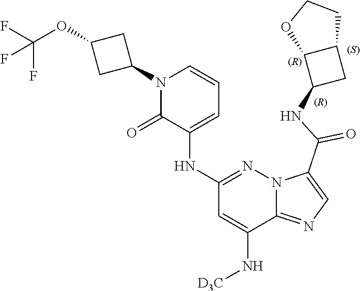 | LC-MS (ESI): m/z 537.3 [M + H]⁺; ¹H NMR (500 MHz, DMSO-d₆) δ 8.75 (d, J = 7.4 Hz, 1H), 8.57 (s, 1H), 8.05 (dd, J = 7.4, 1.7 Hz, 1H), 7.85 (s, 1H), 7.48-7.37 (m, 2H), 6.39-6.31 (m, 2H), 5.37-5.27 (m, 1H), 5.19-5.09 (m, 1H), 4.45 (dd, J = 6.8, 3.2 Hz, 1H), 4.32-4.24 (m, 1H), 4.07 (td, J = 8.4, 2.4 Hz, 1H), 3.95 (td, J = 9.5, 6.4 Hz, 1H), 2.96-2.86 (m, 3H), 2.81-2.71 (m, 2H), 2.05- 1.97 (m, 1H), 1.96-1.89 (m, 1H), 1.88-1.78 (m, 1H), 1.77-1.69 (m, 1H). |
| 90 | 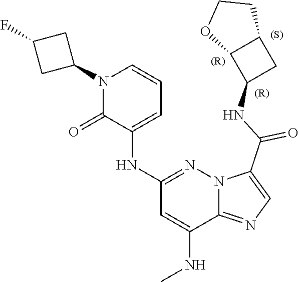 | LC-MS (ESI): m/z 468.2 [M + H]⁺; ¹H NMR (300 MHz, CDCl₃) δ 8.76-8.74 (m, 1H), 8.06 (s, 1H), 7.96-7.93 (m, 1H), 7.74 (s, 1H), 6.99- 6.96 (m, 1H), 6.42-6.37 (m, 1H), 6.03-6.01 (m, 1H), 5.67 (s, 1H), 5.45-5.21 (m, 2H), 4.61-4.58 (m, 1H), 4.46-4.33 (m, 1H), 4.25-4.01 (m, 2H), 3.04-3.02 (m, 3H), 2.93-2.73 (m, 5H), 2.14-1.93 (m, 3H), 1.81-1.79 (m, 1H). |
| 91 | 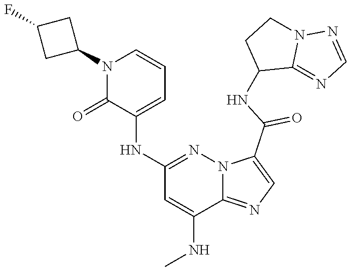 [SMILES: CNc1cc(Nc2cccn([C@H]3C[C@H](F)C3)c2=O)nn2c(C(=O)NC3CCn4ncnc43)cnc12] Racemic mixture | LC-MS (ESI): m/z 479.22 [M + H]⁺; ¹H NMR (500 MHz, DMSO-d₆) δ 9.05 (d, J = 8.6 Hz, 1H), 8.53 (s, 1H), 8.02 (s, 1H), 7.92 (s, 1H), 7.78 (dd, J = 7.4, 1.7 Hz, 1H), 7.52-7.42 (m, 1H), 7.31 (dd, J = 7.1, 1.7 Hz, 1H), 6.36 (s, 1H), 5.81 (t, J = 7.2 Hz, 1H), 5.68-5.58 (m, 1H), 5.45-5.26 (m, 2H), 4.34- 4.24 (m, 1H), 4.20-4.07 (m, 1H), 3.22-3.15 (m, 1H), 2.86 (d, J = 4.9 Hz, 3H), 2.82-2.61 (m, 4H), 2.58-2.53 (m, 1H). |
| 92a | 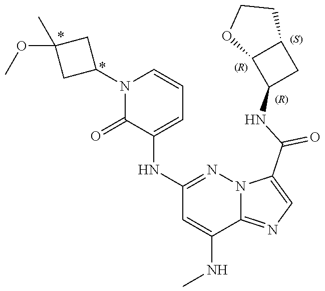 Diastereoisomer 1 (earlier eluting diastereoisomer) | LC-MS (ESI): m/z 494.24 [M + H]⁺; ¹H NMR (300 MHz, DMSO-d₆) δ 8.76 (d, J = 7.5 Hz, 1H), 8.55 (s, 1H), 8.05-8.02 (m, 1H), 7.84 (s, 1H), 7.46-7.37 (m, 2H), 6.36-6.32 (m, 2H), 4.81- 4.76 (m, 1H), 4.46-4.43 (dd, J = 6.7, 3.2 Hz, 1H), 4.32-4.25 (m, 1H), 4.13-4.02 (m, 1H), 4.02-3.87 (m, 1H), 3.13 (s, 3H), 2.98-2.87 (m, 4H), 2.49- 2.43 (m, 2H), 2.45-2.35 (m, 2H), 2.05-1.68 (m, 4H), 1.39 (s, 3H). de: 99%; Retention time: 7.37 min (column: chiral art cellulose-SB, 2*25 cm, 5 μm; mobile phase A: MtBE (10 mM NH₃—MeOH), mobile phase B: MeOH--HPLC; flow rate: 20 mL/min; gradient: 20% B to 20% B in 12 min; wave length: 248/262 nm). |
| 92b | 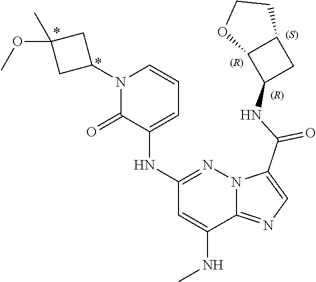 Diastereoisomer 2 (later eluting diastereoisomer) | LC-MS (ESI): m/z 494.24 [M + H]⁺; ¹H NMR (300 MHz, DMSO-d₆) δ 8.76 (d, J = 7.5 Hz, 1H), 8.55 (s, 1H), 8.05-8.02 (m, 1H), 7.84 (s, 1H), 7.47-7.44 (m, 2H), 6.37-6.31 (m, 2H), 5.15- 5.09 (m, 1H), 4.45 (dd, J = 6.7, 3.2 Hz, 1H), 4.32- 4.25 (m, 1H), 4.13-4.02 (m, 1H), 4.02-3.87 (m, 1H), 3.13 (s, 3H), 2.98-2.85 (m, 4H), 2.61-2.55 (m, 2H), 2.32-2.22 (m, 2H), 2.05-1.68 (m, 4H), 1.35 (s, 3H). de: 97%; Retention time: 9.245 min (column: chiral art cellulose-SB, 2*25 cm, 5 μm; mobile phase A: MtBE (10 mM NH₃—MeOH), mobile phase B: MeOH--HPLC; flow rate: 20 mL/min; gradient: 20% B to 20% B in 12 min; wave length: 248/262 nm). |
| 93a | 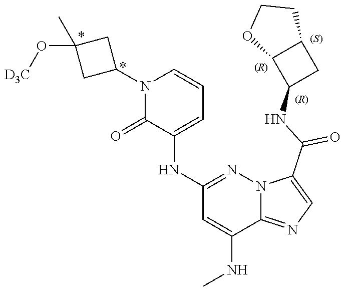 Diastereoisomer 1 (earlier eluting diastereoisomer) | LC-MS (ESI): m/z 497.30 [M + H]⁺; ¹H NMR (400 MHz, CDCl₃) δ 8.70 (d, J = 6.6 Hz, 1H), 7.99 (s, 1H), 7.85 (dd, J = 7.4, 1.7 Hz, 1H), 7.69 (s, 1H), 7.10 (dd, J = 7.1, 1.7 Hz, 1H), 6.34 (t, J = 7.2 Hz, 1H), 6.20 (d, J = 5.3 Hz, 1H), 5.61 (s, 1H), 5.03-4.91 (m, 1H), 4.57-4.48 (m, 1H), 4.42-4.30 (m, 1H), 4.17-4.12 (m, 1H), 4.04-3.94 (m, 1H), 3.01-2.95 (m, 4H), 2.60-2.51 (m, 2H), 2.33-2.28 (m, 2H), 2.08-1.86 (m, 3H), 1.76-1.67 (m, 1H), 1.40 (s, 3H). de: 100%; Retention time: 7.80 min (column: chiral art cellulose-SB, 2*25 cm, 5 μm; mobile phase A: MtBE (10 mM NH₃—MeOH), mobile phase B: MeOH--HPLC; flow rate: 20 mL/min; gradient: 20% B to 20% B in 12 min; wave length: 248/262 nm). |
| 93b | 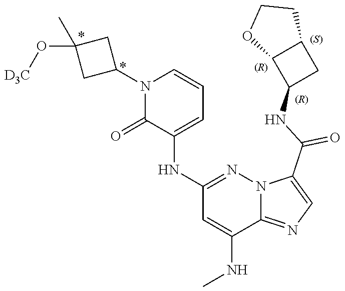 Diastereoisomer 2 (later eluting diastereoisomer) | LC-MS (ESI): m/z 497.30 [M + H]⁺; ¹H NMR (300 MHz, CDCl₃) δ 8.80 (d, J = 6.6 Hz, 1H), 8.09 (s, 1H), 7.94 (dd, J = 7.1, 1.4 Hz, 1H), 7.81 (s, 1H), 7.08-7.01 (m, 1H), 6.40 (t, J = 7.2 Hz, 1H), 6.25 (d, J = 5.6 Hz, 1H), 5.70 (s, 1H), 5.29- 5.13 (m, 1H), 4.66-4.58 (m, 1H), 4.52-4.38 (m, 1H), 4.27-4.17 (m, 1H), 4.15-4.02 (m, 1H), 3.10- 2.95 (m, 4H), 2.89-2.72 (m, 2H), 2.25 (t, J = 11.0 Hz, 2H), 2.18-1.75 (m, 4H), 1.43 (s, 3H). de: 98%; Retention time: 9.87 min (column: chiral art cellulose-SB, 2*25 cm, 5 μm; mobile phase A: MtBE (10 mM NH₃—MeOH), mobile phase B: MeOH--HPLC; flow rate: 20 mL/min; gradient: 20% B to 20% B in 12 min; wave length: 248/262 nm). |
| 94a | 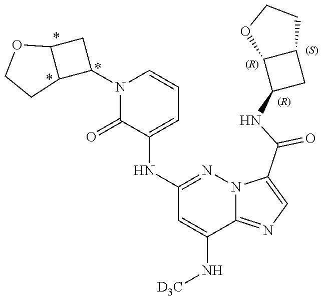 Diastereoisomer 1 (earlier eluting diastereoisomer) relative stereochemistry of the 4,5-fused bicyclic structure on the left hand side is the same as that of 3.3 | LC-MS (ESI): m/z 495.25 [M + H]⁺; ¹H NMR (400 MHz, DMSO-d₆) δ 8.75 (d, J = 7.5 Hz, 1H), 8.58 (s, 1H), 8.04 (dd, J = 7.3, 1.7 Hz, 1H), 7.84 (s, 1H), 7.48 (dd, J = 7.1, 1.7 Hz, 1H), 7.42 (s, 1H), 6.39-6.31 (m, 2H), 4.86-4.76 (m, 1H), 4.71- 4.70 (m, 1H), 4.45 (dd, J = 6.7, 3.2 Hz, 1H), 4.28 (d, J = 6.9 Hz, 1H), 4.13-4.04 (m, 2H), 4.03-3.89 (m, 2H), 3.30-3.21 (m, 1H), 2.98-2.87 (m, 1H), 2.75- 2.64 (m, 1H), 2.38-2.27 (m, 1H), 2.07-1.89 (m, 3H), 1.87-1.78 (m, 1H), 1.78-1.68 (m, 2H). de: 98%; Retention time: 16.0 min (column: chiralpak IH, 3*25 cm, 5 μm; mobile phase A: Hex: DCM = 5:1 (0.1% Et₂NH)--HPLC, mobile phase B: EtOH-- HPLC; flow rate: 40 mL/min; gradient: 5% B to 5% B in 24 min; wave length: 213.240 nm). |
| 94b | 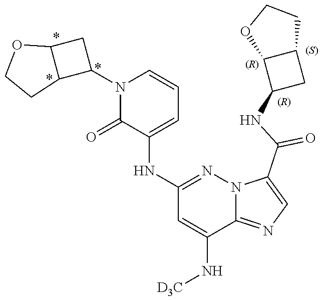 Diastereoisomer 2 (later eluting diastereoisomer), relative stereochemistry of the 4,5-fused bicyclic structure on the left hand side is the same as that of 3,3 | LC-MS (ESI): m/z 495.25 [M + H]⁺; ¹H NMR (400 MHz, DMSO-d₆) δ 8.76 (d, J = 7.5 Hz, 1H), 8.58 (s, 1H), 8.04 (d, J = 6.7 Hz, 1H), 7.84 (s, 1H), 7.48 (d, J = 7.0 Hz, 1H), 7.42 (s, 1H), 6.39- 6.31 (m, 2H), 4.86-4.76 (m, 1H), 4.71 (t, J = 6.4 Hz, 1H), 4.45 (dd, J = 6.9, 3.2 Hz, 1H), 4.32-4.24 (m, 1H), 4.13-3.89 (m, 4H), 3.30-3.21 (m, 1H), 2.98-2.85 (m, 1H), 2.75-2.67 (m, 1H), 2.37- 2.27 (m, 1H), 2.06-1.88 (m, 3H), 1.86-1.68 (m, 3H). de: 99%; Retention time: 20.0 min (column: chiralpak IH, 3*25 cm, 5 μm; mobile phase A: Hex: DCM = 5:1 (0.1% Et₂NH)--HPLC, mobile phase B: EtOH-- HPLC; flow rate: 40 mL/min; gradient: 5% B to 5% B in 24 min; wave length: 213/240 nm). |
| 95a | 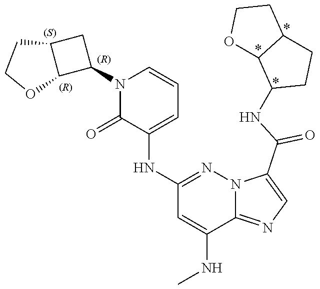 Diastereoisomer 1 (earlier eluting diastereoisomer), relative stereochemistry of the 5,5-fused bicyclic structure is the same as that of Compound 10 | LC-MS (ESI): m/z 506.20 [M + H]⁺; ¹H NMR (300 MHz, DMSO-d6) δ 8.53 (s, 1H), 8.46 (d, J = 7.3 Hz, 1H), 7.92 (d, J = 7.4 Hz, 1H), 7.85 (s, 1H), 7.50-7.40 (m, 2H), 6.36-6.27 (m, 2H), 5.04- 4.93 (m, 1H), 4.91-4.85 (m, 1H), 4.24-3.94 (m, 4H), 3.82-3.59 (m, 2 H), 3.19-3.07 (m, 1H), 2.86 (d, J = 4.8 Hz, 3H), 2.70-2.61 (m, 1H), 2.32-2.24 (m, 1H), 2.08-1.78 (m, 6H), 1.60-1.47 (m, 2H), 1.45- 1.30 (m, 1H). de: 100%; Retention time: 12.64 min (column: chiral art cellulose-SB, 2*25 cm 5 μm; mobile phase A: MtBE (10 mM NH₃—MeOH), mobile phase B: MeOH--HPLC; flow rate: 20 mL/min; gradient: 20% B to 20% B in 18 min; wave length: 250/262 nm). |
| 95b | 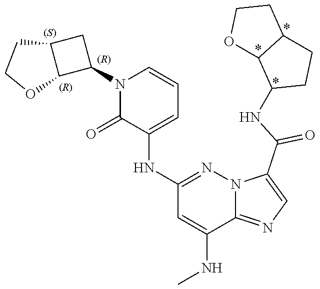 Diastereoisomer 2 (later eluting diastereoisomer) relative stereochemistry of the 5,5-fused bicyclic structure is the same as that of Compound 10 | LC-MS (ESI): m/z 506.20 [M + H]⁺; ¹H NMR (300 MHz, DMSO-d6) δ 8.53 (s, 1H), 8.45 (d, J = 7.3 Hz, 1H), 7.91 (d, J = 7.2 Hz, 1H), 7.85 (s, 1H), 7.53-7.34 (m, 2H), 6.38-6.24 (m, 2H), 5.06- 4.83 (m, 2H), 4.30-3.91 (m, 4H), 3.80-3.62 (m, 2H), 3.20-3.04 (m, 1H), 2.86 (d, J = 4.8 Hz, 3H), 2.75-2.62 (m, 1H), 2.45-2.34 (m, 1H), 2.09-1.73 (m, 6H), 1.60-1.32 (m, 3H). de: 97%; Retention time: 14.475 min (column: chiral art cellulose-SB, 2*25 cm, 5 μm; mobile phase A: MtBE (10 mM NH₃—MeOH), mobile phase B: MeOH--HPLC; flow rate: 20 mL/min; gradient: 20% B to 20% B in 18 min; wave length: 250/262 nm). |
| 96a | 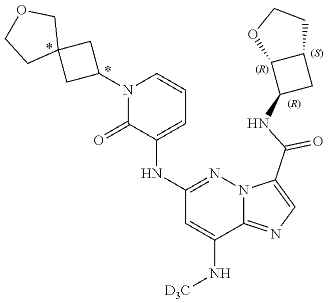 Diastereoisomer 1 (earlier eluting diastereoisomer) | LC-MS (ESI): m/z 509.30 [M + H]⁺; ¹H NMR (400 MHz, CDCl₃) δ 8.75 (d, J = 6.5 Hz, lH), 8.09 (s, 1H), 7.92 (dd, J = 7.4, 1.7 Hz, 1H), 7.79 (s, 1H), 7.07 (dd, J = 7.4, 1.7 Hz, 1H), 6.63 (s, 1H) 6.41 (t, J = 7.2 Hz, 1H), 5.72 (s, 1H), 5.26- 5.14 (m, 1H), 4.59 (dd, J = 6.9, 3.4 Hz, 1H), 4.45- 4.36 (m, 1H), 4.25-4.15 (m, 1H), 4.06-4.05 (m, 1H), 3.91 (t, J = 6.7 Hz, 2H), 3.75 (s, 2H), 3.06- 2.95 (m, 1H), 2.65-2.60 (m, 2H), 2.39-2.36 (m, 2H), 2.20-2.11 (m, 3H), 2.07-1.94 (m, 2H), 1.82-1.75 (m, 1H). de: 100%; Retention time: 8.9525 min (column: chiral art amylose-SA, 2*25 cm, 5 μm; mobile phase A: MtBE (10 mM NH₃—MeOH), mobile phase B: MeOH--HPLC; flow rate: 20 mL/min; gradient: 50% B to 50% B in 14 min; wave length: 338/262 nm). |
| 96b | 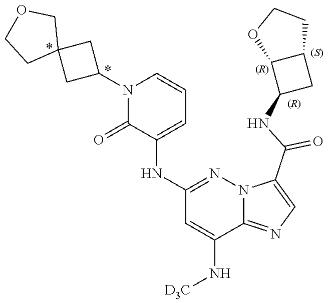 Diastereoisomer 2 (later eluting diasteroisomer) | LC-MS (ESI): m/z 509.30 [M + H]⁺; ¹H NMR (400 MHz, CDCl₃) δ 8.76 (d, J = 6.5 Hz, 1H), 8.08 (s, 1H), 7.93 (dd, J = 7.3, 1.7 Hz, 1H), 7.77 (s, 1H), 7.09 (dd, J = 7.1, 1.7 Hz, 1H), 6.41 (t, J = 7.2 Hz, 2H), 5.70 (s, 1H), 5.16-5.05 (m, 1H), 4.60 (dd, J = 6.9 , 3.3 Hz, 1H), 4.47-4.36 (m, 1H), 4.20-4.19 (m, 1H), 4.60-4.05 (m, 1H), 3.91-3.81 (m, 4H), 3.06-2.96 (m, 1H), 2.75-2.64 (m, 2H), 2.47-2.37 (m, 2H), 2.20-1.90 (m, 5H), 1.82- 1.75 (m, 1H). de: 99%; Retention time: 11.034 min (column: chiral art amylose-SA, 2*25 cm, 5 μm; mobile phase A: MtBE (10 mM NH₃—MeOH), mobile phase B: MeOH--HPLC; flow rate: 20 mL/min; gradient: 50% B to 50% B in 14 min; wave length: 338/262 nm). |
| 97a | 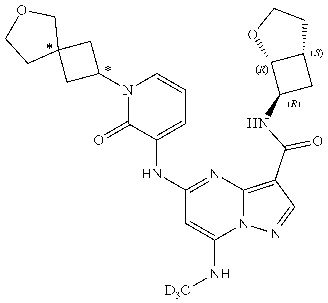 Diastereoisomer 1 (earlier eluting diastereoisomer) | LC-MS (ESI): m/z 509.30 [M + H]⁺; ¹H NMR (400 MHz, CDCl₃) δ 8.38-8.31 (m, 2H), 8.04-7.96 (m, 2H), 7.10 (dd, J = 7.1, 1.7 Hz, 1H), 6.38 (t, J = 7.2 Hz, 1H), 6.22 (s, 1H), 5.43 (s, 1H), 5.17-5.04 (m, 1H), 4.60 (dd, J = 6.7, 3.2 Hz, 1H), 4.45-4.36 (m, 1H), 4.210-4.17 (m, 1H), 4.07-4.06 (m, 1H), 3.90-3.81 (m, 4H), 3.08-2.99 (m, 1H), 2.74-2.64 (m, 2H), 2.46-2.37 (m, 2H), 2.21- 2.11 (m, 1H), 2.09-1.90 (m, 4H), 1.82-1.74 (m, 1H). de: 99%; Retention time: 25.0 min (column: chiralpak ID, 3*25 cm, 5 μm; mobile phase A: MtBE (10 mM NH₃—MeOH), mobile phase B: MeOH--HPLC; flow rate: 20 mL/min; gradient: 20% B to 20% B in 40 min; wave length: 214/235 nm). |
| 97b | 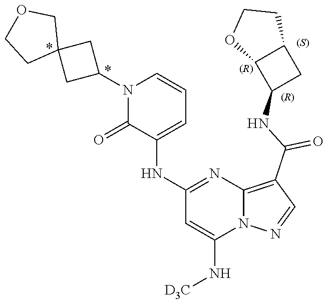 Diastereoisomer 2 (later eluting diastereoisomer) | LC-MS (ESI): m/z 509.30 [M + H]⁺; ¹H NMR (400 MHz, CDCl₃) δ 8.33 (s, 1H), 8.31 (d, J = 8.1 Hz, 1H), 7.90 (br, 1H), 7.12 (d, J = 6.9 Hz, 1H), 6.39 (t, J = 7.1 Hz, 1H), 6.32 (s, 1H), 5.54 (br, 1H), 5.19 (p, J = 8.6 Hz, 1H), 4.64-4.58 (m, 1H), 4.44-4.35 (m, 1H), 4.24-4.15 (m, 1H), 4.11- 4.00 (m, 1H), 3.90 (t, J = 6.7 Hz, 2H), 3.75 (s, 2H), 3.07-2.95 (m, 1H), 2.69-2.58 (m, 2H), 2.39 (t, J = 10.3 Hz, 2H), 2.20-2.04 (m, 4H), 1.98-1.89 (m, 1H), 1.82-1.74 (m, 1H). de: 99%; Retention time: 35.0 min (column: chiralpak ID, 3*25 cm, 5 μm; mobile phase A: MtBE (10 mM NH₃—MeOH), mobile phase B: MeOH--HPLC; flow rate: 20 mL/min; gradient: 20% B to 20% B in 40 min; wave length: 214/235 nm). |
| 98a | 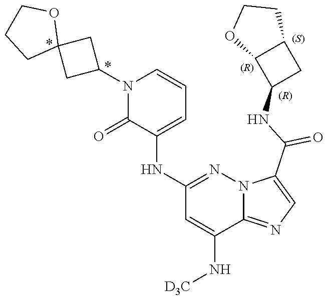 Diastereoisomer 1 (earlier eluting diastereoisomer) | LC-MS (ESI): m/z 509.30 [M + H]⁺; ¹H NMR (400 MHz, Methanol-d₄) δ 7.99 (d, J = 7.4 Hz, 1H), 7.91 (s, 1H), 7.39 (d, J = 6.8 Hz, 1H), 6.50- 6.46 (m, 1H), 6.06-6.03 (m, 1H), 4.85-4.74 (m, 1H), 4.51 (dd, J = 6.8, 3.2 Hz, 1H), 4.37-4.30 (m, 1H), 4.20-4.11 (m, 1H), 4.10-4.01 (m, 1H), 3.83 (t, J = 6.6 Hz, 2H), 3.09-2.96 (m, 1H), 2.74-2.65 (m, 2H), 2.53-2.42 (m, 2H), 2.15-2.05 (m, 3H), 2.05-1.89 (m, 4H), 1.83-1.76 (m, 1H). de: 99%; Retention time: 10.255 min (column: chiral art cellulose-SB, 2*25 cm 5 μm; mobile phase A: MtBE (10 mM NH₃—MeOH), mobile phase B: EtOH--HPLC; flow rate: 20 mL/min; gradient: 30% B to 30% B in 20 min; wave length: 262/238 nm). |
| 98b | 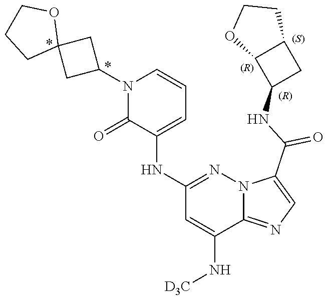 Diastereoisomer 2 (later eluting diastereoisomer) | LC-MS (ESI): m/z 509.30 [M + H ]⁺; ¹H NMR (400 MHz, Metahnol-d₄) δ 8.01 (d, J = 7.4 Hz, 1H), 7.92 (s, 1H), 7.38 (dd, J = 7.0 Hz, 1H), 6.49 (t, J = 7.2 Hz, 1H), 6.07 (s, 1H), 5.24-5.20 (m, 1H), 4.51 (dd, J = 6.7, 3.2 Hz, 1H), 4.39-4.30 (m, 1H), 4.15-4.13 (m, 1H), 4.06-4.04 (m, 1H), 3.85 (t, J = 6.6 Hz, 2H), 3.07-2.97 (m, 1H), 2.76- 2.66 (m, 2H), 2.58-2.45 (m, 2H), 2.15-1.86 (m, 7H), 1.86-1.75 (m, 1H). de: 98%; Retention time: 13.185 min (column: chiral art cellulose-SB, 2*25 cm, 5 μm; mobile phase A: MtBE (10 mM NH₃—MeOH), mobile phase B: EtOH--HPLC; flow rate: 20 mL/min; gradient: 30% B to 30% B in 20 min; wave length: 262/238 nm). |
| 99 | 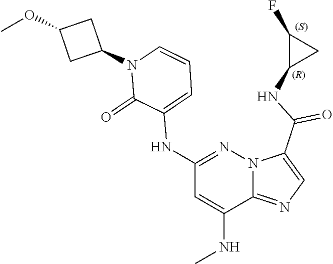 | LC-MS (ESI): m/z 442.25 [M + H]⁺; ¹H NMR (400 MHz, CDCl₃) δ 8.62 (d, J = 4.4 Hz, 1H), 8.12 (s, 1H), 7.81-7.79 (m, 1H), 7.76 (s, 1H), 7.04 (dd, J = 7.1, 1.7 Hz, 1H), 6.34 (t, J = 7.2 Hz, 1H), 6.16 (d, J = 5.1 Hz, 1H), 5.67 (s, 1H), 5.36 (p, J = 8.1 Hz, 1H), 4.90-4.67 (m, 1H), 4.17-4.09 (m, 1H), 3.33 (s, 3H), 3.21-3.11 (m, 1H), 3.02 (d, J = 5.1 Hz, 3H), 2.75-2.63 (m, 2H), 2.61-2.50 (m, 2H), 1.34-1.22 (m, 1H), 1.16-1.03 (m, 1H). |
| 100 | 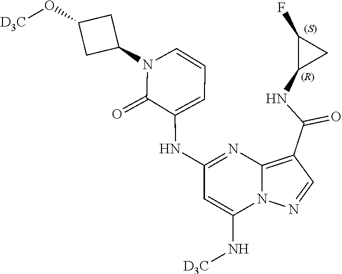 | LC-MS (ESI): m/z 448.2 [M + H]⁺; ¹H NMR (400 MHz, DMSO-d₆) δ 8.84 (s, 1H), 8.23 (s, 1H), 8.12 (dd, J = 7.4, 1.7 Hz, 1H), 7.85 (s, 1H), 7.81 (d, J = 4.8 Hz, 1H), 7.47 (dd, J = 7.1, 1.7 Hz, 1H), 6.30 (t, J = 7.2 Hz, 1H), 6.19 (s, 1H), 5.31- 5.22 (m, 1H), 4.98-4.77 (m, 1H), 4.08-4.02 (m, 1H), 3.04-2.95 (m, 1H), 2.57-2.52 (m, 1H), 2.48- 2.41 (m, 3H), 1.28-1.17 (m, 1H), 0.93-0.79 (m, 1H). |
| 101a | 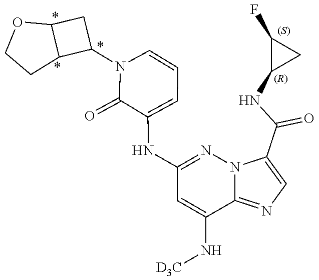 Diastereoisomer 1 (earlier eluting diastereoisomer), relative stereochemistry of the 4,5-fused bicyclic structure on the left hand side is the same as that of 3.3 | LC-MS (ESI): m/z 457.25 [M + H]⁺; ¹H NMR (300 MHz, DMSO-d₆) δ 8.63 (d, J = 4.2 Hz, 1H), 8.57 (s, 1H), 7.92-7.85 (m, 2H), 7.50- 7.43 (m, 2H), 6.38 (s, 1H), 6.32 (t, J = 7.1 Hz, 1H), 5.04-4.64 (m, 3H), 4.15-3.93 (m, 2H), 3.30- 3.21 (m, 1H), 3.04-2.93 (m, 1H), 2.78-2.63 (m, 1H), 2.40-2.26 (m, 1H), 2.02 (dd, J = 12.4, 5.3 Hz, 1H), 1.85-1.66 (m, 1H), 1.33-1.17 (m, 1H), 1.06- 0.88 (m, 1H). |
| 101b | 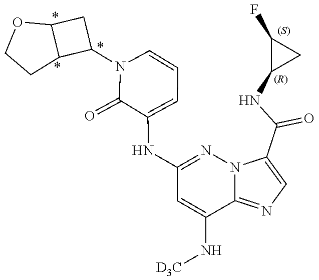 Diastereoisomer 2 (later eluting diastereoisomer), relative stereochemistry of the 4,5-fused bicyclic structure on the left hand side is the same as that of 3.3 | LC-MS (ESI): m/z 457.25 [M + H]⁺; ¹H NMR (300 MHz, DMSO-d₆) δ 8.63 (d, J = 4.2 Hz, 1H), 8.57 (s, 1H), 7.93-7.85 (m, 2H), 7.50- 7.43 (m, 2H), 6.41-6.27 (m, 2H), 5.03-4.67 (m, 3H), 4.15-3.93 (m, 2H), 3.31-3.20 (m, 1H), 3.06- 2.90 (m, 1H), 2.78-2.63 (m, 1H), 2.40-2.26 (m, 1H), 2.02 (dd, J = 12.6, 5.4 Hz, 1H), 1.85-1.66 (m, 1H), 1.34-1.15 (m, 1H), 1.04-0.85 (m, 1H). de: 99%; Retention time: 34.25 min (column: chiralpak ID, 2*25 cm, 5 μm; mobile phase A: Hex: MtBE = 1:1 (0.5% 2 M NH₃—MeOH), mobile phase B: EtOH-- HPLC; flow rate: 20 mL/min; gradient: 30% B to 30% B in 49 min; wave length: 262/338 nm). |
| 102a | 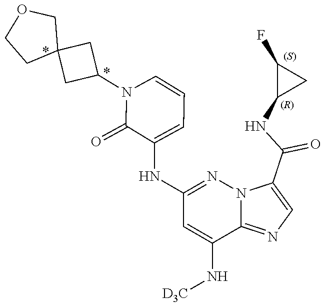 Diastereoisomer 1 (earlier eluting diastereoisomer) | LC-MS (EIS): m/z 471.25 [M + H]⁺; ¹H NMR (300 MHz, CDCl₃) δ 8.60 (d, J = 4.4 Hz, 1H), 8.17 (s, 1H), 7.85-7.76 (m, 2H), 7.07 (dd, J = 7.1, 1.7 Hz, 1H), 6.86 (s, 1H), 6.37 (t, J = 7.2 Hz, 1H), 5.76 (s, 1H), 5.30-5.12 (m, 1H), 4.96-4.67 (m, 1H), 3.93 (t, J = 6.8 Hz, 2H), 3.77 (s, 2H), 3.25- 3.13 (m, 1H), 2.71-2.59 (m, 2H), 2.44-2.40 (m, 2H), 2.18-2.15 (m, 2H), 1.41-1.22 (m, 1H), 1.19- 1.03 (m, 1H). de: 100%; Retention time: 12.96 min (column: chiralpak ID, 2*25 cm 5 μm; mobile phase A: MtBE (10 mM NH₃—MeOH), mobile phase B: EtOH--HPLC; flow rate: 20 mL/min; gradient: 50% B to 50% B in 24 min; wave length: 262/338 nm). |
| 102b | 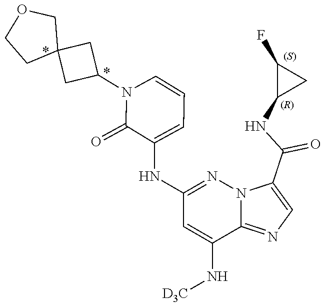 Diastereoisomer 2 (later eluting diastereoisomer) | LC-MS (ESI): m/z 471.25 [M + H]⁺; ¹H NMR (300 MHz, CDCl₃) δ 8.58 (d, J = 4.5 Hz, 1H), 8.21 (s, 1H), 7.88 (s, 1H), 7.78 (d, J = 7.2 Hz, 1H), 7.13 (dd, J = 7.1, 1.6 Hz, 1H), 6.38 (t, J = 7.2 Hz, 1H), 5.85 (s, 1H), 5.11-5.05 (m, 1H), 4.98- 4.65 (m, 1H), 3.93-3.82 (m, 4H), 3.23-3.13 (m, 1H), 2.77-2.66 (m, 2H), 2.51-2.38 (m, 2H), 2.05 (t, J = 7.2 Hz, 2H), 1.59-1.52 (m, 1H), 1.23-1.02 (m, 1H). de: 100%; Retention time: 18.13 min (column: chiralpak ID, 2*25 cm, 5 μm; mobile phase A: MtBE (10 mM NH₃—MeOH), mobile phase B: EtOH--HPLC; flow rate: 20 mL/min; gradient: 50% B to 50% B in 24 min; wave length: 262/338 nm). |
| 103a | 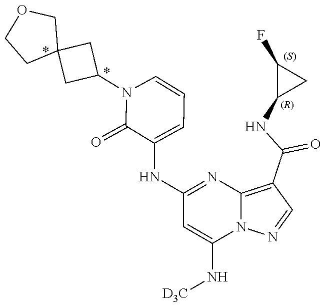 Diastereoisomer 1 (earlier eluting diastereoisomer) | LC-MS (ESI): m/z 471.20 [M + H]⁺; ¹H NMR (400 MHz, DMSO-d₆) δ 8.84 (s, 1H), 8.22 (s, 1H), 8.12 (dd, J = 7.4, 1.7 Hz, 1H), 7.87 (s, 1H), 7.80 (d, J = 4.8 Hz, 1H), 7.44 (dd, J = 7.1, 1.7 Hz, 1H), 6.28 (t, J = 7.1 Hz, 1H), 6.21 (s, 1H), 5.17- 5.06 (m, 1H), 4.99-4.75 (m, 1H), 3.74 (t, J = 6.7 Hz, 2H), 3.63 (s, 2H), 3.05-2.92 (m, 1H), 2.43 (d, J = 8.8 Hz, 4H), 2.06 (t, J = 6.8 Hz, 2H), 1.29-1.15 (m, 1H), 0.94-0.79 (m, 1H). de: 99%; Retention time: 11.708 min (column: chiralpak ID, 2*25 cm, 5 μm; mobile phase A: MtBE (10 mM NH₃—MeOH), mobile phase B: EtOH--HPLC; flow rate: 18 mL/min; gradient: 50% B to 50% B in 23 min; wave length: 210/268 nm). |
| 103b | 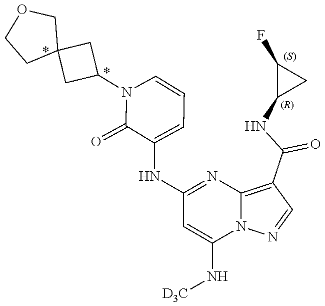 Diastereoisomer 2 (later eluting diastereoisomer) | LC-MS (ESI): m/z 471.15 [M + H]⁺; ¹H NMR (400 MHz, DMSO-d₆) δ 8.85 (s, 1H), 8.22 (s, 1H), 8.13 (dd, J = 7.3, 1.7 Hz, 1H), 7.87 (s, 1H), 7.80 (d, J = 4.8 Hz, 1H), 7.45 (dd, J = 7.1, 1.7 Hz, 1H), 6.29 (t, J = 7.1 Hz, 1H), 6.21 (s, 1H), 5.10- 4.74 (m, 2H), 3.75 (s, 2H), 3.68 (t, J = 7.1 Hz, 2H), 3.04-2.95 (m, 1H), 2.48-2.38 (m, 4H), 1.97 (t, J = 7.1 Hz, 2H), 1.30-1.16 (m, 1H), 0.93-0.80 (m, 1H). de: 99%; Retention time: 17.595 min (column: chiralpak ID, 2*25 cm, 5 μm; mobile phase A: MtBE (10 mM NH₃—MeOH), mobile phase B: EtOH--HPLC; flow rate: 18 mL/min; gradient: 50% B to 50% B in 23 min; wave length: 210/268 nm). |
| 104a | 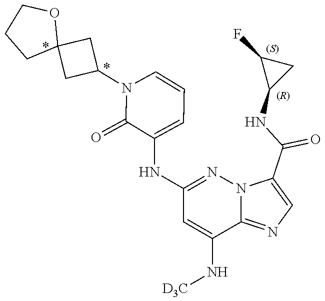 Diastereoisomer 1 (earlier eluting diastereoisomer) | LC-MS (ESI): m/z 471.25 [M + H]⁺; ¹H NMR (400 MHz, CDCl₃) δ 8.60 (d, J = 4.4 Hz, 1H), 8.14 (s, 1H), 7.82-7.77 (m, 1H), 7.75 (s, 1H), 7.18 (d, J = 1.2 Hz, 1H), 6.51 (s, 1H), 6.36 (t, J = 7.6 Hz, 1H), 5.71 (s, 1H), 4.98-4.94 (m, 1H), 4.86- 4.70 (m, 1H), 3.86 (t, J = 6.4 Hz, 2H), 3.17-3.13 (m, 1H), 2.73-2.68 (m, 2H), 2.52-2.45 (m, 2H), 2.08-2.05 (m, 2H), 2.05-2.00 (m, 2H), 1.32-1.26 (m, 1H), 1.12-1.02 (m, 1H). de: 100%; Retention time: 12.8025 min (column: chiralpak ID, 2*25 cm, 5 μm; mobile phase A: MtBE (10 mM NH₃—MeOH), mobile phase B: EtOH--HPLC; flow rate: 18 mL/min; gradient: 50% B to 50% B in 22 min; wave length: 262/338 nm). |
| 104b | 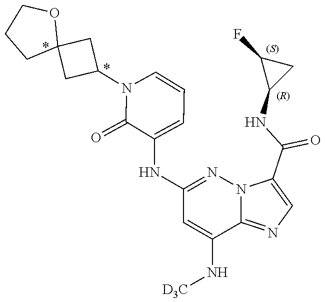 Diastereoisomer 2 (later eluting diastereoisomer) | LC-MS (ESI): m/z 471.25 [M + H]⁺; ¹H NMR (400 MHz, CDCl₃) δ 8.60 (d, J = 4.5 Hz, 1H), 8.14 (s, 1H), 7.82 (s, 1H), 7.80-7.73 (m, 1H), 7.03 (dd, J = 7.1, 1.7 Hz, 1H), 6.79 (s, 1H), 6.33 (t, J = 7.2 Hz, 1H), 5.72 (s, 1H), 5.23 (p, J = 8.5 Hz, 1H), 4.89-4.68 (m, 1H), 3.91-3.83 (m, 2H), 3.22- 3.10 (m, 1H), 2.84-2.74 (m, 2H), 2.54-2.44 (m, 2H), 2.00-1.86 (m, 4H), 1.36-1.22 (m, 1H), 1.15- 1.03 (m, 1H). de: 100%; Retention time: 21.5925 min (column: chiralpak ID, 2*25 cm, 5 μm; mobile phase A: MtBE (10 mM NH₃—MeOH), mobile phase B: EtOH--HPLC; flow rate: 18 mL/min; gradient: 50% B to 50% B in 22 min; wave length: 262/338 nm). |
| 105a | 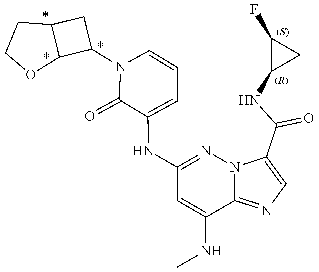 Diastereoisomer 1 (earlier eluting diastereoisomer), relative stereochemistry of the 4,5-fused bicyclic structure on the left hand side is the same as that of 3.2a or 3.2b | LC-MS (ESI): m/z 454.2 [M + H]⁺; ¹H NMR (400 MHz, DMSO-d₆) δ 8.65 (d, J = 4.3 Hz, 1H), 8.50 (s, 1H), 7.95-7.81 (m, 2H), 7.44 (s, 1H), 7.32 (dd, J = 7.1, 1.7 Hz, 1H), 6.33 (d, J = 1.8 Hz, 1H), 6.28 (t, J = 7.1 Hz, 1H), 4.96-4.93 (m, 2H), 4.77-4.76 (m, 1H), 4.04-3.95 (m, 1H), 3.89- 3.79 (m, 1H), 3.05-2.94 (m, 2H), 2.86 (d, J = 4.8 Hz, 3H), 2.65-2.55 (m, 1H), 2.33-2.19 (m, 1H), 1.88-1.69 (m, 2H), 1.28-1.20 (m, 1H), 1.03- 0.87 (m, 1H). de: 100%; Retention time: 7.32 min (column: chiral art cellulose-SB, 3*25 cm, 5 μm; mobile phase A: CO₂, mobile phase B: IPA: DCM = 2:1 (0.1% 2 M NH₃—MeOH); flow rate: 70 mL/min; gradient: isocratic 50% B; column temperature (° C.): back pressure (bar): 100; wave length: 260 nm). |
| 105b | 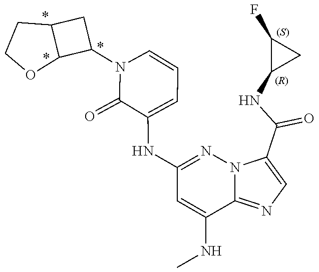 Diastereoisomer 2 (later eluting diastereoisomer) relative stereochemistry of the 4,5-fused bicyclic structure on the left hand side is the same as that of 3.2a or 3.2b | LC-MS (ESI): m/z 454.2 [M + H]⁺; ¹H NMR (400 MHz, DMSO-d₆) δ 8.66 (d, J = 4.2 Hz, 1H), 8.52 (s, 1H), 7.93-7.83 (m, 2H), 7.46 (q, J = 4.9 Hz, 1H), 7.33 (dd, J = 7.1, 1.7 Hz, 1H), 6.35 (s, 1H), 6.28 (t, J = 7.1 Hz, 1H), 5.00-4.75 (m, 3H), 4.05-3.96 (m, 1H), 3.92-3.78 (m, 1H), 3.05- 2.93 (m, 2H), 2.87 (d, J = 4.9 Hz, 3H), 2.66-2.56 (m, 1H), 2.35-2.21 (m, 1H), 1.86-1.72 (m, 2H), 1.27-1.22 (m, 1H), 1.00-0.89 (m, 1H). de: 99%; Retention time: 12.18 min (column: chiral art cellulose-SB, 3*25 cm, 5 μm; mobile phase A: CO₂, mobile phase B: IPA:DCM = 2:1 (0.1% 2 M NH₃—MeOH); flow rate: 70 mL/ min; gradient: isocratic 50% B; column temperature (° C.): 35; back pressure (bar): 100; wave length: 260 nm). |
| 105c | 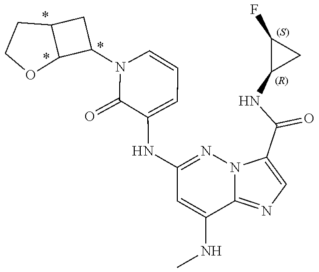 Diastereoisomer 3 (earlier sluting diastereoisomer), relative stereochemistry of the 4,5-fused bicyclic structure on the oeft hand side is the same as that of 3.2a or 3.2b | LC-MS (ESI): m/z 454.2 [M + H]⁺; ¹H NMR (400 MHz, DMSO-d₆) δ 8.62 (d, J = 4.0 Hz, 1H), 8.55 (s, 1H), 7.93-7.83 (m, 2H), 7.48 (d, J = 5.1 Hz, 1H), 7.40 (dd, J = 7.0, 1.7 Hz, 1H), 6.37 (s, 1H), 6.30 (t, J = 7.1 Hz, 1H), 5.00-4.76 (m, 3H), 4.17-3.94 (m, 1H), 4.04-3.96 (m, 1H), 3.12 (s, 1H), 3.02-2.92 (m, 1H), 2.86 (d, J = 4.8 Hz, 3H), 2.48-2.40 (m, 1H), 2.09-1.98 (m, 1H), 1.89- 1.74 (m, 2H), 1.27-1.21 (m, 1H), 1.00-0.90 (m, 1H). de: 99%; Retention time: 10.65 min (column: chiral art cellulose-SB, 3*25 cm, 5 μm; mobile phase A: CO₂, mobile phase B: IPA:DCM = 2:1 (0.1% 2 M NH₃—MeOH); flow rate: 70 mL/min; gradient: isocratic 45% B; column temperature (° C.): 35; back pressure (bar): 100; wave length: 260 nm). |
| 105d | 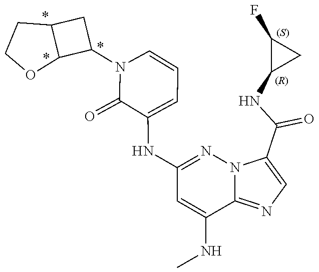 Diastereoisomer 4 (later eluting diastereoisomer), relative stereochemistry of the 4,5-fused bicyclic structure on the left hand side is the same as that of 3.2a or 3.2b | LC-MS (ESI): m/z 454.2 [M + H]⁺; ¹H NMR (400 MHz, DMSO-d₆) δ 8.62 (d, J = 4.2 Hz, 1H), 8.55 (s, 1H), 7.97-7.81 (m, 2H), 7.48 (d, J = 5.1 Hz, 1H), 7.41 (dd, J = 7.0, 1.7 Hz, 1H), 6.37 (s, 1H), 6.31 (t, J = 7.1 Hz, 1H), 5.01-4.76 (m, 3H), 4.17-4.09 (m, 1H), 4.06-3.95 (m, 1H), 3.13- 3.05 (m, 1H), 3.03-2.95 (m, 1H), 2.86 (d, J = 4.8 Hz, 3H), 2.49-2.40 (m, 1H), 2.10-1.98 (m, 1H), 1.90-1.74 (m, 2H), 1.27-1.20 (m, 1H), 1.01- 0.88 (m, 1H). de: 98%; Retention time: 12.45 min (column: chiral art cellulose-SB 3*25 cm, 5 μm; mobile phase A: CO₂, mobile phase B: IPA:DCM = 2:1 (0.1% 2 M NH₃—MeOH); flow rate: 70 mL/min; gradient: isocratic 45% B; column temperature (° C.); 35; back pressure (bar): 100; wave length: 260 nm). |
| 106 | 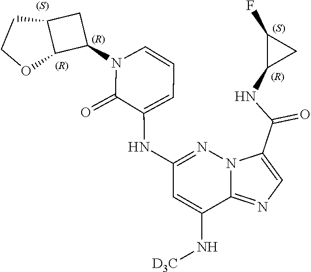 | LC-MS (ESI): m/z 457.2 [M + H]⁺; ¹H NMR (400 MHz, CDCl₃) δ 8.53 (s, 1H), 8.06 (s, 1H), 7.77-7.71 (m, 2H), 6.99-6.91 (m, 1H), 6.43- 6.40 (m, 1H), 6.14 (s, 1H), 5.61 (s, 1H), 4.98-4.55 (m, 3H), 4.12-4.10 (m, 2H), 3.19-3.10 (m, 2H), 2.48-2.43 (m, 1H), 2.16-1.85 (m, 3H), 1.29- 1.24 (m, 1H), 1.08-0.94 (m, 1H). |
| 107 | 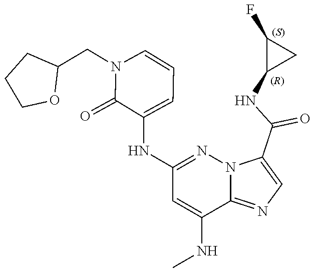 Diastereoisomer mixture | LC-MS (ESI): m/z 442.25 [M + H]⁺; ¹H NMR (400 MHz, CDCl₃) δ 8.68-8.52 (m, 1H), 8.14 (s, 1H), 7.87-7.71 (m, 2H), 7.11 (dd, J = 7.0, 1.7 Hz, 1H), 6.73 (s, 1H), 6.34-6.21 (m, 1H), 5.72 (s, 1H), 4.98-4.63 (m, 1H), 4.53-4.36 (m, 1H), 4.33-4.14 (m, 1H), 3.94-3.84 (m, 2H), 3.82-3.73 (m, 1H), 3.20-3.08 (m, 1H), 3.03 (d, J = 5.0 Hz, 3H), 2.19-2.04 (m, 1H), 1.96-1.85 (m, 2H), 1.70- 1.57 (m, 1H), 1.37-1.20 (m, 1H), 1.16-1.03 (m, 1H). |

### Biological Example 1. TYK2 JH2 Domain Binding Assay

Assays were performed in 384-well plates with a final assay volume of 20 μL containing copper-polyvinyltoluene scintillation proximity assay (SPA) beads (PerkinElmer Life Sciences, catalogue no. RPNQ0095) at 80 g/ml, H3 probe (20 nM), the N-terminal His-tagged TYK2 pseudokinase domain (2.5 nM, purified by Pharmaron), and test compounds in assay buffer (50 mM HEPES, pH 7.5, 100 g ml-1 BSA, 500 DMSO). After incubating at room temperature for 30 min, the inhibition was calculated by the displacement of [3H] 3 binding as determined by scintillation counting. Dose-response curves were generated to determine the concentration required to inhibit H3 probe binding by 5000 (IC50).

### Biological Example 2. IL-23 Induced STAT3 QUANTI-Blue in HEK-Blue™ IL-23 Cells

HEK-Blue™ IL-23 cells were prepared at 2×10⁵ cells/ml for adding 5000 cell/well and incubate plates containing compounds+cells for 1 hour at 37° C. Prepare IL-23 (treatment at final conc. 0.1 ng/mL) during incubation and add 2 ul per well of prepared cytokine in media to appropriate wells. The plate is incubated for overnight at 37° C. The next day, QUANTI-Blue solution is prepared. 18 ul of QUANTI-Blue solution per well is added into a new 384-well clear flat bottom plate, then add 2 ul of induced and treated HEK-Blue cell supernatant from overnight plate. Incubate the plate at 37° C. for 2 hours and determine SEAP levels.

Biological activity data for representative compounds tested according to the procedures described in Biological Examples 1 and 2 are provided in Table 3 below. In Table 3, “A” represents IC₅₀ values of less than 10 nM; “B” represents IC₅₀ values of greater than or equal to 10 nM and less than 100 nM; “C” represents IC₅₀ values of greater than or equal to 100 nM and less than 1 μM; and “D” represents IC₅₀ values of 1 μM or greater. A control compound, BMS—986165 (J. Med. Chem. 2019, 62, 20, 8973-8995), was also tested following Biological Examples 1 and 2. BMS986165 is believed to have the structure shown below:

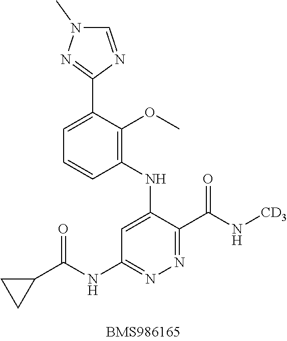 [SMILES: [2H]C([2H])([2H])NC(=O)c1nnc(NC(=O)C2CC2)cc1Nc1cccc(-c2ncn(C)n2)c1OC]

TABLE 3
Examplary Biological Data

| Compound | TYK2 JH2 SPA Binding assay | IL23 reporter assay |
|---|---|---|
| No. | (IC50 nM) | (IC50 nM) |
| Control (BMS- | 4.99 | 5.93 |
| 986165) | | |
| 1 | B | A |
| 2 | / | B |
| 3a | / | A |
| 3b | / | C |
| 4a | / | B |
| 4b | / | D |
| 5 | B | A |
| 6 | A | A |
| 7 | A | A |
| 8 | A | A |
| 9 | A | A |
| 10a | B | B |
| 10b | D | D |
| 11 | A | / |
| 12 | A | A |
| 13 | B | A |
| 14 | A | A |
| 15 | B | B |
| 16 | B | A |
| 17 | B | B |
| 18 | A | A |
| 19 | B | B |
| 20 | A | A |
| 21 | B | A |
| 22a | D | D |
| 22b | A | A |
| 23a | C | D |
| 23b | A | B |
| 24 | B | B |
| 25 | A | A |
| 26 | B | B |
| 27 | A | A |
| 28 | B | A |
| 29 | A | A |
| 30 | / | B |
| 31 | A | A |
| 32 | B | A |
| 33 | B | A |
| 34 | B | B |
| 35 | B | B |
| 36a | / | D |
| 36b | B | B |
| 37 | B | A |
| 38 | B | A |
| 39 | A | A |
| 40 | D | C |
| 41 | B | B |
| 42 | D | C |
| 43 | B | B |
| 44 | B | A |
| 45 | C | D |
| 46 | B | A |
| 47 | B | A |
| 48 | / | B |
| 49a | B | A |
| 49b | D | D |
| 50 | / | C |
| 51 | C | B |
| 52 | A | A |
| 53 | B | A |
| 54 | B | A |
| 55 | B | B |
| 56 | / | B |
| 57 | B | B |
| 58 | B | B |
| 59 | B | A |
| 60 | / | B |
| 61 | B | A |
| 62a | C | B |
| 62b | B | B |
| 63 | A | A |
| 64 | B | B |
| 65 | B | A |
| 66 | B | A |
| 67 | / | B |
| 68a | B | B |
| 68b | B | A |
| 69 | B | A |
| 70 | B | B |
| 71 | / | B |
| 72 | B | A |
| 73 | B | A |
| 74 | / | B |
| 75a | C | C |
| 75b | B | B |
| 76 | B | B |
| 77 | / | B |
| 78 | / | B |
| 79a | B | B |
| 79b | B | A |
| 80 | B | B |
| 81 | B | B |
| 82 | B | A |
| 83 | B | B |
| 84 | B | B |
| 85 | B | B |
| 86 | B | B |
| 87 | B | B |
| 88a | B | A |
| 88b | C | C |
| 89 | B | B |
| 90 | B | B |
| 91 | B | B |
| 92a | B | B |
| 92b | B | B |
| 93a | B | B |
| 93b | B | B |
| 94a | B | B |
| 94b | B | B |
| 95a | B | A |
| 95b | D | D |
| 96a | B | B |
| 96b | B | B |
| 97a | B | B |
| 97b | B | B |
| 98a | B | B |
| 98b | B | B |
| 99 | B | B |
| 100 | B | B |
| 101a | B | B |
| 101b | B | B |
| 102a | B | B |
| 102b | B | B |
| 103a | B | B |
| 103b | C | B |
| 104a | B | B |
| 104b | B | B |
| 105a | C | B |
| 105b | C | B |
| 105c | B | B |
| 105d | B | A |
| 106 | A | A |
| 107 | B | B |

### Biological Example 3. IL-12 Induced pSTAT4 in Human PBMC

Frozen Human PBMC are thawed and resuspended in complete media containing serum, then cells are diluted to 1.6×10⁶ cells/ml. 2.5 uL of compound or DMSO is added to the well at the desired concentrations and incubated at 1 hr at 37° C. 2.5 uL of stimulus (final concentration of 1.7 ng/mL IL-12) is added for 30 minutes prior to pSTAT4 analysis using cell lysates prepared and analyzed by AlphaLISA assay as per manufacturer protocol. The final DMSO concentration of compound in the assay is 0.1%.

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A compound of Formula I or II, or a pharmaceutically acceptable salt thereof: 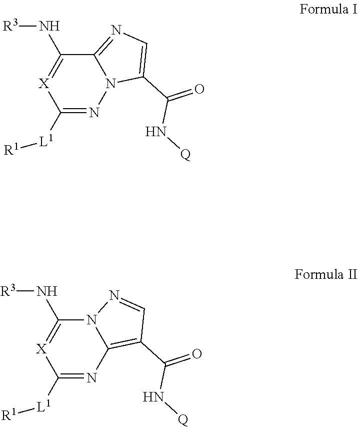
wherein:
L¹ is a bond, NR¹⁰, O, optionally substituted C₁₋₄ alkylene or optionally substituted C₁₋₄ heteroalkylene;
R¹ is an optionally substituted carbocyclic ring, optionally substituted heterocyclic ring, optionally substituted aryl or optionally substituted heteroaryl ring;
X is N or CR², wherein R² is hydrogen, hydroxyl, halogen, optionally substituted C₁₋₆ alkyl, optionally substituted C₁₋₆ heteroalkyl, optionally substituted C₁₋₆ alkoxy, optionally substituted C₃₋₆ cycloalkyl, or optionally substituted 4-8 membered heterocyclyl;
R³ is hydrogen, optionally substituted C₁₋₆ alkyl, optionally substituted C₁₋₆ heteroalkyl, optionally substituted C₃₋₆ cycloalkyl, or optionally substituted 4-8 membered heterocyclyl; or R² and R³, together with the intervening atoms, form a 5-8 membered heterocyclic or a 5 or 6 membered heteroaryl ring, each of which is optionally substituted and has one ring nitrogen atom as required by Formula I or II, and optionally 1-2 additional ring heteroatoms independently selected from nitrogen, oxygen, and sulfur; Q is 6-14 membered heterocyclyl or 5-10 membered heteroaryl, each of which is optionally substituted, or Q is 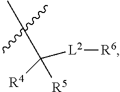
wherein R⁴ and R⁵ are each independently hydrogen or an optionally substituted C₁₋₆ alkyl, or R⁴ and R⁵ are joined to form a 3-8 membered carbocyclic or heterocyclic ring, each of which is optionally substituted;
L² is a bond, NR^{11A}, O, optionally substituted C₁₋₄ alkylene or optionally substituted C₁₋₄ heteroalkylene;
R⁶ is hydrogen, a 3-8 membered heterocyclic ring, or a 5 or 6 membered heteroaryl ring, each of which is optionally substituted; and
wherein:
R¹⁰ and R^{11A} are each independently hydrogen, optionally substituted C₁₋₆ alkyl, optionally substituted C₃₋₆ cycloalkyl, optionally substituted aryl, optionally substituted 5 or 6 membered heteroaryl, or optionally substituted 4-8 membered heterocyclyl.

2. (canceled)

3. The compound of claim 1, or a pharmaceutically acceptable salt thereof, wherein Q is a fused 4,5-bicyclic heterocyclyl, fused 4,6-bicyclic heterocyclyl, fused 5,6-bicyclic heterocyclyl, fused 5,5-bicyclic heterocyclyl, or fused 6,6-bicyclic heterocyclyl.

4. The compound of claim 1, or a pharmaceutically acceptable salt thereof, wherein Q has the structure of F-1, F-2, or F-3: 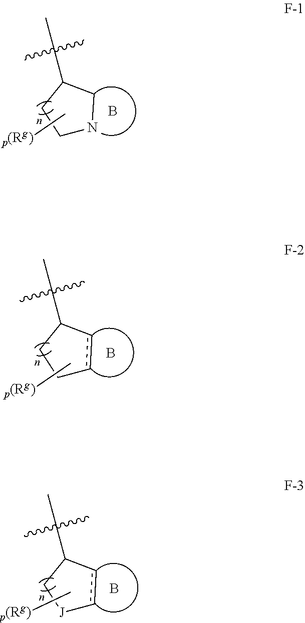
wherein:
ring B in F-1 is an optionally substituted 5-membered heteroaryl having 1-4 ring heteroatoms independently selected from nitrogen, oxygen, and sulfur, or an optionally substituted 4-7 membered heterocyclyl having 1-2 ring heteroatoms independently selected from nitrogen, oxygen, and sulfur, ring B in F-2 or F-3 is an optionally substituted 5- or 6-membered heteroaryl having 1-3 ring heteroatoms independently selected from nitrogen, oxygen, and sulfur, or an optionally substituted 4-7 membered heterocyclyl having 1-2 ring heteroatoms independently selected from nitrogen, oxygen, and sulfur,
J is O or NR¹¹, wherein R¹¹ is a C₁₋₄ alkyl optionally substituted with 1-3 R^{s1},
n is 0, 1, or 2,
p is 0, 1, 2, or 3, as valency permits,
R^{g} at each occurrence is independently OH, halogen, CN, oxo (as valency permits), C₁₋₄ alkyl optionally substituted with one or more R^{s2}, C₁₋₄ heteroalkyl optionally substituted with one or more R^{s2}, C₃₋₆ cycloalkyl optionally substituted with one or more R^{s2}, or 4-7 membered heterocyclyl optionally substituted with one or more R^{s2},
wherein R^{s1} at each occurrence is independently F, OH, oxo (as valency permits), methoxy, or methyl; and
R^{s2} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.

5. The compound of claim 4, or a pharmaceutically acceptable salt thereof, wherein ring B in F-1, F-2, or F-3 is a 5-membered heteroaryl having 1-4 ring heteroatoms independently selected from nitrogen, oxygen, and sulfur, wherein the 5-membered heteroaryl is optionally substituted with one or more R^{s3}, wherein R^{s3} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F; or ring B in F-2, or F-3 is a 6-membered heteroaryl having 1-3 ring nitrogen atoms, wherein the 6-membered heteroaryl is optionally substituted, with one or more R^{s3}, wherein R^{s3} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.

6. (canceled)

7. The compound of claim 1, or a pharmaceutically acceptable salt thereof, wherein n in F-1, F-2, or F-3 is 0 or 1; or
wherein p in F-1, F-2, or F-3 is 0: or wherein J in F-3 is O or NCH₃: or any combination thereof.

8. (canceled)

9. (canceled)

10. The compound of claim 1, or a pharmaceutically acceptable salt thereof, wherein Q is selected from:

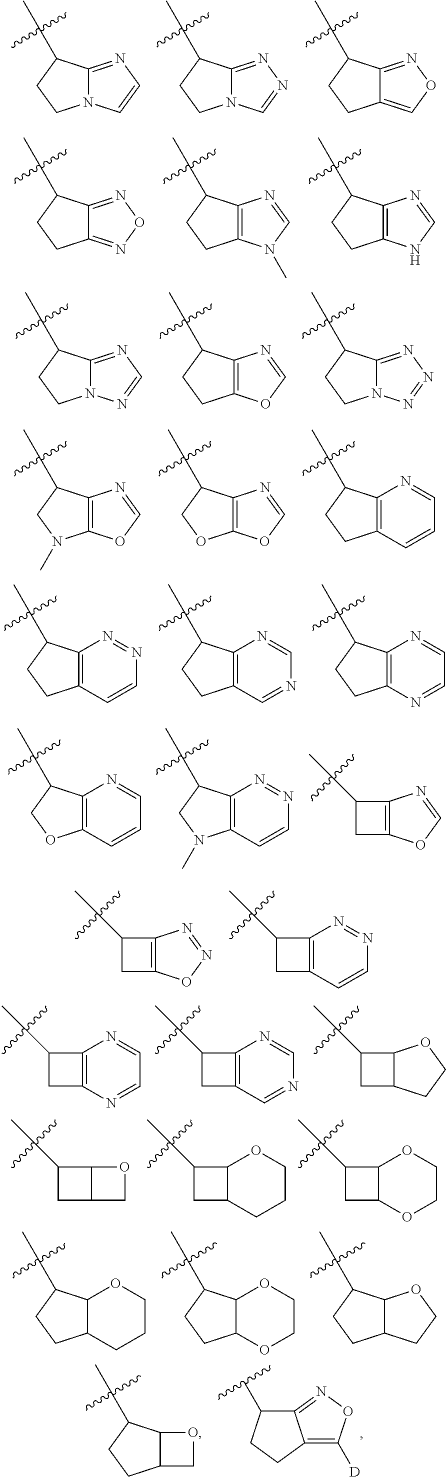

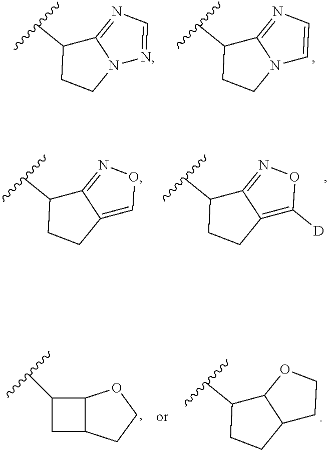 [SMILES: *C1CCn2ncnc21 ; *C1CCn2ccnc21 ; *C1CCc2conc21 ; *C1CCc2c1noc2[2H] ; *C1CC2CCOC12 ; *C1CCC2CCOC12]

11. The compound of claim 1, or a pharmaceutically acceptable salt thereof, wherein Q is selected from: 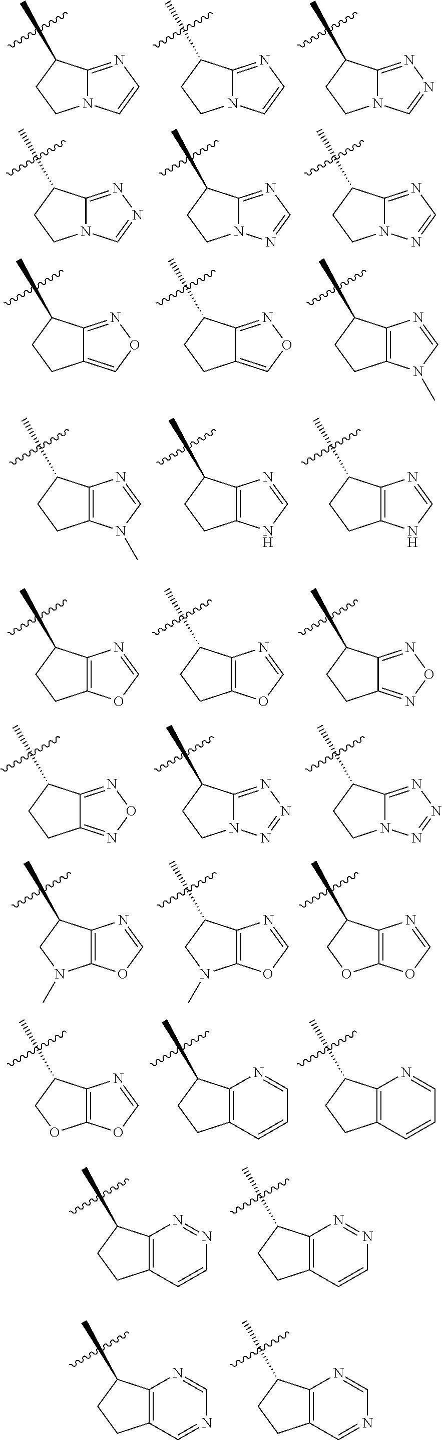 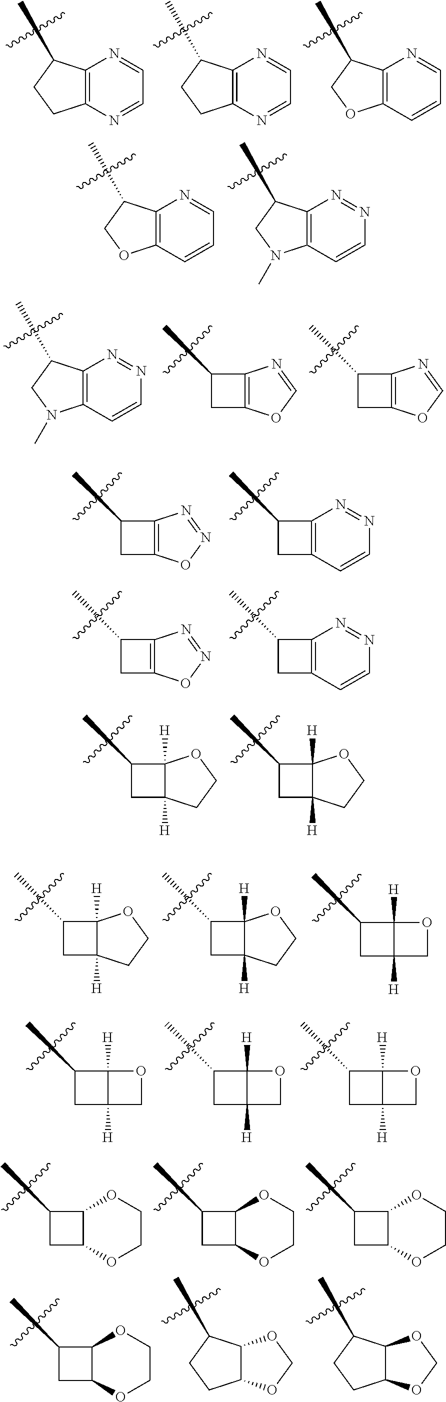 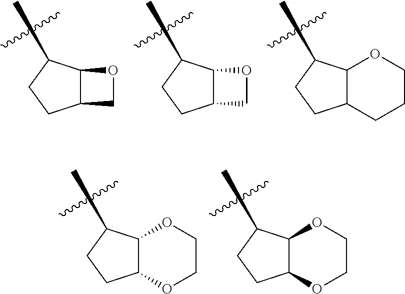
or

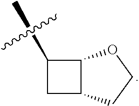

12. The compound of claim 1, or a pharmaceutically acceptable salt thereof, wherein Q is 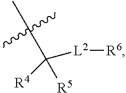
selected from

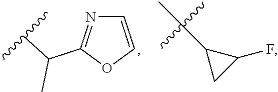

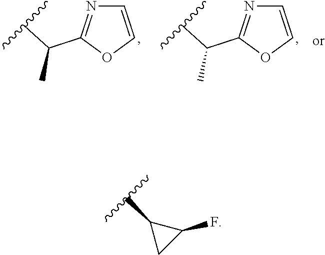

13. The compound of claim 1, or a pharmaceutically acceptable salt thereof, wherein L¹ is —NH—, a bond, or O.

14. (canceled)

15. (canceled)

16. The compound of claim 1, or a pharmaceutically acceptable salt thereof, wherein R¹ is a phenyl, 5- or 6-membered heterocyclic or heteroaryl ring selected from: 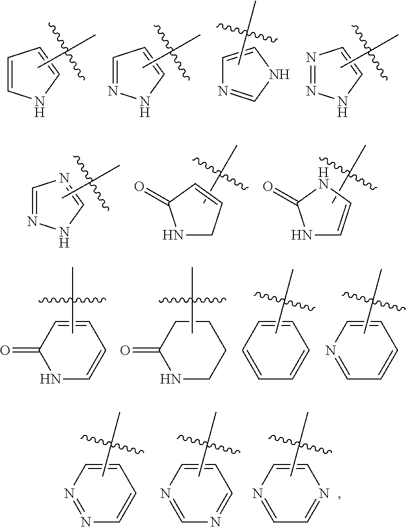
wherein each of which is optionally substituted with one or more substituents each independently:
1) halogen, cyano (CN), —C(O)(C₁₋₄ alkyl), —C(O)NH₂, —COOH, —C(O)—N(H)(C₁₋₄ alkyl), —C(O)—N(C₁₋₄ alkyl)(C₁₋₄ alkyl), —N(H)—C(O)—(C₁₋₄ alkyl), or —N(C₁₋₄ alkyl)-C(O)—(C₁₋₄ alkyl),
2) C₁₋₆ alkyl, —O—C₁₋₆ alkyl, —N(H)—C₁₋₆ alkyl, or —(C₁₋₄ heteroalkylene)-C₁₋₆ alkyl, wherein each of the C₁₋₆ alkyl is optionally substituted with one or more substituents independently selected from oxo, C₁₋₄ heteroalkyl, hydroxyl, N(C₁₋₄ alkyl)(C₁₋₄ alkyl), N(H)(C₁₋₄ alkyl), —C(O)—N(C₁₋₄ alkyl)(C₁₋₄ alkyl), —C(O)NH₂, —COOH, —C(O)—N(H)(C₁₋₄ alkyl), —N(H)—C(O)—(C₁₋₄ alkyl), —N(C₁₋₄ alkyl)-C(O)—(C₁₋₄ alkyl), —C(O)-G¹, F, C₁₋₄ alkoxy optionally substituted with 1-3 F, 5- or 6-membered heteroaryl having 1-3 ring heteroatoms independently selected from nitrogen, oxygen, and sulfur, and 4-8 membered saturated heterocyclyl having 1-3 ring heteroatoms independently selected from nitrogen, oxygen, and sulfur, wherein the 5- or 6-membered heteroaryl or 4-8 membered saturated heterocyclyl is optionally substituted with 1-3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
3) C₃₋₁₀ cycloalkyl, —(C₁₋₄ alkylene)-C₃₋₁₀ cycloalkyl, —O—C₃₋₁₀ cycloalkyl, —N(H)—C₃₋₁₀ cycloalkyl, or —(C₁₋₄ heteroalkylene)-C₃₋₁₀ cycloalkyl, each of the C₃₋₁₀ cycloalkyl is monocyclic or polycyclic which is optionally substituted with one or more substituents independently selected from C₁₋₄ alkyl optionally substituted with 1-3 F, C₁₋₄ heteroalkyl, hydroxyl, F, and C₁₋₄ alkoxy optionally substituted with 1-3 F, wherein two of the optional substituents of the C₃₋₁₀ cycloalkyl can be joined to form a 4-8 membered saturated heterocyclic ring containing 1 or 2 ring heteroatoms independently selected from nitrogen, oxygen, and sulfur, wherein the 4-8 membered saturated heterocyclic ring is optionally substituted with one or more, substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F, 4) 4-8 membered heterocyclyl, —(C₁₋₄ alkylene)-(4-8 membered heterocyclyl), —O-(4-8 membered heterocyclyl), —N(H)-(4-8 membered heterocyclyl), or —(C₁₋₄ heteroalkylene)-(4-8 membered heterocyclyl), wherein each of the 4-8 membered heterocyclyl is a heterocyclyl having 1-3 ring heteroatoms independently selected from nitrogen, oxygen, and sulfur, which is optionally substituted with 1-3 substituents independently selected from oxo, F, C₁₋₄ alkyl optionally substituted with 1-3 F, C₁₋₄ heteroalkyl, hydroxyl, G¹, and C₁₋₄ alkoxy optionally substituted with 1-3 F, wherein two optional substituents of the 4-8 membered heterocyclyl can be joined together to form a spirocycloalkyl,
5) 5 or 6 membered heteroaryl, —(C₁₋₄ alkylene)-(5 or 6 membered heteroaryl), —O-(5 or 6 membered heteroaryl), —N(H)-(5 or 6 membered heteroaryl), or —(C₁₋₄ heteroalkylene)-(5 or 6 membered heteroaryl), wherein each of the 5 or 6 membered heteroaryl is a heteroaryl having 1-4 ring heteroatoms independently selected from nitrogen, oxygen, and sulfur, which is optionally substituted with one or more substituents independently selected from C₁₋₄ alkyl optionally substituted with 1-3 F, C₁₋₄ heteroalkyl, hydroxyl, F, Cl, CN, and C₁₋₄ alkoxy optionally substituted with 1-3 F, wherein two optional substituents of the 5 or 6 membered heteroaryl can be joined together to form a ring structure, or
6) phenyl, —(C₁₋₄ alkylene)-phenyl, —O-phenyl, —N(H)-phenyl, or —(C₁₋₄ heteroalkylene)-phenyl, each of the phenyl is optionally substituted with 1-3 substituents independently selected from F, Cl, CN, C₁₋₄ alkyl optionally substituted with 1-3 F, C₁₋₄ heteroalkyl, hydroxyl, and C₁₋₄ alkoxy optionally substituted with 1-3 F, wherein two optional substituents of the phenyl can be joined together to form a ring structure, and wherein G¹ is a 4-8 membered saturated heterocyclyl containing 1 or 2 ring heteroatoms independently selected from nitrogen, oxygen, and sulfur, wherein the heterocyclyl is optionally substituted with one or more substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F, and wherein two of the optional substituents of the phenyl, 5- or 6-membered heterocyclic or heteroaryl ring as drawn above can be joined together to form a spiro, fused, or bridged ring structure).

17. The compound of claim 1, or a pharmaceutically acceptable salt thereof, wherein R¹ is selected from: 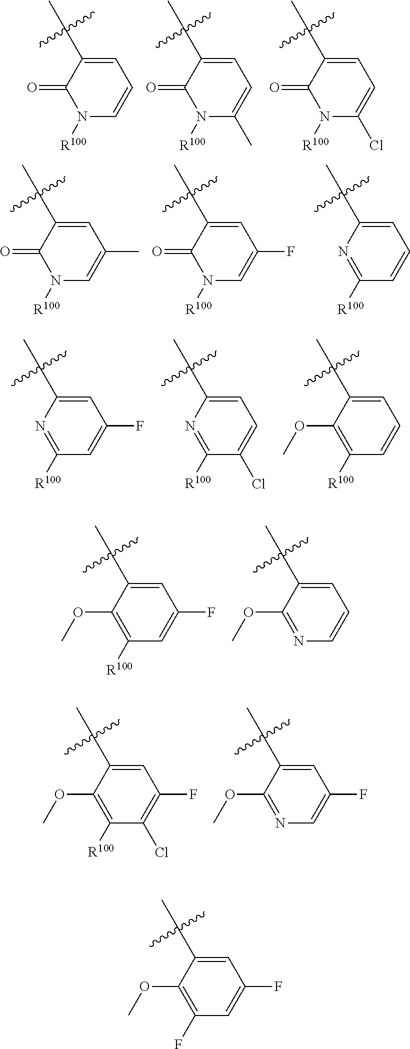
wherein each R¹⁰⁰ is independently selected from:
1) C₁₋₆ alkyl, which is optionally substituted with one or more substituents independently selected from oxo, C₁₋₄ heteroalkyl, hydroxyl, N(C₁₋₄ alkyl)(C₁₋₄ alkyl), N(H)(C₁₋₄ alkyl), —C(O)—N(C₁₋₄ alkyl)(C₁₋₄ alkyl), —C(O)NH₂, —COOH, —C(O)—N(H)(C₁₋₄ alkyl), —N(H)—C(O)—(C₁₋₄ alkyl), —N(C₁₋₄ alkyl)-C(O)—(C₁₋₄ alkyl), —C(O)-G¹, F, C₁₋₄ alkoxy optionally substituted with 1-3 F, 5- or 6-membered heteroaryl having 1-3 ring heteroatoms independently selected from nitrogen, oxygen, and sulfur, and 4-8 membered saturated heterocyclyl having 1-3 ring heteroatoms independently selected from nitrogen, oxygen, and sulfur, wherein the 5- or 6-membered heteroaryl or 4-8 membered saturated heterocyclyl is optionally substituted with 1-3 substituents independently selected from F, C₁₋₄ alkyl optionally substituted with 1-3 F, C₁₋₄ heteroalkyl, hydroxyl, and C₁₋₄ alkoxy optionally substituted with 1-3 F,
2) C₃₋₆ cycloalkyl, which is optionally substituted with one or more substituents independently selected from C₁₋₄ alkyl optionally substituted with 1-3 F, C₁₋₄ heteroalkyl, hydroxyl, F, 5- or 6-membered heteroaryl having 1-3 ring heteroatoms independently selected from nitrogen, oxygen, and sulfur, 4-8 membered saturated heterocyclyl having 1-3 ring heteroatoms independently selected from nitrogen, oxygen, and sulfur, and C₁₋₄ alkoxy optionally substituted with 1-3 F, wherein two of the optional substituents of the C₃₋₆ cycloalkyl can be joined to form a 4-8 membered saturated heterocyclic ring containing 1 or 2 ring heteroatoms independently selected from nitrogen, oxygen, and sulfur, wherein the 5- or 6-membered heteroaryl or 4-8 membered saturated heterocyclic ring is optionally substituted with one or more substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
3) 4-8 membered heterocyclyl having 1-3 ring heteroatoms independently selected from nitrogen, oxygen, and sulfur, which is optionally substituted with 1-3 substituents independently selected from oxo, F, C₁₋₄ alkyl optionally substituted with 1-3 F, C₁₋₄ heteroalkyl, hydroxyl, G¹, and C₁₋₄ alkoxy optionally substituted with 1-3 F,
4) 5 or 6 membered heteroaryl having 1-4 ring heteroatoms independently selected from nitrogen, oxygen, and sulfur, which is optionally substituted with one or more substituents independently selected from C₁₋₄ alkyl optionally substituted with 1-3 F, C₁₋₄ heteroalkyl, hydroxyl, F, Cl, CN, and C₁₋₄ alkoxy optionally substituted with 1-3 F, and
5) phenyl, which is optionally substituted with 1-3 substituents independently selected from F, Cl, CN, C₁₋₄ alkyl optionally substituted with 1-3 F, C₁₋₄ heteroalkyl, hydroxyl, and C₁₋₄ alkoxy optionally substituted with 1-3 F,
wherein G¹ is a 4-8 membered saturated heterocyclyl containing 1 or 2 ring heteroatoms independently selected from nitrogen, oxygen, and sulfur, wherein the heterocyclyl is optionally substituted with one or more, such as 1, 2, or 3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F, or R¹⁰⁰ is a 7-10 membered spiro, bridged, or fused bicyclic ring structure containing 1 or 2 ring heteroatoms independently selected from nitrogen, oxygen, and sulfur, which is optionally substituted.

18. (canceled)

19. (canceled)

20. The compound of claim 1, or a pharmaceutically acceptable salt thereof, wherein R¹⁰⁰ is selected from: 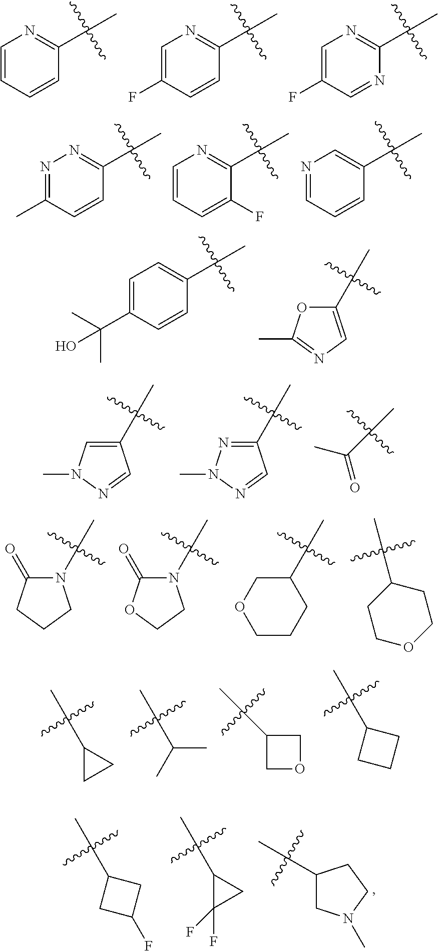
or R¹⁰⁰ is selected from: 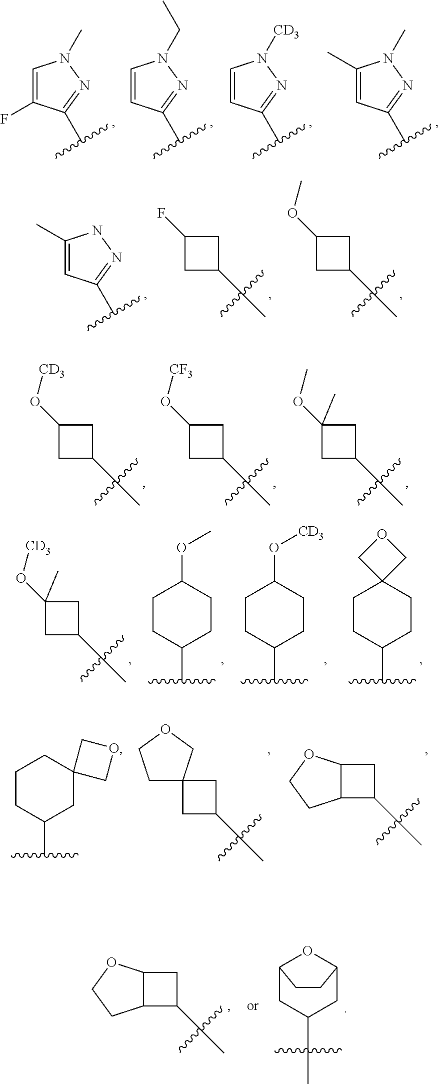
or R¹⁰⁰ is selected from: 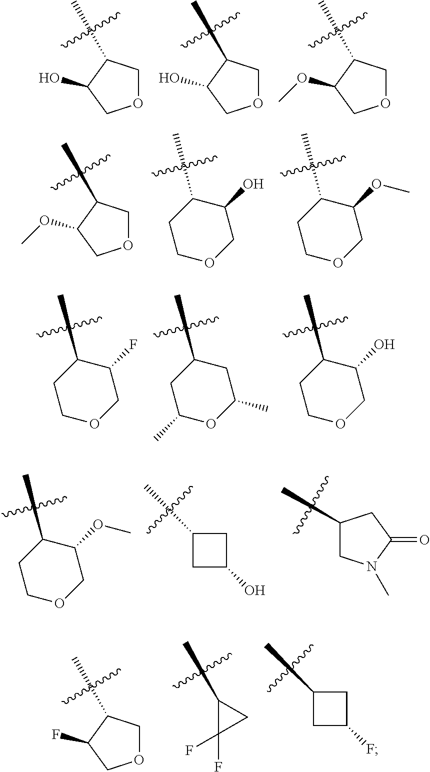
or R¹⁰⁰ is selected from: 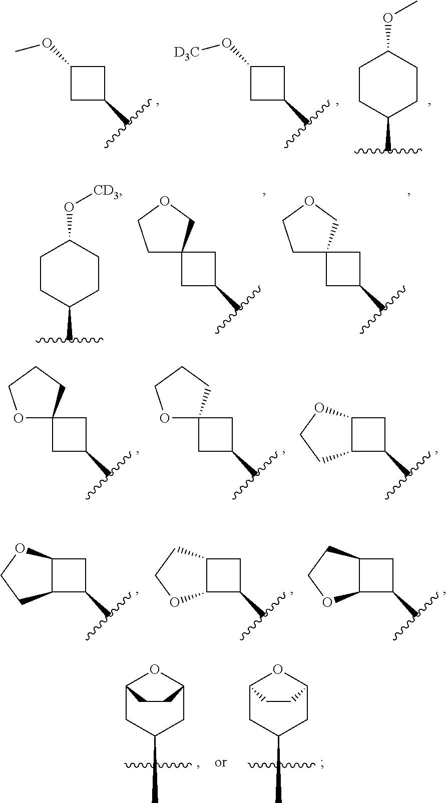
or R¹⁰⁰ is selected from: 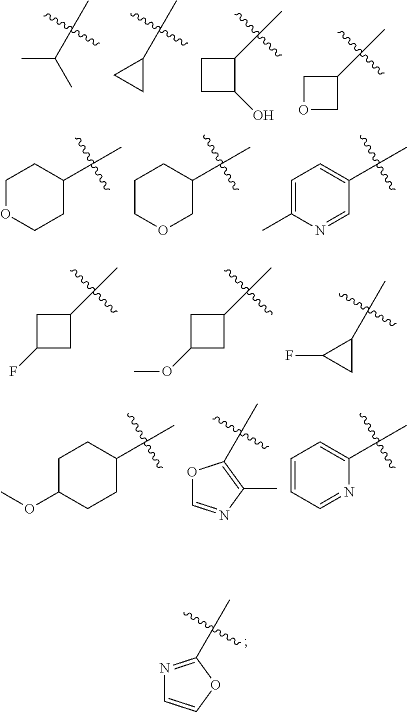
or R¹⁰⁰ is selected from:

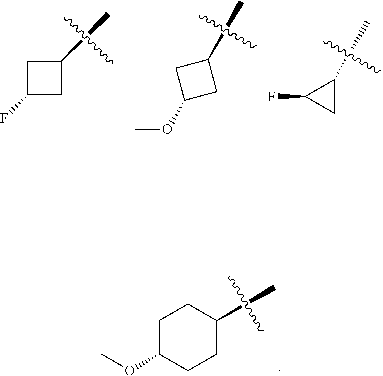

21. (canceled)

22. (canceled)

23. (canceled)

24. (canceled)

25. (canceled)

26. (canceled)

27. (canceled)

28. (canceled)

29. The compound of claim 1, or a pharmaceutically acceptable salt thereof, wherein R¹ is selected from: 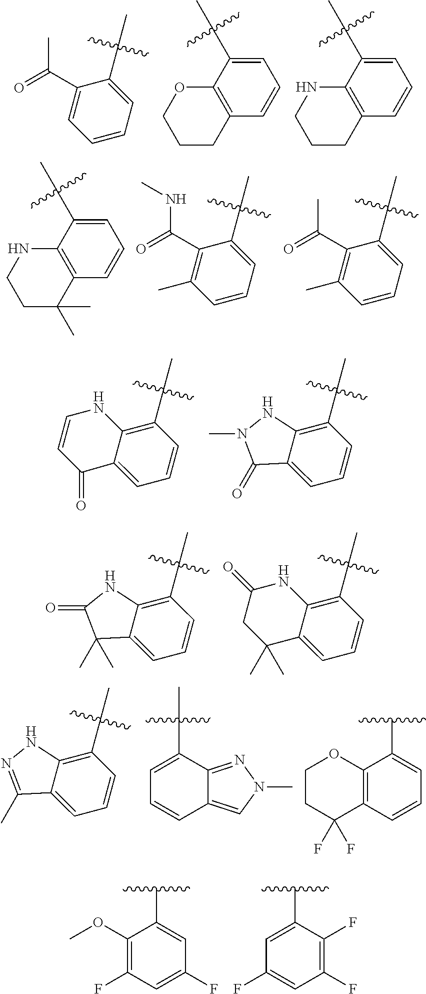
or R¹ is 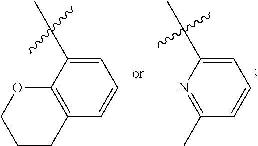
or wherein R¹ is a 5,6-bicyclic heteroaryl ring selected from: 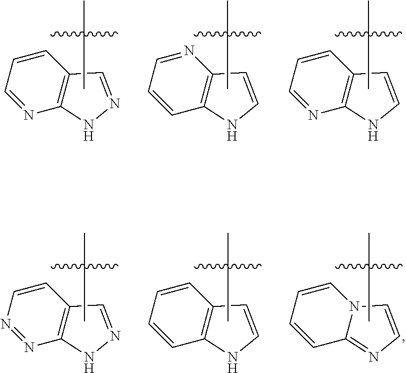
or a tautomeric form thereof, wherein each of which is substituted with one or more substituents each independently:
1) halogen, CN, —C(O)(C₁₋₄ alkyl), —C(O)NH₂, —COOH, —C(O)—N(H)(C₁₋₄ alkyl), —C(O)—N(C₁₋₄ alkyl)(C₁₋₄ alkyl), —N(H)—C(O)—(C₁₋₄ alkyl), or —N(C₁₋₄ alkyl)-C(O)—(C₁₋₄ alkyl),
2) C₁₋₆ alkyl, —O—C₁₋₆ alkyl, —N(H)—C₁₋₆ alkyl, or —(C₁₋₄ heteroalkylene)-C₁₋₆ alkyl, each of the C₁₋₆ alkyl is optionally substituted with one or more substituents independently selected from oxo, C₁₋₄ heteroalkyl, hydroxyl, N(C₁₋₄ alkyl)(C₁₋₄ alkyl), N(H)(C₁₋₄ alkyl), —C(O)—N(C₁₋₄ alkyl)(C₁₋₄ alkyl), —C(O)NH₂, —COOH, —C(O)—N(H)(C₁₋₄ alkyl), —N(H)—C(O)—(C₁₋₄ alkyl), —N(C₁₋₄ alkyl)-C(O)—(C₁₋₄ alkyl), —C(O)-G¹, F, C₁₋₄ alkoxy optionally substituted with 1-3 F, and 4-8 membered saturated heterocyclyl having 1-3 ring heteroatoms independently selected from nitrogen, oxygen, and sulfur, wherein the 4-8 membered saturated heterocyclyl is optionally substituted with 1-3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
3) C₃₋₁₀ cycloalkyl, —(C₁₋₄ alkylene)-C₃₋₁₀ cycloalkyl, —O—C₃₋₁₀ cycloalkyl, —N(H)—C₃₋₁₀ cycloalkyl, or —(C₁₋₄ heteroalkylene)-C₃₋₁₀ cycloalkyl, each of the C₃₋₁₀ cycloalkyl is monocyclic or polycyclic, preferably monocyclic C₃₋₆ cycloalkyl, which is optionally substituted with one or more substituents independently selected from C₁₋₄ alkyl optionally substituted with 1-3 F, C₁₋₄ heteroalkyl, hydroxyl, F, and C₁₋₄ alkoxy optionally substituted with 1-3 F, wherein two of the optional substituents of the cycloalkyl can be joined to form a 4-8 membered saturated heterocyclic ring containing 1 or 2 ring heteroatoms independently selected from nitrogen, oxygen, and sulfur, wherein the heterocyclic ring is optionally substituted with one or more substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
4) 4-8 membered heterocyclyl, —(C₁₋₄ alkylene)-(4-8 membered heterocyclyl), —O-(4-8 membered heterocyclyl), —N(H)-(4-8 membered heterocyclyl), or —(C₁₋₄ heteroalkylene)-(4-8 membered heterocyclyl), wherein each of the 4-8 membered heterocyclyl is a heterocyclyl having 1-3 ring heteroatoms independently selected from nitrogen, oxygen, and sulfur, which is optionally substituted with 1-3 substituents independently selected from oxo, F, C₁₋₄ alkyl optionally substituted with 1-3 F, C₁₋₄ heteroalkyl, hydroxyl, G¹, and C₁₋₄ alkoxy optionally substituted with 1-3 F, wherein two optional substituents of the heterocyclyl can be joined together to form a ring structure,
5) 5 or 6 membered heteroaryl, —(C₁₋₄ alkylene)-(5 or 6 membered heteroaryl), —O-(5 or 6 membered heteroaryl), —N(H)-(5 or 6 membered heteroaryl), or —(C₁₋₄ heteroalkylene)- (5 or 6 membered heteroaryl), wherein each of the 5 or 6 membered heteroaryl is a heteroaryl having 1-4 ring heteroatoms independently selected from nitrogen, oxygen, and sulfur, which is optionally substituted with one or more substituents independently selected from C₁₋₄ alkyl optionally substituted with 1-3 F, C₁₋₄ heteroalkyl, hydroxyl, F, Cl, CN, and C₁₋₄ alkoxy optionally substituted with 1-3 F, wherein two optional substituents of the heteroaryl can be joined together to form a ring structure, or
6) phenyl, —(C₁₋₄ alkylene)-phenyl, —O-phenyl, —N(H)-phenyl, or —(C₁₋₄ heteroalkylene)-phenyl, each of the phenyl is optionally substituted with 1-3 substituents independently selected from F, Cl, CN, C₁₋₄ alkyl optionally substituted with 1-3 F, C₁₋₄ heteroalkyl, hydroxyl, and C₁₋₄ alkoxy optionally substituted with 1-3 F, wherein two optional substituents of the phenyl can be joined together to form a ring structure, and wherein G¹ is a 4-8 membered saturated heterocyclyl containing 1 or 2 ring heteroatoms independently selected from nitrogen, oxygen, and sulfur, wherein the heterocyclyl is optionally substituted with one or more substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F.

30. The compound of claim 1, or a pharmaceutically acceptable salt thereof, wherein R¹ is selected from: 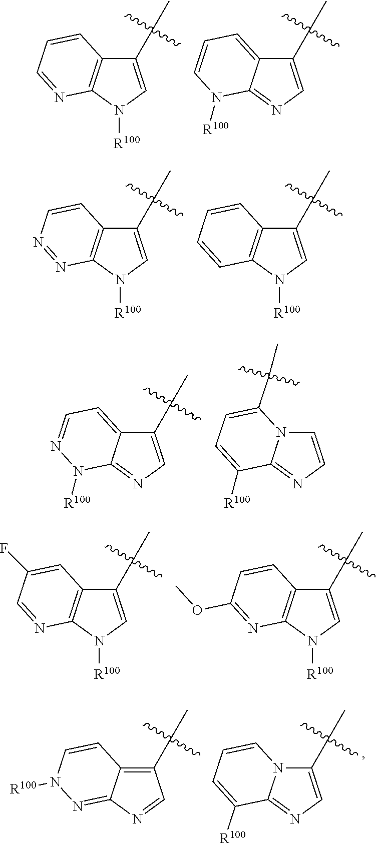
wherein each R¹⁰⁰ is independently selected from:
1) C₁₋₆ alkyl, which is optionally substituted with one or more substituents independently selected from oxo, C₁₋₄ heteroalkyl, hydroxyl, N(C₁₋₄ alkyl)(C₁₋₄ alkyl), N(H)(C₁₋₄ alkyl), —C(O)—N(C₁₋₄ alkyl)(C₁₋₄ alkyl), —C(O)NH₂, —COOH, —C(O)—N(H)(C₁₋₄ alkyl), —N(H)—C(O)—(C₁₋₄ alkyl), —N(C₁₋₄ alkyl)-C(O)—(C₁₋₄ alkyl), —C(O)-G¹, F, C₁₋₄ alkoxy optionally substituted with 1-3 F, and 4-8 membered saturated heterocyclyl having 1-3 ring heteroatoms independently selected from nitrogen, oxygen, and sulfur, wherein the 4-8 membered saturated heterocyclyl is optionally substituted with 1-3 substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
2) C₃₋₆ cycloalkyl, which is optionally substituted with one or more substituents independently selected from C₁₋₄ alkyl optionally substituted with 1-3 F, C₁₋₄ heteroalkyl, hydroxyl, F, and C₁₋₄ alkoxy optionally substituted with 1-3 F, wherein two of the optional substituents of the cycloalkyl can be joined to form a ring structure, such as a 4-8 membered saturated heterocyclic ring containing 1 or 2 ring heteroatoms independently selected from nitrogen, oxygen, and sulfur, wherein the heterocyclic ring is optionally substituted with one or more substituents independently selected from oxo, F, C₁₋₄ alkyl optionally substituted with 1-3 F, C₁₋₄ heteroalkyl, hydroxyl, and C₁₋₄ alkoxy optionally substituted with 1-3 F,
3) 4-8 membered heterocyclyl having 1-3 ring heteroatoms independently selected from nitrogen, oxygen, and sulfur, which is optionally substituted with 1-3 substituents independently selected from oxo, F, C₁₋₄ alkyl optionally substituted with 1-3 F, C₁₋₄ heteroalkyl, hydroxyl, G¹, and C₁₋₄ alkoxy optionally substituted with 1-3 F,
4) 5 or 6 membered heteroaryl having 1-4 ring heteroatoms independently selected from nitrogen, oxygen, and sulfur, which is optionally substituted with one or more substituents independently selected from C₁₋₄ alkyl optionally substituted with 1-3 F, Cl, CN, C₁₋₄ heteroalkyl, hydroxyl, F, and C₁₋₄ alkoxy optionally substituted with 1-3 F, and
5) phenyl, which is optionally substituted with 1-3 substituents independently selected from F, Cl, CN, C₁₋₄ alkyl optionally substituted with 1-3 F, C₁₋₄ heteroalkyl, hydroxyl, and C₁₋₄ alkoxy optionally substituted with 1-3 F, and
wherein G¹ is a 4-8 membered saturated heterocyclyl containing 1 or 2 ring heteroatoms independently selected from nitrogen, oxygen, and sulfur, wherein the heterocyclyl is optionally substituted with one or more substituents independently selected from oxo, F, C₁₋₄ alkyl optionally substituted with 1-3 F, C₁₋₄ heteroalkyl, hydroxyl, and C₁₋₄ alkoxy.

31. (canceled)

32. The compound of claim 30, or a pharmaceutically acceptable salt thereof, wherein R¹⁰⁰ is selected from: 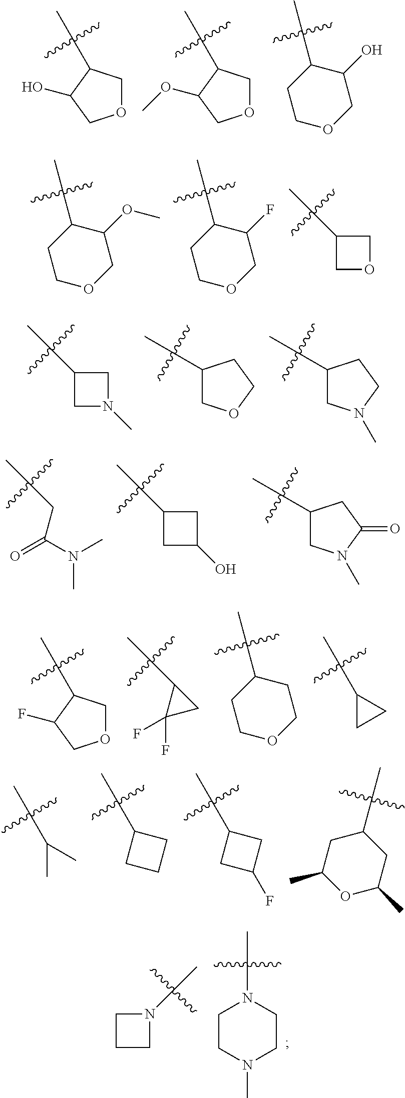
or R¹⁰⁰ is selected from: 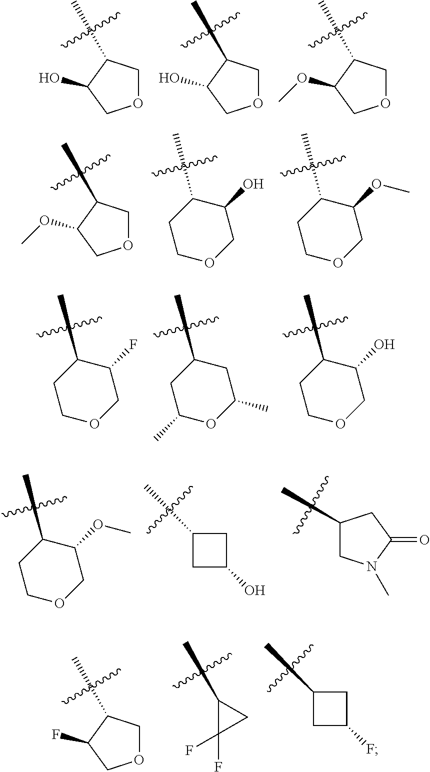
or R¹⁰⁰ is selected from: 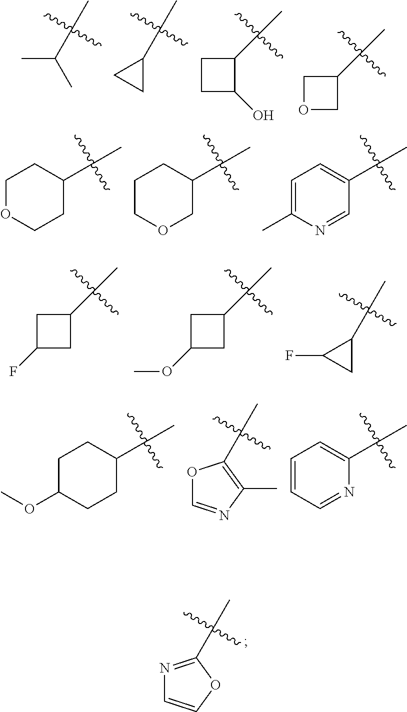
or R¹⁰⁰ is selected from:

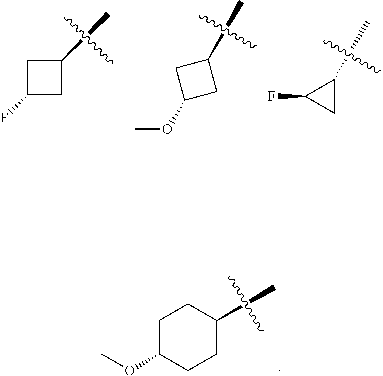

33. (canceled)

34. (canceled)

35. (canceled)

36. The compound of claim 1, or a pharmaceutically acceptable salt thereof, wherein L¹-R¹ is selected from: 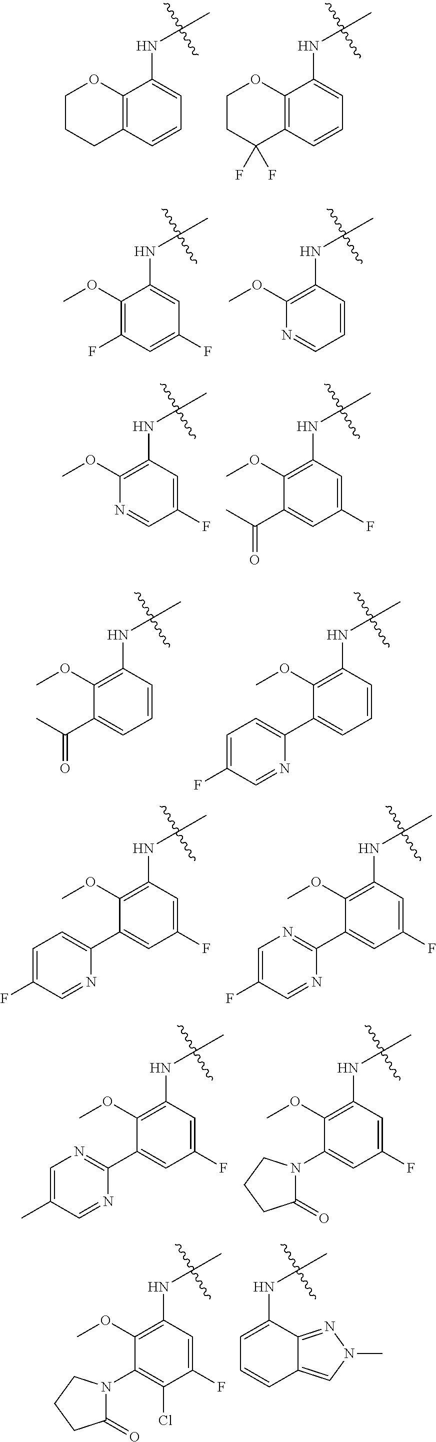 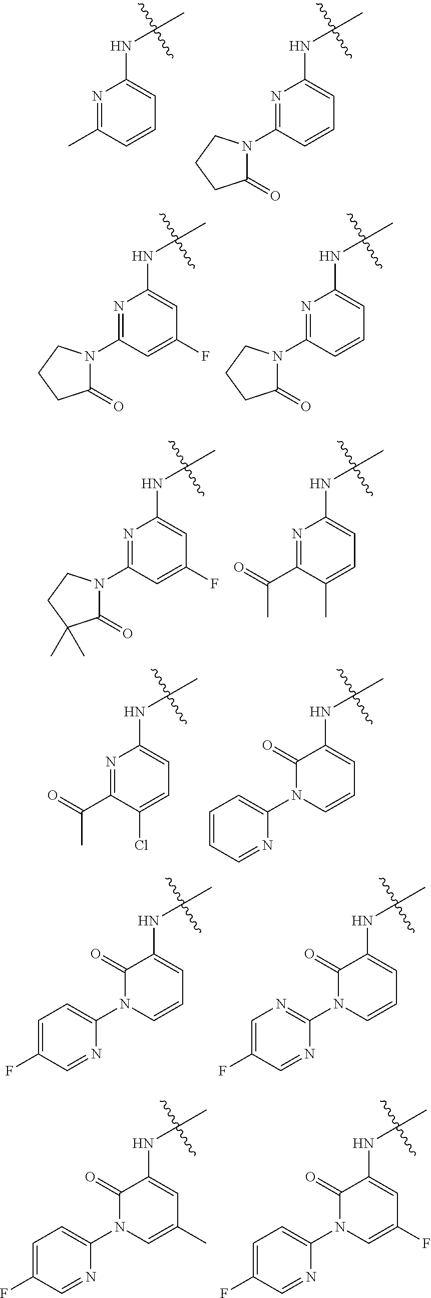 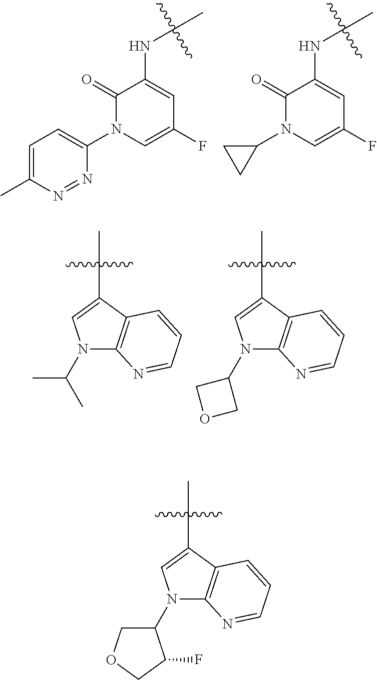
or L¹-R¹ is selected from: 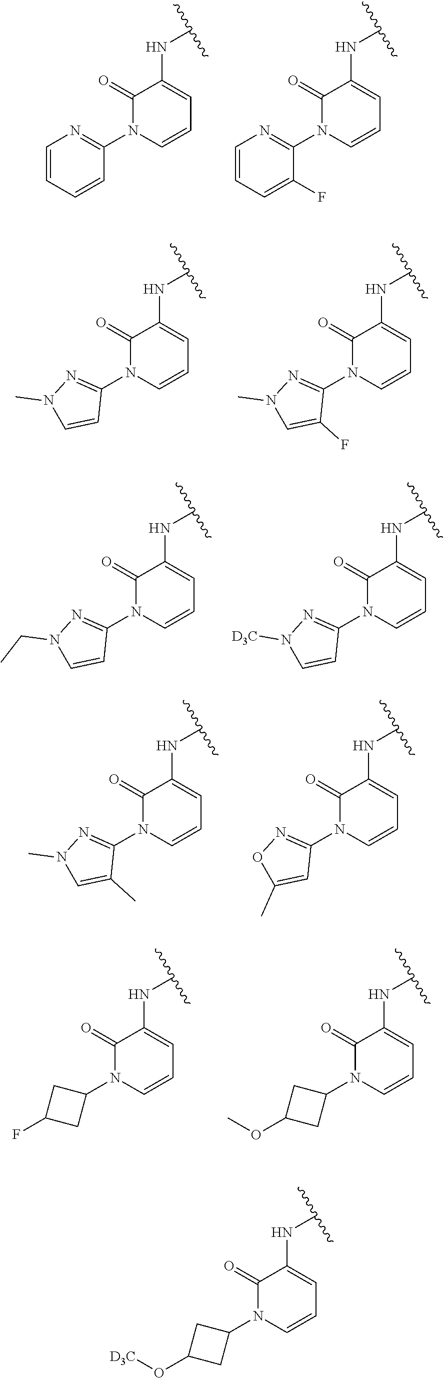 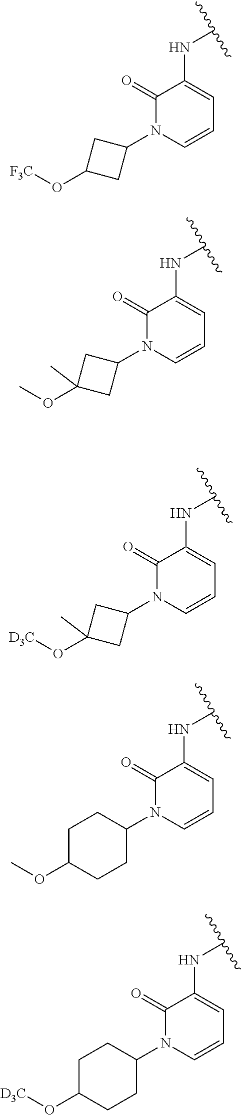 [SMILES: *Nc1cccn(C2CC(OC(F)(F)F)C2)c1=O ; *Nc1cccn(C2CC(C)(OC)C2)c1=O ; *Nc1cccn(C2CC(C)(OC([2H])([2H])[2H])C2)c1=O ; *Nc1cccn(C2CCC(OC)CC2)c1=O ; *Nc1cccn(C2CCC(OC([2H])([2H])[2H])CC2)c1=O]
or L¹-R¹ is selected from: 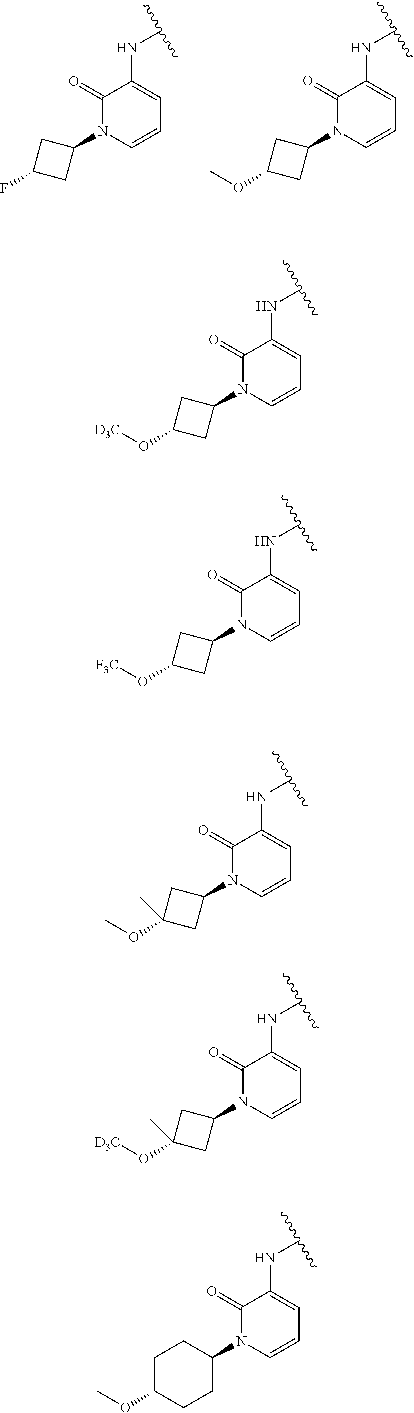 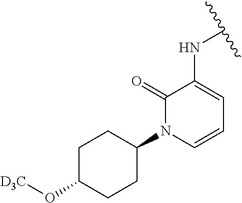 [SMILES: *Nc1cccn([C@H]2CC[C@H](OC([2H])([2H])[2H])CC2)c1=O]
or L¹-R¹ is selected from: 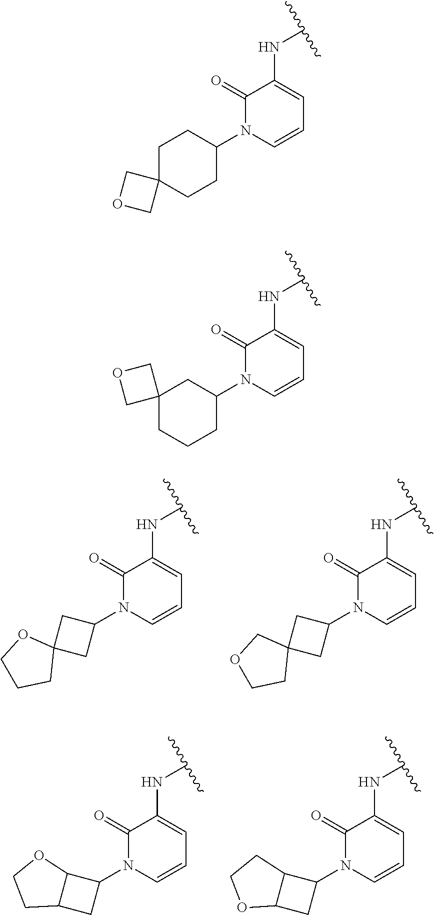
or L¹-R¹ is selected from:

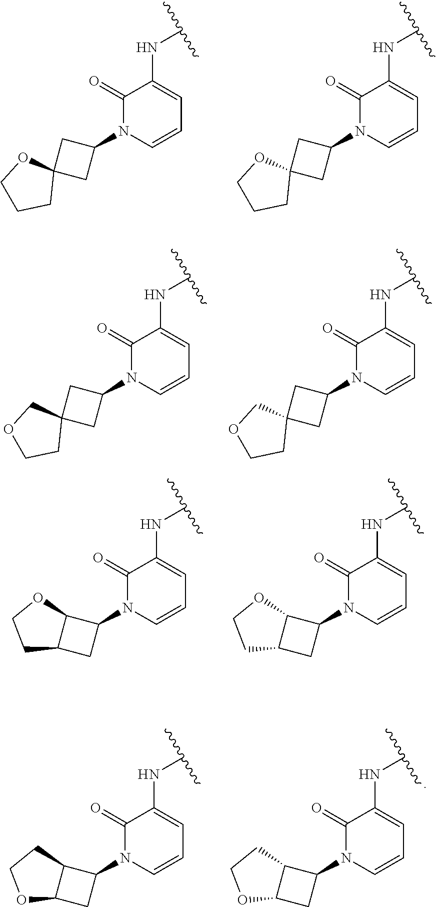

37. The compound of claim 1, or a pharmaceutically acceptable salt thereof, wherein X is CH or N.

38. (canceled)

39. The compound of claim 1, or a pharmaceutically acceptable salt thereof, wherein R³ is hydrogen or a C₁₋₄ alkyl, or R³ is CD₃.

40. (canceled)

41. A compound selected from 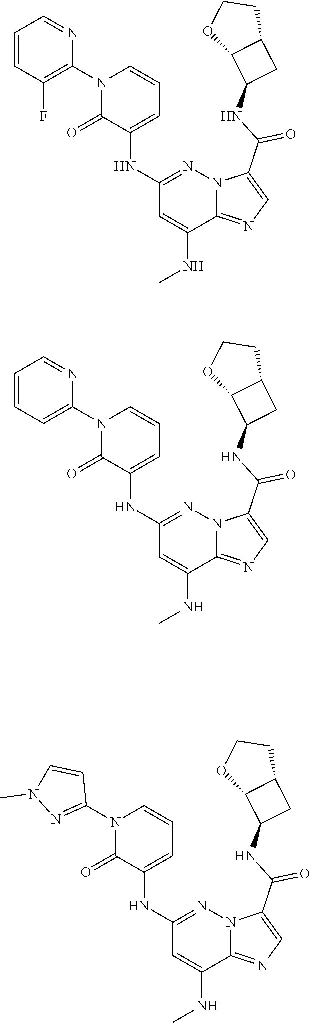 [SMILES: CNc1cc(Nc2cccn(-c3ncccc3F)c2=O)nn2c(C(=O)N[C@@H]3C[C@H]4CCO[C@H]43)cnc12 ; CNc1cc(Nc2cccn(-c3ccccn3)c2=O)nn2c(C(=O)N[C@@H]3C[C@H]4CCO[C@H]43)cnc12 ; CNc1cc(Nc2cccn(-c3ccn(C)n3)c2=O)nn2c(C(=O)N[C@@H]3C[C@H]4CCO[C@H]43)cnc12] 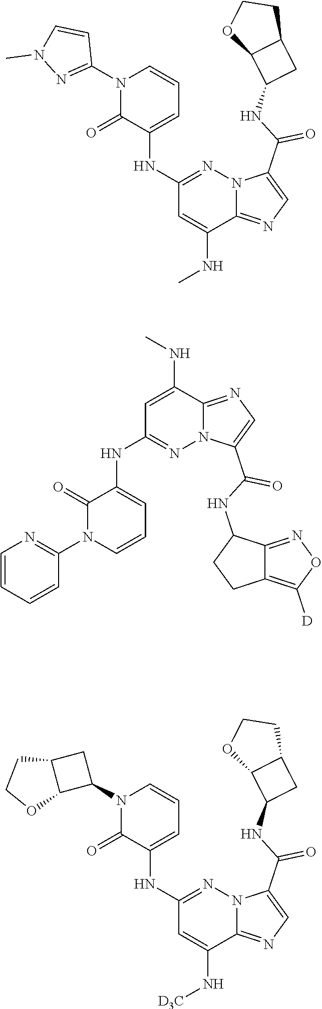 [SMILES: CNc1cc(Nc2cccn(-c3ccn(C)n3)c2=O)nn2c(C(=O)N[C@H]3C[C@@H]4CCO[C@@H]43)cnc12 ; [2H]c1onc2c1CCC2NC(=O)c1cnc2c(NC)cc(Nc3cccn(-c4ccccn4)c3=O)nn12 ; [2H]C([2H])([2H])Nc1cc(Nc2cccn([C@@H]3C[C@H]4CCO[C@H]43)c2=O)nn2c(C(=O)N[C@@H]3C[C@H]4CCO[C@H]43)cnc12] 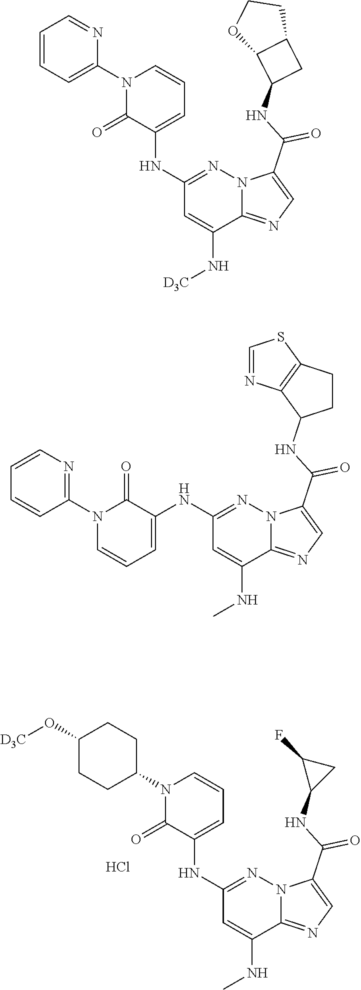 [SMILES: [2H]C([2H])([2H])Nc1cc(Nc2cccn(-c3ccccn3)c2=O)nn2c(C(=O)N[C@@H]3C[C@H]4CCO[C@H]43)cnc12 ; CNc1cc(Nc2cccn(-c3ccccn3)c2=O)nn2c(C(=O)NC3CCc4scnc43)cnc12 ; [2H]C([2H])([2H])O[C@H]1CC[C@@H](n2cccc(Nc3cc(NC)c4ncc(C(=O)N[C@@H]5C[C@@H]5F)n4n3)c2=O)CC1 ; Cl] 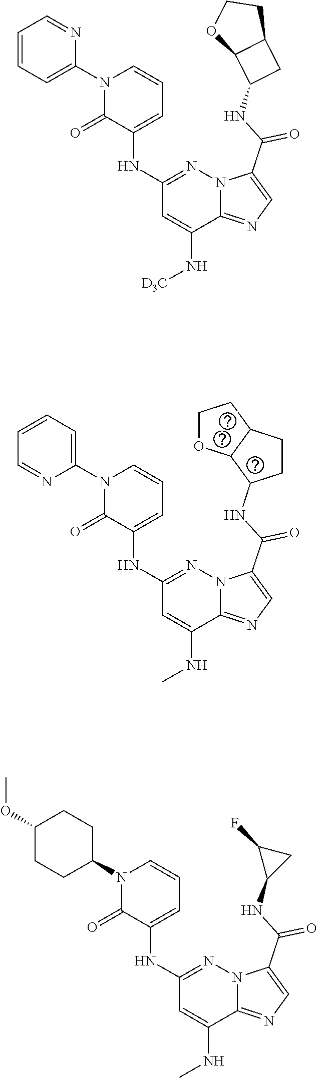 [SMILES: [2H]C([2H])([2H])Nc1cc(Nc2cccn(-c3ccccn3)c2=O)nn2c(C(=O)N[C@H]3C[C@@H]4CCO[C@@H]43)cnc12 ; CNc1cc(Nc2cccn(-c3ccccn3)c2=O)nn2c(C(=O)NC3CCC4CCOC43)cnc12 ; CNc1cc(Nc2cccn([C@H]3CC[C@H](OC)CC3)c2=O)nn2c(C(=O)N[C@@H]3C[C@@H]3F)cnc12] 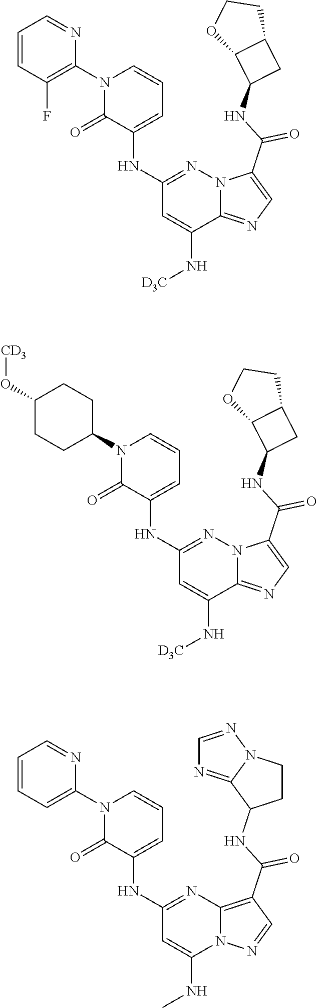 [SMILES: [2H]C([2H])([2H])Nc1cc(Nc2cccn(-c3ncccc3F)c2=O)nn2c(C(=O)N[C@@H]3C[C@H]4CCO[C@H]43)cnc12 ; [2H]C([2H])([2H])Nc1cc(Nc2cccn([C@H]3CC[C@H](OC([2H])([2H])[2H])CC3)c2=O)nn2c(C(=O)N[C@@H]3C[C@H]4CCO[C@H]43)cnc12 ; CNc1cc(Nc2cccn(-c3ccccn3)c2=O)nc2c(C(=O)NC3CCn4ncnc43)cnn12] 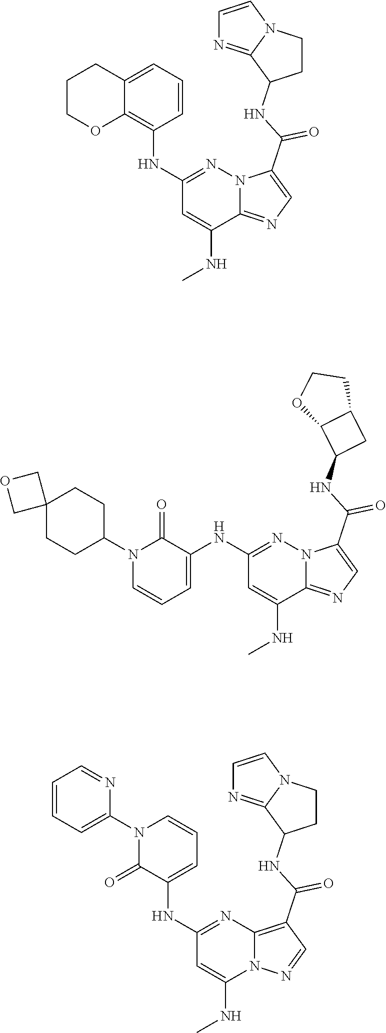 [SMILES: CNc1cc(Nc2cccc3c2OCCC3)nn2c(C(=O)NC3CCn4ccnc43)cnc12 ; CNc1cc(Nc2cccn(C3CCC4(CC3)COC4)c2=O)nn2c(C(=O)N[C@@H]3C[C@H]4CCO[C@H]43)cnc12 ; CNc1cc(Nc2cccn(-c3ccccn3)c2=O)nc2c(C(=O)NC3CCn4ccnc43)cnn12] 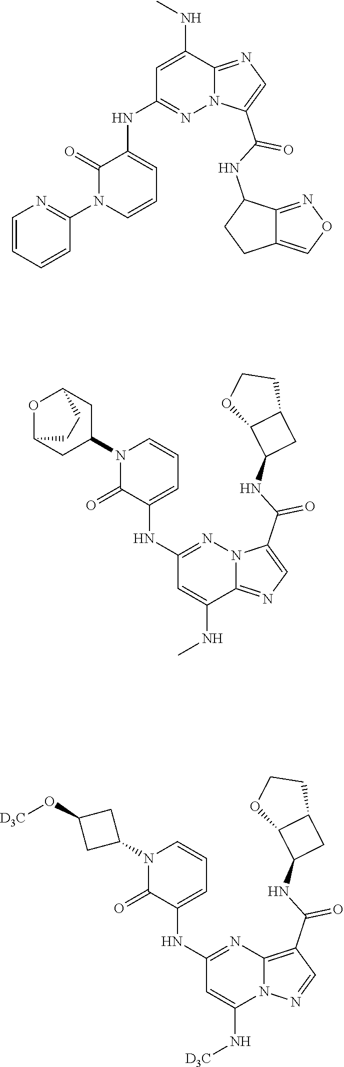 [SMILES: CNc1cc(Nc2cccn(-c3ccccn3)c2=O)nn2c(C(=O)NC3CCc4conc43)cnc12 ; CNc1cc(Nc2cccn([C@@H]3C[C@H]4CC[C@@H](C3)O4)c2=O)nn2c(C(=O)N[C@@H]3C[C@H]4CCO[C@H]43)cnc12 ; [2H]C([2H])([2H])Nc1cc(Nc2cccn([C@H]3C[C@H](OC([2H])([2H])[2H])C3)c2=O)nc2c(C(=O)N[C@@H]3C[C@H]4CCO[C@H]43)cnn12] 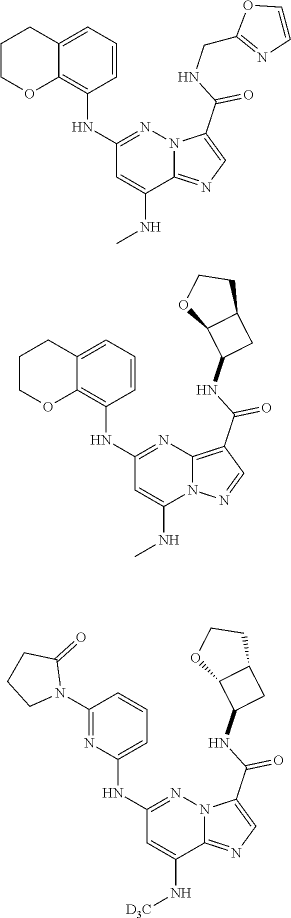 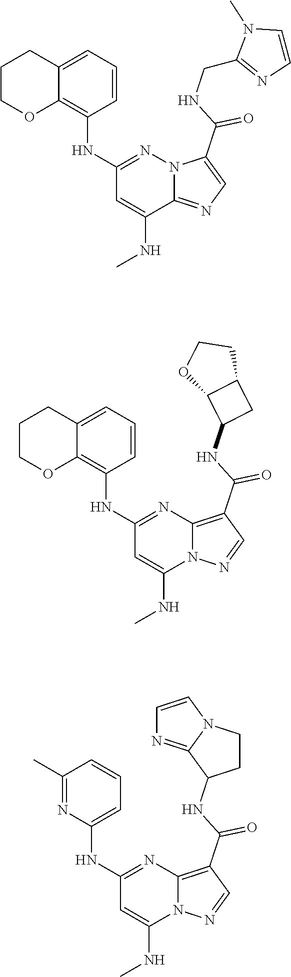 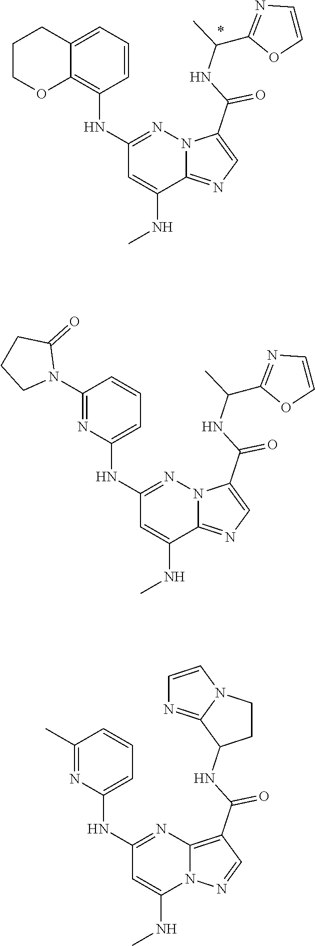 [SMILES: CNc1cc(Nc2cccc3c2OCCC3)nn2c(C(=O)NC(C)c3ncco3)cnc12 ; CNc1cc(Nc2cccc(N3CCCC3=O)n2)nn2c(C(=O)NC(C)c3ncco3)cnc12 ; CNc1cc(Nc2cccc(C)n2)nc2c(C(=O)NC3CCn4ccnc43)cnn12] 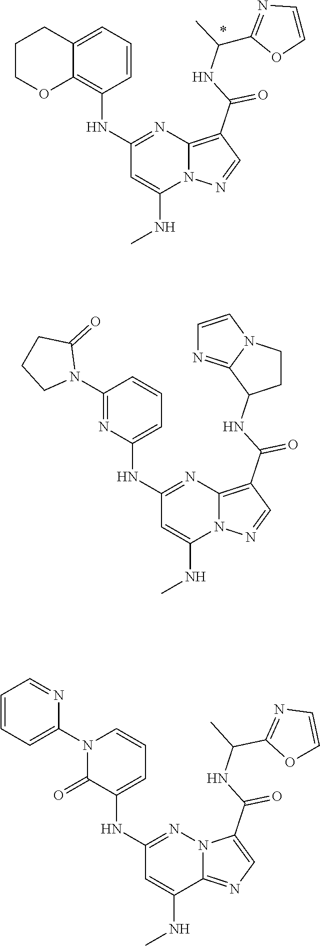 [SMILES: CNc1cc(Nc2cccc3c2OCCC3)nc2c(C(=O)NC(C)c3ncco3)cnn12 ; CNc1cc(Nc2cccc(N3CCCC3=O)n2)nc2c(C(=O)NC3CCn4ccnc43)cnn12 ; CNc1cc(Nc2cccn(-c3ccccn3)c2=O)nn2c(C(=O)NC(C)c3ncco3)cnc12] 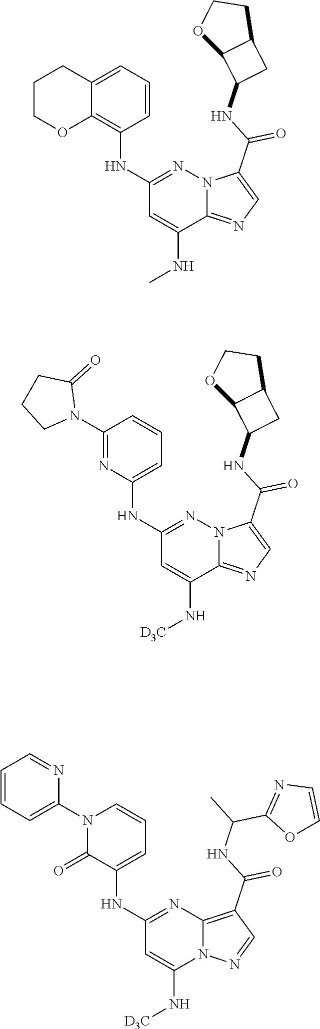 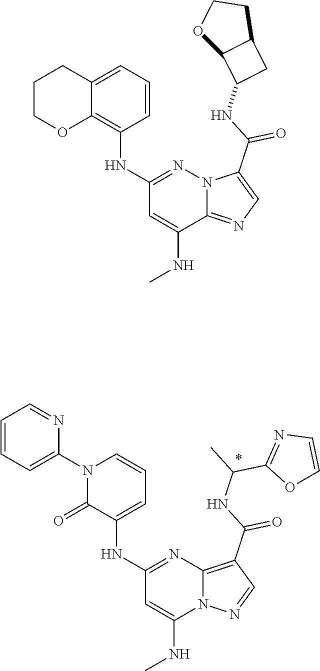 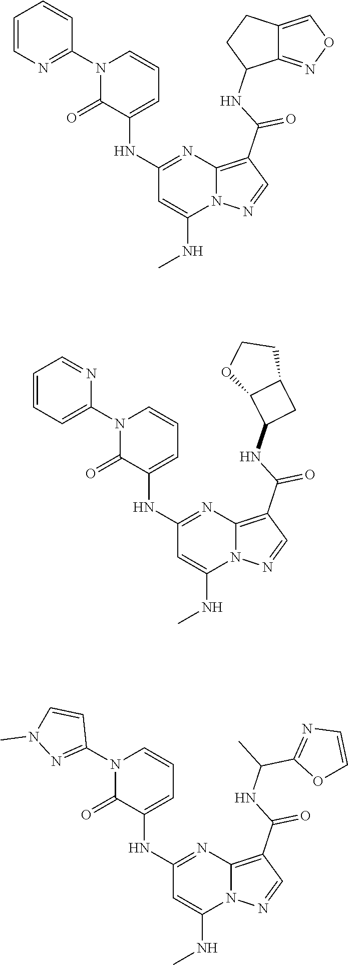 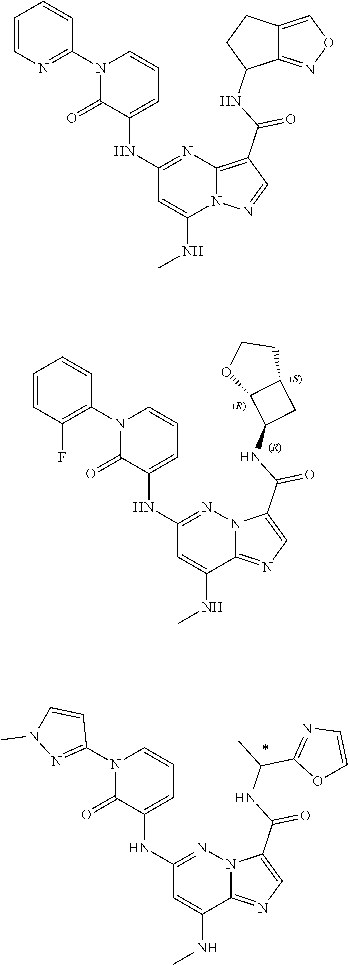 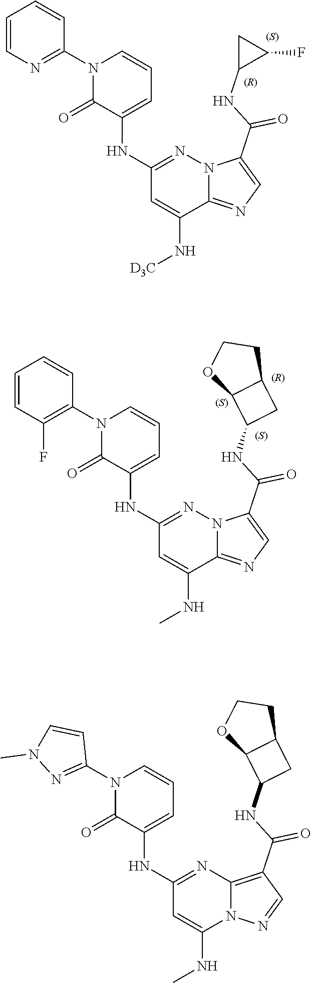 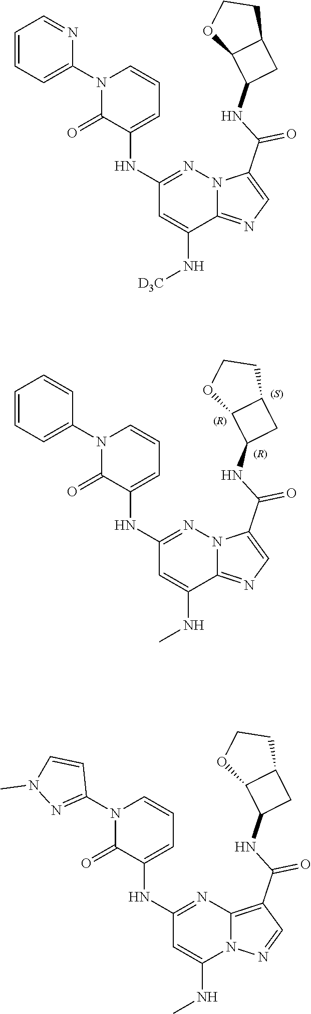 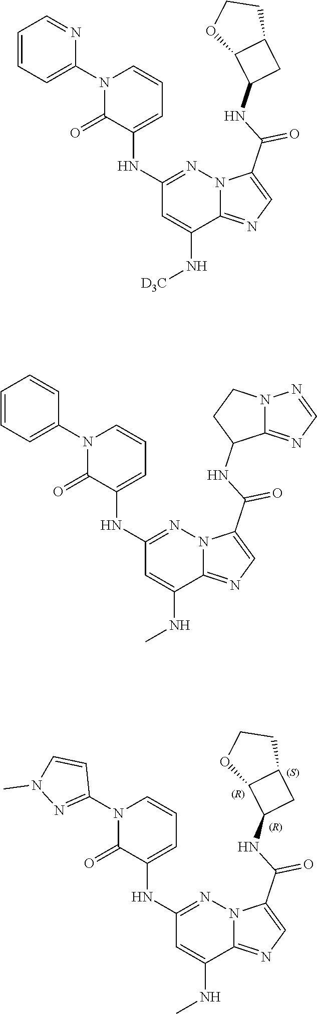 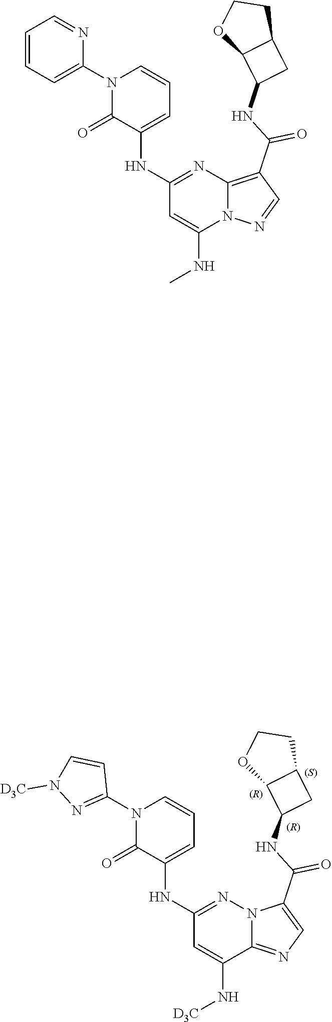 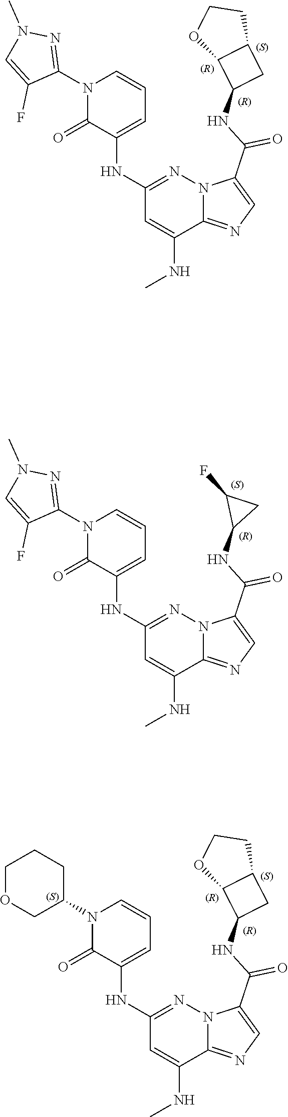 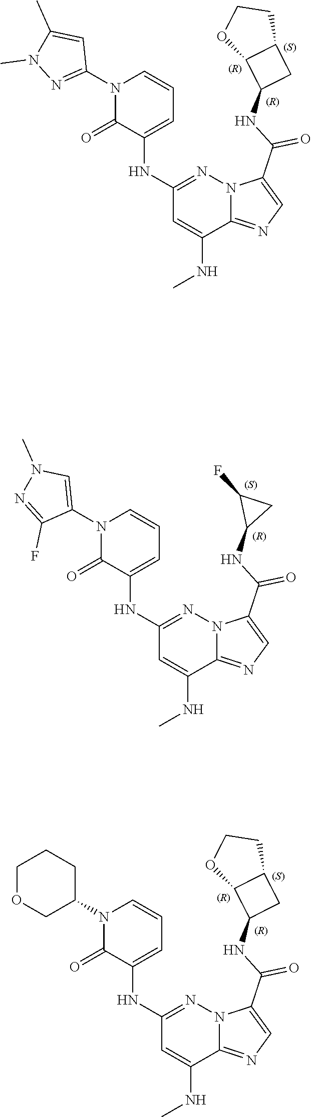 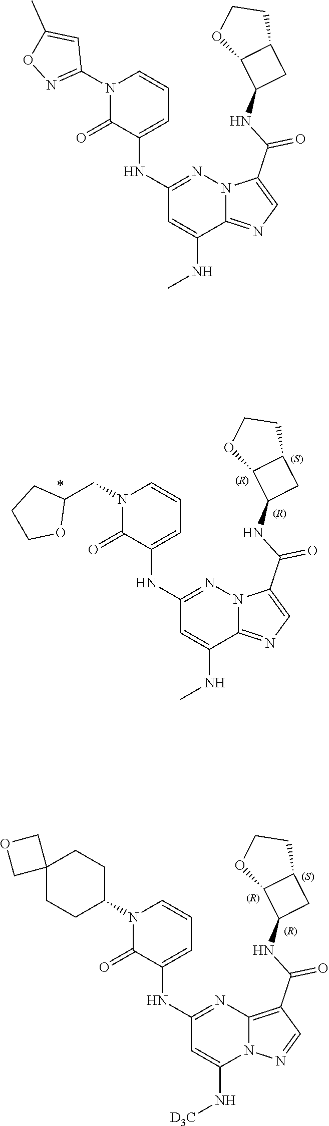 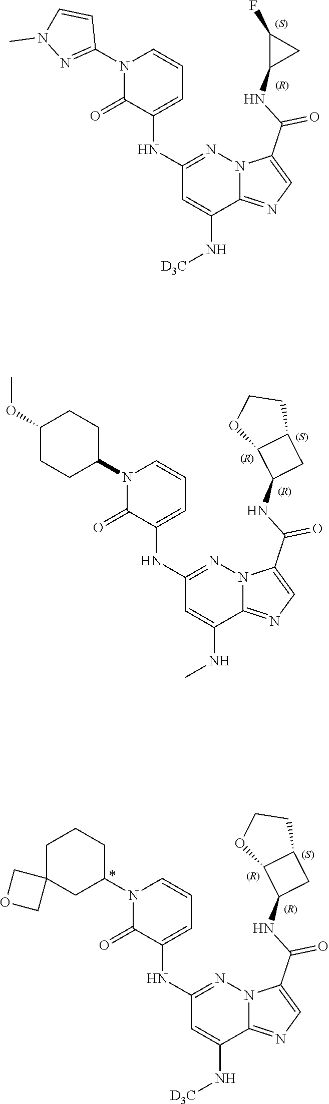 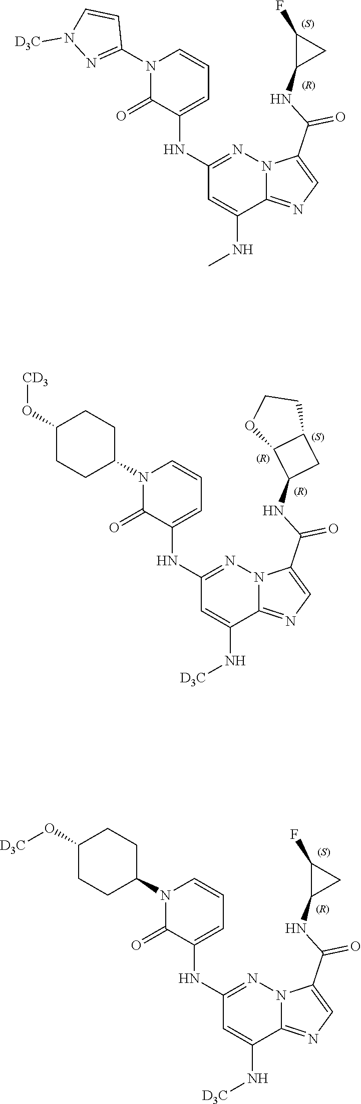 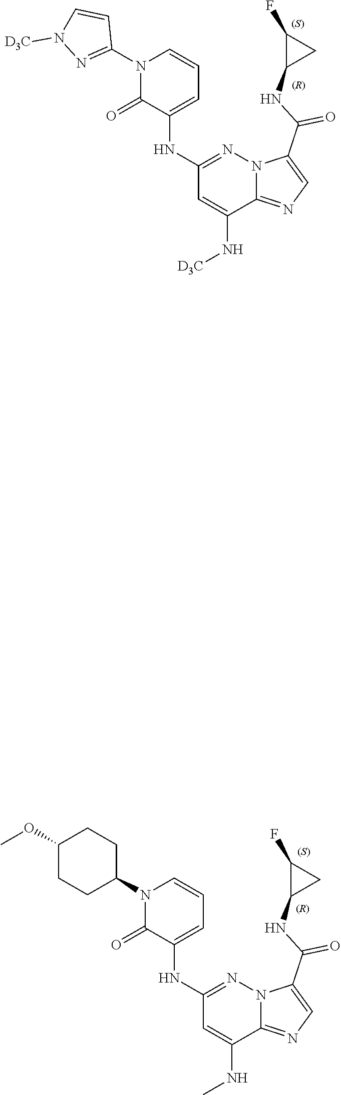 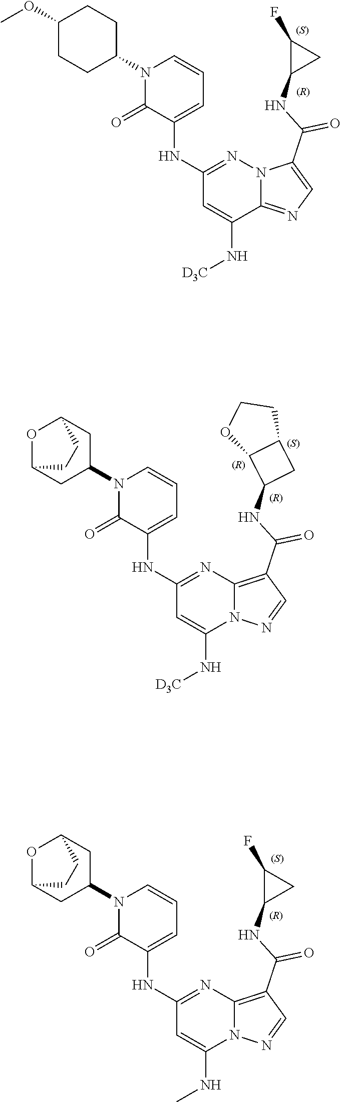 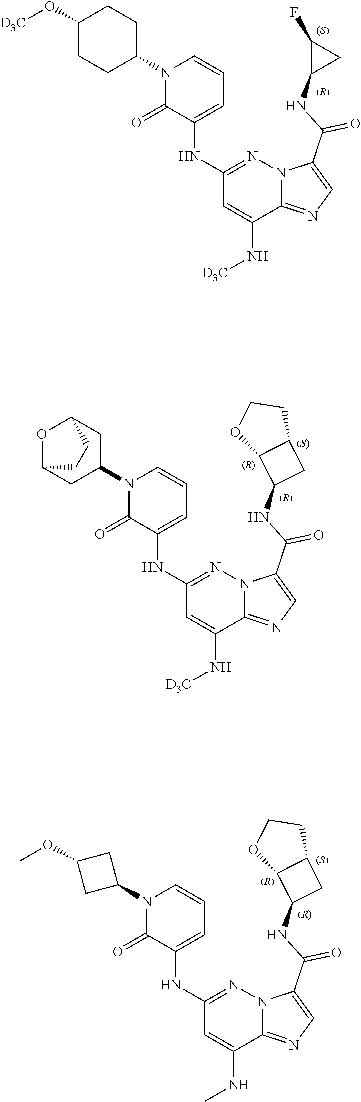 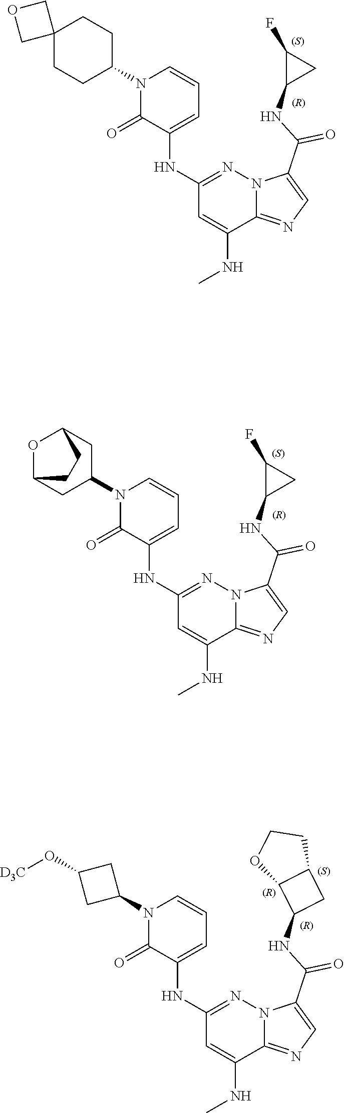 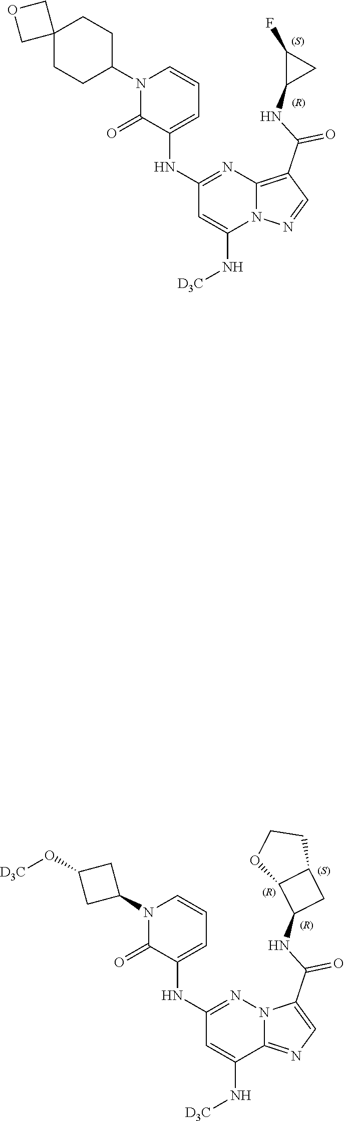 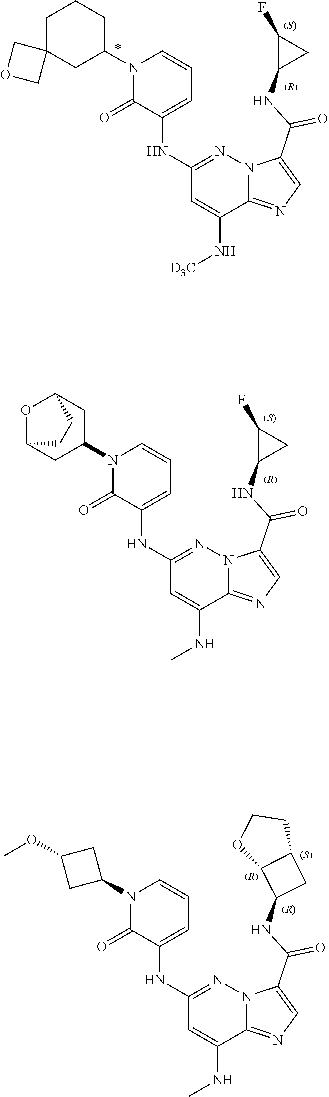 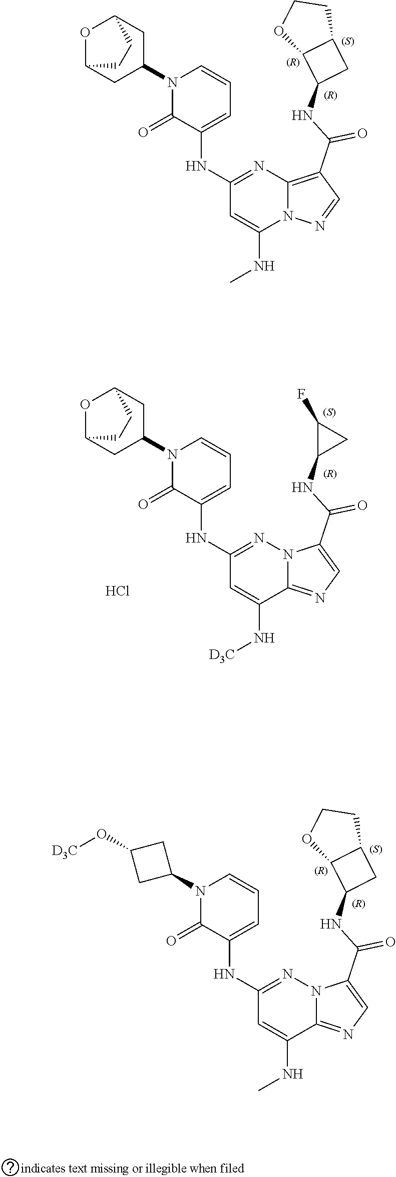 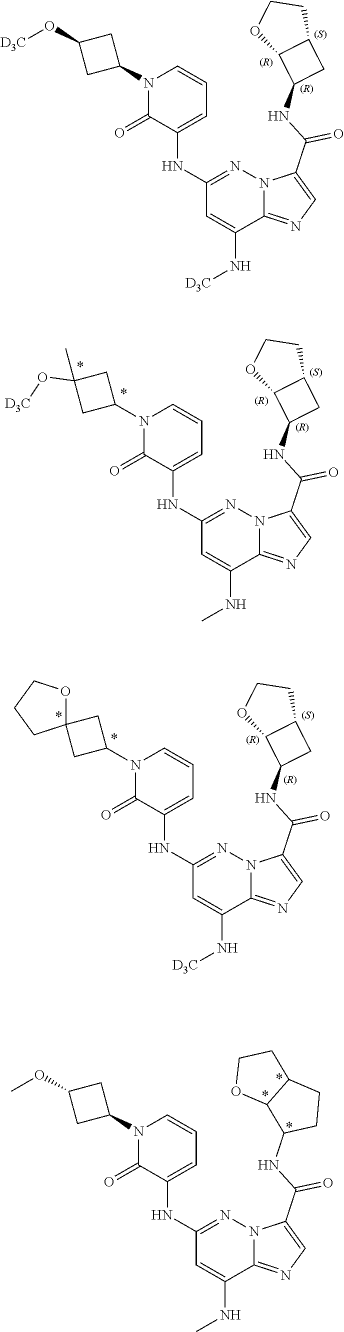 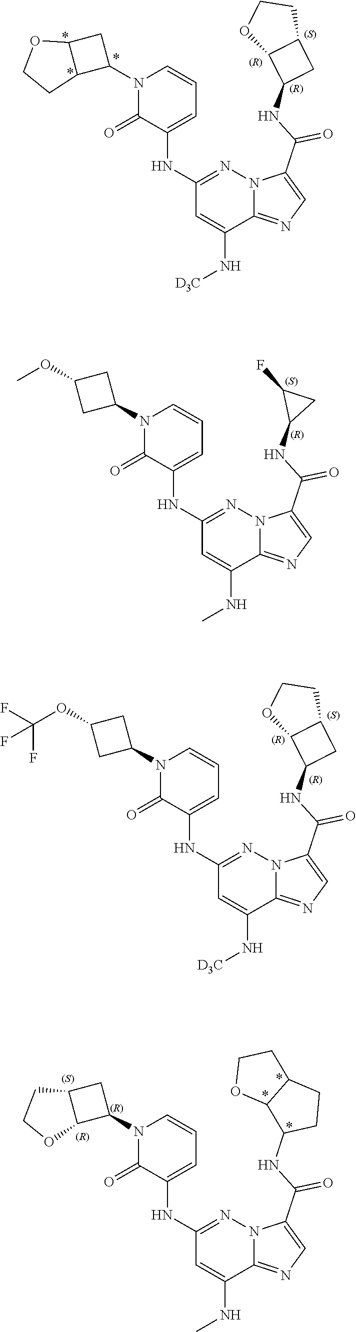 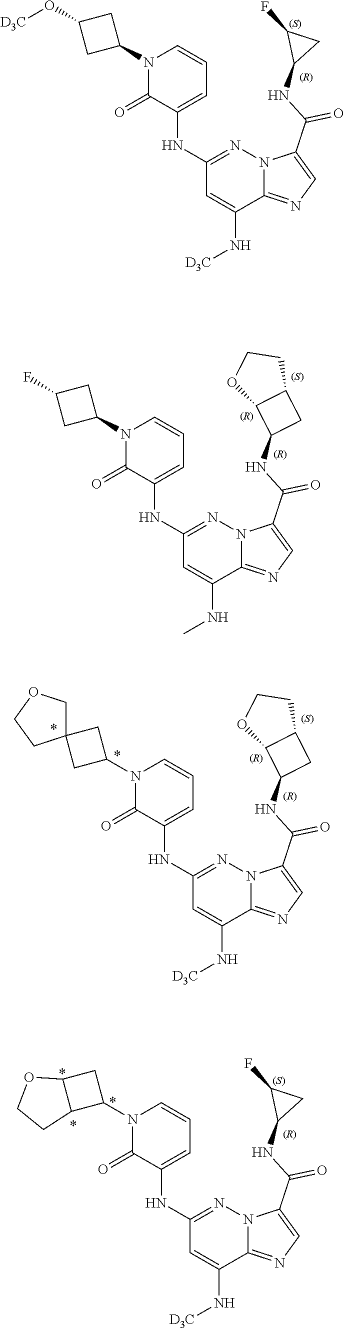 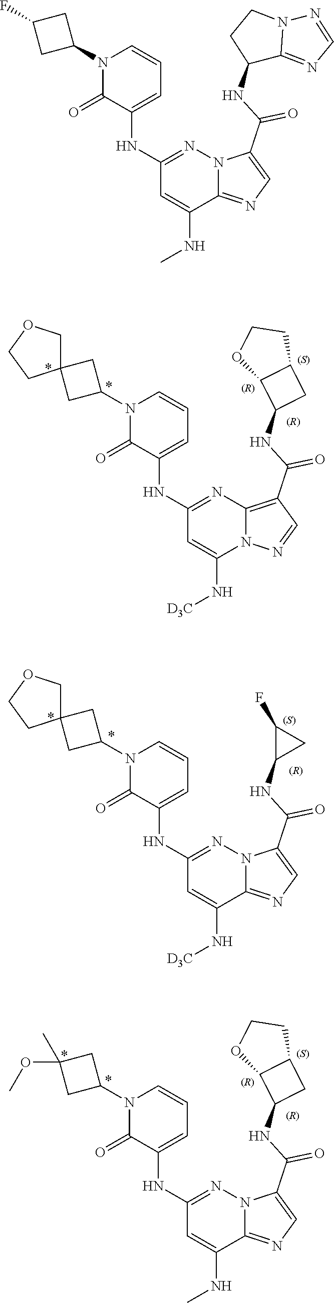 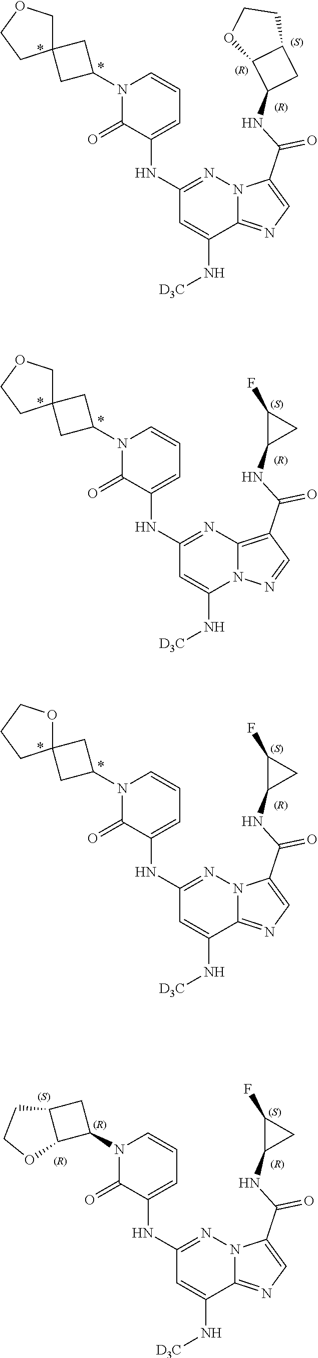 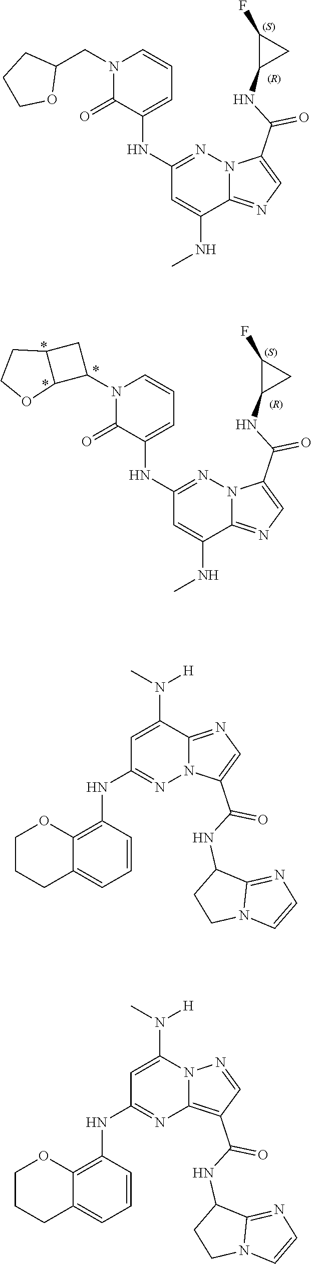 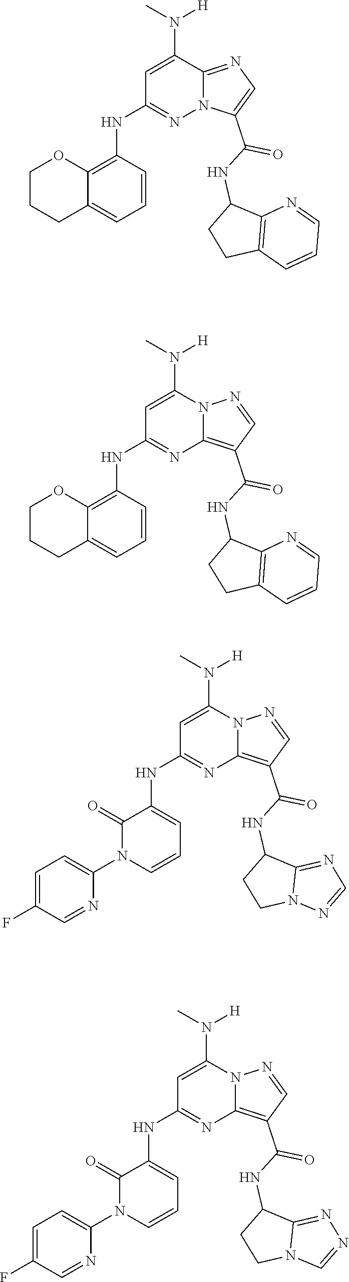 [SMILES: CNc1cc(Nc2cccc3c2OCCC3)nn2c(C(=O)NC3CCc4cccnc43)cnc12 ; CNc1cc(Nc2cccc3c2OCCC3)nc2c(C(=O)NC3CCc4cccnc43)cnn12 ; CNc1cc(Nc2cccn(-c3ccc(F)cn3)c2=O)nc2c(C(=O)NC3CCn4ncnc43)cnn12 ; CNc1cc(Nc2cccn(-c3ccc(F)cn3)c2=O)nc2c(C(=O)NC3CCn4cnnc43)cnn12] 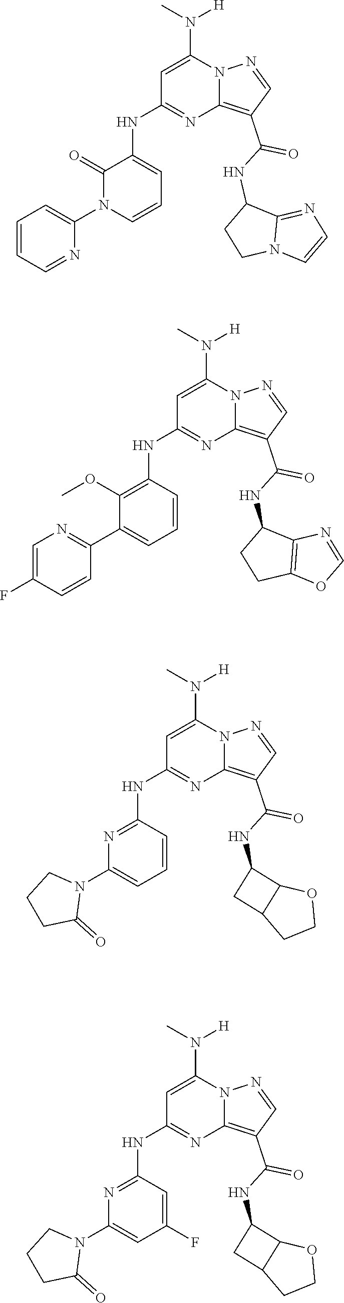 [SMILES: CNc1cc(Nc2cccn(-c3ccccn3)c2=O)nc2c(C(=O)NC3CCn4ccnc43)cnn12 ; CNc1cc(Nc2cccc(-c3ccc(F)cn3)c2OC)nc2c(C(=O)N[C@@H]3CCc4ocnc43)cnn12 ; CNc1cc(Nc2cccc(N3CCCC3=O)n2)nc2c(C(=O)N[C@@H]3CC4CCOC43)cnn12 ; CNc1cc(Nc2cc(F)cc(N3CCCC3=O)n2)nc2c(C(=O)N[C@@H]3CC4CCOC43)cnn12] 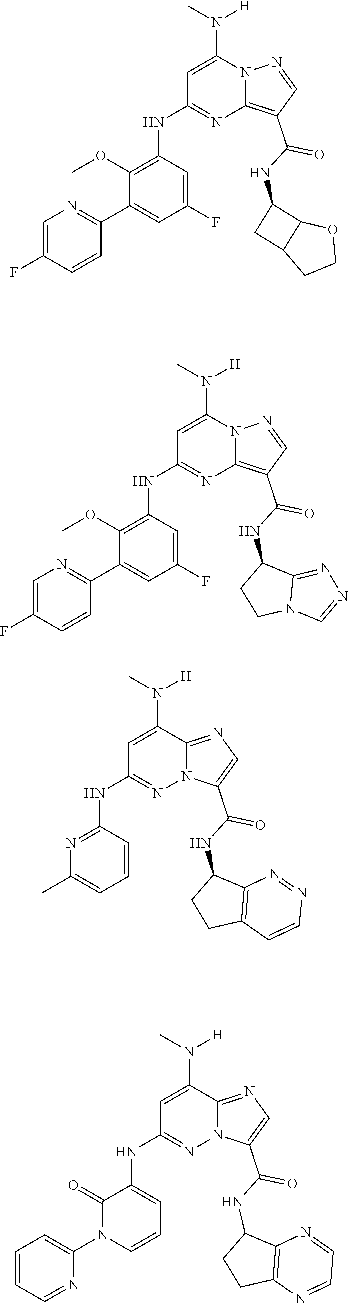 [SMILES: CNc1cc(Nc2cc(F)cc(-c3ccc(F)cn3)c2OC)nc2c(C(=O)N[C@@H]3CC4CCOC43)cnn12 ; CNc1cc(Nc2cc(F)cc(-c3ccc(F)cn3)c2OC)nc2c(C(=O)N[C@@H]3CCn4cnnc43)cnn12 ; CNc1cc(Nc2cccc(C)n2)nn2c(C(=O)N[C@@H]3CCc4ccnnc43)cnc12 ; CNc1cc(Nc2cccn(-c3ccccn3)c2=O)nn2c(C(=O)NC3CCc4nccnc43)cnc12] 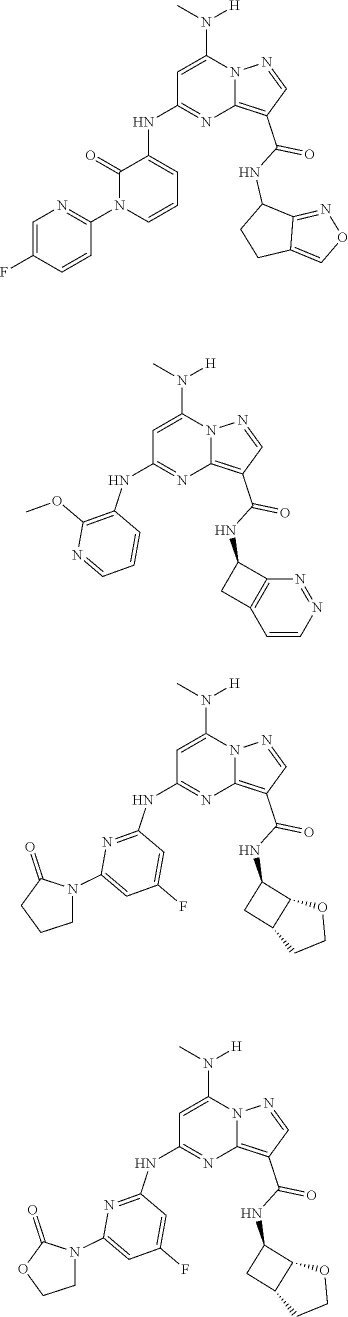 [SMILES: CNc1cc(Nc2cccn(-c3ccc(F)cn3)c2=O)nc2c(C(=O)NC3CCc4conc43)cnn12 ; CNc1cc(Nc2cccnc2OC)nc2c(C(=O)N[C@@H]3Cc4ccnnc43)cnn12 ; CNc1cc(Nc2cc(F)cc(N3CCCC3=O)n2)nc2c(C(=O)N[C@@H]3C[C@H]4CCO[C@H]43)cnn12 ; CNc1cc(Nc2cc(F)cc(N3CCOC3=O)n2)nc2c(C(=O)N[C@@H]3C[C@H]4CCO[C@H]43)cnn12] 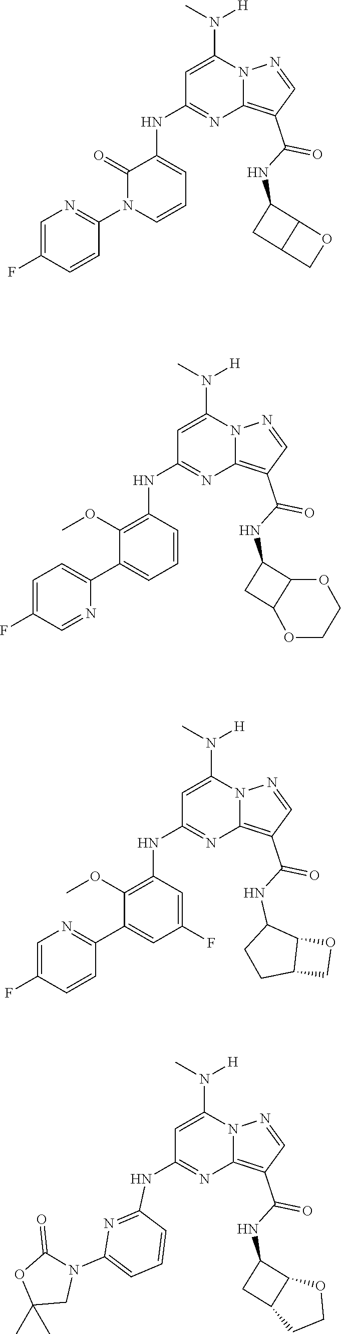 [SMILES: CNc1cc(Nc2cccn(-c3ccc(F)cn3)c2=O)nc2c(C(=O)N[C@@H]3CC4COC43)cnn12 ; CNc1cc(Nc2cccc(-c3ccc(F)cn3)c2OC)nc2c(C(=O)N[C@@H]3CC4OCCOC43)cnn12 ; CNc1cc(Nc2cc(F)cc(-c3ccc(F)cn3)c2OC)nc2c(C(=O)NC3CC[C@H]4CO[C@@H]34)cnn12 ; CNc1cc(Nc2cccc(N3CC(C)(C)OC3=O)n2)nc2c(C(=O)N[C@@H]3C[C@H]4CCO[C@H]43)cnn12] 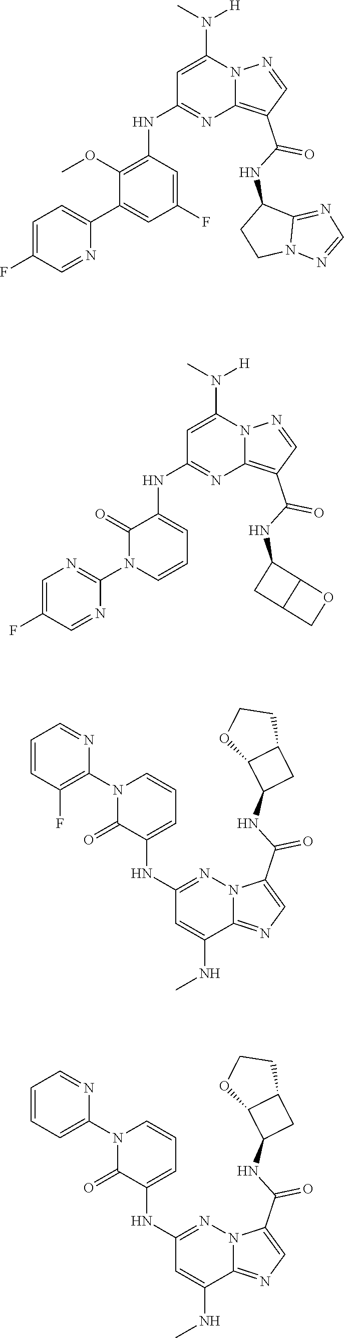 [SMILES: CNc1cc(Nc2cc(F)cc(-c3ccc(F)cn3)c2OC)nc2c(C(=O)N[C@@H]3CCn4ncnc43)cnn12 ; CNc1cc(Nc2cccn(-c3ncc(F)cn3)c2=O)nc2c(C(=O)N[C@@H]3CC4COC43)cnn12 ; CNc1cc(Nc2cccn(-c3ncccc3F)c2=O)nn2c(C(=O)N[C@@H]3C[C@H]4CCO[C@H]43)cnc12 ; CNc1cc(Nc2cccn(-c3ccccn3)c2=O)nn2c(C(=O)N[C@@H]3C[C@H]4CCO[C@H]43)cnc12] 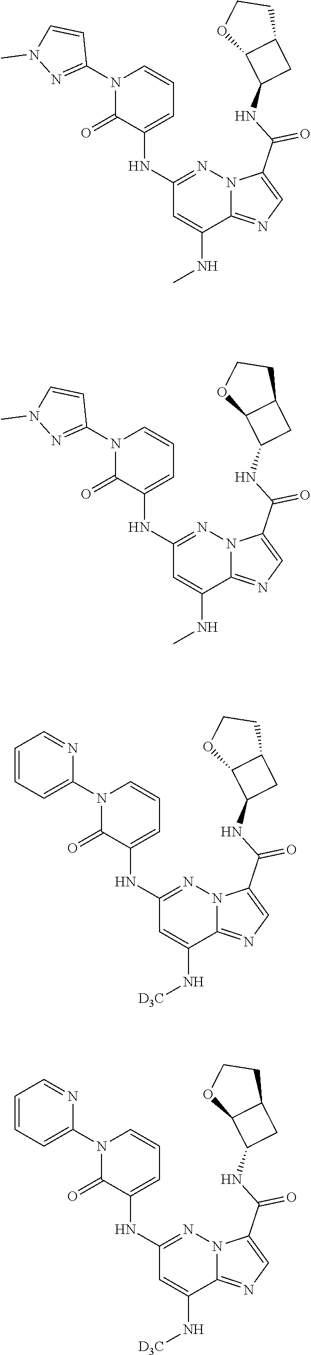 [SMILES: CNc1cc(Nc2cccn(-c3ccn(C)n3)c2=O)nn2c(C(=O)N[C@@H]3C[C@H]4CCO[C@H]43)cnc12 ; CNc1cc(Nc2cccn(-c3ccn(C)n3)c2=O)nn2c(C(=O)N[C@H]3C[C@@H]4CCO[C@@H]43)cnc12 ; [2H]C([2H])([2H])Nc1cc(Nc2cccn(-c3ccccn3)c2=O)nn2c(C(=O)N[C@@H]3C[C@H]4CCO[C@H]43)cnc12 ; [2H]C([2H])([2H])Nc1cc(Nc2cccn(-c3ccccn3)c2=O)nn2c(C(=O)N[C@H]3C[C@@H]4CCO[C@@H]43)cnc12] 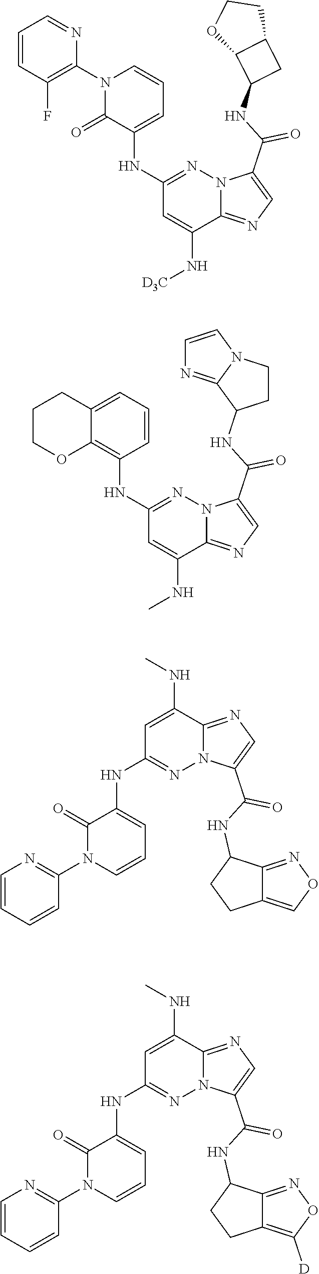 [SMILES: [2H]C([2H])([2H])Nc1cc(Nc2cccn(-c3ncccc3F)c2=O)nn2c(C(=O)N[C@@H]3C[C@H]4CCO[C@H]43)cnc12 ; CNc1cc(Nc2cccc3c2OCCC3)nn2c(C(=O)NC3CCn4ccnc43)cnc12 ; CNc1cc(Nc2cccn(-c3ccccn3)c2=O)nn2c(C(=O)NC3CCc4conc43)cnc12 ; [2H]c1onc2c1CCC2NC(=O)c1cnc2c(NC)cc(Nc3cccn(-c4ccccn4)c3=O)nn12] 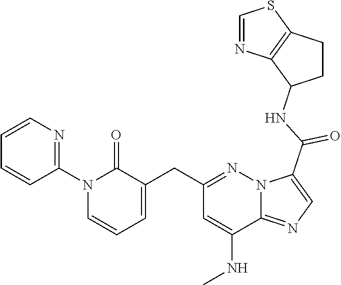 [SMILES: CNc1cc(Cc2cccn(-c3ccccn3)c2=O)nn2c(C(=O)NC3CCc4scnc43)cnc12] 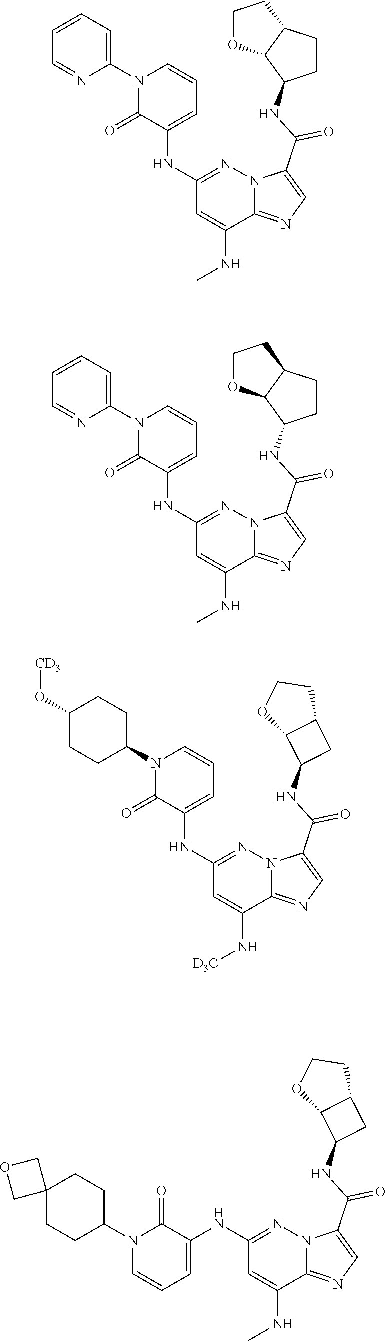 [SMILES: CNc1cc(Nc2cccn(-c3ccccn3)c2=O)nn2c(C(=O)N[C@@H]3CC[C@H]4CCO[C@H]43)cnc12 ; CNc1cc(Nc2cccn(-c3ccccn3)c2=O)nn2c(C(=O)N[C@H]3CC[C@@H]4CCO[C@@H]43)cnc12 ; [2H]C([2H])([2H])Nc1cc(Nc2cccn([C@H]3CC[C@H](OC([2H])([2H])[2H])CC3)c2=O)nn2c(C(=O)N[C@@H]3C[C@H]4CCO[C@H]43)cnc12 ; CNc1cc(Nc2cccn(C3CCC4(CC3)COC4)c2=O)nn2c(C(=O)N[C@@H]3C[C@H]4CCO[C@H]43)cnc12] 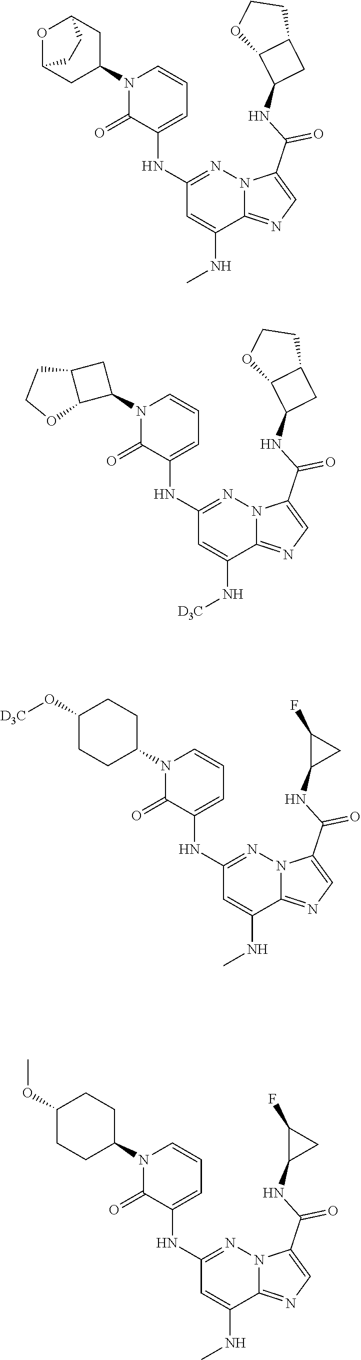 [SMILES: CNc1cc(Nc2cccn([C@@H]3C[C@H]4CC[C@@H](C3)O4)c2=O)nn2c(C(=O)N[C@@H]3C[C@H]4CCO[C@H]43)cnc12 ; [2H]C([2H])([2H])Nc1cc(Nc2cccn([C@@H]3C[C@H]4CCO[C@H]43)c2=O)nn2c(C(=O)N[C@@H]3C[C@H]4CCO[C@H]43)cnc12 ; [2H]C([2H])([2H])O[C@H]1CC[C@@H](n2cccc(Nc3cc(NC)c4ncc(C(=O)N[C@@H]5C[C@@H]5F)n4n3)c2=O)CC1 ; CNc1cc(Nc2cccn([C@H]3CC[C@H](OC)CC3)c2=O)nn2c(C(=O)N[C@@H]3C[C@@H]3F)cnc12] 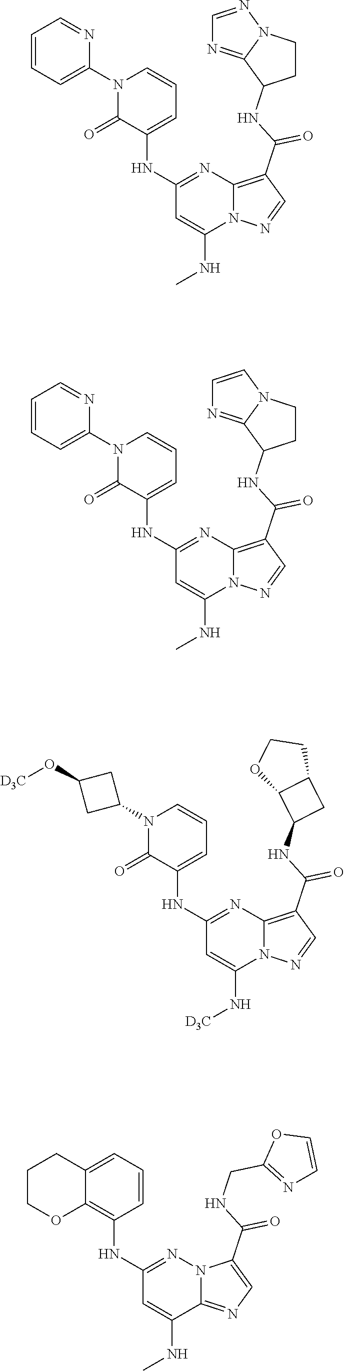 [SMILES: CNc1cc(Nc2cccn(-c3ccccn3)c2=O)nc2c(C(=O)NC3CCn4ncnc43)cnn12 ; CNc1cc(Nc2cccn(-c3ccccn3)c2=O)nc2c(C(=O)NC3CCn4ccnc43)cnn12 ; [2H]C([2H])([2H])Nc1cc(Nc2cccn([C@H]3C[C@H](OC([2H])([2H])[2H])C3)c2=O)nc2c(C(=O)N[C@@H]3C[C@H]4CCO[C@H]43)cnn12 ; CNc1cc(Nc2cccc3c2OCCC3)nn2c(C(=O)NCc3ncco3)cnc12] 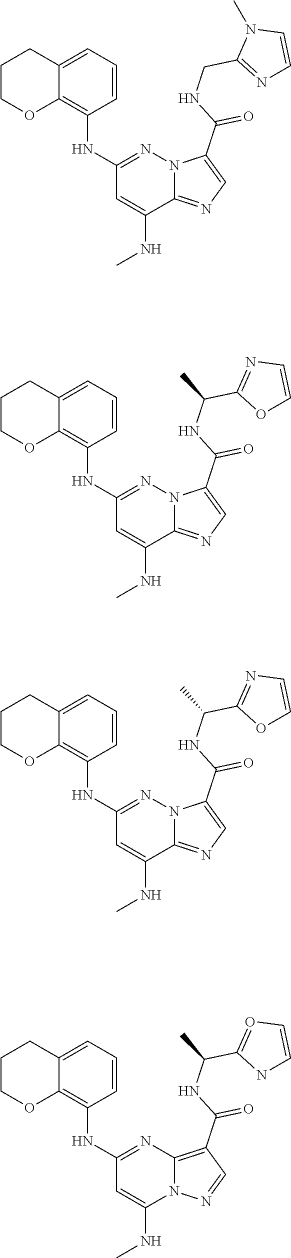 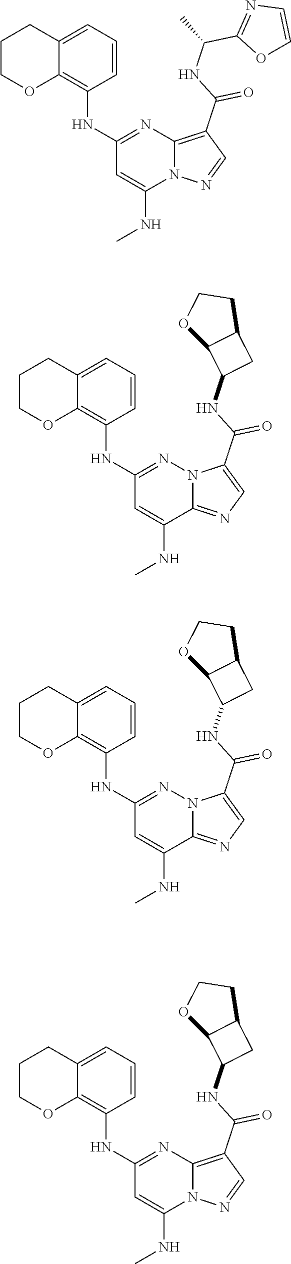 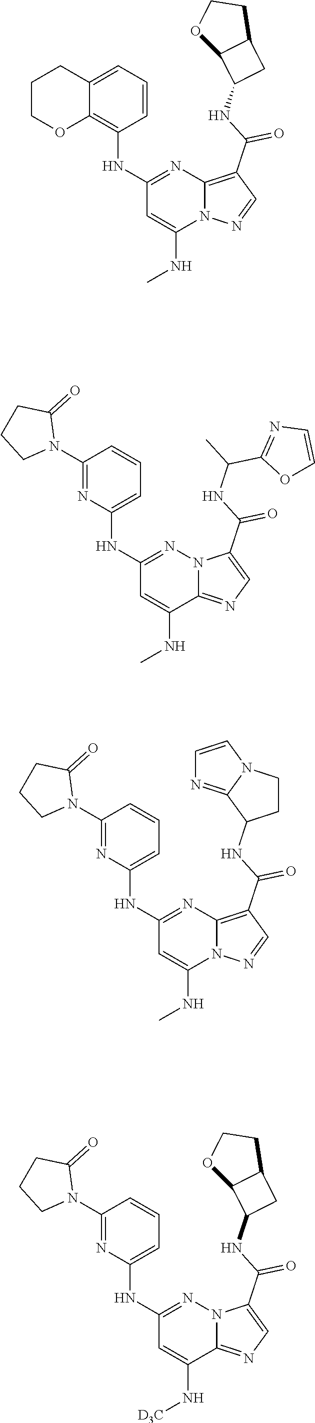 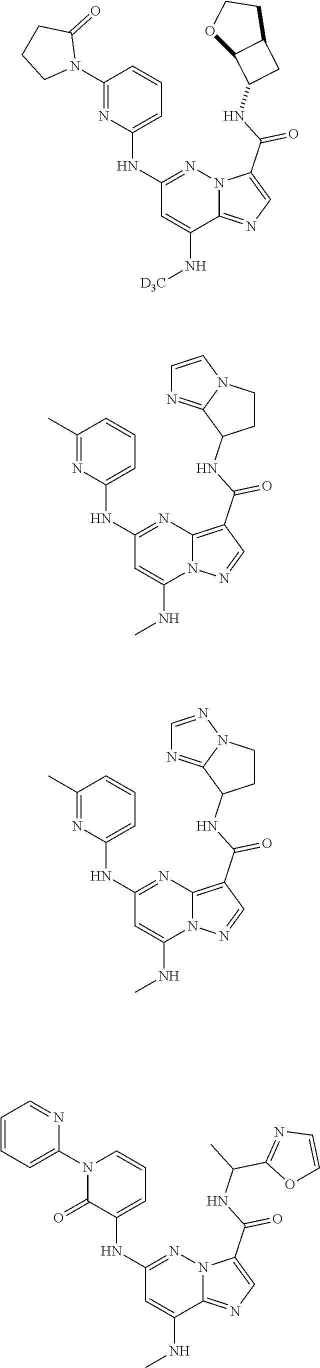 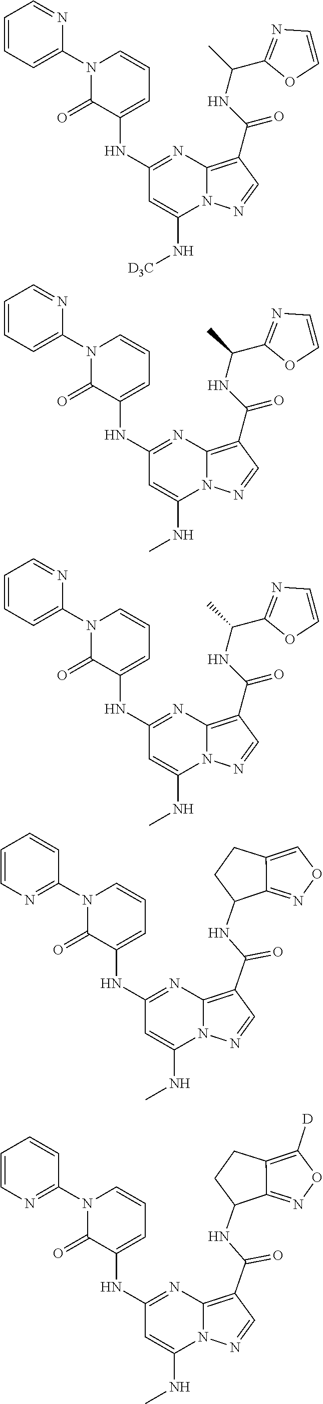 [SMILES: [2H]C([2H])([2H])Nc1cc(Nc2cccn(-c3ccccn3)c2=O)nc2c(C(=O)NC(C)c3ncco3)cnn12 ; CNc1cc(Nc2cccn(-c3ccccn3)c2=O)nc2c(C(=O)N[C@@H](C)c3ncco3)cnn12 ; CNc1cc(Nc2cccn(-c3ccccn3)c2=O)nc2c(C(=O)N[C@H](C)c3ncco3)cnn12 ; CNc1cc(Nc2cccn(-c3ccccn3)c2=O)nc2c(C(=O)NC3CCc4conc43)cnn12 ; [2H]c1onc2c1CCC2NC(=O)c1cnn2c(NC)cc(Nc3cccn(-c4ccccn4)c3=O)nc12] 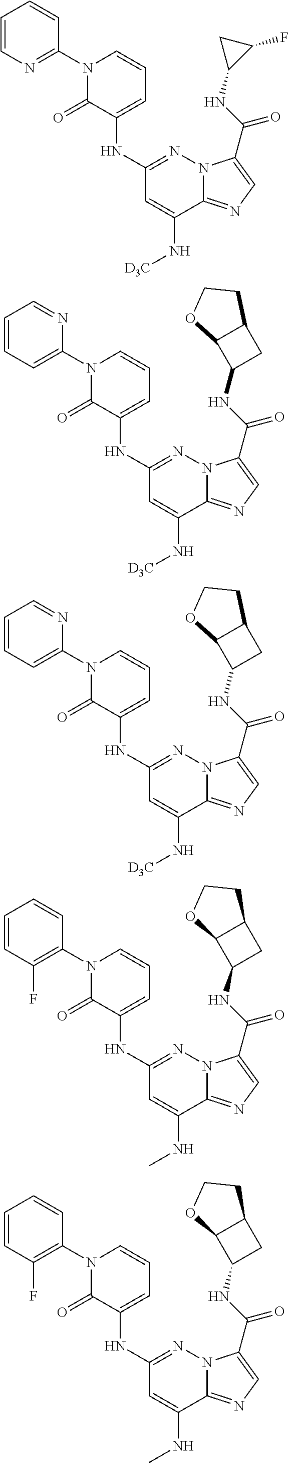 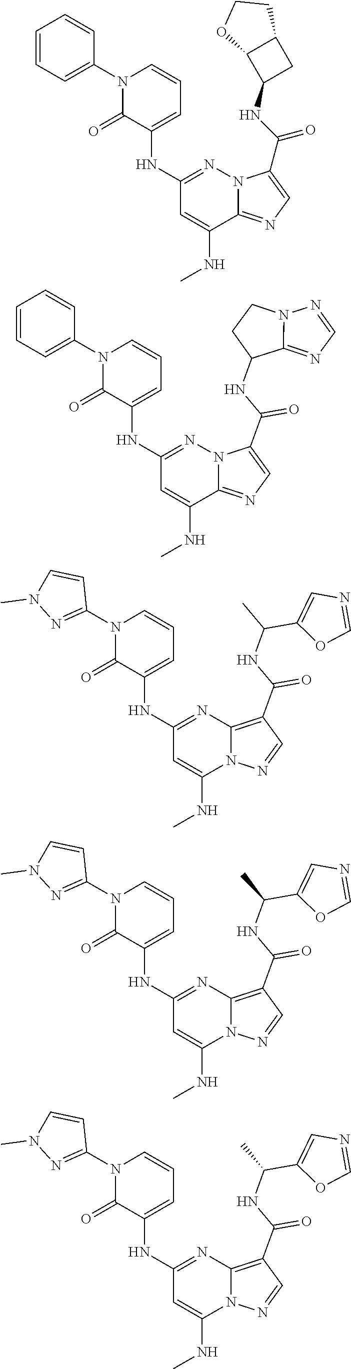 [SMILES: CNc1cc(Nc2cccn(-c3ccccc3)c2=O)nn2c(C(=O)N[C@@H]3C[C@H]4CCO[C@H]43)cnc12 ; CNc1cc(Nc2cccn(-c3ccccc3)c2=O)nn2c(C(=O)NC3CCn4ncnc43)cnc12 ; CNc1cc(Nc2cccn(-c3ccn(C)n3)c2=O)nc2c(C(=O)NC(C)c3cnco3)cnn12 ; CNc1cc(Nc2cccn(-c3ccn(C)n3)c2=O)nc2c(C(=O)N[C@@H](C)c3cnco3)cnn12 ; CNc1cc(Nc2cccn(-c3ccn(C)n3)c2=O)nc2c(C(=O)N[C@H](C)c3cnco3)cnn12] 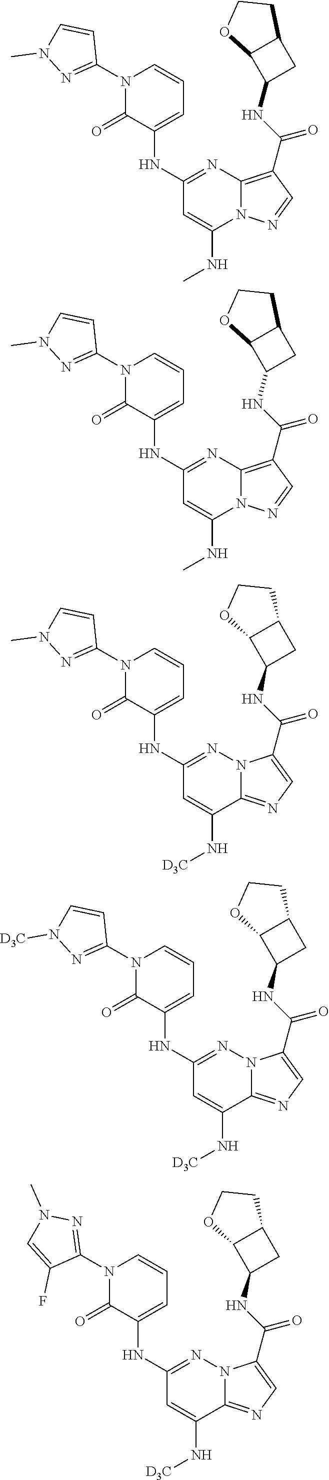 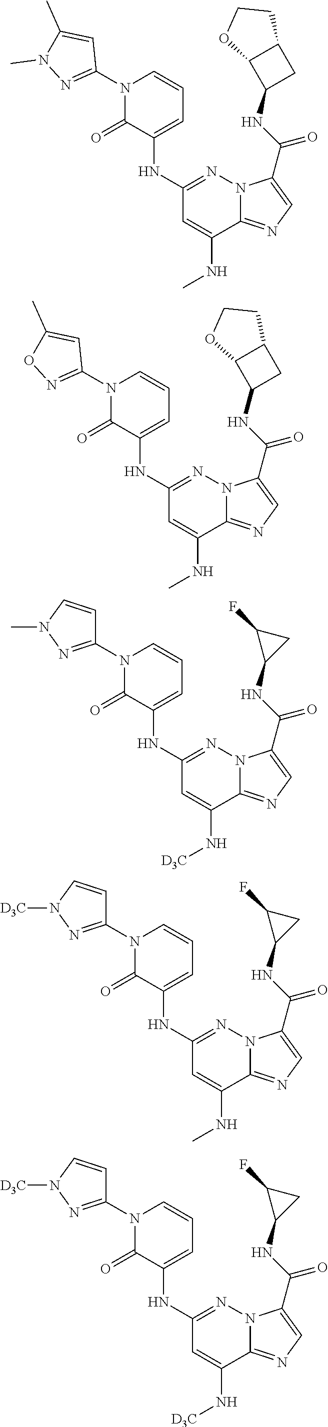 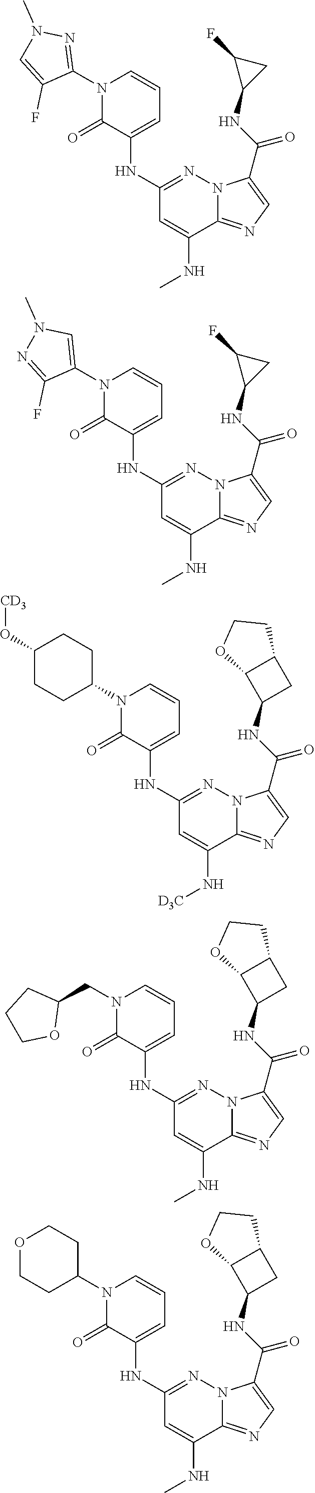 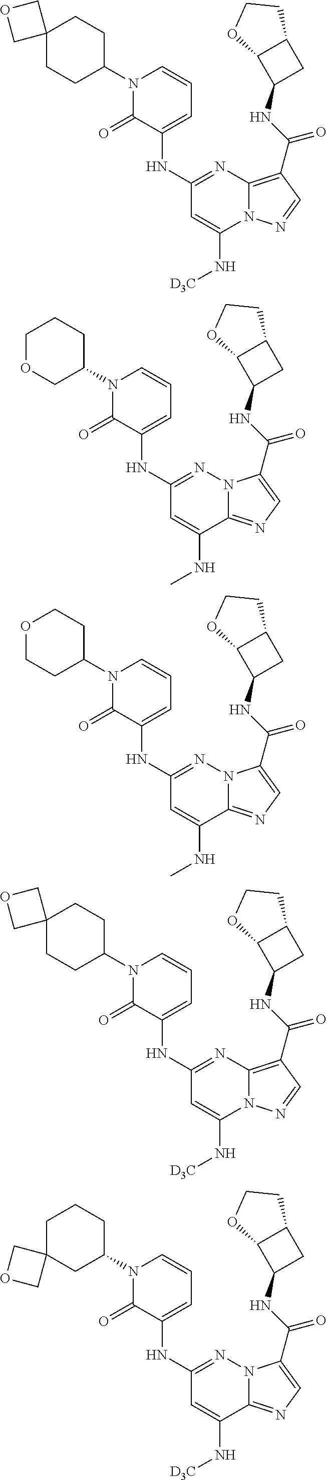 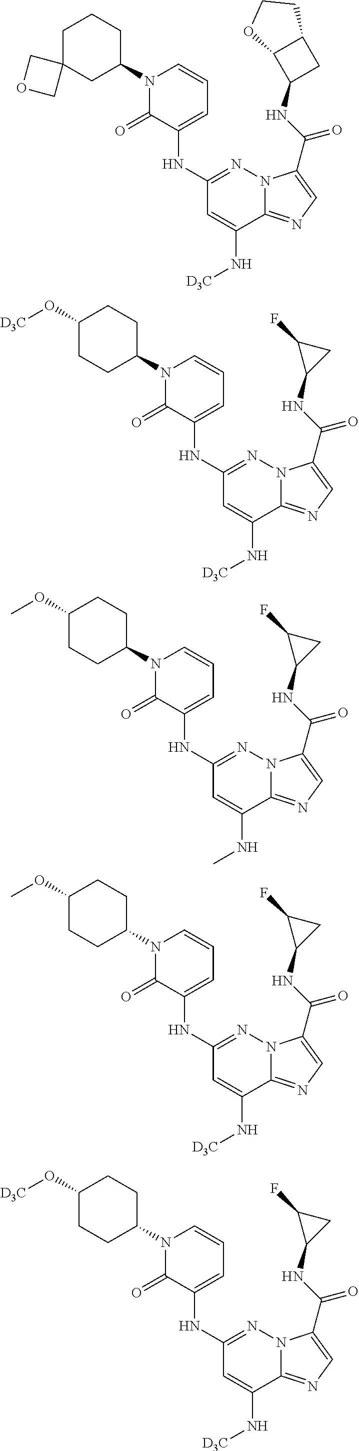 [SMILES: [2H]C([2H])([2H])Nc1cc(Nc2cccn([C@@H]3CCCC4(COC4)C3)c2=O)nn2c(C(=O)N[C@@H]3C[C@H]4CCO[C@H]43)cnc12 ; [2H]C([2H])([2H])Nc1cc(Nc2cccn([C@H]3CC[C@H](OC([2H])([2H])[2H])CC3)c2=O)nn2c(C(=O)N[C@@H]3C[C@@H]3F)cnc12 ; CNc1cc(Nc2cccn([C@H]3CC[C@H](OC)CC3)c2=O)nn2c(C(=O)N[C@@H]3C[C@@H]3F)cnc12 ; [2H]C([2H])([2H])Nc1cc(Nc2cccn([C@H]3CC[C@@H](OC)CC3)c2=O)nn2c(C(=O)N[C@@H]3C[C@@H]3F)cnc12 ; [2H]C([2H])([2H])Nc1cc(Nc2cccn([C@H]3CC[C@@H](OC([2H])([2H])[2H])CC3)c2=O)nn2c(C(=O)N[C@@H]3C[C@@H]3F)cnc12] 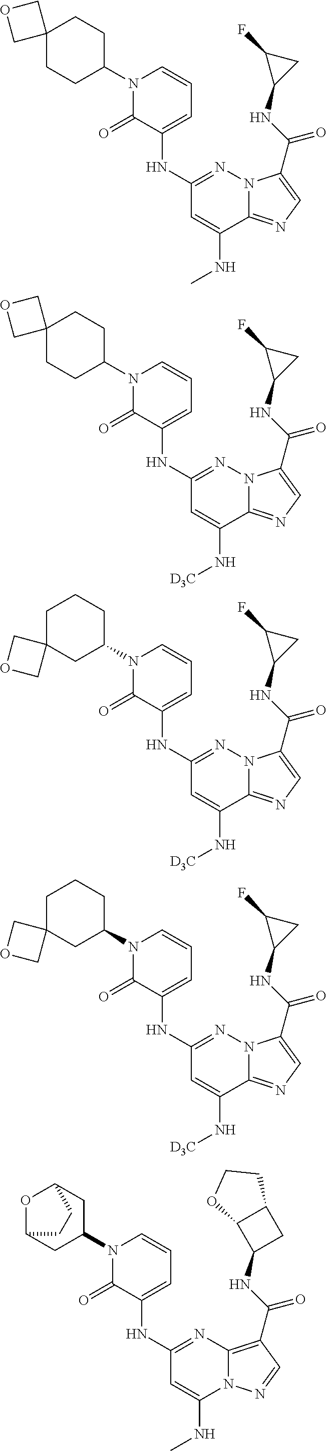 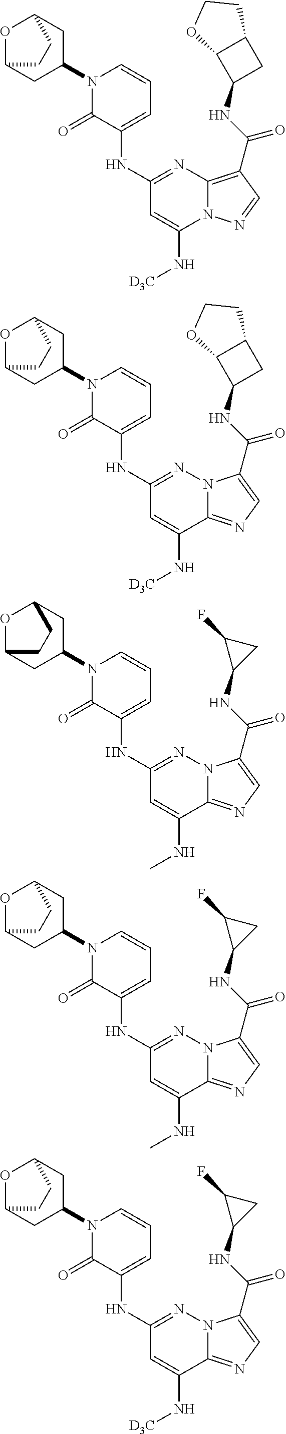 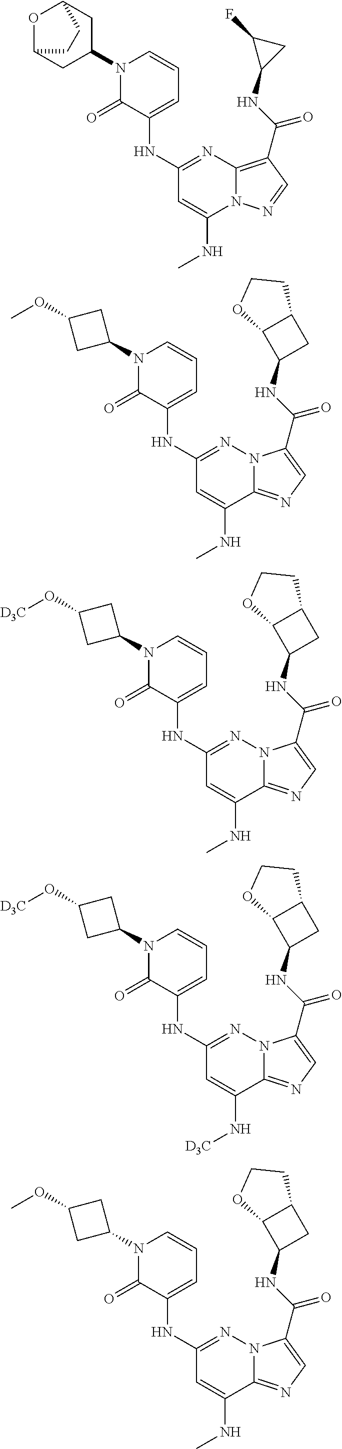 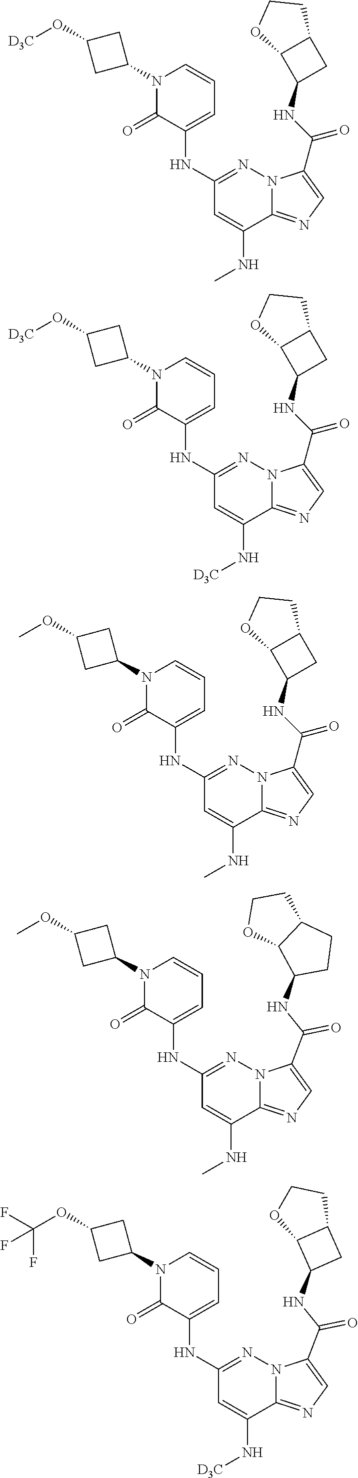 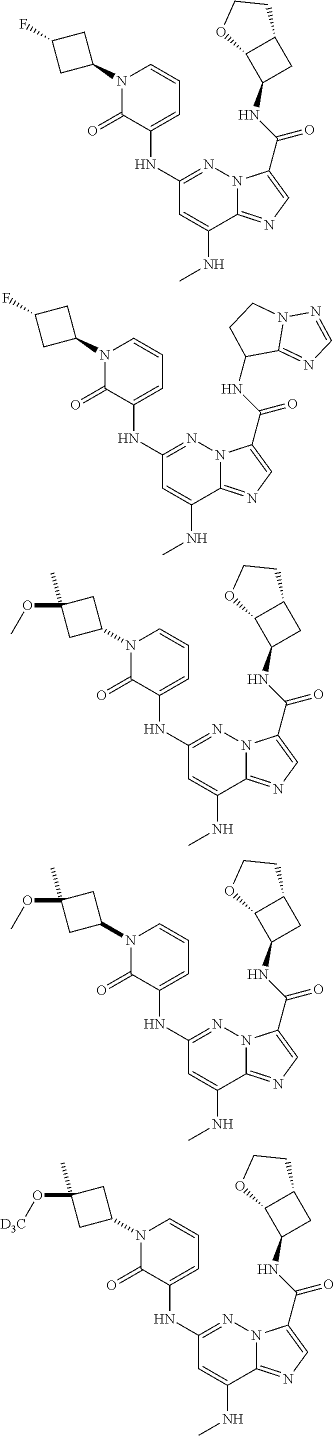 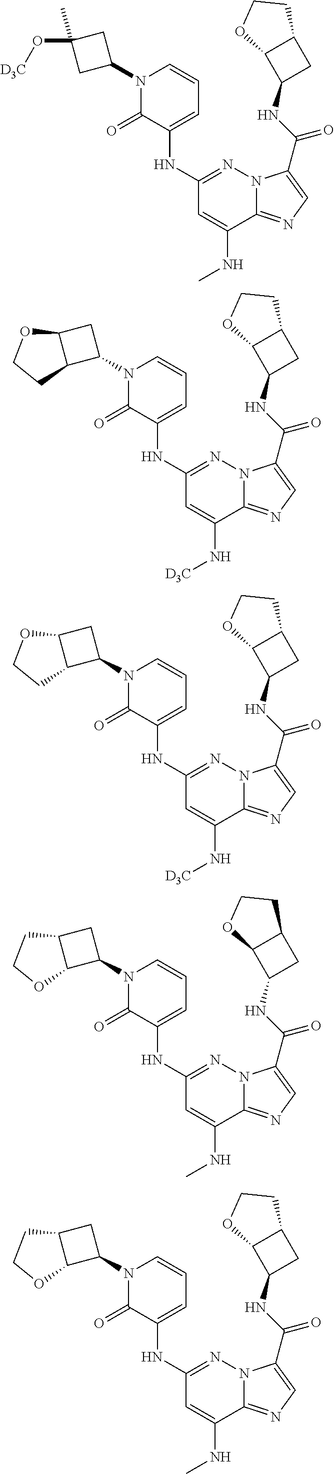 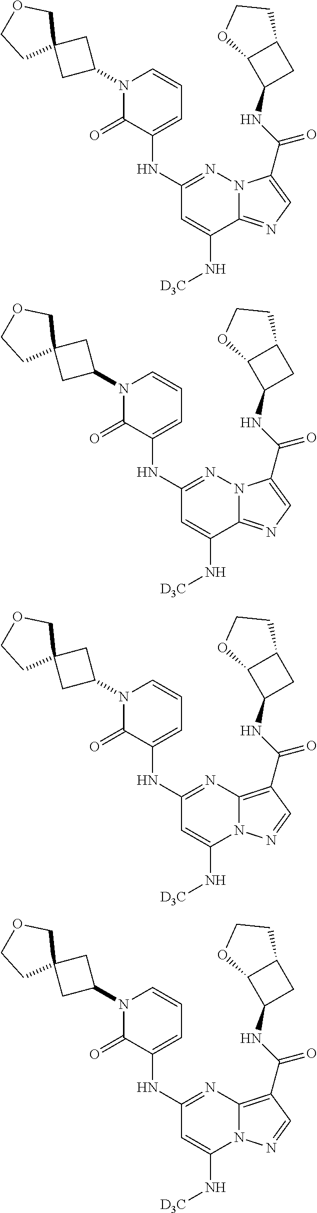 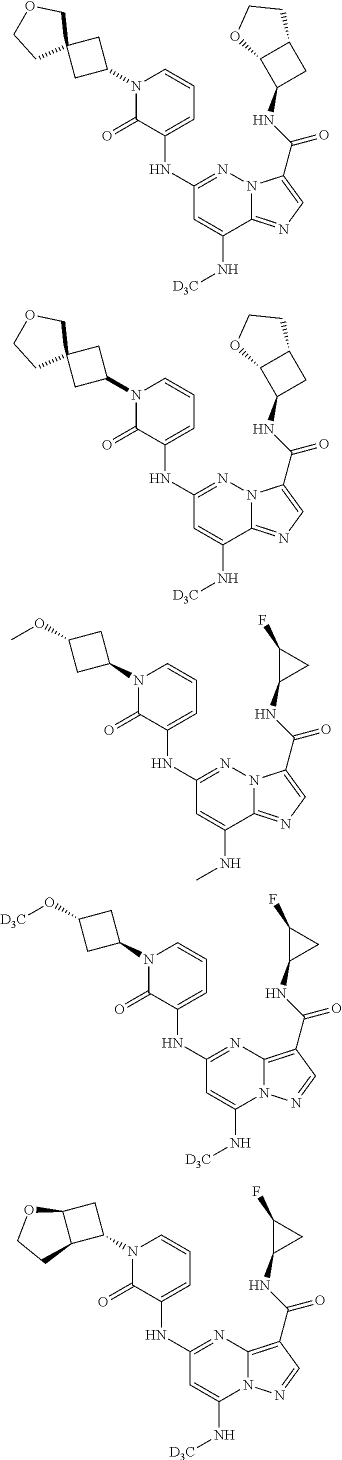 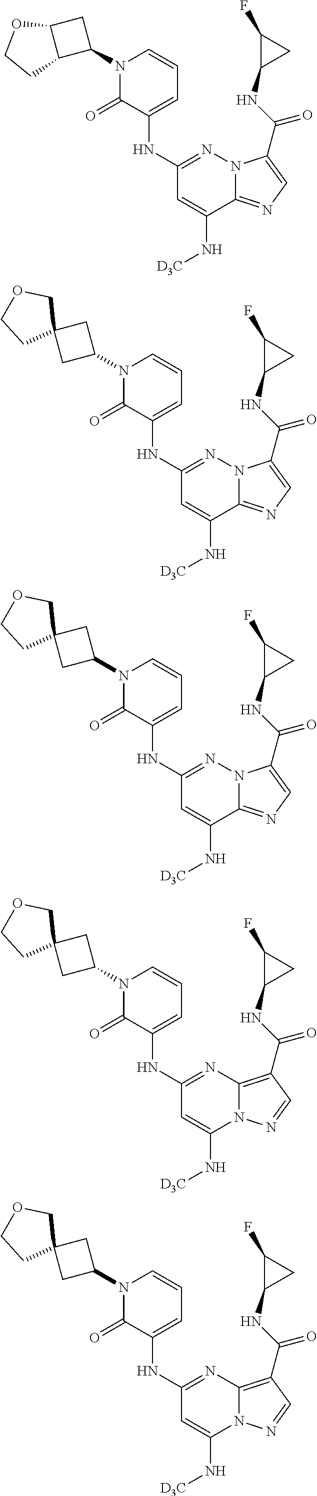 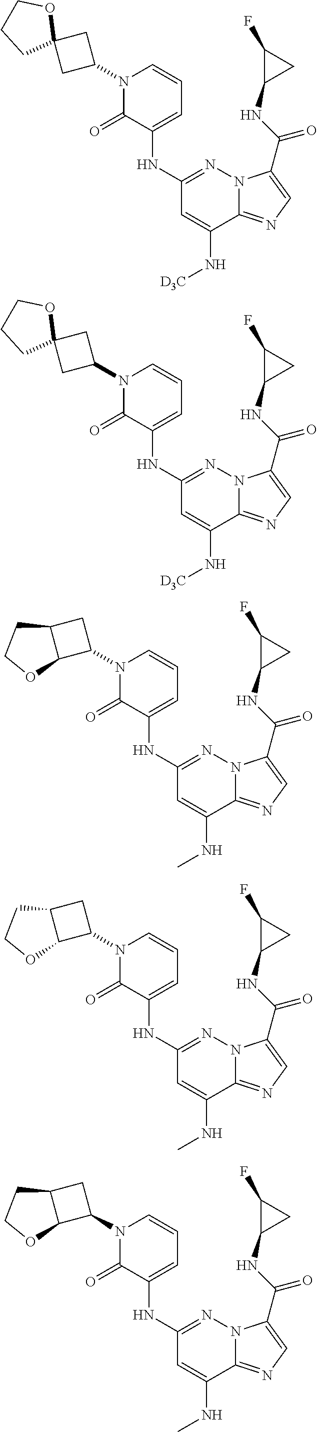 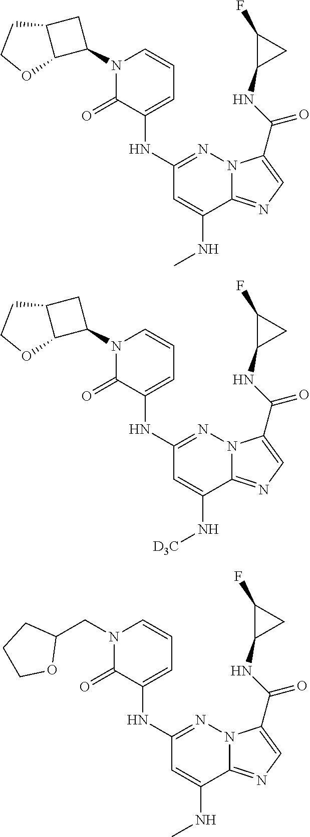 [SMILES: CNc1cc(Nc2cccn([C@@H]3C[C@H]4CCO[C@H]43)c2=O)nn2c(C(=O)N[C@@H]3C[C@@H]3F)cnc12 ; [2H]C([2H])([2H])Nc1cc(Nc2cccn([C@@H]3C[C@H]4CCO[C@H]43)c2=O)nn2c(C(=O)N[C@@H]3C[C@@H]3F)cnc12 ; CNc1cc(Nc2cccn(CC3CCCO3)c2=O)nn2c(C(=O)N[C@@H]3C[C@@H]3F)cnc12]
or an enantiomer or diastereomer thereof, or a pharmaceutically acceptable salt thereof, wherein “*” indicates a chiral center.

42. A pharmaceutical composition comprising the compound of claim 1, or a pharmaceutically acceptable salt thereof, and a pharmaceutically acceptable excipient.

43. A method of inhibiting TYK2 in a subject or biological sample comprising contacting the subject or biological sample with an effective amount of the compound of claim 1, or a pharmaceutically acceptable salt thereof.

44. A method of treating a TYK2-mediated disease or disorder in a subject in need thereof, the method comprising administering to the subject a therapeutically effective amount of the compound of claim 1, or a pharmaceutically acceptable salt thereof.

45. The method of claim 44, wherein the TYK2-mediated disease or disorder is selected from systemic lupus erythematosus, psoriasis, Crohn's disease, ulcerative colitis, inflammatory bowel disease, rheumatoid arthritis, asthma, chronic obstructive pulmonary disease, hematological cancer, an endocrine disease or disorder, a neurological disease or disorder, a disease or disorder associated with transplantation, or a combination thereof.

46-55. (canceled)
